(19) United States
(12) Patent Application Publication
(10) Pub. No.: US 2026/0092276 A1
(43) Pub. Date: Apr. 2, 2026

(54) Carbohydrate-oligonucleotide conjugates, pharmaceutical compositions, and therapeutic applications

(71) Applicant: KYLONOVA (XIAMEN) BIOPHARMA CO., LTD., Xiamen, Fujian (CN)

(72) Inventors: Kunyuan CUI, Xiamen, Fujian (CN); Xueqin LU, Xiamen, Fujian (CN)

(21) Appl. No.: 19/112,959

(22) PCT Filed: Sep. 18, 2023
(86) PCT No.: PCT/CN2023/119377

§ 371 Date: Mar. 18, 2025

(30) Foreign Application Priority Data
Sep. 19, 2022 (WO) PCT/CN2022/119522
May 23, 2023 (WO) PCT/CN2023/095768

(51) Int. Cl. C12N 15/113; A61K 47/54

(52) U.S. Cl. CPC C12N 15/113; A61K 47/549; C12N 2310/14; C12N 2310/351; C12N 2310/51

(57) ABSTRACT

Carbohydrate-oligonucleotide conjugates are compounds of Formula (I). A pharmaceutical composition comprises the conjugate. The methods of their therapeutic application are treating, preventing, or ameliorating one or more symptoms of a disorder, disease, or condition.

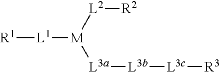

### CROSS REFERENCE TO RELATED APPLICATION

This application claims the benefit of the priority of International Application Nos. PCT/CN2022/119522, filed Sep. 19, 2022, and PCT/CN2023/095768, filed May 23, 2023, under 35 U.S.C. 119(a); the disclosure of each of which is incorporated herein by reference in its entirety.

### FIELD

Provided herein are carbohydrate-oligonucleotide conjugates and pharmaceutical compositions thereof. Also provided herein are methods of their therapeutic applications for treating, preventing, or ameliorating one or more symptoms of a disorder, disease, or condition.

### REFERENCE TO A SEQUENCE LISTING

The present specification is being filed with a Sequence Listing entitled 413A003WO01_SEQLIST_ST26.XML of 23,144 bytes in size and created Sep. 16, 2023; the content of which is incorporated herein by reference in its entirety.

### BACKGROUND

A therapeutic oligonucleotide such as an antisense oligonucleotide (ASO) and small interfering RNA (siRNA) is designed to modulate a disease gene expression. Roberts et al., Nat. Rev. Drug Dis. 2020, 19, 673-94. An ASO is a single-stranded oligonucleotide, which specifically binds to a target mRNA via the Watson-Crick base pairing to modulate the production of a disease-causing protein. Dhuri et al., J. Clin. Med. 2020, 9, 2004. A siRNA is a double-stranded RNA molecule that induces gene silencing by targeting a complementary mRNA for degradation. Of the two strands of a siRNA, one is an antisense strand (also known as a guide strand) and the other is a sense strand (also known as a passenger strand). Shukla et al., Chem Med Chem 2010, 5, 328-49. In silencing a target gene, a siRNA first binds to an RNA-induced silencing complex (RISC). Hammond et al., Nature 2000, 404, 293-6. Within the RISC, the two strands of the siRNA are separated, and the antisense strand and the RISC form an activated RISC, which binds to a target mRNA molecule for cleavage. Nykanen et al., Cell 2001, 107, 309-21; Martinez et al., Cell 2002, 110, 563-74. This gene-silencing process is mediated through the sequence-specific hybridization of the guide RNA to the mRNA target site and brings RISC into close proximity to its target mRNA molecule, which is then cleaved by the RISC nuclease Argonaute 2 (Ago 2). Liu et al., Science 2004, 305, 1437-41; Rand et al., Proc. Natl. Acad. Sci. USA 2004, 101, 14385-9; Rivas et al., Nat. Struct. Mol. Biol. 2005, 12, 340-9. Since RISC is recovered for subsequent rounds, this represents a catalytical process leading to the selective reduction in a specific mRNA molecule and thus resulting in decreased expression of the targeted gene.

In general, therapeutic oligonucleotides are specific and efficient in modulating a disease-related gene. Hu et al., Signal. Transduct. Target Ther. 2020, 5, 101; Alshaer et al., Eur. J. Pharmacol. 2021, 905, 174178. However, their therapeutic applications are limited by their poor cellular uptake and susceptibility to nuclease-mediated degradation. Roberts et al., Nat. Rev. Drug Dis. 2020, 19, 673-94; Gagliardi and Ashizawa, Biomedicines 2021, 9, 433; Friedrich and Aigner, BioDrugs 2022, 1-23. Therefore, there is a need for therapeutic oligonucleotides with pharmaceutical properties suitable for therapeutic applications.

### SUMMARY OF THE DISCLOSURE

Provided herein is a carbohydrate-oligonucleotide conjugate comprising an asialoglycoprotein receptor (ASGPR) binding moiety and two oligonucleotides.

Also provided herein is a carbohydrate-oligonucleotide conjugate comprising an ASGPR binding moiety, a first oligonucleotide duplex comprising first sense and antisense sequences, a second oligonucleotide duplex comprising second sense and antisense sequences, and a trivalent linker or first and second divalent linkers; wherein the ASGPR binding moiety is connected to a terminus of the first sense or antisense sequence and to a terminus of the second sense or antisense sequence via the trivalent linker; or wherein the ASGPR binding moiety is connected to a terminus of the first sense or antisense sequence via the first divalent linker, and one of remaining termini of the first sense or antisense sequence is connected to a terminus of the second sense or antisense sequence via the second divalent linker.

Additionally, provided herein is a carbohydrate-oligonucleotide conjugate comprising an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a terminus of the first sense or antisense sequence and to a terminus of the second sense or antisense sequence via the trivalent linker.

Furthermore, provided herein is a carbohydrate-oligonucleotide conjugate comprising an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to a terminus of the first sense or antisense sequence via the first divalent linker, and one of remaining termini of the first sense or antisense sequence is connected to a terminus of the second sense or antisense sequence via the second divalent linker.

Provided herein is a carbohydrate-oligonucleotide conjugate comprising an ASGPR binding moiety, first and second single-stranded oligonucleotides, and a trivalent linker or first and second divalent linkers; wherein the ASGPR binding moiety is connected to a terminus of the first single-stranded oligonucleotide and to a terminus of the second single-stranded oligonucleotide via the trivalent linker; or wherein the ASGPR binding moiety is connected to one terminus of the first single-stranded oligonucleotide via the first divalent linker, and the other terminus of the first single-stranded oligonucleotide is connected to a terminus of the second single-stranded oligonucleotide via the second divalent linker.

Provided herein is a carbohydrate-oligonucleotide conjugate comprising an ASGPR binding moiety, first and second single-stranded oligonucleotides, and a trivalent linker; wherein the ASGPR binding moiety is connected to a terminus of the first single-stranded oligonucleotide and to a terminus of the second single-stranded oligonucleotide via the trivalent linker.

Provided herein is a carbohydrate-oligonucleotide conjugate comprising an ASGPR binding moiety, first and second single-stranded oligonucleotides, and first and second divalent linkers; wherein the ASGPR binding moiety is connected to one terminus of the first single-stranded oligonucleotide via the first divalent linker, and the other terminus of the first single-stranded oligonucleotide is connected to a terminus of the second single-stranded oligonucleotide via the second divalent linker.

Provided herein is a pharmaceutical composition comprising a carbohydrate-oligonucleotide conjugate provided herein and a pharmaceutically acceptable excipient.

Provided herein is a method of treating, preventing, or ameliorating one or more symptoms of a disorder, disease, or condition in a subject, comprising administering to the subject in need thereof a therapeutically effective amount of a carbohydrate-oligonucleotide conjugate provided herein.

### BRIEF DESCRIPTION OF THE DRAWINGS

FIG. 1 shows the effects of GalNAc-siRNA conjugates on hPCSK9 and mANGPTL3 protein levels in hPCSK9-UTR mice.

FIG. 2 shows the effects of GalNAc-siRNA conjugates on LDL-C and TG levels in hPCSK9-UTR mice.

FIG. 3 shows the effects of GalNAc-siRNA conjugates on C3, C5, and CFB protein levels in mice.

FIG. 4 shows the effect of GalNAc-siRNA conjugate A18f on the plasma HBsAg level in HBV infected mice.

FIG. 5 shows the effect of GalNAc-siRNA conjugate A18f on the number of HBV infected mice with a plasma HBsAb level >10 mIV/mL.

FIG. 6 shows the effect of GalNAc-siRNA conjugate A18f on the plasma HBV DNA level in HBV infected mice.

### ILLUSTRATIVE EMBODIMENTS

Embodiment 1. A carbohydrate-oligonucleotide conjugate comprising an ASGPR binding moiety and two oligonucleotides.
Embodiment 2. The carbohydrate-oligonucleotide conjugate of embodiment 1, comprising an ASGPR binding moiety, two oligonucleotides, and a trivalent linker.
Embodiment 3. The carbohydrate-oligonucleotide conjugate of embodiment 1 or 2, comprising an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a terminus of the first sense or antisense sequence and to a terminus of the second sense or antisense sequence via the trivalent linker.
Embodiment 4. The carbohydrate-oligonucleotide conjugate of embodiment 2 or 3, wherein the ASGPR binding moiety is connected to the 5′-terminus of the first sense or antisense sequence and to the 5′-terminus of the second sense or antisense sequence via the trivalent linker.
Embodiment 5. The carbohydrate-oligonucleotide conjugate of any one of embodiments 2 to 4, wherein the ASGPR binding moiety is connected to the 5′-terminus of the first sense sequence and to the 5′-terminus of the second sense sequence via the trivalent linker.
Embodiment 6. The carbohydrate-oligonucleotide conjugate of any one of embodiments 2 to 4, wherein the ASGPR binding moiety is connected to the 3′-terminus of the first sense sequence and to the 5′-terminus of the second sense sequence via the trivalent linker.
Embodiment 7. The carbohydrate-oligonucleotide conjugate of any one of embodiments 2 to 4, wherein the ASGPR binding moiety is connected to the 3′-terminus of the first sense sequence and to the 3′-terminus of the second sense sequence via the trivalent linker.
Embodiment 8. The carbohydrate-oligonucleotide conjugate of any one of embodiments 2 to 4, wherein the ASGPR binding moiety is connected to the 5′-terminus of the first antisense sequence and to the 5′-terminus of the second antisense sequence via the trivalent linker.
Embodiment 9. The carbohydrate-oligonucleotide conjugate of any one of embodiments 2 to 4 wherein the ASGPR binding moiety is connected to the 3′-terminus of the first antisense sequence and to the 5′-terminus of the second antisense sequence via the trivalent linker.
Embodiment 10. The carbohydrate-oligonucleotide conjugate of any one of embodiments 2 to 4, wherein the ASGPR binding moiety is connected to the 3′-terminus of the first antisense sequence and to the 3′-terminus of the second antisense sequence via the trivalent linker.
Embodiment 11. The carbohydrate-oligonucleotide conjugate of embodiment 1, comprising an ASGPR binding moiety, two oligonucleotides, and two divalent linkers.
Embodiment 12. The carbohydrate-oligonucleotide conjugate of embodiment 1 or 11, comprising an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to a terminus of the first sense or antisense sequence via the first divalent linker, and one of remaining termini of the first sense or antisense sequence is connected to a terminus of the second sense or antisense sequence via the second divalent linker.
Embodiment 13. The carbohydrate-oligonucleotide conjugate of any one of embodiments 3 to 10 and 12, wherein the first oligonucleotide duplex is a double-stranded siRNA.
Embodiment 14. The carbohydrate-oligonucleotide conjugate of any one of embodiments 3 to 10, 12, and 13, wherein each strand of the first oligonucleotide duplex independently comprises from about 10 to about 50, from about 10 to about 30, or from about 15 to about 25 nucleotides.
Embodiment 15. The carbohydrate-oligonucleotide conjugate of any one of embodiments 3 to 10 and 12 to 14, wherein the second oligonucleotide duplex is a double-stranded siRNA.
Embodiment 16. The carbohydrate-oligonucleotide conjugate of any one of embodiments 3 to 10 and 12 to 15, wherein each strand of the second oligonucleotide duplex independently comprises from about 10 to about 50, from about 10 to about 30, or from about 15 to about 25 nucleotides.
Embodiment 17. The carbohydrate-oligonucleotide conjugate of embodiment 14 or 16, wherein each nucleotide is independently a natural nucleotide or modified nucleotide.
Embodiment 18. The carbohydrate-oligonucleotide conjugate of embodiment 14, 16, or 17, wherein each nucleotide is independently adenylate, cytidylate, guanylate, uridylate, 2′-fluoroadenosine, 2′-fluorocytidine, 2′-fluorogunaosine, 2′-fluorouridine, 2′-deoxyadenosine, 2′-deoxycytidine, 2′-deoxygunaosine, 2′-deoxythymidine, 2′-O-methyladenosine, 2′-O-methylcytidine, 2′-O-methylguanosine, or 2′-O-methyluridine.
Embodiment 19. The carbohydrate-oligonucleotide conjugate of any one of embodiments 14 and 16 to 18, wherein each strand has one or more phosphate linkage groups each independently replaced with phosphorothioate or phosphorodithiate.
Embodiment 20. The carbohydrate-oligonucleotide conjugate of embodiment 1 or 2, comprising an ASGPR binding moiety, first and second single-stranded oligonucleotides, and a trivalent linker; wherein the ASGPR binding moiety is connected to a terminus of the first single-stranded oligonucleotide and to a terminus of the second single-stranded oligonucleotide via the trivalent linker.
Embodiment 21. The carbohydrate-oligonucleotide conjugate of embodiment 20, wherein the ASGPR binding moiety is connected to the 5′-terminus of the first single-stranded oligonucleotide and to the 5′-terminus of the second single-stranded oligonucleotide via the trivalent linker.
Embodiment 22. The carbohydrate-oligonucleotide conjugate of embodiment 20, wherein the ASGPR binding moiety is connected to the 3′-terminus of the first single-stranded oligonucleotide and to the 5′-terminus of the second single-stranded oligonucleotide via the trivalent linker.
Embodiment 23. The carbohydrate-oligonucleotide conjugate of embodiment 20, wherein the ASGPR binding moiety is connected to the 3′-terminus of the first single-stranded oligonucleotide and to the 3′-terminus of the second single-stranded oligonucleotide via the trivalent linker.
Embodiment 24. The carbohydrate-oligonucleotide conjugate of embodiment 1 or 11, comprising an ASGPR binding moiety, first and second single-stranded oligonucleotides, and first and second divalent linkers; wherein the ASGPR binding moiety is connected to one terminus of the first single-stranded oligonucleotide via the first divalent linker, and the other terminus of the first single-stranded oligonucleotide is connected to a terminus of the second single-stranded oligonucleotide via the second divalent linker.
Embodiment 25. The carbohydrate-oligonucleotide conjugate of any one of embodiments 20 to 24, wherein the first single-stranded oligonucleotide is a single-stranded RNA.
Embodiment 26. The carbohydrate-oligonucleotide conjugate of any one of embodiments 20 to 25, wherein the first single-stranded oligonucleotide comprises from about 10 to about 50, from about 10 to about 30, or from about 15 to about 25 nucleotides.
Embodiment 27. The carbohydrate-oligonucleotide conjugate of any one of embodiments 20 to 26, wherein the second single-stranded oligonucleotide is a single-stranded RNA.
Embodiment 28. The carbohydrate-oligonucleotide conjugate of any one of embodiments 20 to 27, wherein the second single-stranded oligonucleotide comprises from about 10 to about 50, from about 10 to about 30, or from about 15 to about 25 nucleotides.
Embodiment 29. The carbohydrate-oligonucleotide conjugate of embodiment 26 or 28, wherein each nucleotide is independently a natural nucleotide or modified nucleotide.
Embodiment 30. The carbohydrate-oligonucleotide conjugate of embodiment 26, 28, or 29, wherein each nucleotide is independently adenylate, cytidylate, guanylate, uridylate, 2′-fluoroadenosine, 2′-fluorocytidine, 2′-fluorogunaosine, 2′-fluorouridine, 2′-deoxyadenosine, 2′-deoxycytidine, 2′-deoxygunaosine, 2′-deoxythymidine, 2′-O-methyladenosine, 2′-O-methylcytidine, 2′-O-methylguanosine, or 2′-O-methyluridine.
Embodiment 31. The carbohydrate-oligonucleotide conjugate of any one of embodiments 26 and 28 to 30, wherein each single-stranded oligonucleotide has one or more phosphate linkage groups independently replaced with phosphorothioate or phosphorodithiate.
Embodiment 32. The carbohydrate-oligonucleotide conjugate of any one of embodiments 20 to 31, wherein the single-stranded oligonucleotide is an ASO sequence or a sense or antisense sequence of a siRNA.
Embodiment 33. The carbohydrate-oligonucleotide conjugate of any one of embodiments 1 to 32, wherein the ASGPR binding moiety comprises from about 1 to about 10 N-acetylgalactosamines.
Embodiment 34. The carbohydrate-oligonucleotide conjugate of any one of embodiments 1 to 33, wherein the ASGPR binding moiety comprises about 1, about 2, about 3, about 4, or about 5 N-acetylgalactosamines.
Embodiment 32. The carbohydrate-oligonucleotide conjugate of any one of embodiments 1 to 34, wherein the ASGPR binding moiety comprises about 2, about 3, or about 4 N-acetylgalactosamines.
Embodiment 36. The carbohydrate-oligonucleotide conjugate of any one of embodiments 1 to 35, wherein the ASGPR binding moiety has the structure of Formula (A-I):

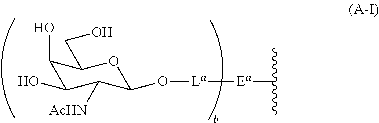

or an enantiomer, a mixture of enantiomers, a diastereomer, a mixture of two or more diastereomers, a tautomer, or a mixture of two or more tautomers thereof; wherein:
E^{a} and b are:
(i) b is an integer of 1; and E^{a} is a bond, CH₂, or NH;
(ii) b is an integer of 2; and E^{a} is a trivalent linker; or
(iii) b is an integer of 3; and E^{a} is a tetravalent linker; and
each L^{a} is independently a linker.
Embodiment 37. The carbohydrate-oligonucleotide conjugate of embodiment 36, wherein b is an integer of 2; and E^{a} is a trivalent linker.
Embodiment 38. The carbohydrate-oligonucleotide conjugate of embodiment 37, wherein E^{a} is CH.
Embodiment 39. The carbohydrate-oligonucleotide conjugate of embodiment 37, wherein E^{a} is N.
Embodiment 40. The carbohydrate-oligonucleotide conjugate of embodiment 36, wherein b is an integer of 3 and E^{a} is a tetravalent linker.
Embodiment 41. The carbohydrate-oligonucleotide conjugate of embodiment 40, wherein E^{a} is C.
Embodiment 42. The carbohydrate-oligonucleotide conjugate of any one of embodiments 36 to 41, wherein each L^{a} is independently a linker having the structure of —Zⁿ—(Rⁿ—Zⁿ)_{z}—, wherein:
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —C(R^{1a})═NO—, —O—, —OC(O)O—, —OC(O)NR^{1b}—, —OC(O)S—, —OC(NR^{1a})NR^{1b}—, —OC(S)O—, —OC(S)NR^{1b}, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —P(O₂)O—, —P(O)(S)O—, —S—, —S(O)—, —S(O)₂—, —S(O)NR^{1b}—, or —S(O)₂NR^{1b}—;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
z is an integer of 0, 1, 2, 3, 4, 5, 6, 7, 8, 9, or 10;
wherein each alkyl, alkylene, heteroalkyl, alkenyl, alkenylene, alkynyl, alkynylene, cycloalkyl, cycloalkylene, aryl, arylene, heteroaryl, heteroarylene, heterocyclyl, or heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₄ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{c}C(O)R^{h}, —NR^{e}C(O)OR^{f}, —NR^{c}C(O)NR^{f}R^{g}, —NR^{c}C(O)SR^{f}, —NR^{c}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.
Embodiment 43. The carbohydrate-oligonucleotide conjugate of embodiment 42, wherein each Rⁿ is independently C₁₋₁₀ alkylene, C₆₋₁₄ arylene, heteroarylene, or heterocyclylene, each of which is optionally substituted with one or more substituents Q.
Embodiment 44. The carbohydrate-oligonucleotide conjugate of embodiment 42 or 43, wherein each Rⁿ is independently methanediyl, ethanediyl, propanediyl, butanediyl, pentanediyl, hexanediyl, phendiyl, 1,2,3-triazoldiyl, pyrrolidindiyl, or piperidindiyl, each optionally substituted with one, two, or three substituents Q.
Embodiment 45. The carbohydrate-oligonucleotide conjugate of any one of embodiments 42 to 44, wherein each Rⁿ is independently methanediyl, ethane-1,2-diyl, propane-1,3-diyl, butane-1,4-diyl, pentane-1,5-diyl, hexane-1,6-diyl, phen-1,4-diyl, 1,2,3-triazol-1,4-diyl, pyrrolidin-1,3-diyl, or piperidin-1,4-diyl, each optionally substituted with one or more substituents Q.
Embodiment 46. The carbohydrate-oligonucleotide conjugate of any one of embodiments 42 to 45, wherein each Zⁿ is independently a bond, —C(O)—, —C(O)O—, —C(O)NH—, —OC(O)NH—, —C(CH₃)═NO—, —O—, —OP(O₂)O—, —OP(O₂)S—, —NH—, —N(CH₃)—, —S—, or —S(O)₂—.
Embodiment 47. The carbohydrate-oligonucleotide conjugate of any one of embodiments 42 to 46, wherein each Zⁿ is independently a bond, —C(O)NH—, —O—, —OP(O₂)O—, —OP(O₂)S—, or —NH—.
Embodiment 48. The carbohydrate-oligonucleotide conjugate of any one of embodiments 42 to 47, wherein z is an integer of 0, 1, 2, 3, 4, 5, 6, 7, or 8.
Embodiment 49. The carbohydrate-oligonucleotide conjugate of any one of embodiments 42 to 48, wherein z is an integer of 0, 1, 2, 3, 4, or 5.
Embodiment 50. The carbohydrate-oligonucleotide conjugate of any one of embodiments 36 to 42, wherein each L^{a} is independently:

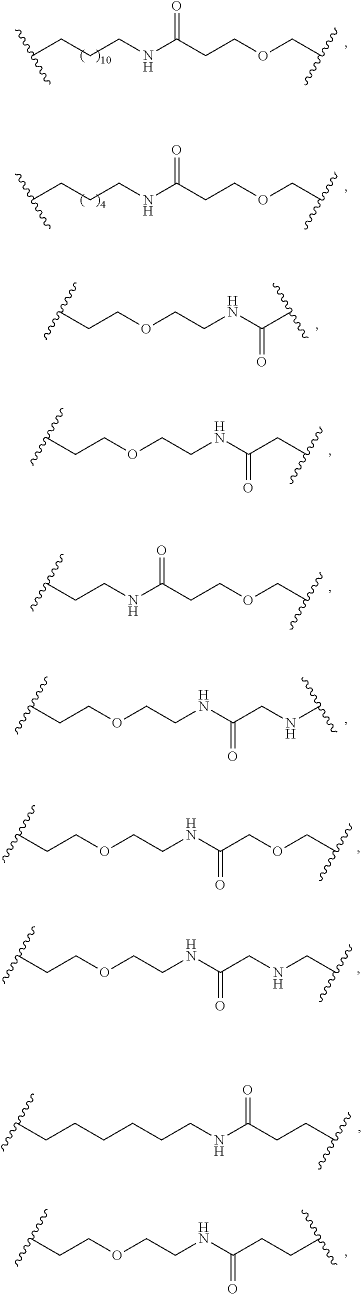

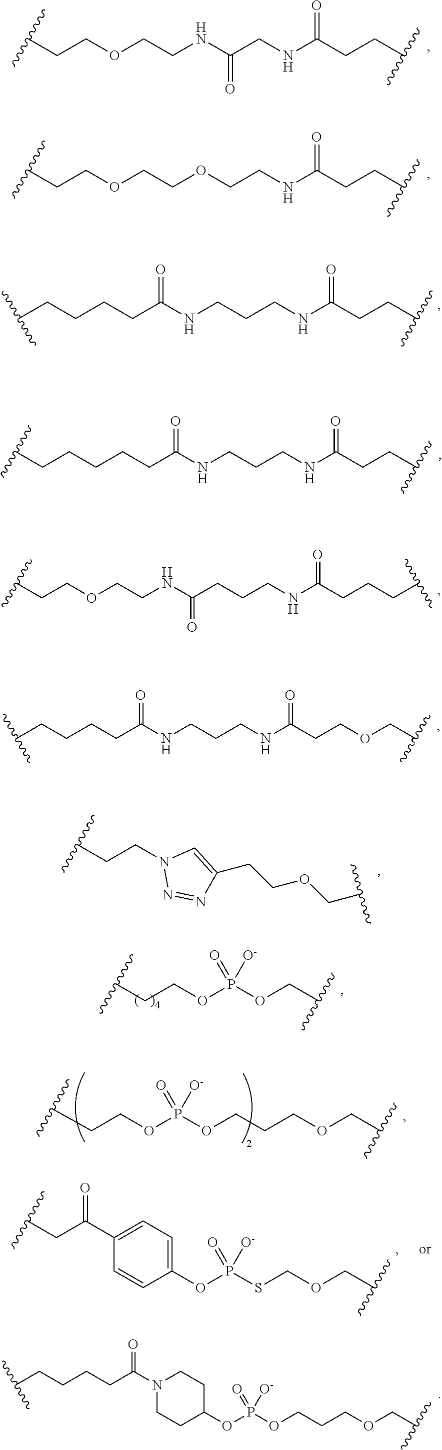

Embodiment 51. The carbohydrate-oligonucleotide conjugate of any one of embodiments 1 to 35, wherein the ASGPR binding moiety has the structure of Formula (A-V):

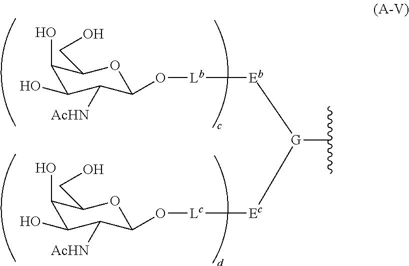

or an enantiomer, a mixture of enantiomers, a diastereomer, a mixture of two or more diastereomers, a tautomer, or a mixture of two or more tantomers thereof; wherein:
E^{b} and c are:
(i) c is an integer of 1; and E^{b} is a bond;
(ii) c is an integer of 2; and E^{b} is a trivalent linker; or
(iii) c is an integer of 3; and E^{b} is a tetravalent linker;
E^{c} and d are:
(i) d is an integer of 1; and E^{c} is bond;
(ii) d is an integer of 2; and Ee is a trivalent linker; or
(iii) d is an integer of 3; and E^{c} is a tetravalent linker;
G is a trivalent linker; and
each L^{b} and L^{c} is independently a divalent linker.
Embodiment 52. The carbohydrate-oligonucleotide conjugate of embodiment 51, wherein c is an integer of 1; and E^{b} is a bond.
Embodiment 53. The carbohydrate-oligonucleotide conjugate of embodiment 51, wherein c is an integer of 2; and E^{b} is a trivalent linker.
Embodiment 54. The carbohydrate-oligonucleotide conjugate of embodiment 53, wherein E^{b} is

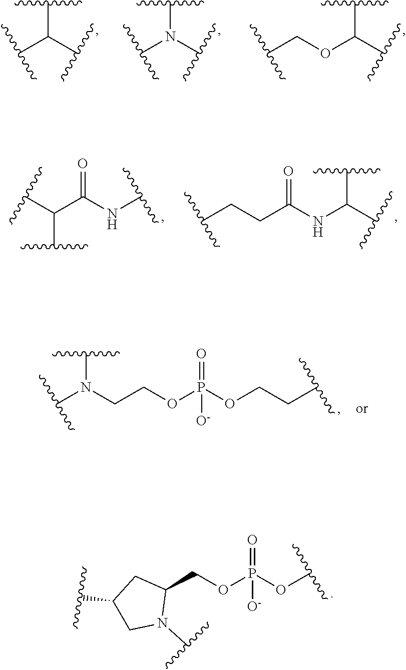

Embodiment 55. The carbohydrate-oligonucleotide conjugate of any one of embodiments 51 to 53, wherein d is an integer of 2; and E^{c} is a trivalent linker.
Embodiment 56. The carbohydrate-oligonucleotide conjugate of embodiment 55, wherein E^{c} is

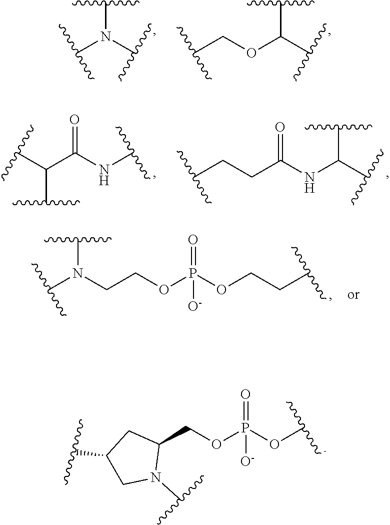

Embodiment 57. The carbohydrate-oligonucleotide conjugate of any one of embodiments 51 to 56, wherein each L^{b} and L^{c} is independently a linker having the structure of —Zⁿ—(Rⁿ—Zⁿ)_{z}—, wherein:
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —C(R^{1a})═NO—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —P(O₂)O—, —P(O)(S)O—, —S—, —S(O)—, —S(O)₂—, —S(O)NR^{1b}—, or —S(O)₂NR^{1b}—;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
z is an integer of 0, 1, 2, 3, 4, 5, 6, 7, 8, 9, or 10;
wherein each alkyl, alkylene, heteroalkyl, alkenyl, alkenylene, alkynyl, alkynylene, cycloalkyl, cycloalkylene, aryl, arylene, heteroaryl, heteroarylene, heterocyclyl, or heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{c}, —OS(O)₂R^{c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.
Embodiment 58. The carbohydrate-oligonucleotide conjugate of embodiment 57, wherein each Rⁿ is independently C₁₋₁₀ alkylene, C₆₋₁₄ arylene, heteroarylene, or heterocyclylene each of which is optionally substituted with one or more substituents Q.
Embodiment 59. The carbohydrate-oligonucleotide conjugate of embodiment 57 or 58, wherein each Rⁿ is independently methanediyl, ethanediyl, propanediyl, butanediyl, pentanediyl, hexanediyl, phendiyl, 1,2,3-triazoldiyl, pyrrolidindiyl, or piperidindiyl, each optionally substituted with one, two, or three substituents Q.
Embodiment 60. The carbohydrate-oligonucleotide conjugate of any one of embodiments 57 to 59, wherein each Rⁿ is independently methanediyl, ethane-1,2-diyl, propane-1,3-diyl, butane-1,4-diyl, pentane-1,5-diyl, hexane-1,6-diyl, phen-1,4-diyl, 1,2,3-triazol-1,4-diyl, pyrrolidin-1,3-diyl, or piperidin-1,4-diyl, each optionally substituted with one or more substituents Q.
Embodiment 61. The carbohydrate-oligonucleotide conjugate of any one of embodiments 57 to 60, wherein each Zⁿ is independently a bond, —C(O)—, —C(O)O—, —C(O)NH—, —OC(O)NH—, —C(CH₃)═NO—, —O—, —OP(O₂)O—, —OP(O₂)S—, —NH—, —N(CH₃)—, —P(O₂)O—, —P(O)(S)O—, —S—, or —S(O)₂—.
Embodiment 62. The carbohydrate-oligonucleotide conjugate of any one of embodiments 57 to 61, wherein each Zⁿ is independently a bond, —C(O)NH—, —O—, —OP(O₂)O—, —OP(O₂)S—, or —NH—.
Embodiment 63. The carbohydrate-oligonucleotide conjugate of any one of embodiments 57 to 62, wherein z is an integer of 0, 1, 2, 3, 4, 5, 6, 7, or 8.
Embodiment 64. The carbohydrate-oligonucleotide conjugate of any one of embodiments 57 to 63, wherein z is an integer of 0, 1, 2, 3, 4, or 5.
Embodiment 65. The carbohydrate-oligonucleotide conjugate of embodiment 51 or 57, wherein each L^{b} and L^{c} is independently:

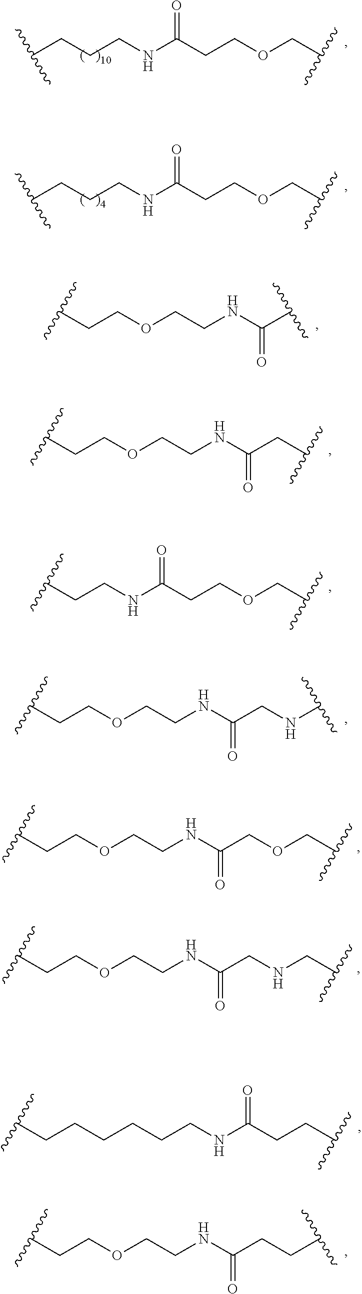

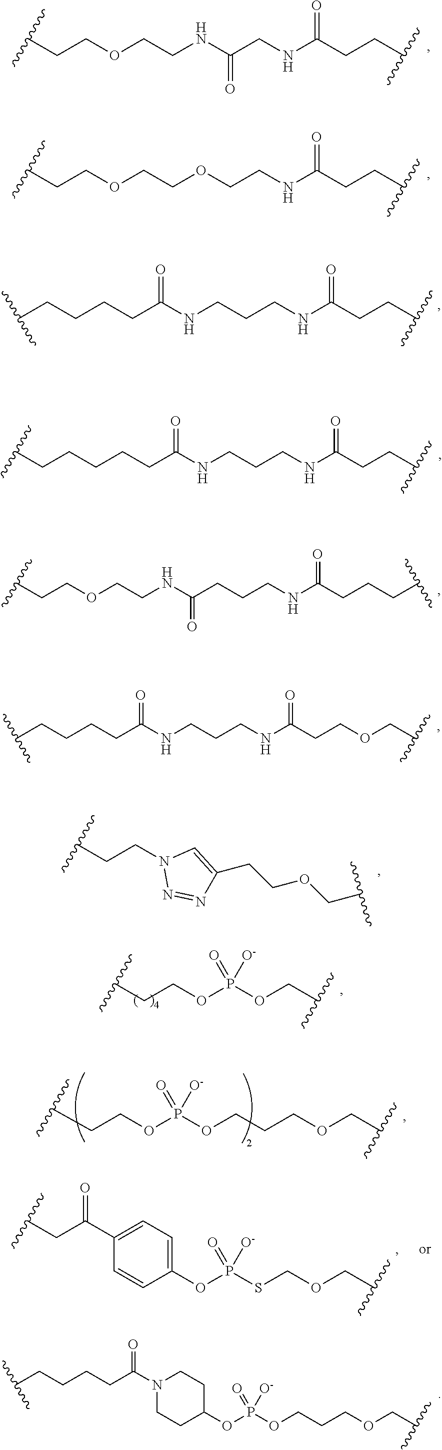

Embodiment 66. The carbohydrate-oligonucleotide conjugate of any one of embodiments 1 to 35, wherein the ASGPR binding moiety has the structure of:

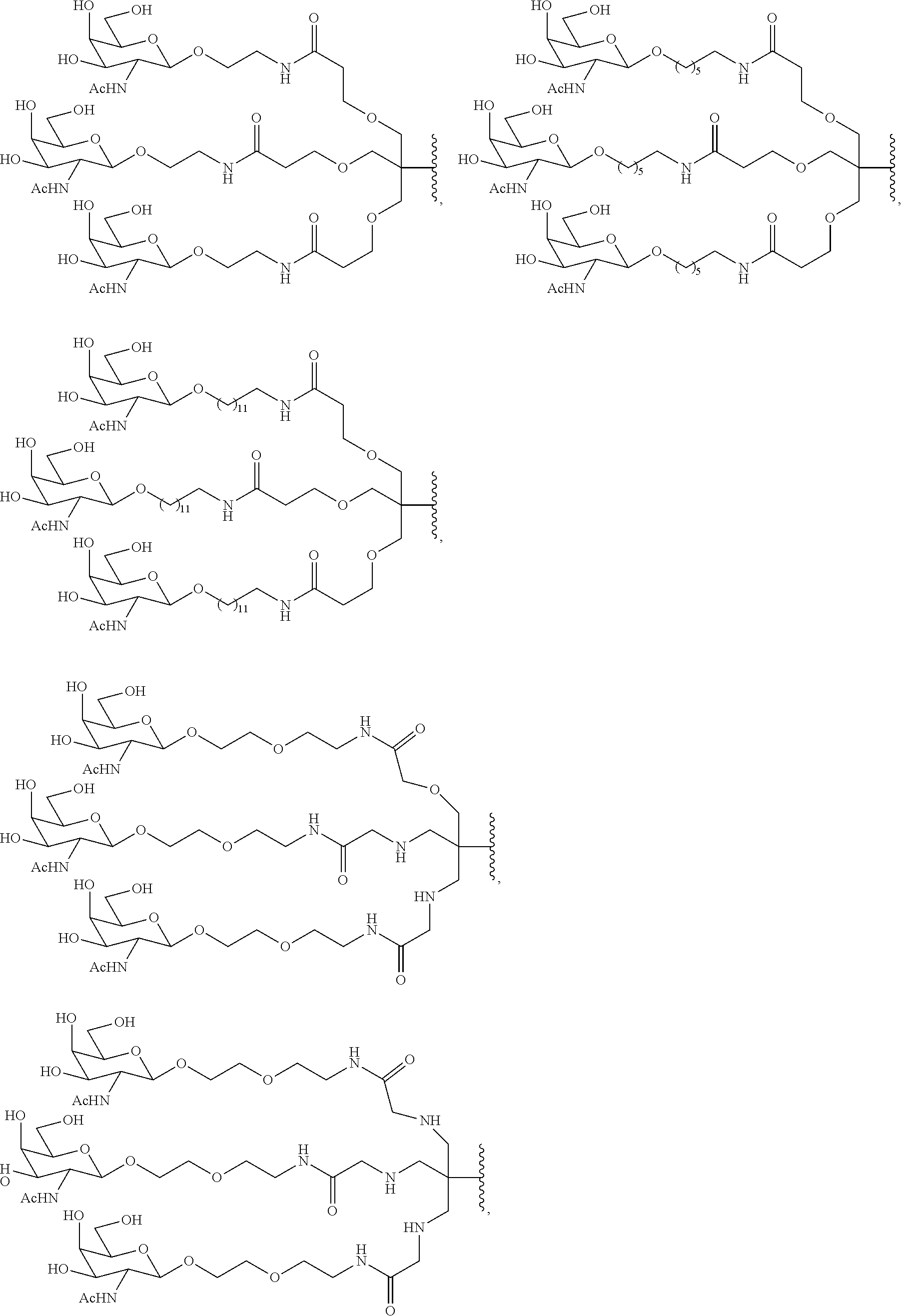

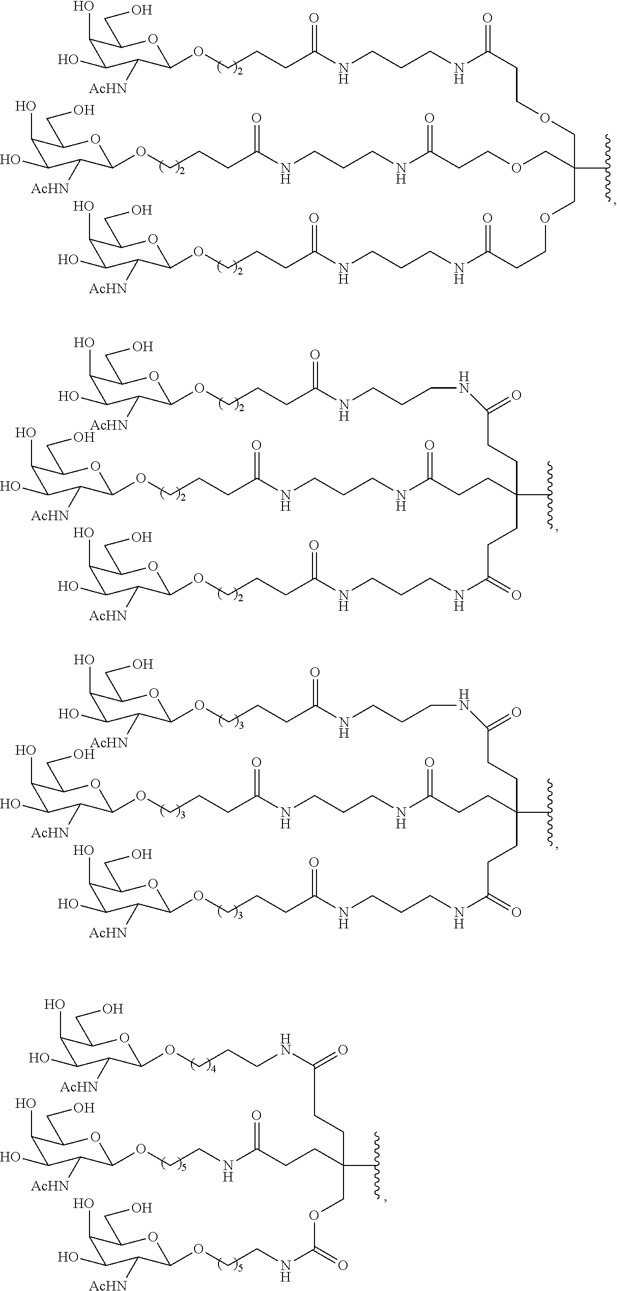

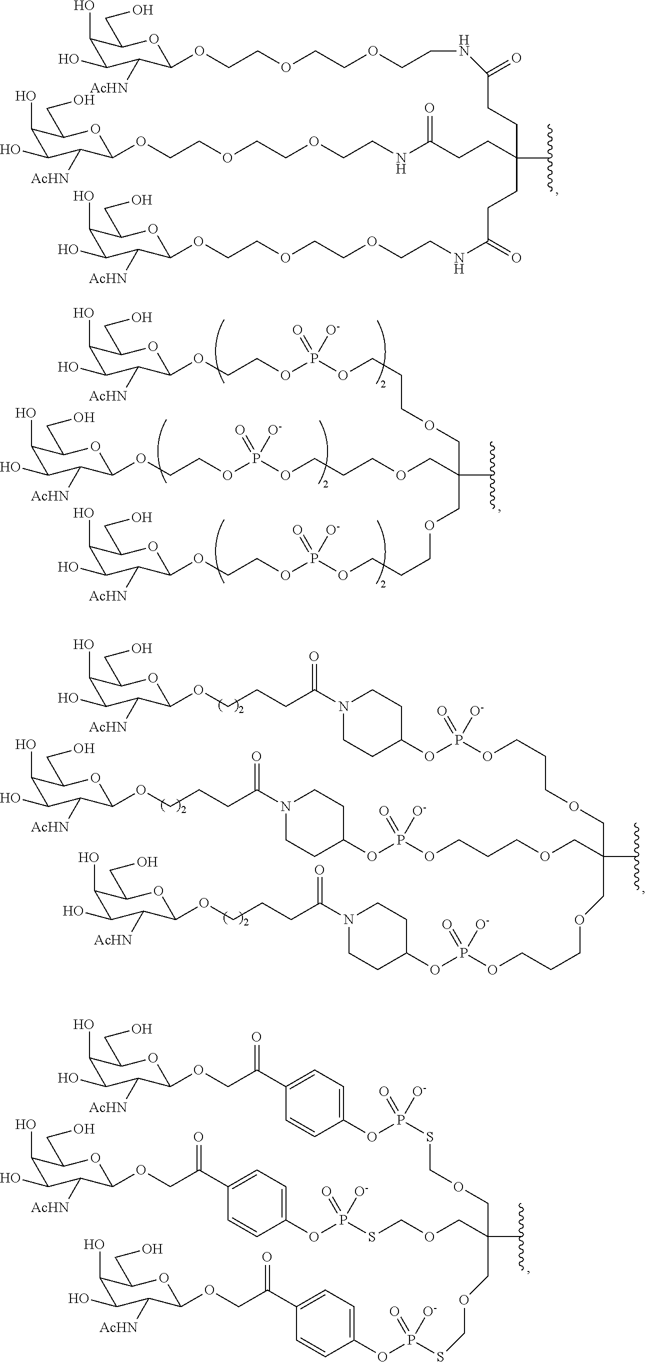

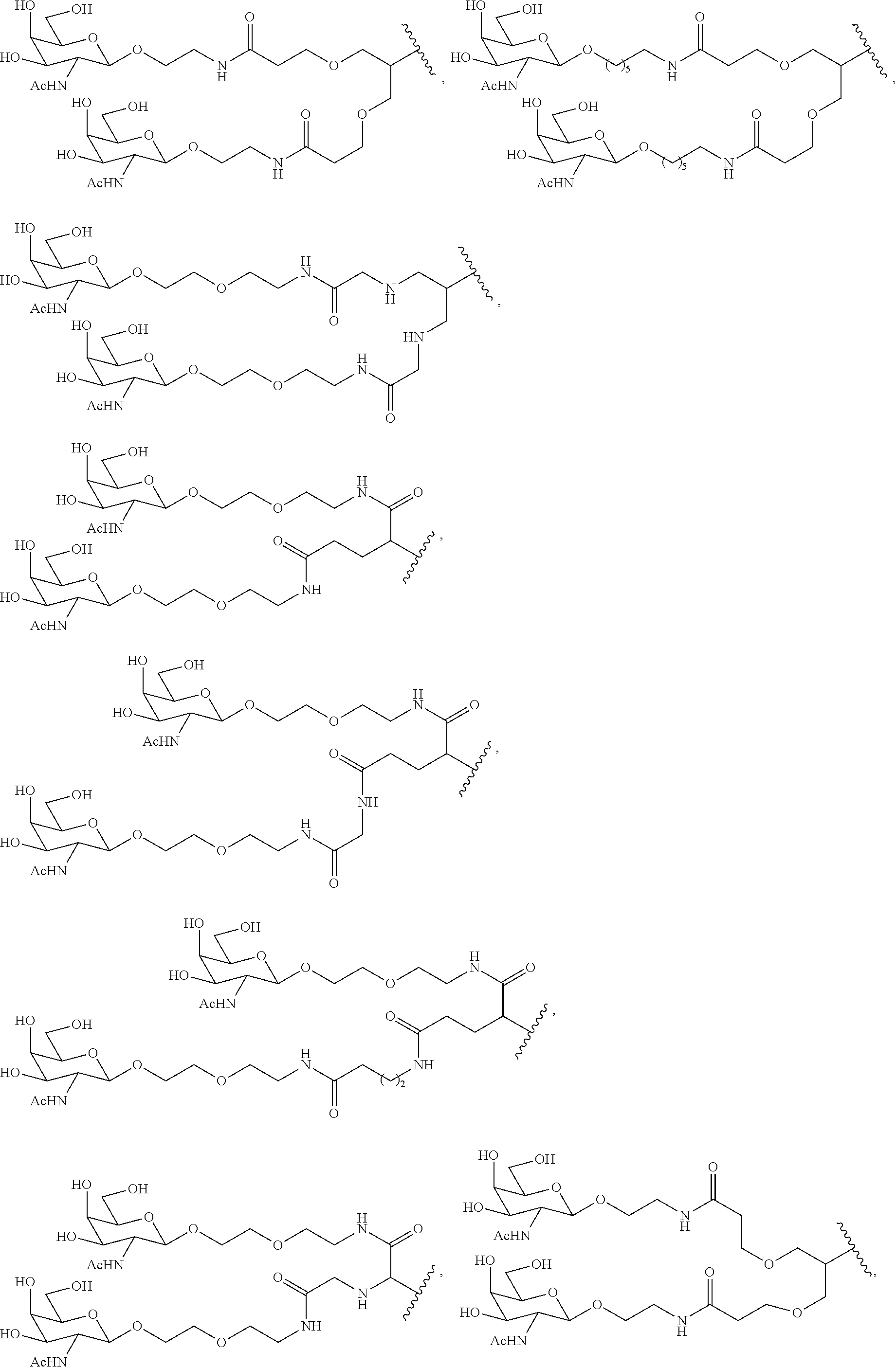

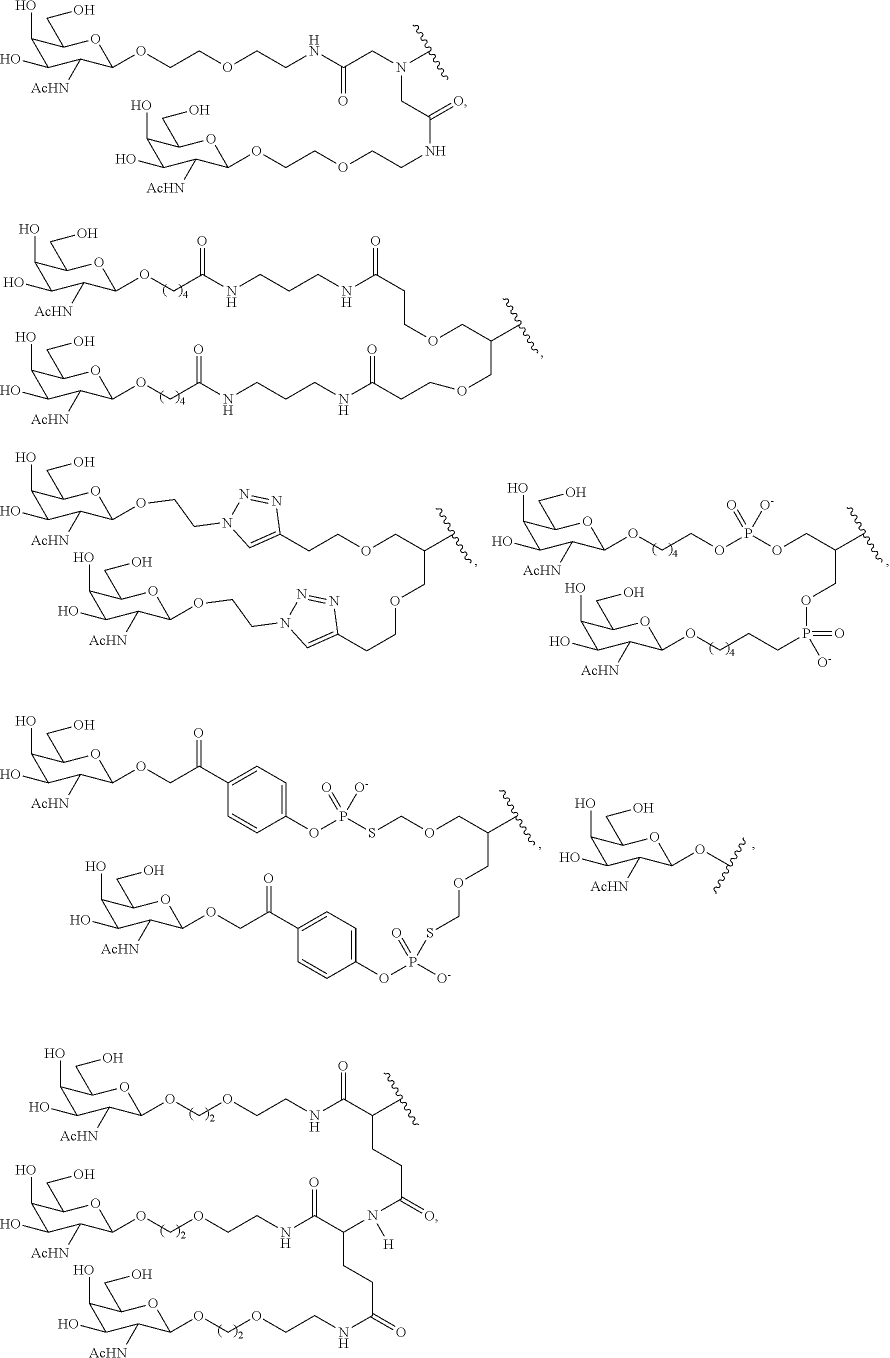

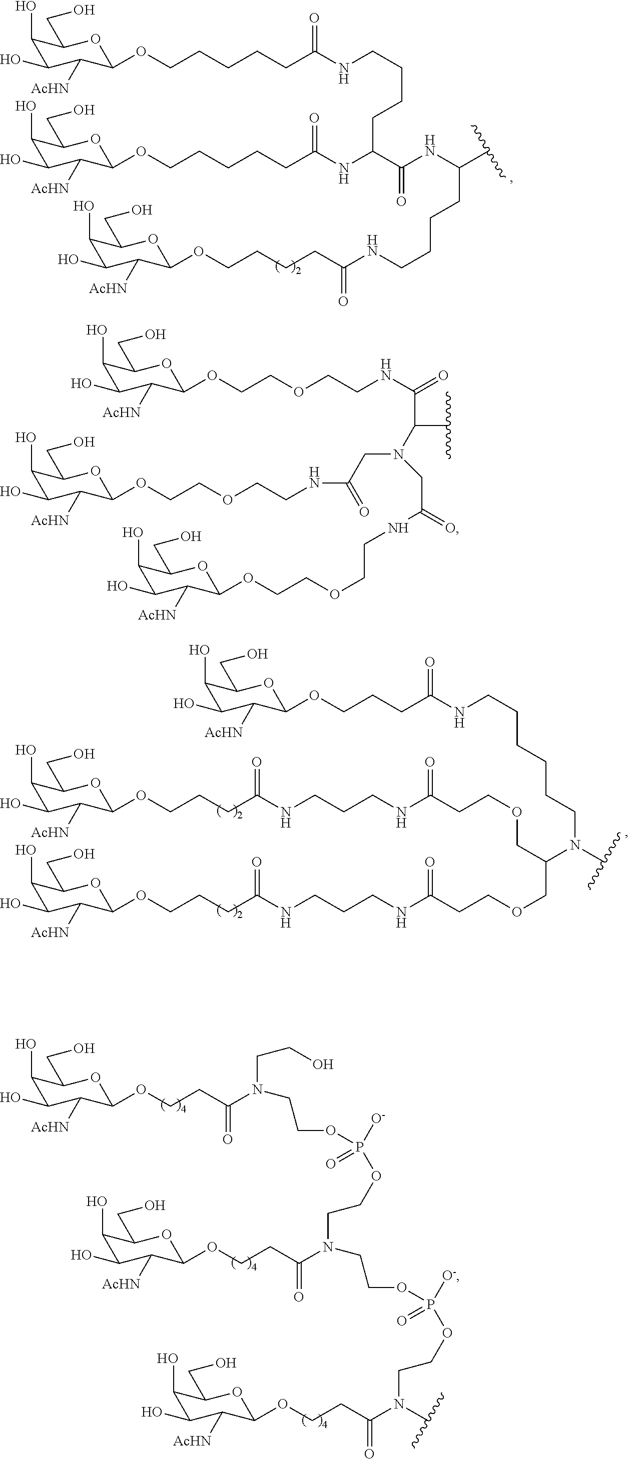

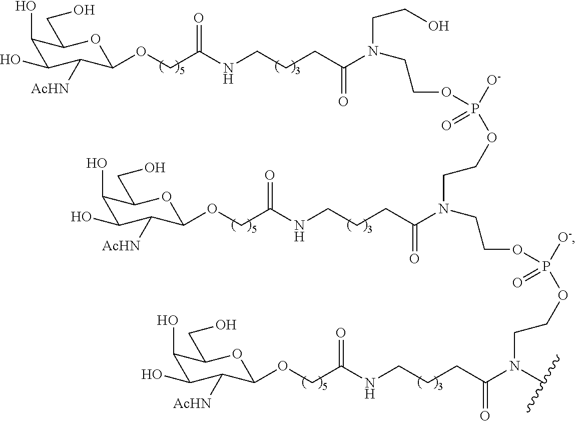

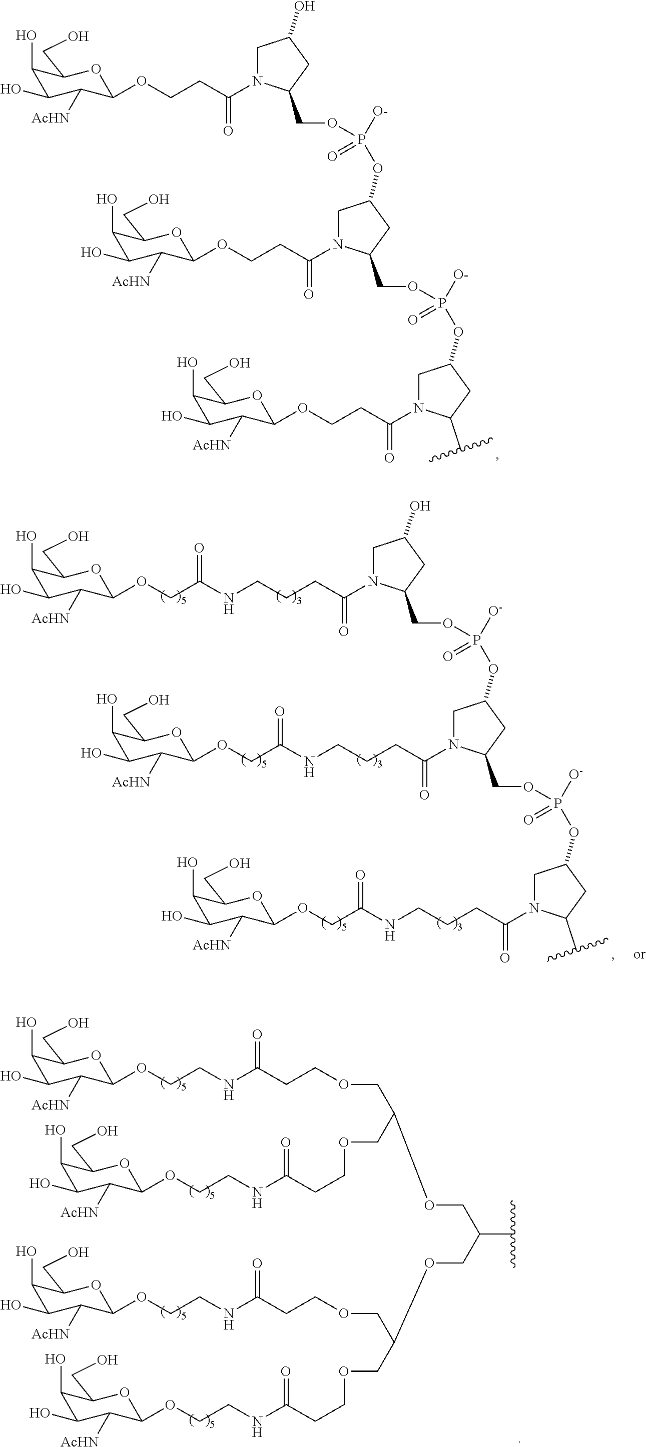

Embodiment 67. The carbohydrate-oligonucleotide conjugate of any one of embodiments 2 to 10, 13 to 23, and 25 to 66, wherein the trivalent linker is independently a linker having the structure of

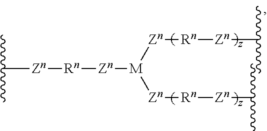

wherein:
M is (i) N or CH; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl;
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —C(R^{1a})═NO—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —P(O₂)O—, —P(O)(S)O—, —S—, —S(O)—, —S(O)₂—, —S(O)NR^{1b}—, or —S(O)₂NR^{1b}—;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
z is an integer of 0, 1, 2, 3, 4, 5, 6, 7, 8, 9, or 10;
wherein each alkyl, alkylene, heteroalkyl, alkenyl, alkenylene, alkynyl, alkynylene, cycloalkyl, cycloalkylene, aryl, arylene, heteroaryl, heteroarylene, heterocyclyl, or heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR)NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{c}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{c}S(O)₂NR^{f}R^{g}, —SR^{c}, —S(O)R^{c}, —S(O)₂R^{c}, —S(O)NR^{f}R^{g}, and —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.
Embodiment 68. The carbohydrate-oligonucleotide conjugate of embodiment 67, wherein M is N.
Embodiment 69. The carbohydrate-oligonucleotide conjugate of embodiment 67, wherein M is CH, or C(CH₃).
Embodiment 70. The carbohydrate-oligonucleotide conjugate of any one of embodiments 67 to 69, wherein each Rⁿ is independently C₁₋₁₀ alkylene, C₆₋₁₄ arylene, heteroarylene, or heterocyclylene, each of which is optionally substituted with one or more substituents Q.
Embodiment 71. The carbohydrate-oligonucleotide conjugate of any one of embodiments 67 to 70, wherein each Rⁿ is independently methanediyl, ethanediyl, propanediyl, butanediyl, pentanediyl, hexanediyl, heptanediyl, octanediyl, nonanediyl, decanediyl, phendiyl, 1,2,3-triazoldiyl, pyrrolidindiyl, tetrahydrothiendiyl, or tetrahydropyrandiyl, each optionally substituted with one, two, or three substituents Q.
Embodiment 72. The carbohydrate-oligonucleotide conjugate of any one of embodiments 67 to 71, wherein each Rⁿ is independently methanediyl, ethane-1,2-diyl, acetamidoethane-1,2-diyl, 1-acetamidoethane-1,2-diyl, propane-1,3-diyl, 2-hydroxylpropane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, phen-1,4-diyl, 1,2,3-triazol-1,4-diyl, or 2,5-dioxopyrrolidin-1,3-diyl.
Embodiment 73. The carbohydrate-oligonucleotide conjugate of any one of embodiments 67 to 72, wherein each Zⁿ is independently a bond, —C(O)—, —C(O)O—, —C(O)NH—, —C(CH₃)═NO—, —O—, —OC(O)NH—, —NH—, —N(CH₃), —P(O₂)O)—, —P(O)(S)O—, —P(O₂)O—, —P(O)(S)O—, —S—, or —S(O)₂—.
Embodiment 74. The carbohydrate-oligonucleotide conjugate of any one of embodiments 67 to 73, wherein each Zⁿ is independently a bond, —C(O)—, —C(O)NH—, —C(CH₃)═NO—, —O—, —NH—, —P(O₂)O—, —P(O)(S)O)—, or —S—.
Embodiment 75. The carbohydrate-oligonucleotide conjugate of any one of embodiments 67 to 74, wherein each z is independently an integer of 0, 1, 2, 3, 4, or 5.
Embodiment 76. The carbohydrate-oligonucleotide conjugate of any one of embodiments 67 to 75, wherein each z is independently an integer of 0, 1, 2, 3, or 4.
Embodiment 77. The carbohydrate-oligonucleotide conjugate of any one of embodiments 2 to 10, 13 to 23, and 25 to 67, wherein the trivalent linker has the structure of:

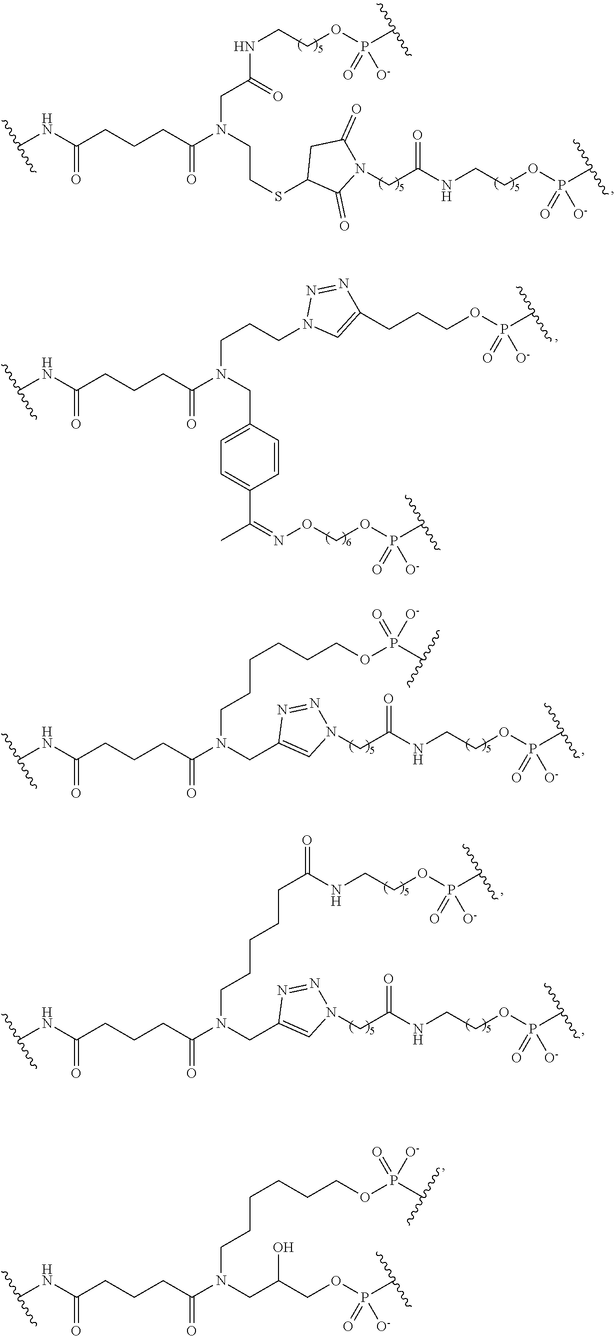

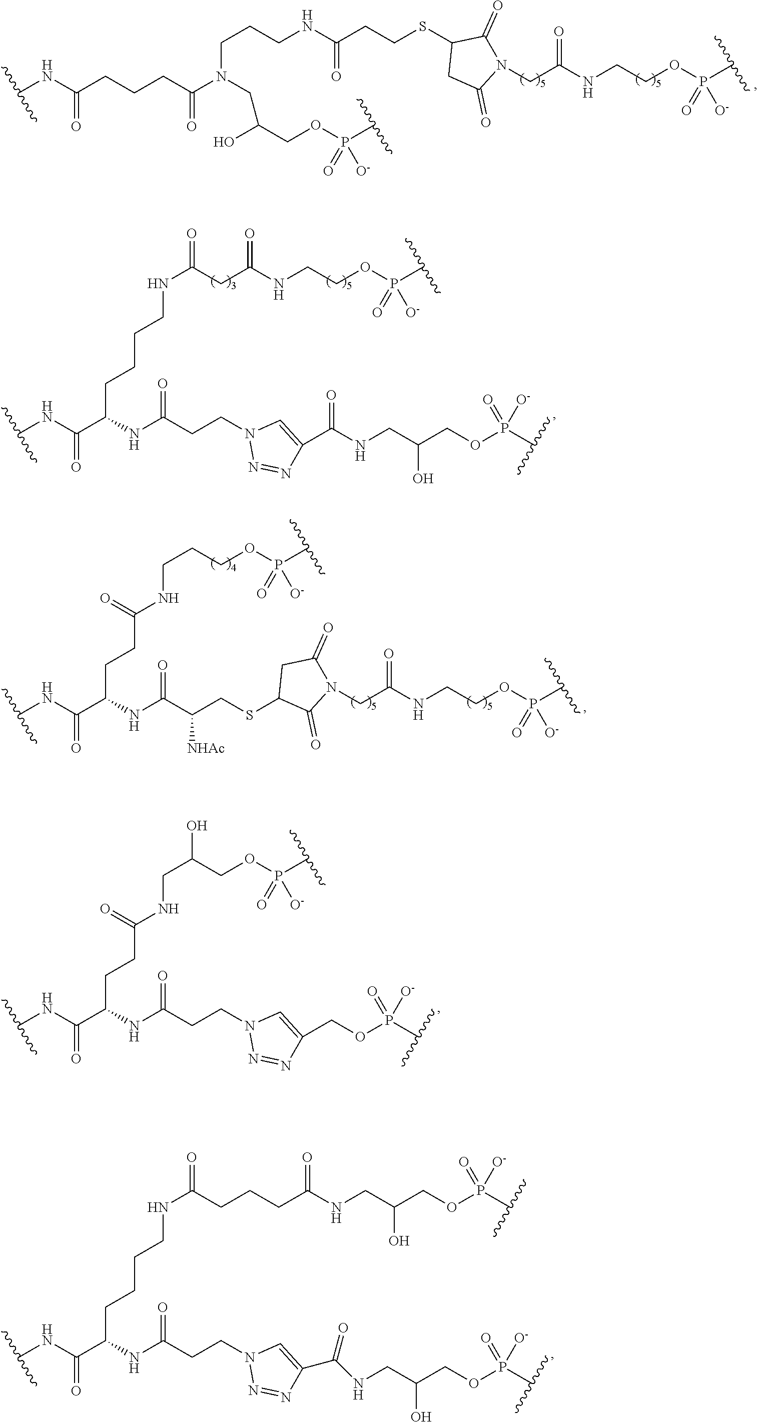

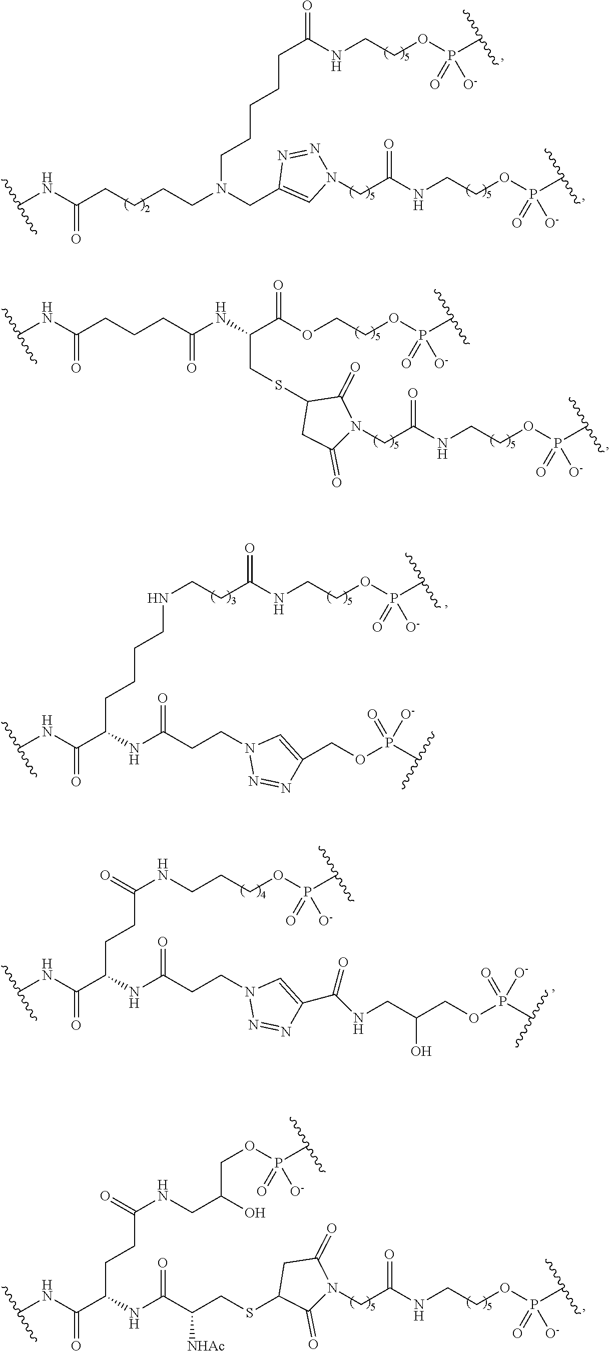

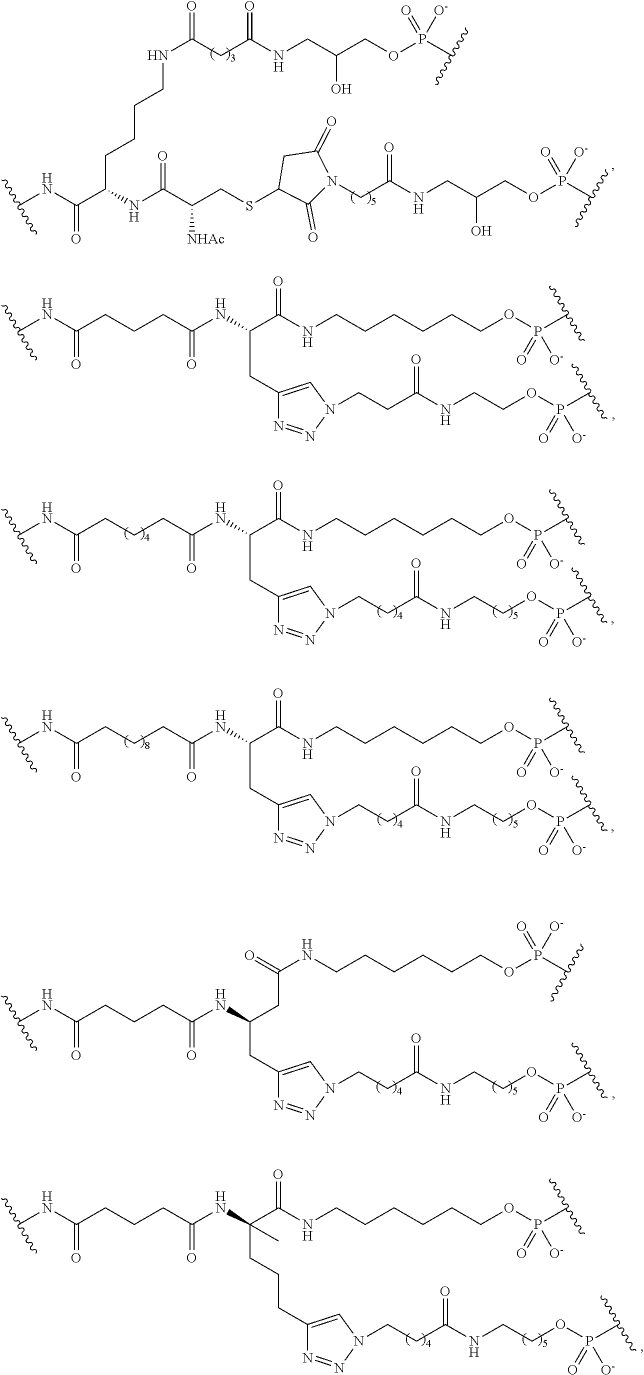

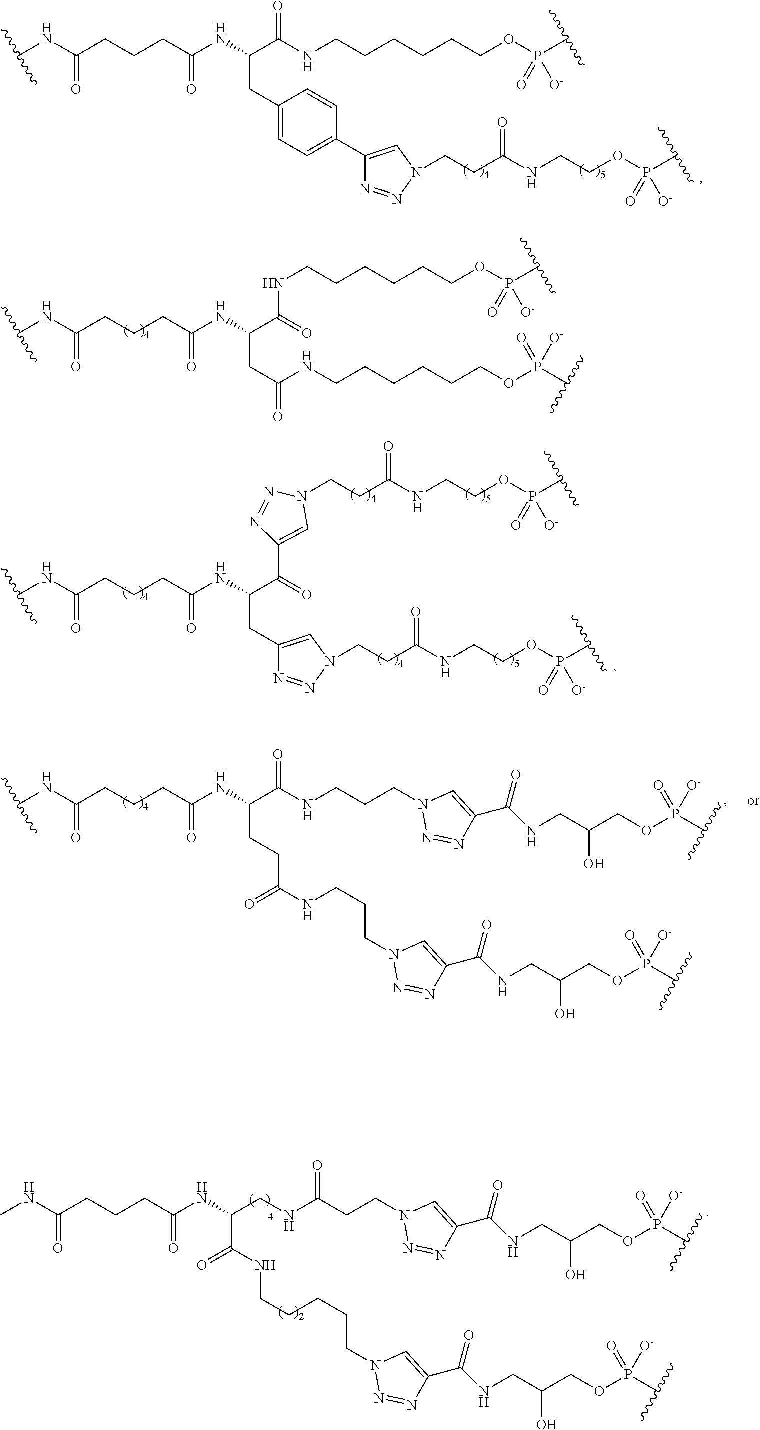

Embodiment 78. The carbohydrate-oligonucleotide conjugate of any one of embodiments 1, 11 to 19, and 24 to 66, wherein each divalent linker is independently a linker having the structure of —Zⁿ—(Rⁿ—Zⁿ)_{z}—, wherein:
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —C(R^{1a})—NO—, —O—, —OC(O)O—, —OC(O)NR^{1b}, —OC(O)S—, —OC(NR^{1a})NR^{1b}—, —OC(S)O—, —OC(S)NR^{1b}—, —OS(O), —OS(O)₂, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —P(O₂)O—, —P(O)(S)O—, —S—, —S(O)—, —S(O)₂—, —S(O)NR^{1b}—, or —S(O)₂NR^{1b}—;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
z is an integer of 0, 1, 2, 3, 4, 5, 6, 7, 8, 9, or 10;
wherein each alkyl, alkylene, heteroalkyl, alkenyl, alkenylene, alkynyl, alkynylene, cycloalkyl, cycloalkylene, aryl, arylene, heteroaryl, heteroarylene, heterocyclyl, or heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{c}, —C(O)NR^{f}R^{g}, —C(O)SR^{c}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{c}, —OC(NR^{c})NR^{f}R^{g}, —OC(S)R^{c}, —OC(S)OR^{c}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{c}S(O)₂NR^{f}R^{g}, —SR^{c}, —S(O)R^{c}, —S(O)₂R^{c}, —S(O)NR^{f}R^{g}, and —S(O)₂NR^{f}R^{g}; wherein each R^{c}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.
Embodiment 79. The carbohydrate-oligonucleotide conjugate of embodiment 78, wherein each Rⁿ is independently C₁₋₁₀ alkylene, C₆₋₁₄ arylene, heteroarylene, or heterocyclylene, each of which is optionally substituted with one or more substituents Q.
Embodiment 80. The carbohydrate-oligonucleotide conjugate of embodiment 78 or 79, each Rⁿ is independently methanediyl, ethanediyl, propanediyl, butanediyl, pentanediyl, hexanediyl, phendiyl, 1,2,3-triazoldiyl, or pyrrolidindiyl, each optionally substituted with one, two, or three substituents Q.
Embodiment 81. The carbohydrate-oligonucleotide conjugate of any one of embodiments 78 to 80, wherein each Rⁿ is independently each Rⁿ is independently methanediyl, ethane-1,2-diyl, acetamidoethane-1,2-diyl, 1-acetamidoethane-1,2-diyl, propane-1,3-diyl, 2-hydroxylpropane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, phen-1,4-diyl, 1,2,3-triazol-1,4-diyl, or 2,5-dioxopyrrolidin-1,3-diyl.
Embodiment 82. The carbohydrate-oligonucleotide conjugate of any one of embodiments 78 to 81, wherein each Zⁿ is independently a bond, —C(O)—, —C(O)O—, —C(O)NH—, —C(CH₃)═NO—, —O—, —OC(O)NH—, —NH—, —N(CH₃)—, —P(O₂)O—, —P(O)(S)O—, —S—, or —S(O)₂—.
Embodiment 83. The carbohydrate-oligonucleotide conjugate of any one of embodiments 78 to 82, wherein each Zⁿ is independently a bond, —C(O)—, —C(O)NH—, —C(CH₃)═NO—, —O—, —NH—, —P(O₂)O—, —P(O)(S)O—, or —S—.
Embodiment 84. The carbohydrate-oligonucleotide conjugate of any one of embodiments 78 to 83, wherein each z is independently an integer of 0, 1, 2, 3, 4, 5, 6, 7, or 8.
Embodiment 85. The carbohydrate-oligonucleotide conjugate of any one of embodiments 78 to 84, wherein each z is independently an integer of 0, 1, 2, 3, 4, or 5.
Embodiment 86. The carbohydrate-oligonucleotide conjugate of any one of embodiments 1, 11 to 19, 24 to 66, and 78, wherein each divalent linker is independently a linker having the structure of:

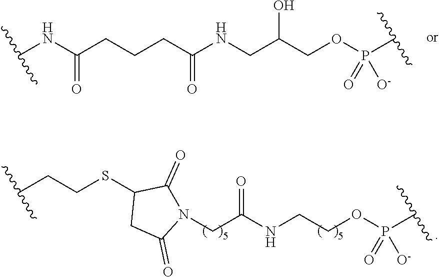

Embodiment 87. The carbohydrate-oligonucleotide conjugate of embodiment 1, having the structure of:

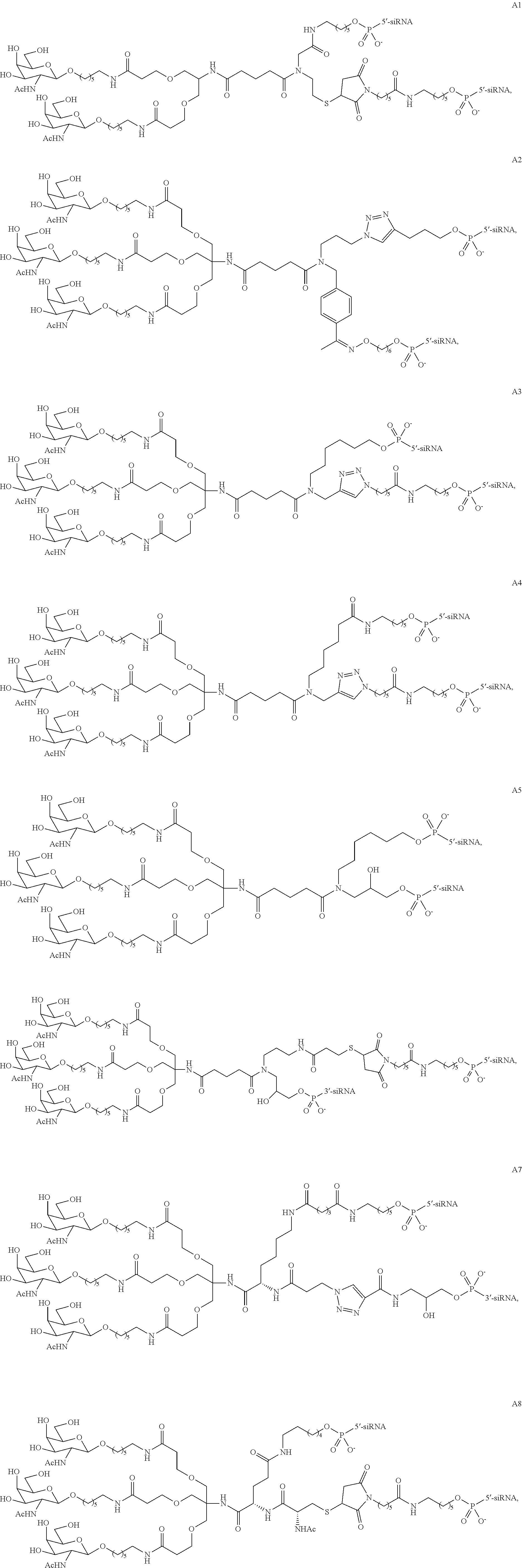

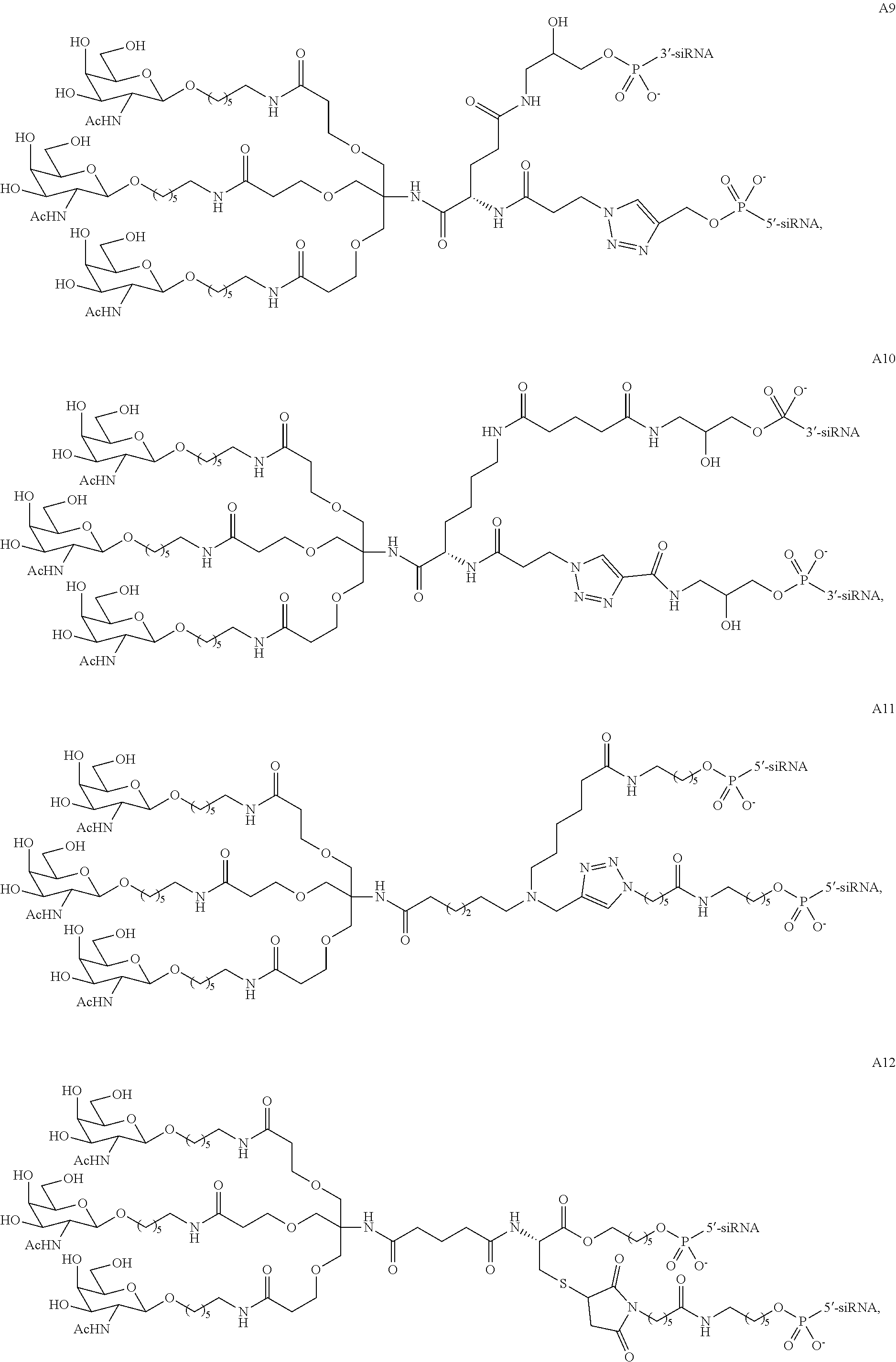

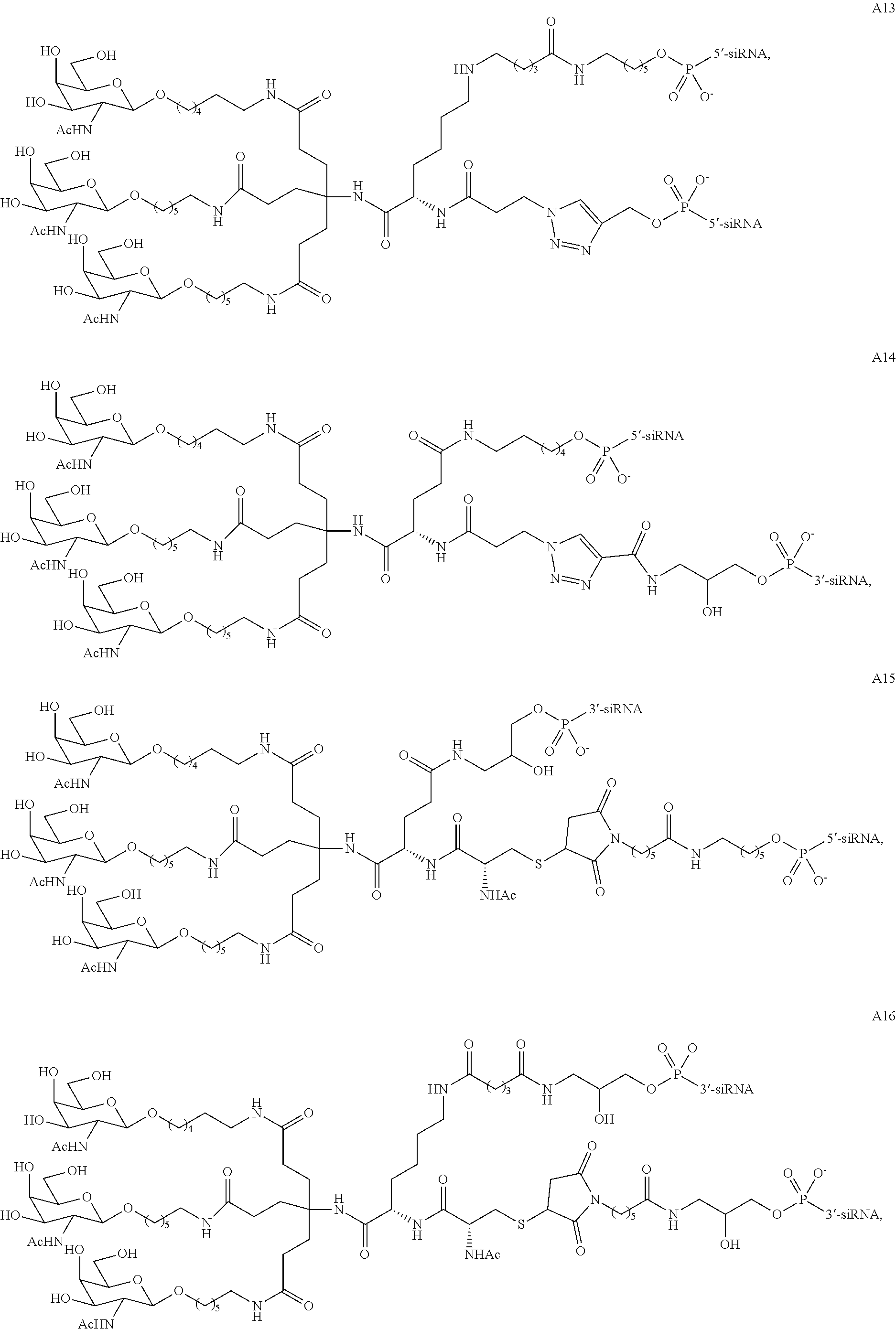

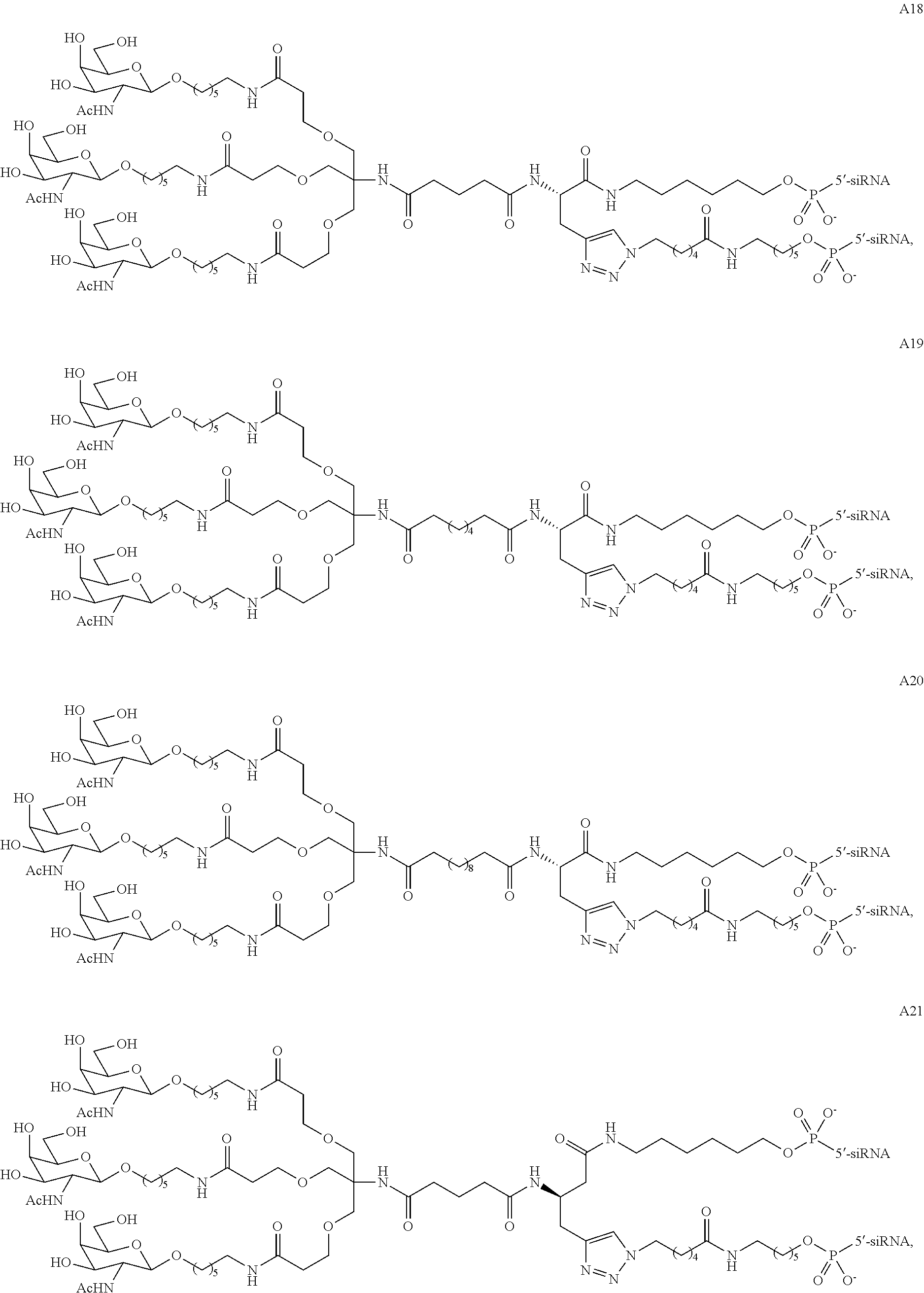

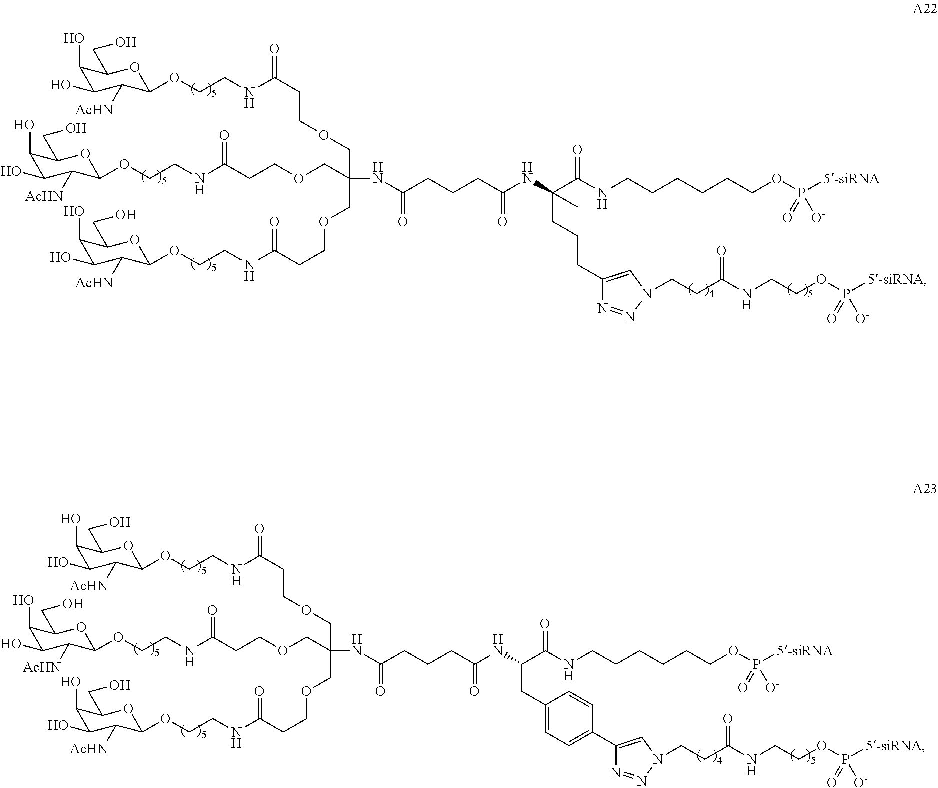

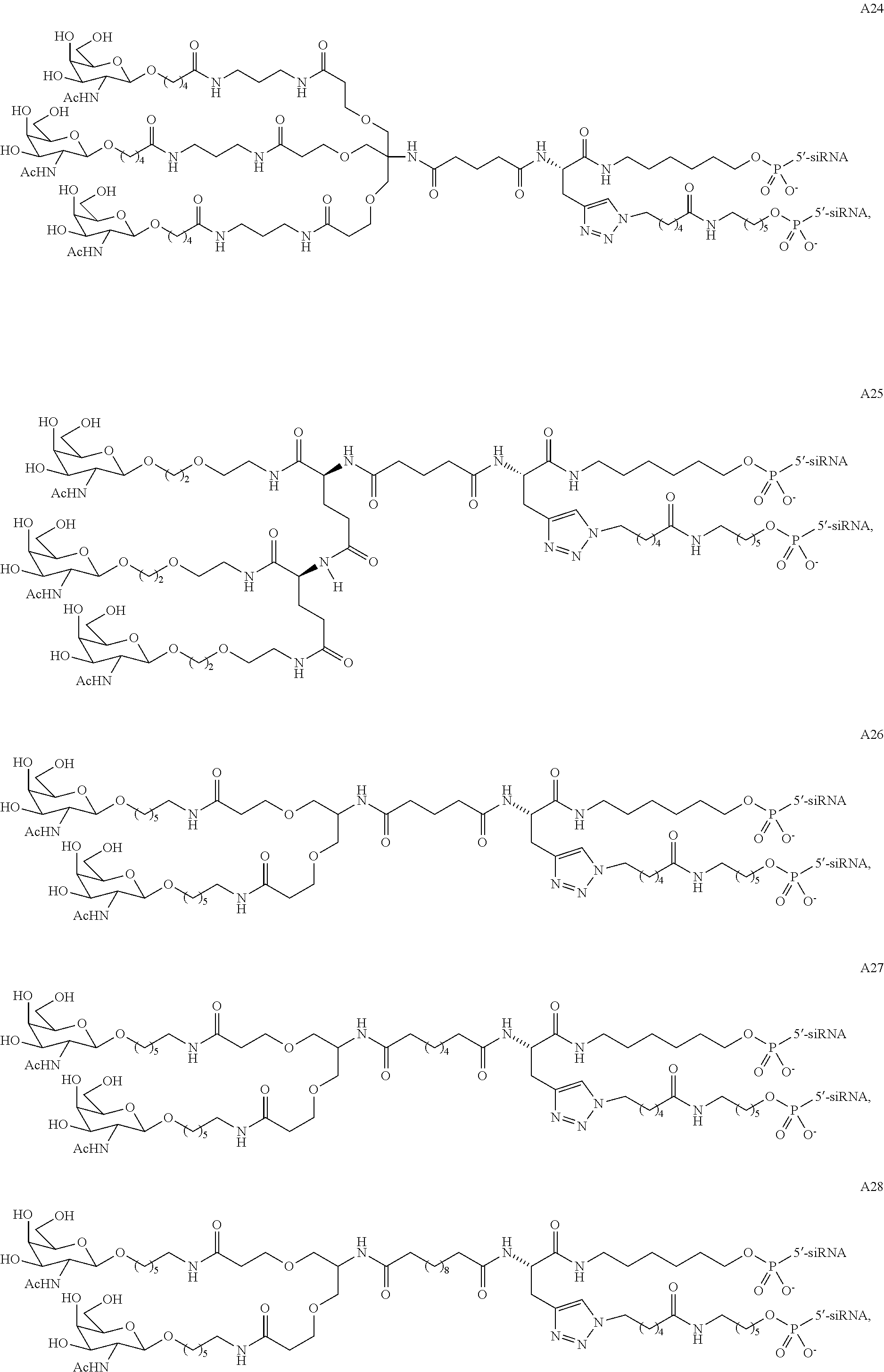

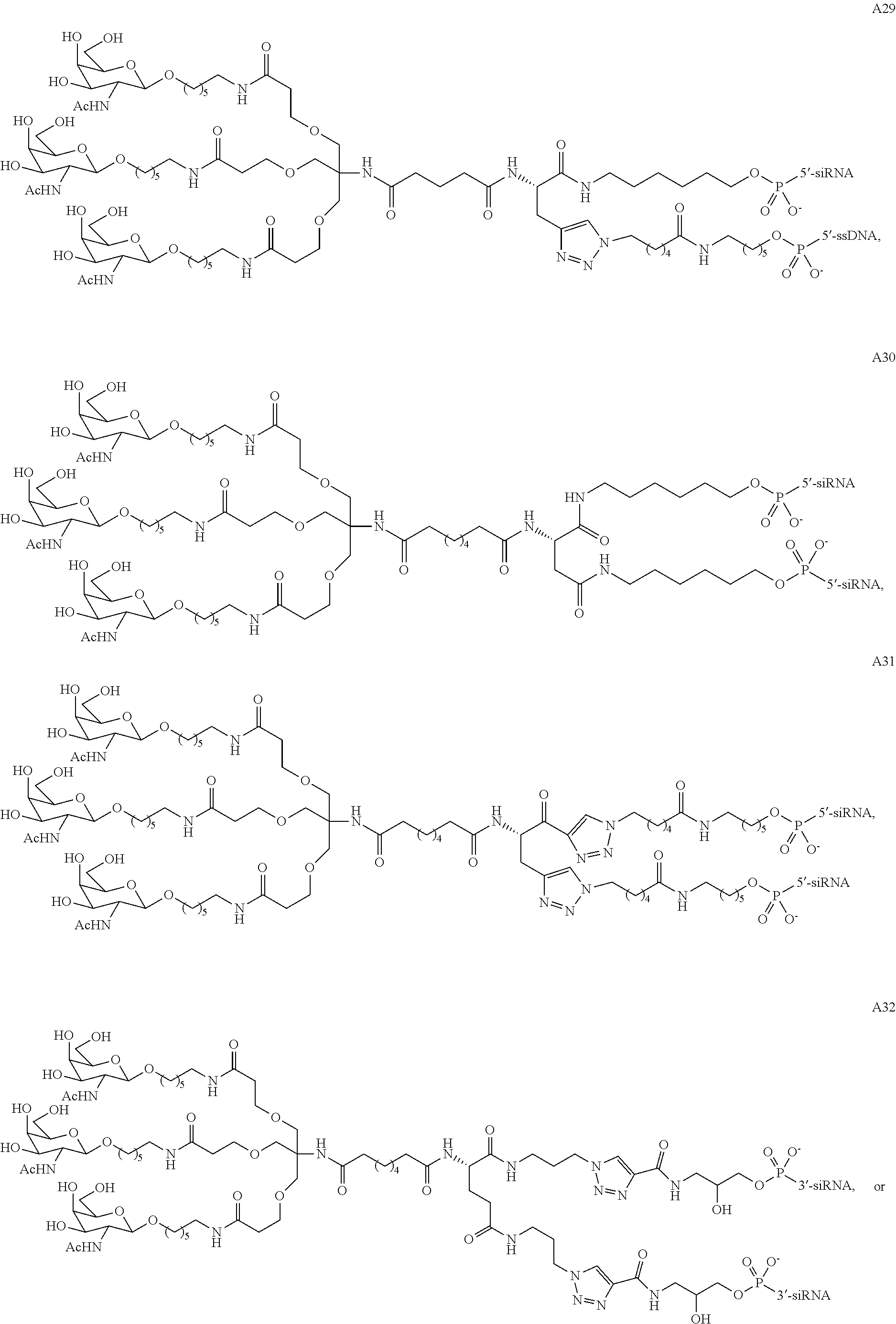

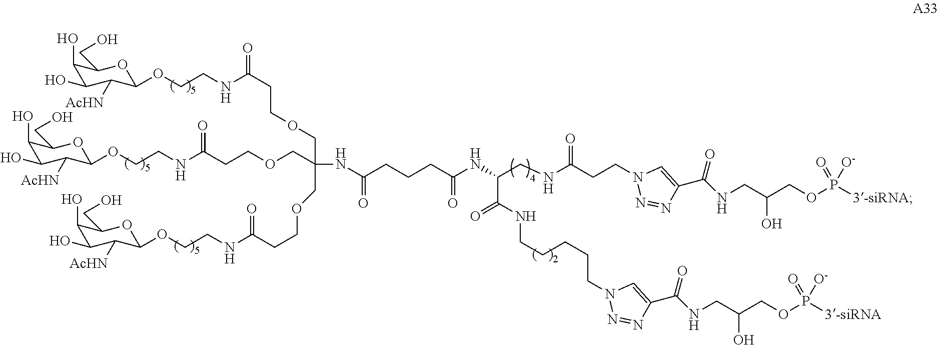

wherein each 3′-siRNA independently represents a siRNA with one of its 3′-termini connected to a trivalent linker; each 5′-siRNA represents a siRNA with one of its 5′-termini connected to a trivalent linker; and 5′-ssDNA represents a single stranded DNA with its 5′-terminus connected to a trivalent linker.
Embodiment 88. The carbohydrate-oligonucleotide conjugate of embodiment 1, having the structure of:

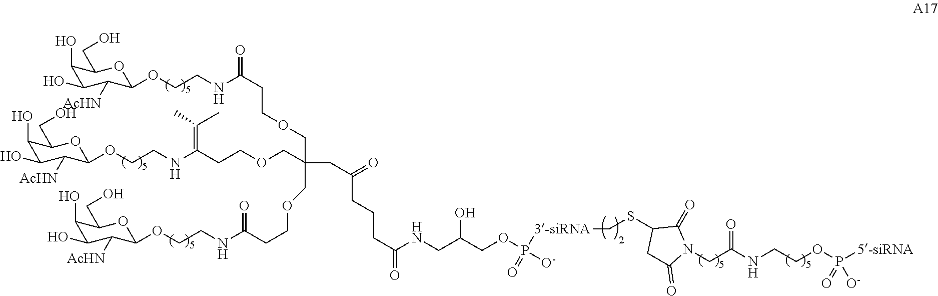

wherein 3′-siRNA represents a siRNA with one of its 3′-termini connected to a first divalent linker and one of its 5′-termini connected to a second divalent linker; and 5′-siRNA represents a siRNA with one of its 5′-termini connected to the second divalent linker.
Embodiment 89. The carbohydrate-oligonucleotide conjugate of any of embodiments 1 to 19 and 33 to 88, wherein one oligonucleotide is an oligonucleotide duplex comprising comprises a pair of nucleotide sequences of SEQ ID NOs: 1 and 2, 3 and 4, 5 and 6, 7 and 8, 9 and 10, 11 and 12, 13 and 14, 15 and 16, 17 and 18, 19 and 20, 21 and 22, or 23 and
Embodiment 90. The carbohydrate-oligonucleotide conjugate of embodiment 1, having the structure of:

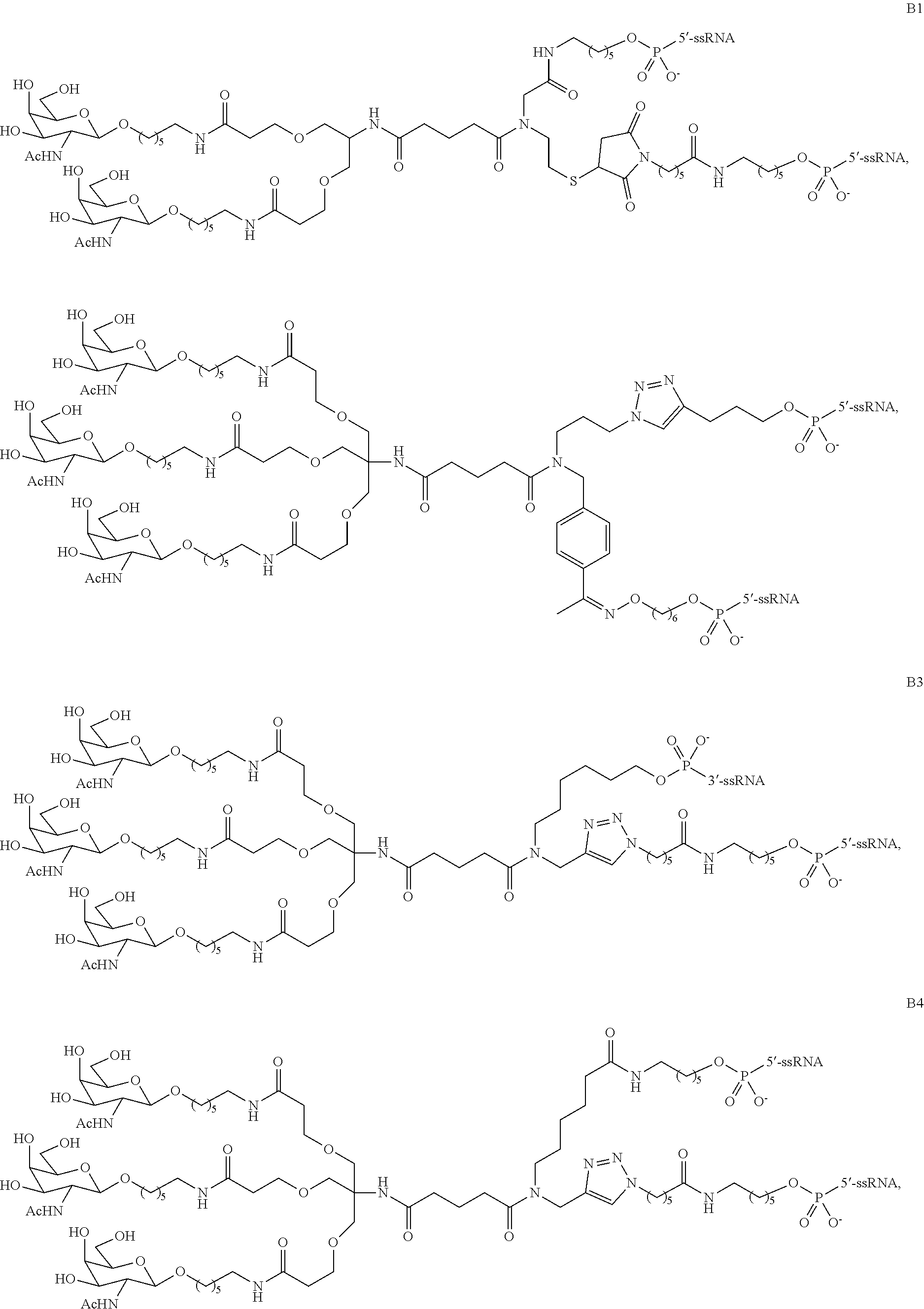

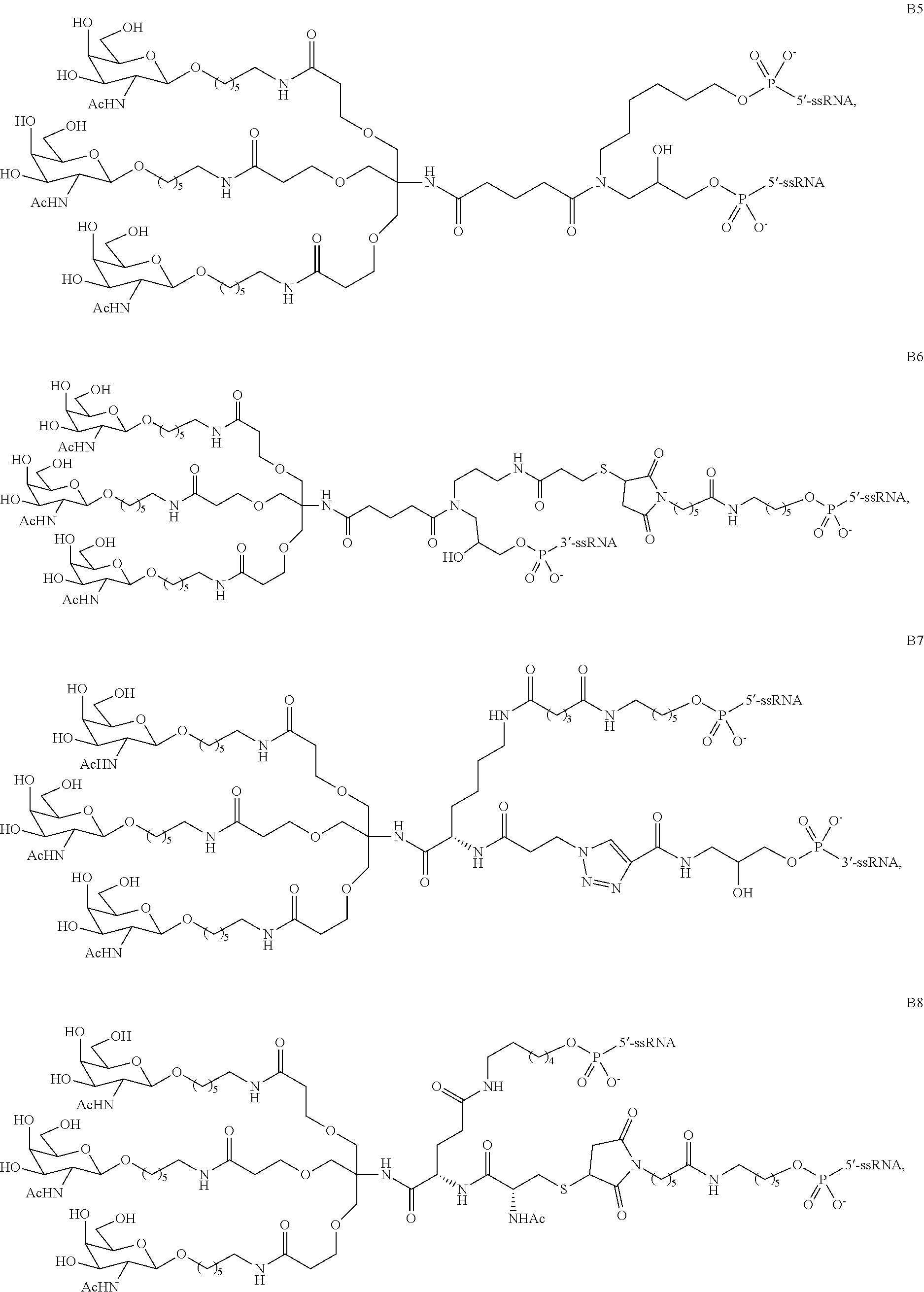

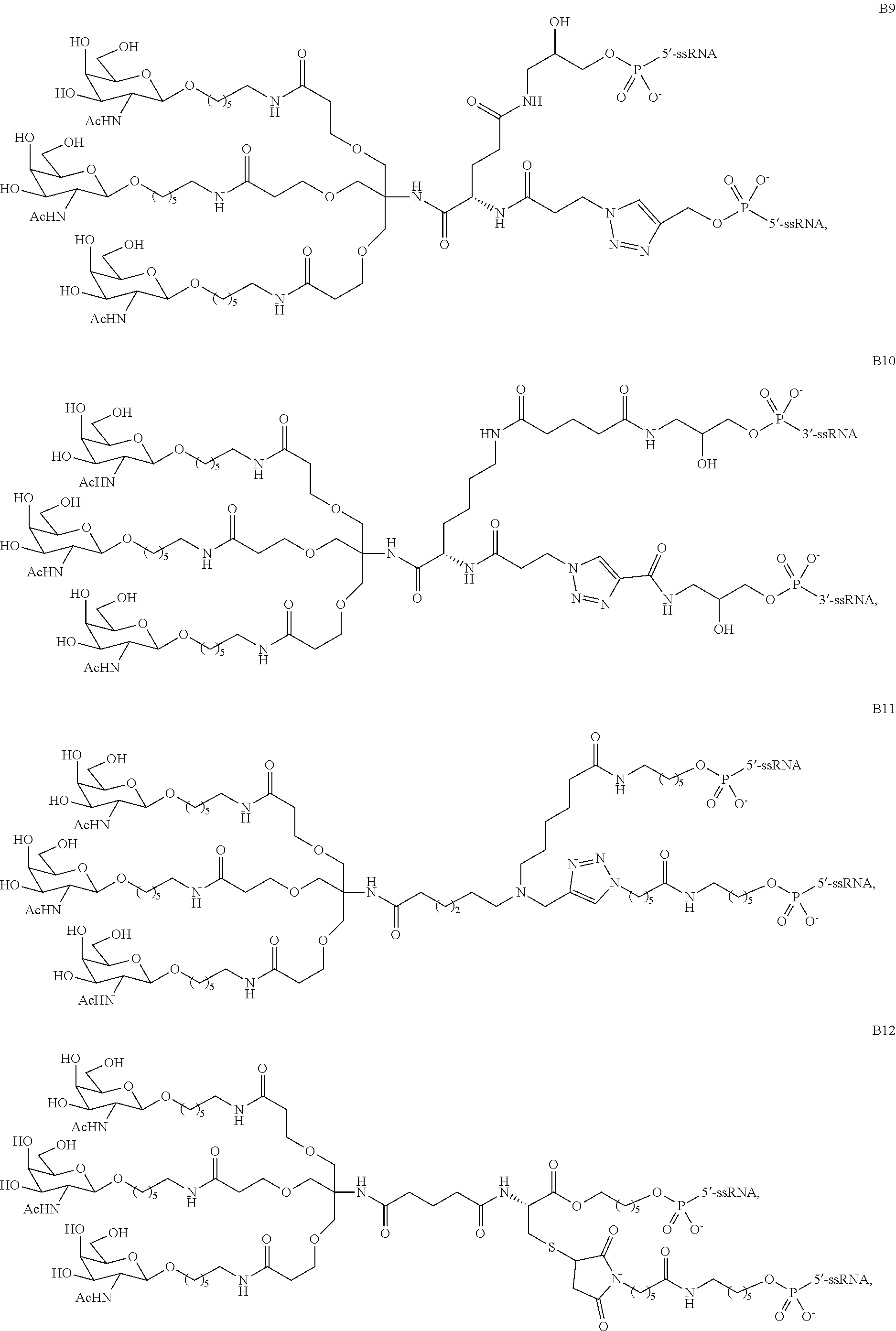

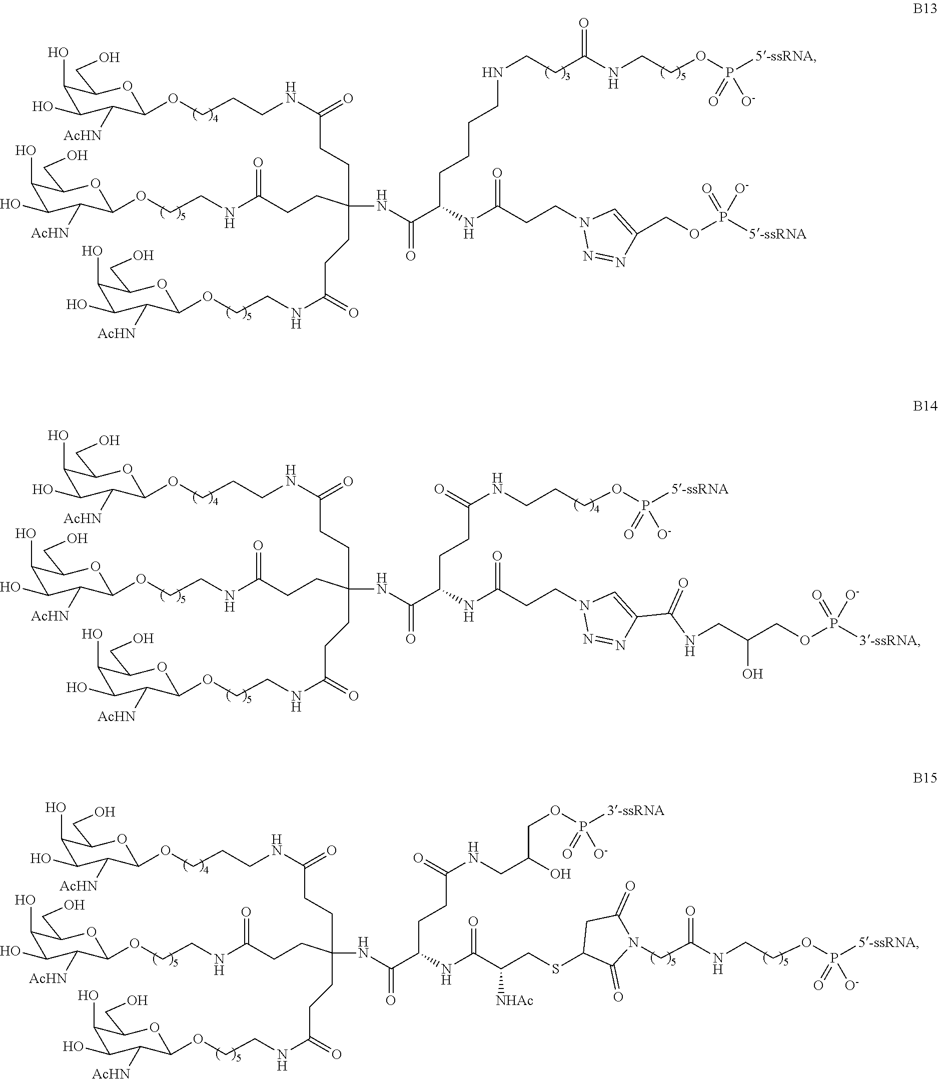

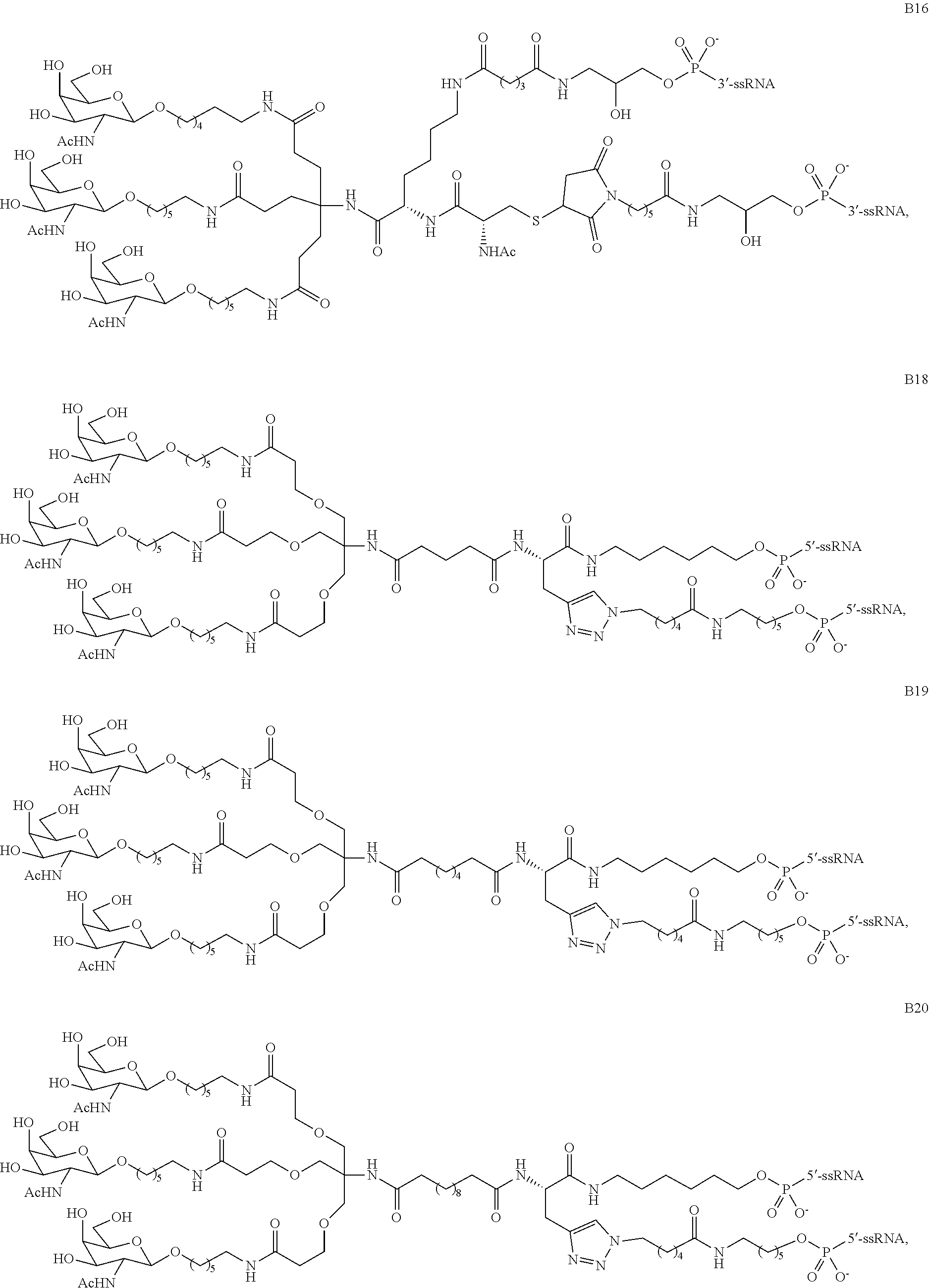

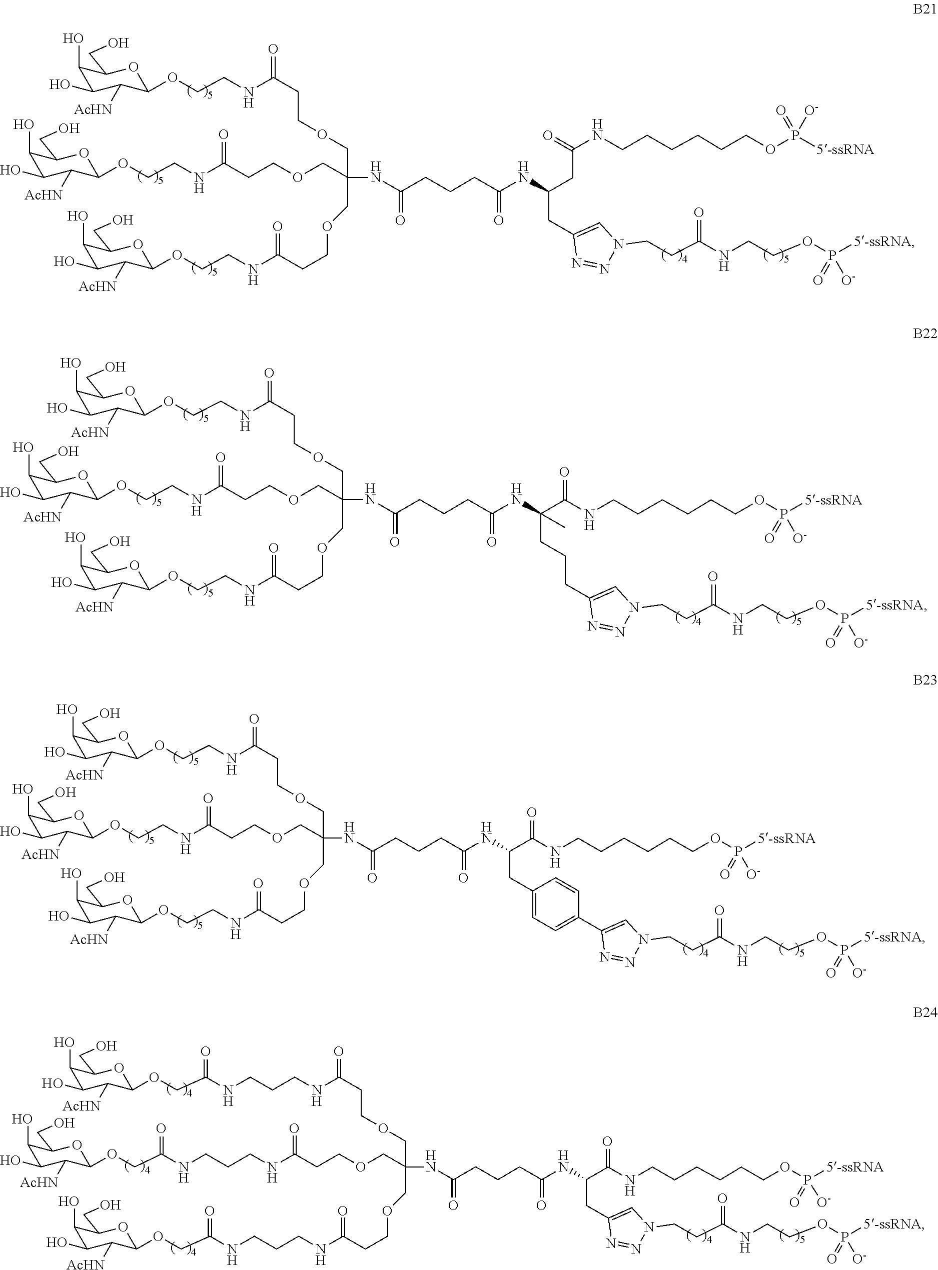

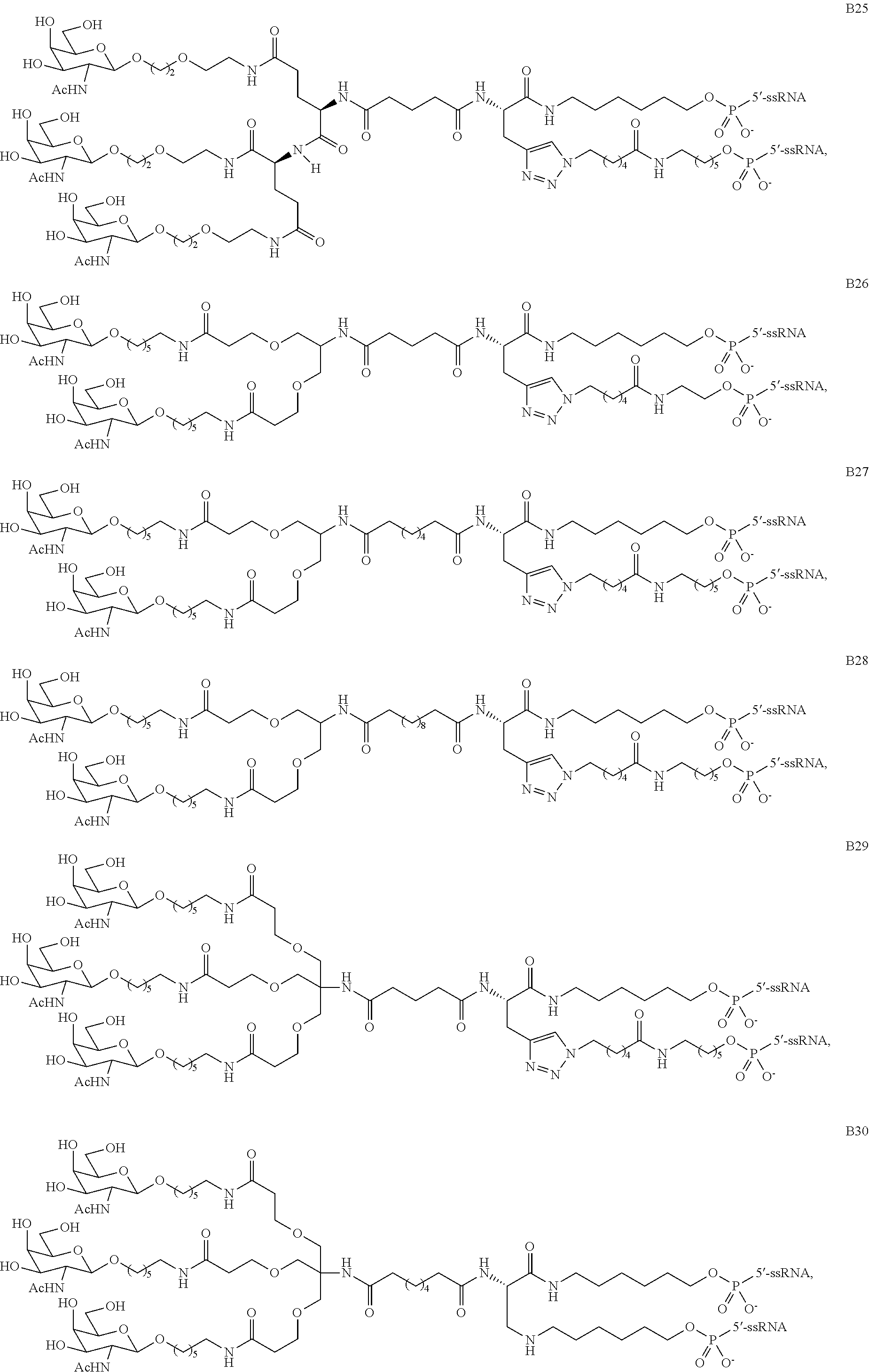

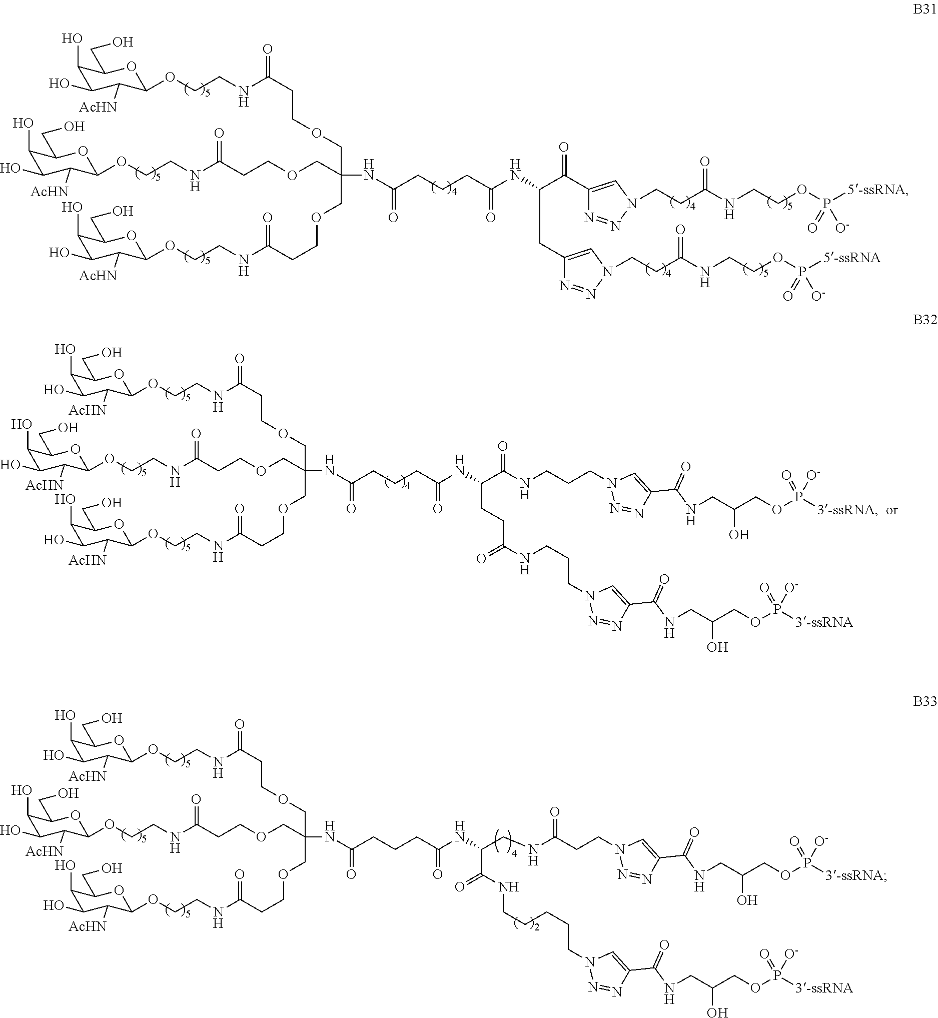

wherein each 3′-ssRNA represents independently a single-stranded RNA with its 3′-terminus, connected to a trivalent linker; each 5′-ssRNA independently represents a single-stranded RNA with its 5′-terminus connected to a trivalent linker; and 5′-ssDNA represents a single stranded DNA with its 5′-terminus connected to a trivalent linker.
Embodiment 91. The carbohydrate-oligonucleotide conjugate of embodiment 1, having the structure of:

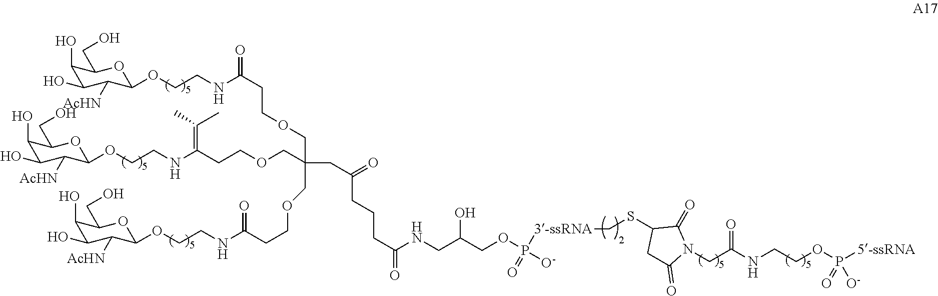

wherein 3′-ssRNA represents a single-stranded RNA with its 3′-terminus connected to a first divalent linker and its 5′-terminus to a second divalent linker; and 5′-ssRNA represents a single-stranded RNA with its 5′-terminus to the second divalent linker.
Embodiment 92. The carbohydrate-oligonucleotide conjugate of any of embodiments 1, 2, 20 to 86, 90, and 91, wherein each oligonucleotide independently comprises a nucleotide sequence of any one of SEQ ID NOs: 1 to 25.
Embodiment 93. A pharmaceutical composition comprising the carbohydrate-oligonucleotide conjugate of any one of embodiments 1 to 92 and a pharmaceutically acceptable excipient.
Embodiment 94. The pharmaceutical composition of embodiment 93, wherein the composition is in single dosage form.
Embodiment 95. The pharmaceutical composition of embodiment 93 or 94, wherein the composition is in a parenteral or intravenous dosage form.
Embodiment 96. The pharmaceutical composition of embodiment 95, wherein the composition is formulated in an intravenous dosage form. Provided herein is a carbohydrate-oligonucleotide conjugate comprising an asialoglycoprotein receptor (ASGPR) binding moiety and two oligonucleotides.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from aequiring a disorder, disease, or condition; or reducing a subject's risk of aequiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The terms “alkylene” and “alkanediyl” are used interchangeably herein in reference to a linear or branched saturated divalent hydrocarbon radical, wherein the alkanediyl is optionally substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)₄C(O)—, or —C(O)(CH₂)₅C(O)—.

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, ONHCH₃, NHOCH₃, SCH₃, CH₂NHCH₂CH₃, and NHCH₂CH₂CH₃. Examples of substituted heteroalkyl groups include, but are not limited to, —CH₂NHC(O)CH₃ and —NHC(O)CH₂CH₃.

The terms “heteroalkylene” and “heteroalkanediyl” are used interchangeably herein in reference to a linear or branched saturated divalent hydrocarbon radical that contains one or more heteroatoms in its main chain, each independently selected from O, S, and N. The heteroalkylene is optionally substituted with one or more substituents Q as described herein. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —(CH₂)₂O—, —(CH₂)₃O—, —(CH₂)₄O—, —(CH₂)₅O—, —(CH₂)₆O—, —(CH₂)₇O—, —(CH₂)₈O—, —(CH₂)₉O—, —(CH₂)₁₀O—, —CH₂OCH₂—, —CH₂CH₂O—, —(CH₂CH₂O)₂—, —(CH₂CH₂O)₃, —(CH₂CH₂O)₄, —(CH₂CH₂O)₅, —CH₂NH—, —CH₂NHCH₂—, —CH₂CH₂NH—, —CH₂S—, —CH₂SCH₂—, and —CH—CH₂S—. Examples of substituted heteroalkylene groups include, but are not limited to, —C(O)CH₂O—, —C(O)(CH₂)₂O—, —C(O)(CH₂)₃O—, —C(O)(CH₂)₄O—, —C(O)(CH₂)₅O—, —C(O)(CH₂)₆O—, —C(O)(CH₂)₇O—, —C(O)(CH₂)₈O—, —C(O)(CH₂)₉O—, —C(O)(CH₂)₁₀O—, —C(O)CH₂OCH₂CH₂O—, —C(O)CH₂O(CH₂CH₂O)₂—, —C(O)CH₂O(CH₂CH₂O)₃, —C(O)CH₂O(CH₂CH₂O)₄, —C(O)CH₂O(CH₂CH₂O)₅—, —CH₂NHC(O)CH₂—, or —CH₂CH₂C(O)NH—.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The terms “alkenylene” and “alkenediyl” are used interchangeably herein in reference to a linear or branched divalent hydrocarbon radical, which contains one or more, in one embodiment, one, two, three, or four, in another embodiment, one, carbon-carbon double bond(s). The alkenediyl is optionally substituted with one or more substituents Q as described herein. The term “alkenediyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenediyl refers to a linear unsaturated divalent hydrocarbon radical of 2 to 6 carbon atoms or a branched unsaturated divalent hydrocarbon radical of 3 to 6 carbon atoms. In certain embodiments, the alkenediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. Examples of alkenediyl groups include, but are not limited to, ethenediyl (including all isomeric forms, e.g., ethene-1,1-diyl and ethene-1,2-diyl), propenediyl (including all isomeric forms, e.g., 1-propene-1,1-diyl, 1-propene-1,2-diyl, and 1-propene-1,3-diyl), butenediyl (including all isomeric forms, e.g., 1-butene-1,1-diyl, 1-butene-1,2-diyl, and 1-butene-1,4-diyl), pentenediyl (including all isomeric forms, e.g., 1-pentene-1,1-diyl, 1-pentene-1,2-diyl, and 1-pentene-1,5-diyl), and hexenediyl (including all isomeric forms, e.g., 1-hexene-1,1-diyl, 1-hexene-1,2-diyl, 1-hexene-1,3-diyl, 1-hexene-1,4-diyl, 1-hexene-1,5-diyl, and 1-hexene-1,6-diyl).

The terms “heteroalkenylene” and “heteroalkenediyl” are used interchangeably herein in reference to a linear or branched divalent hydrocarbon radical, which contains one or more, in one embodiment, one, two, three, or four, in another embodiment, one, carbon-carbon double bond(s), and which contains one or more heteroatoms each independently selected from O, S, and N in the hydrocarbon chain. The heteroalkenylene is optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, or four, in another embodiment, one, carbon-carbon triple bond(s). An alkynyl group does not contain a carbon-carbon double bond. The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The terms “alkynylene” and “alkynediyl” are used interchangeably herein in reference to a linear or branched divalent hydrocarbon radical, which contains one or more, in one embodiment, one, two, three, or four, in another embodiment, one, carbon-carbon triple bond(s). An alkynylene group does not contain a carbon-carbon double bond. The alkynediyl is optionally substituted with one or more substituents Q as described herein. For example, C₂₋₆ alkynediyl refers to a linear unsaturated divalent hydrocarbon radical of 2 to 6 carbon atoms or a branched unsaturated divalent hydrocarbon radical of 4 to 6 carbon atoms. In certain embodiments, the alkynediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of alkynediyl groups include, but are not limited to, ethynediyl, propynediyl (including all isomeric forms, e.g., 1-propyne-1,3-diyl and 1-propyne-3,3-diyl), butynediyl (including all isomeric forms, e.g., 1-butyne-1,3-diyl, 1-butyne-1,4-diyl, and 2-butyne-1,1-diyl), pentynediyl (including all isomeric forms, e.g., 1-pentyne-1,3-diyl, 1-pentyne-1,4-diyl, and 2-pentyne-1,1-diyl), and hexynediyl (including all isomeric forms, e.g., 1-hexyne-1,3-diyl, 1-hexyne-1,4-diyl, and 2-hexyne-1,1-diyl).

The terms “heteroalkynylene” and “heteroalkynediyl” are used interchangeably herein in reference to a linear or branched divalent hydrocarbon radical, which contains one or more, in one embodiment, one, two, three, or four, in another embodiment, one, carbon-carbon triple bond(s), and which contains one or more heteroatoms in its main chain, each independently selected from O, S, and N. A heteroalkynylene group does not contain a carbon-carbon double bond. The heteroalkynylene is optionally substituted with one or more substituents Q as described herein. For example, C₂₋₆ heteroalkynylene refers to a linear unsaturated divalent hydrocarbon radical of 2 to 6 carbon atoms or a branched unsaturated divalent hydrocarbon radical of 4 to 6 carbon atoms. In certain embodiments, the heteroalkynylene is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of heteroalkynylene groups include, but are not limited to, —C≡CCH₂O—, —C≡CCH₂S—, or —C≡CCH₂NH—.

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The terms “cycloalkylene” and “cycloalkanediyl” are used interchangeably herein in reference to a cyclic divalent hydrocarbon radical, which may be optionally substituted with one or more substituents Q as described herein. In one embodiment, cycloalkanediyl groups may be saturated or unsaturated but non-aromatic, and/or bridged, and/or non-bridged, and/or fused bicyclic groups. In certain embodiments, the cycloalkanediyl has from 3 to 30 (C₃₋₃₀), 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkanediyl groups include, but are not limited to, cyclopropanediyl (including all isomeric forms, e.g., cyclopropane-1,1-diyl and cyclopropane-1,2-diyl), cyclobutanediyl (including all isomeric forms, e.g., cyclobutane-1,1-diyl, cyclobutane-1,2-diyl, and cyclobutane-1,3-diyl), cyclopentanediyl (including all isomeric forms, e.g., cyclopentane-1,1-diyl, cyclopentane-1,2-diyl, and cyclopentane-1,3-diyl), cyclohexanediyl (including all isomeric forms, e.g., cyclohexane-1,1-diyl, cyclohexane-1,2-diyl, cyclohexane-1,3-diyl, and cyclohex-1,4-diyl), cycloheptanediyl (including all isomeric forms, e.g., cycloheptane-1,1-diyl, cycloheptane-1,2-diyl, cycloheptane-1,3-diyl, and cycloheptane-1,4-diyl), decalinediyl (including all isomeric forms, e.g., decaline-1,1-diyl, decaline-1,2-diyl, and decaline-1,8-diyl), and adamantdiyl (including all isomeric forms, e.g., adamant-1,2-diyl, adamant-1,3-diyl, and adamant-1,8-diyl).

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The terms “arylene” and “arenediyl” are used interchangeably herein in reference to a divalent monocyclic aromatic hydrocarbon radical or divalent polycyclic aromatic hydrocarbon radical that contains at least one aromatic hydrocarbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene (including all isomeric forms, e.g., phen-1,2-diyl, phen-1,3-diyl, and phen-1,4-diyl), naphthylene (including all isomeric forms, e.g., naphth-1,2-diyl, naphth-1,3-diyl, and naphth-1,8-diyl), fluorenylene (including all isomeric forms, e.g., fluoren-1,2-diyl, fluoren-1,3-diyl, and fluoren-1,8-diyl), azulenylene (including all isomeric forms, e.g., azulen-1,2-diyl, azulen-1,3-diyl, and azulen-1,8-diyl), anthrylene (including all isomeric forms, e.g., anthr-1,2-diyl, anthr-1,3-diyl, and anthr-1,8-diyl), phenanthrylene (including all isomeric forms, e.g., phenanthr-1,2-diyl, phenanthr-1,3-diyl, and phenanthr-1,8-diyl), pyrenylene (including all isomeric forms, e.g., pyren-1,2-diyl, pyren-1,3-diyl, and pyren-1,8-diyl), biphenylene (including all isomeric forms, e.g., biphen-2,3-diyl, biphen-3,4′-diyl, and biphen-4,4′-diyl), and terphenylene (including all isomeric forms, e.g., terphen-2,3-diyl, terphen-3,4′-diyl, and terphen-4,4′-diyl). Arylene also refers to bicyclic or tricyclic carbon rings, where one of the rings is aromatic and the others of which may be saturated, partially unsaturated, or aromatic, for example, dihydronaphthylene (including all isomeric forms, e.g., dihydronaphth-1,2-diyl and dihydronaphth-1,8-diyl), indenylene (including all isomeric forms, e.g., inden-1,2-diyl, inden-1,5-diyl, and inden-1,7-diyl), indanylene (including all isomeric forms, e.g., indan-1,2-diyl, indan-1,5-diyl, and indan-1,7-diyl), or tetrahydronaphthylene (tetralinylene) (including all isomeric forms, e.g., tetrahydronaphth-1,2-diyl, tetrahydronaphth-1,5-diyl, and tetrahydronaphth-1,8-diyl). In certain embodiments, arylene is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “aralkylene” or “arylalkylene” refers to a divalent alkyl group substituted with one or more aryl groups. In certain embodiments, the aralkylene has from 7 to 30 (C₇₋₃₀), from 7 to 20 (C₇₋₂₀), or from 7 to 16 (C₇₋₁₆) carbon atoms. Examples of aralkylene groups include, but are not limited to, benzylene (including all isomeric forms, e.g., phenylmethdiyl), phenylethylene (including all isomeric forms, e.g., 2-phenyl-ethan-1,1-diyl and 2-phenyl-ethan-1,2-diyl), and phenylpropylene (including all isomeric forms, e.g., 3-phenyl-propan-1,1-diyl, 3-phenyl-propan-1,2-diyl, and 3-phenyl-propan-1,3-diyl). In certain embodiments, the aralkylene is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. For a heteroaryl group containing a heteroaromatic ring and a nonaromatic heterocyclic ring, the heteroaryl group is not bonded to the rest of a molecule through its nonaromatic heterocyclic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The terms “heteroarylene” and “heteroarenediyl” are used interchangeably herein in reference to a divalent monocyclic aromatic group or divalent polycyclic aromatic group that contains at least one aromatic ring, wherein at least one aromatic ring contains one or more heteroatoms in the ring, each of which is independently selected from O, S, and N. For a heteroarylene group containing a heteroaromatic ring and a nonaromatic heterocyclic ring, the heteroarylene group is not bonded to the rest of a molecule via its nonaromatic heterocyclic ring. Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furandiyl, imidazoldiyl, isothiazoldiyl, isoxazoldiyl, oxadiazoldiyl, oxazoldiyl, pyrazindiyl, pyrazoldiyl, pyridazindiyl, pyridindiyl, pyrimidindiyl, pyrroldiyl, thiadiazoldiyl, thiazoldiyl, thiendiyl, tetrazoldiyl, triazinediyl, and triazoldiyl. Examples of bicyclic heteroarylene groups include, but are not limited to, benzofurandiyl, benzimidazoldiyl, benzoisoxazoldiyl, benzopyrandiyl, benzothiadiazoldiyl, benzothiazoldiyl, benzothiendiyl, benzotriazoldiyl, benzoxazoldiyl, furopyridindiyl (including all isomeric forms, e.g., furo[2,3-b]pyridindiyl, furo[2,3-c]pyridindiyl, furo[3,2-b]pyridindiyl, furo[3,2-c]-pyridindiyl, furo[3,4-b]pyridindiyl, and furo[3,4-c]pyridindiyl), imidazopyridindiyl (including all isomeric forms, e.g., imidazo[1,2-a]pyridindiyl, imidazo[4,5-b]pyridindiyl, and imidazo[4,5-c]-pyridindiyl), imidazothiazoldiyl (including all isomeric forms, e.g., imidazo[2,1-b]thiazoldiyl and imidazo[4,5-d]thiazoldiyl), indazoldiyl, indolizindiyl, indoldiyl, isobenzofurandiyl, isobenzothiendiyl (i.e., benzo[c]thiendiyl), isoindoldiyl, isoquinolindiyl, naphthyridindiyl (including all isomeric forms, e.g., 1,5-naphthyridindiyl, 1,6-naphthyridindiyl, 1,7-naph-thyridindiyl, and 1,8-naphthyridindiyl), oxazolopyridindiyl (including all isomeric forms, e.g., oxazolo[4,5-b]pyridindiyl, oxazolo[4,5-c]pyridindiyl, oxazolo[5,4-b]pyridindiyl, and oxazolo[5,4-c]pyridindiyl), phthalazindiyl, pteridindiyl, purindiyl, pyrrolopyridindiyl (including all isomeric forms, e.g., pyrrolo[2,3-b]pyridindiyl, pyrrolo[2,3-c]pyridindiyl, pyrrolo[3,2-b]-pyridindiyl, and pyrrolo[3,2-c]pyridindiyl), quinolindiyl, quinoxalindiyl, quinazolindiyl, thiadiazolopyrimidindiyl (including all isomeric forms, e.g., [1,2,5]thiadiazolo[3,4-d]-pyrimidindiyl and [1,2,3]thiadiazolo[4,5-d]pyrimidindiyl), and thienopyridindiyl (including all isomeric forms, e.g., thieno[2,3-b]pyridindiyl, thieno[2,3-c]pyridindiyl, thieno[3,2-b]pyridindiyl, and thieno[3,2-c]pyridindiyl). Examples of tricyclic heteroarylene groups include, but are not limited to, acridindiyl, benzindoldiyl, carbazoldiyl, dibenzofurandiyl, perimidindiyl, phenanthrolindiyl (including all isomeric forms, e.g., 1,5-phenanthrolindiyl, 1,6-phenanthrolindiyl, 1,7-phenanthrolindiyl, 1,9-phenanthrolindiyl, and 2,10-phenanthrolindiyl), phenanthridindiyl, phenarsazindiyl, phenazindiyl, phenothiazindiyl, phenoxazindiyl, and xanthendiyl. In certain embodiments, heteroarylene is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. For a heterocyclyl group containing a heteroaromatic ring and a nonaromatic heterocyclic ring, the heterocyclyl group is not bonded to the rest of a molecule through the heteroaromatic ring. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydro-benzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydro-pyranyl, dihydropyrazolyl, dihydropyrazinyl, dihydropyridinyl, dihydropyrimidinyl, dihydro-pyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. For a heterocyclylene group containing a heteroaromatic ring and a nonaromatic heterocyclic ring, the heterocyclylene group has at least one bond to the rest of a molecule via its nonaromatic heterocyclic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1,4-dihydrobenzo[d][1,3]oxazindiyl, 3,4-dihydrobenzo[c][1,2]oxazindiyl, and 3,4-dihydrobenzo[d][1,2]oxazindiyl), dihydrobenzothiendiyl, dihydroisobenzofurandiyl, dihydrobenzo[c]thiendiyl, dihydrofurdiyl, dihydroisoindoldiyl, dihydropyrandiyl, dihydro-pyrazoldiyl, dihydropyrazindiyl, dihydropyridindiyl, dihydropyrimidindiyl, dihydropyrroldiyl, dioxolandiyl, 1,4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydro-indoldiyl, octahydroisoindoldiyl, oxazolidinondiyl, oxazolidindiyl, oxirandiyl, piperazindiyl, piperidindiyl, 4-pipcridondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydro-thiendiyl, thiamorpholindiyl, thiazolidindiyl, thiochromandiyl, tetrahydroquinolindiyl, and 1,3,5-trithiandiyl. In certain embodiments, the heterocyclylene is optionally substituted with one or more substituents Q as described herein.

The term “halogen,” “halide,” or “halo” refers to fluoro, chloro, bromo, and/or iodo.

The term “optionally substituted” is intended to mean that a group or substituent, such as an alkyl, heteroalkyl, alkylene, heteroalkylene, alkenyl, alkenylene, heteroalkenylene, alkynyl, alkynylenc, heteroalkynylenc, cycloalkyl, cycloalkylenc, aryl, arylene, aralkyl, aralkylene, heteroaryl, heteroarylene, heterocyclyl, or heterocyclylene group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, NR^{a}C(O)SR^{d}, —NR^{a}C(NR^{d})NR^{b}R^{c}, —NR^{a}C(S)R^{d}, —NR^{a}C(S)O)R^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)R^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{c})NR^{f}R^{g}, —C(S)R^{e}, —C(S)OR^{c}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{c}C(S)R^{h}, —NR^{c}C(S)OR^{f}, —NR^{e}C(S)NR^{f}R^{g}, —NR^{c}S(O)R^{h}, —NR^{c}S(O)₂R^{h}, —NR^{c}S(O)NR^{f}R^{g}, —NR^{e}S(O)₂NR^{f}R^{g}, —P(O)R^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

For a divalent group described herein, no orientation is implied by the direction in which the divalent group is presented. For example, unless a particular orientation is specified, the formula —C(O)NH— represents both —C(O)NH— and —NHC(O)—.

### Carbohydrate-Oligonucleotide Conjugates

In one embodiment, provided herein is a carbohydrate-oligonucleotide conjugate comprising an ASGPR binding moiety and two oligonucleotides.

In another embodiment, provided herein is a carbohydrate-oligonucleotide conjugate comprising an ASGPR binding moiety, two oligonucleotides, and a trivalent linker.

In yet another embodiment, provided herein is a carbohydrate-oligonucleotide conjugate comprising an ASGPR binding moiety, two oligonucleotides, and two divalent linkers.

In certain embodiments, the two oligonucleotides are the same. In certain embodiments, the two oligonucleotides are different.

In certain embodiments, the two oligonucleotides are each a single-stranded oligonucleotide. In certain embodiments, the two oligonucleotides are each a double-stranded oligonucleotide.

In yet another embodiment, provided herein is a carbohydrate-oligonucleotide conjugate comprising an ASGPR binding moiety, a first oligonucleotide duplex comprising first sense and antisense sequences, a second oligonucleotide duplex comprising second sense and antisense sequences, and a trivalent linker or first and second divalent linkers; wherein the ASGPR binding moiety is connected to a terminus of the first sense or antisense sequence and to a terminus of the second sense or antisense sequence via the trivalent linker; or wherein the ASGPR binding moiety is connected to a terminus of the first sense or antisense sequence via the first divalent linker, and one of remaining termini of the first sense or antisense sequence is connected to a terminus of the second sense or antisense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a terminus of the first sense or antisense sequence and to a terminus of the second sense or antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a terminus of the first sense sequence and to a terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a terminus of the first antisense sequence and to a terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a terminus of the first sense sequence and to a terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a terminus of the first antisense sequence and to a terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a 5′-terminus of the first sense or antisense sequence and to a 5′-terminus of the second sense or antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 5′-terminus of the first sense sequence and to the 5′-terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 5′-terminus of the first antisense sequence and to the 5′-terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 5′-terminus of the first sense sequence and to the 5′-terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 5′-terminus of the first antisense sequence and to the 5′-terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a 3′-terminus of the first sense or antisense sequence and to a 5′-terminus of the second sense or antisense sequence via the trivalent linker.

In yet embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first sense sequence and to the 5′-terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first antisense sequence and to the 5′-terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first sense sequence and to the 5′-terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first antisense sequence and to the 5′-terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to a 3′-terminus of the first sense or antisense sequence and to a 3′-terminus of the second sense or antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first sense sequence and to the 3′-terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first antisense sequence and to the 3′-terminus of the second sense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first sense sequence and to the 3′-terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and a trivalent linker; wherein the ASGPR binding moiety is connected to the 3′-terminus of the first antisense sequence and to the 3′-terminus of the second antisense sequence via the trivalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to a terminus of the first sense or antisense sequence via the first divalent linker, and one of the remaining termini of the first sense or antisense sequence is connected to a terminus of the second sense or antisense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to a terminus of the first sense sequence via the first divalent linker, and one of the remaining termini of the first sense sequence is connected to a terminus of the second sense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to a terminus of the first antisense sequence via the first divalent linker, and one of the remaining termini of the first antisense sequence is connected to a terminus of the second sense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to a terminus of the first sense sequence via the first divalent linker, and one of the remaining termini of the first sense sequence is connected to a terminus of the second antisense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to a terminus of the first antisense sequence via the first divalent linker, and one of the remaining termini of the first antisense sequence is connected to a terminus of the second antisense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to the 5′-terminus of the first sense or antisense sequence via the first divalent linker, and the 3′-terminus of the first sense or antisense sequence is connected to the 5′-terminus of the second sense or antisense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to the 5′-terminus of the first sense sequence via the first divalent linker, and the 3′-terminus of the first sense sequence is connected to the 5′-terminus of the second sense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to the 5′-terminus of the first antisense sequence via the first divalent linker, and the 3′-terminus of the first antisense sequence is connected to the 5′-terminus of the second sense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to the 5′-terminus of the first sense sequence via the first divalent linker, and the 3′-terminus of the first sense sequence is connected to the 5′-terminus of the second antisense sequence via the second divalent linker.

In yet another embodiment, the carbohydrate-oligonucleotide conjugate provided herein comprises an ASGPR binding moiety, a first oligonucleotide duplex comprising first sense and antisense sequences, a second oligonucleotide duplex comprising second sense and antisense sequences, and first and second divalent linkers; wherein the ASGPR binding moiety is connected to the 5′-terminus of the first antisense sequence via the first divalent linker, and the 3′-terminus of the first antisense sequence is connected to the 5′-terminus of the second antisense sequence via the second divalent linker.

In certain embodiments, each oligonucleotide duplex in a carbohydrate-oligonucleotide conjugate provided herein is independently a double-stranded oligodeoxy-ribonucleotide or oligoribonucleotide. In certain embodiments, each oligonucleotide duplex in a carbohydrate-oligonucleotide conjugate provided herein is independently a double-stranded oligodeoxyribonucleotide. In certain embodiments, each oligonucleotide duplex in a carbohydrate-oligonucleotide conjugate provided herein is independently a double-stranded oligoribonucleotide. In certain embodiments, each oligonucleotide duplex in a carbohydrate-oligonucleotide conjugate provided herein is independently a siRNA.

In certain embodiments, each strand of the first oligonucleotide duplex in a carbohydrate-oligonucleotide conjugate provided herein independently comprises from about 10 to about 50, from about 10 to about 30, or from about 15 to about 25 nucleotides. In certain embodiments, each strand of the first oligonucleotide duplex in a carbohydrate-oligonucleotide conjugate provided herein independently comprises from about 10 to about 50 nucleotides. In certain embodiments, each strand of the first oligonucleotide duplex in a carbohydrate-oligonucleotide conjugate provided herein independently comprises from about 10 to about 30 nucleotides. In certain embodiments, each strand of the first oligonucleotide duplex in a carbohydrate-oligonucleotide conjugate provided herein independently comprises from about 15 to about 25 nucleotides. In certain embodiments, each strand of the first oligonucleotide duplex in a carbohydrate-oligonucleotide conjugate provided herein independently comprises about 15, about 16, about 17, about 18, about 19, about 20, about 21, about 22, about 23, about 24, or about 25 nucleotides.

In certain embodiments, each strand of the second oligonucleotide duplex in a carbohydrate-oligonucleotide conjugate provided herein independently comprises from about 10 to about 50, from about 10 to about 30, or from about 15 to about 25 nucleotides. In certain embodiments, each strand of the second oligonucleotide duplex in a carbohydrate-oligonucleotide conjugate provided herein independently comprises from about 10 to about 50 nucleotides. In certain embodiments, each strand of the second oligonucleotide duplex in a carbohydrate-oligonucleotide conjugate provided herein independently comprises from about 10 to about 30 nucleotides. In certain embodiments, each strand of the second oligonucleotide duplex in a carbohydrate-oligonucleotide conjugate provided herein independently comprises from about 15 to about 25 nucleotides. In certain embodiments, each strand of the second oligonucleotide duplex in a carbohydrate-oligonucleotide conjugate provided herein independently comprises about 15, about 16, about 17, about 18, about 19, about 20, about 21, about 22, about 23, about 24, or about 25 nucleotides.

In certain embodiments, the first oligonucleotide duplex is a double-stranded siRNA. In certain embodiments, each strand of the first siRNA duplex in a carbohydrate-oligonucleotide conjugate provided herein independently comprises from about 10 to about 50, from about 10 to about 30, or from about 15 to about 25 nucleotides. In certain embodiments, each strand of the first siRNA duplex in a carbohydrate-oligonucleotide conjugate provided herein independently comprises from about 10 to about 50 nucleotides. In certain embodiments, each strand of the first siRNA duplex in a carbohydrate-oligonucleotide conjugate provided herein independently comprises from about 10 to about 30 nucleotides. In certain embodiments, each strand of the first siRNA duplex in a carbohydrate-oligonucleotide conjugate provided herein independently comprises from about 15 to about 25 nucleotides. In certain embodiments, each strand of the first siRNA duplex in a carbohydrate-oligonucleotide conjugate provided herein independently comprises about 15, about 16, about 17, about 18, about 19, about 20, about 21, about 22, about 23, about 24, or about 25 nucleotides.

In certain embodiments, the second oligonucleotide duplex is a double-stranded siRNA. In certain embodiments, each strand of the second siRNA duplex in a carbohydrate-oligonucleotide conjugate provided herein independently comprises from about 10 to about 50, from about 10 to about 30, or from about 15 to about 25 nucleotides. In certain embodiments, each strand of the second siRNA duplex in a carbohydrate-oligonucleotide conjugate provided herein independently comprises from about 10 to about 50 nucleotides. In certain embodiments, each strand of the second siRNA duplex in a carbohydrate-oligonucleotide conjugate provided herein independently comprises from about 10 to about 30 nucleotides. In certain embodiments, each strand of the second siRNA duplex in a carbohydrate-oligonucleotide conjugate provided herein independently comprises from about 15 to about 25 nucleotides. In certain embodiments, each strand of the second siRNA duplex in a carbohydrate-oligonucleotide conjugate provided herein independently comprises about 15, about 16, about 17, about 18, about 19, about 20, about 21, about 22, about 23, about 24, or about 25 nucleotides.

In certain embodiments, each nucleotide in each strand of the first siRNA duplex is independently a natural nucleotide or modified nucleotide. In certain embodiments, a natural nucleotide includes adenylate (a), cytidylate (c), guanylate (g), and uridylate (u). In certain embodiments, a modified nucleotide is a nucleotide with a modification on its nucleobase, a modification on its sugar, and/or a modification on its phosphate linkage group. Examples of modified nucleotides include, but are not limited to, 2′-fluoroadenosine (fA), 2′-fluorocytidine (fC), 2′-fluorogunaosine (fG), 2′-fluorouridine (fU), 2′-deoxyadenosine (dA), 2′-deoxy-gunaosine (dG), 2′-deoxycytidine (dC), 2′-deoxythymidine (dT), 2′-O-methyladenosine (A), 2′-O-methylcytidine (C), 2′-O-methylguanosine (G), or 2′-O-methyluridine (U). In certain embodiments, each nucleotide in each strand of the first siRNA duplex is independently adenylate, cytidylate, guanylate, uridylate, 2′-fluoroadenosine, 2′-fluorocytidine, 2′-fluorogunaosine, 2′-fluorouridine, 2′-deoxyadenosine, 2′-deoxycytidine, 2′-deoxygunaosine, 2′-deoxythymidine, 2′-O-methyladenosine, 2′-O-methylcytidine, 2′-O-methylguanosine, or 2′-O-methyluridine. In certain embodiments, each strand of the first siRNA duplex has one or more phosphate linkage groups replaced with phosphorothioate or phosphorodithiate.

In certain embodiments, each nucleotide in each strand of the second siRNA duplex is independently a natural nucleotide or modified nucleotide. In certain embodiments, each nucleotide in each strand of the second siRNA duplex is independently adenylate, cytidylate, guanylate, uridylate, 2′-fluoroadenosine, 2′-fluorocytidine, 2′-fluorogunaosine, 2′-fluorouridine, 2′-deoxyadenosine, 2′-deoxycytidine, 2′-deoxygunaosine, 2′-deoxythymidine, 2′-O-methyl-adenosine, 2′-O-methylcytidine, 2′-O-methylguanosine, or 2′-O-methyluridine. In certain embodiments, each strand of the second siRNA duplex has one or more phosphate linkage groups replaced with phosphorothioate or phosphorodithiate.

In yet another embodiment, provided herein is a carbohydrate-oligonucleotide conjugate comprising an ASGPR binding moiety, first and second single-stranded oligonucleotides, and a trivalent linker or first and second divalent linkers; wherein the ASGPR binding moiety is connected to a terminus of the first oligonucleotide and to a terminus of the second oligonucleotide via the trivalent linker; or wherein the ASGPR binding moiety is connected to a terminus of the first oligonucleotide via the first divalent linker, and the other terminus of the first oligonucleotide is connected to a terminus of the second oligonucleotide via the second divalent linker.

In yet another embodiment, the carbohydrate-oligonucleotide conjugate provided herein comprises an ASGPR binding moiety, first and second single-stranded oligonucleotides, and a trivalent linker; wherein the ASGPR binding moiety is connected to the 5′-terminus of the first oligonucleotide and to the 5′-terminus of the second oligonucleotide via the trivalent linker.

In yet another embodiment, the carbohydrate-oligonucleotide conjugate provided herein comprises an ASGPR binding moiety, first and second single-stranded oligonucleotides, and a trivalent linker; wherein the ASGPR binding moiety is connected to the 3′-terminus of the first oligonucleotide and to the 5′-terminus of the second oligonucleotide via the trivalent linker.

In yet another embodiment, the carbohydrate-oligonucleotide conjugate provided herein comprises an ASGPR binding moiety, first and second oligonucleotides, and a trivalent linker; wherein the ASGPR binding moiety is connected to the 3′-terminus of the first oligonucleotide and to the 3′-terminus of the second oligonucleotide via the trivalent linker.

In yet another embodiment, the carbohydrate-oligonucleotide conjugate provided herein comprises an ASGPR binding moiety, first and second single-stranded oligonucleotides, and first and second divalent linkers; wherein the ASGPR binding moiety is connected to a terminus of the first oligonucleotide via the first divalent linker, and the other terminus of the first oligonucleotide is connected to a terminus of the second oligonucleotide via the second divalent linker.

In yet another embodiment, the carbohydrate-oligonucleotide conjugate provided herein comprises an ASGPR binding moiety, first and second single-stranded oligonucleotides, and first and second divalent linkers; wherein the ASGPR binding moiety is connected to the 5′-terminus of the first oligonucleotide via the first divalent linker, and the 3′-terminus of the first oligonucleotide is connected to the 5′-terminus of the second oligonucleotide via the second divalent linker.

In yet another embodiment, the carbohydrate-oligonucleotide conjugate provided herein comprises an ASGPR binding moiety, first and second single-stranded oligonucleotides, and first and second divalent linkers; wherein the ASGPR binding moiety is connected to the 3′-terminus of the first oligonucleotide via the first divalent linker, and the 5′-terminus of the first oligonucleotide is connected to the 3′-terminus of the second oligonucleotide via the second divalent linker.

In certain embodiments, each single-stranded oligonucleotide in a carbohydrate-oligonucleotide conjugate provided herein is independently a single-stranded oligodeoxyribo-nucleotide or oligoribonucleotide. In certain embodiments, each single-stranded oligonucleotide in a carbohydrate-oligonucleotide conjugate provided herein is independently a single-stranded oligodeoxyribonucleotide. In certain embodiments, each single-stranded oligonucleotide in a carbohydrate-oligonucleotide conjugate provided herein is independently a single-stranded oligoribonucleotide. In certain embodiments, each single-stranded oligonucleotide in a carbohydrate-oligonucleotide conjugate provided herein is independently an ASO, miRNA, mRNA, or tRNA.

In certain embodiments, the first single-stranded oligonucleotide in a carbohydrate-oligonucleotide conjugate provided herein is a ribonucleotide. In certain embodiments, the first single-stranded oligonucleotide comprises from about 10 to about 50, from about 10 to about 30, or from about 15 to about 25 nucleotides. In certain embodiments, the first single-stranded oligonucleotide comprises from about 10 to about 50 nucleotides. In certain embodiments, the first single-stranded oligonucleotide comprises from about 10 to about 30 nucleotides. In certain embodiments, the first single-stranded oligonucleotide comprises from about 15 to about 25 nucleotides. In certain embodiments, the first single-stranded oligonucleotide comprises about 15, about 16, about 17, about 18, about 19, about 20, about 21, about 22, about 23, about 24, or about 25 nucleotides.

In certain embodiments, the second single-stranded oligonucleotide in a carbohydrate-oligonucleotide conjugate provided herein is a ribonucleotide. In certain embodiments, the second single-stranded oligonucleotide comprises from about 10 to about 50, from about 10 to about 30, or from about 15 to about 25 nucleotides. In certain embodiments, the second single-stranded oligonucleotide comprises from about 10 to about 50 nucleotides. In certain embodiments, the second single-stranded oligonucleotide comprises from about 10 to about 30 nucleotides. In certain embodiments, the second single-stranded oligonucleotide comprises from about 15 to about 25 nucleotides. In certain embodiments, the second single-stranded oligonucleotide comprises about 15, about 16, about 17, about 18, about 19, about 20, about 21, about 22, about 23, about 24, or about 25 nucleotides.

In certain embodiments, each nucleotide in the first single-stranded oligonucleotide is independently a natural nucleotide or modified nucleotide. In certain embodiments, each nucleotide in the first single-stranded oligonucleotide is independently adenylate, cytidylate, guanylate, uridylate, 2′-fluoroadenosine, 2′-fluorocytidine, 2′-fluorogunaosine, 2′-fluorouridine, 2′-deoxyadenosine, 2′-deoxycytidine, 2′-deoxygunaosine, 2′-deoxythymidine, 2′-O-methyl-adenosine, 2′-O-methylcytidine, 2′-O-methylguanosine, or 2′-O-methyluridine. In certain embodiments, the first single-stranded oligonucleotide has one or more phosphate linkage groups replaced with phosphorothioate or phosphorodithiate.

In certain embodiments, each nucleotide in the second single-stranded oligonucleotide is independently a natural nucleotide or modified nucleotide. In certain embodiments, each nucleotide in the second single-stranded oligonucleotide is independently adenylate, cytidylate, guanylate, uridylate, 2′-fluoroadenosine, 2′-fluorocytidine, 2′-fluorogunaosine, 2′-fluorouridine, 2′-deoxyadenosine, 2′-deoxycytidine, 2′-deoxygunaosine, 2′-deoxythymidine, 2′-O-methyl-adenosine, 2′-O-methylcytidine, 2′-O-methylguanosine, or 2′-O-methyluridine. In certain embodiments, the second single-stranded oligonucleotide has one or more phosphate linkage groups replaced with phosphorothioate or phosphorodithiate.

In certain embodiments, in a carbohydrate-oligonucleotide conjugate provided herein, the ASGPR binding moiety comprises one or more moieties, each moiety independently having the structure of:

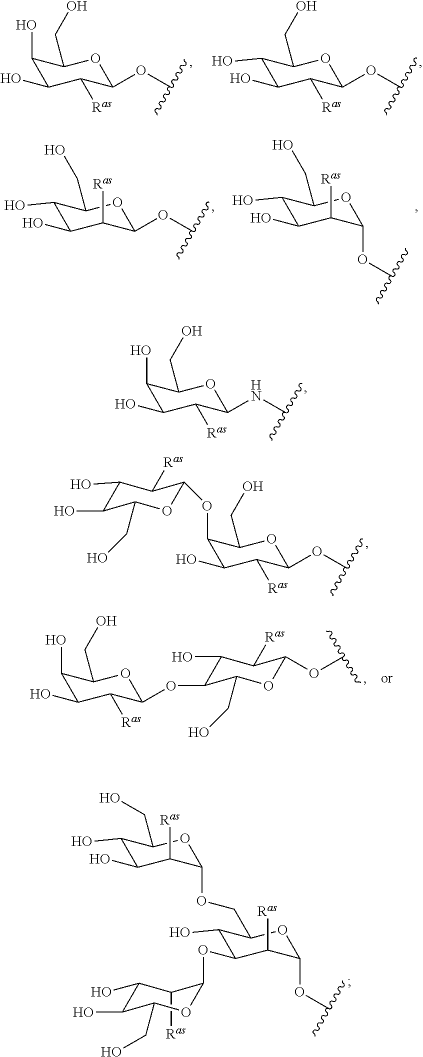

wherein each R^{as} is independently —OH, —NHC(O)H, or —NHCO(CH₂)ₐCH₃; and a is an integer of 0, 1, 2, 3, or 4.

In certain embodiments, in a carbohydrate-oligonucleotide conjugate provided herein, the ASGPR binding moiety comprises one or more N-acetylgalactosamines (GalNAc). In certain embodiments, in a carbohydrate-oligonucleotide conjugate provided herein, the ASGPR binding moiety comprises from about 1 to about 10 or from about 1 to about 6 GalNAc. In certain embodiments, in a carbohydrate-oligonucleotide conjugate provided herein, the ASGPR binding moiety comprises from about 1 to about 10 GalNAc. In certain embodiments, in a carbohydrate-oligonucleotide conjugate provided herein, the ASGPR binding moiety comprises from about 1 to about 6 GalNAc. In certain embodiments, in a carbohydrate-oligonucleotide conjugate provided herein, the ASGPR binding moiety comprises about 1, about 2, about 3, about 4, or about 5 GalNAc. In certain embodiments, in a carbohydrate-oligonucleotide conjugate provided herein, the ASGPR binding moiety comprises about 1 GalNAc. In certain embodiments, in a carbohydrate-oligonucleotide conjugate provided herein, the ASGPR binding moiety comprises about 2 GalNAc. In certain embodiments, in a carbohydrate-oligonucleotide conjugate provided herein, the ASGPR binding moiety comprises about 3 GalNAc. In certain embodiments, in a carbohydrate-oligonucleotide conjugate provided herein, the ASGPR binding moiety comprises about 4 GalNAc. In certain embodiments, in a carbohydrate-oligonucleotide conjugate provided herein, the ASGPR binding moiety comprises about 5 GalNAc.

In certain embodiments, in a carbohydrate-oligonucleotide conjugate provided herein, the ASGPR binding moiety has the structure of Formula (A-I):

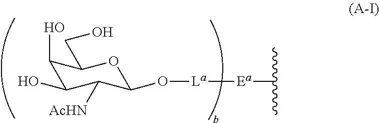

or an enantiomer, a mixture of enantiomers, a diastereomer, a mixture of two or more diastereomers, a tautomer, or a mixture of two or more tautomers thereof; wherein:
   E^{a} and b are:
   (i) b is an integer of 1; and E^{a} is a bond;
   (ii) b is an integer of 2; and E^{a} is (i) CH or N; or (ii) trivalent C₁₋₆ alkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q
   (iii) b is an integer of 3; and E^{a} is (i) C; or (ii) tetravalent C₁₋₆ alkenyl, tetravalent C₃₋₁₀ cycloalkyl, tetravalent C₆₋₁₄ aryl, tetravalent heteroaryl, or tetravalent heterocyclyl, each optionally substituted with one or more substituents Q; and
   each L^{a} is independently a linker.

In one embodiment, in a carbohydrate-oligonucleotide conjugate provided herein, the ASGPR binding moiety has the structure of Formula (A-II):

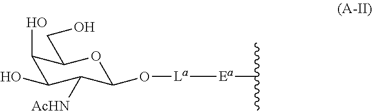

or an enantiomer, a mixture of enantiomers, a diastereomer, a mixture of two or more diastereomers, a tautomer, or a mixture of two or more tautomers thereof; wherein E^{a} is a bond; and L^{a} is a linker.

In another embodiment, in a carbohydrate-oligonucleotide conjugate provided herein, the ASGPR binding moiety has the structure of Formula (A-III):

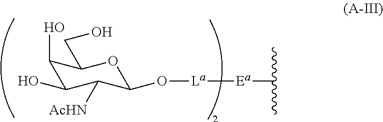

or an enantiomer, a mixture of enantiomers, a diastereomer, a mixture of two or more diastereomers, a tautomer, or a mixture of two or more tautomers thereof; wherein E^{a} is (i) CH or N; or (ii) trivalent C₁₋₆ alkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q; and each L^{a} is independently a linker.

In certain embodiments, in Formula (A-III), E^{a} is CH or N. In certain embodiments, in Formula (A-III), E^{a} is CH. In certain embodiments, in Formula (A-III), E^{a} is N.

In certain embodiments, in Formula (A-III), E^{a} is trivalent C₁₋₆ alkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, in Formula (A-III), E^{a} is trivalent C₁₋₆ alkyl, optionally substituted with one or more substituents Q. In certain embodiments, in Formula (A-III), E^{a} is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, in Formula (A-III), E^{a} is CR^{m}, wherein R^{m} is C₁₋₆ alkyl, optionally substituted with one or more Q. In certain embodiments, in Formula (A-III), E^{a} is C(CH₃). In certain embodiments, in Formula (A-III), E^{a} is trivalent C₁₋₆ alkenyl, optionally substituted with one or more substituents Q. In certain embodiments, in Formula (A-III), E^{a} is trivalent C₃₋₁₀ cycloalkyl, optionally substituted with one or more substituents Q. In certain embodiments, in Formula (A-III), E^{a} is trivalent C₆₋₁₄ aryl, optionally substituted with one or more substituents Q. In certain embodiments, in Formula (A-III), E^{a} is trivalent heteroaryl, optionally substituted with one or more substituents Q. In certain embodiments, in Formula (A-III), E^{a} is trivalent heterocyclyl, optionally substituted with one or more substituents Q.

In yet certain embodiment, in a carbohydrate-oligonucleotide conjugate provided herein, the ASGPR binding moiety has the structure of Formula (A-IV):

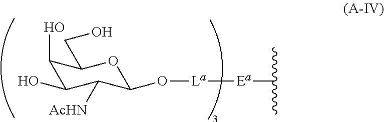

or an enantiomer, a mixture of enantiomers, a diastereomer, a mixture of two or more diastereomers, a tautomer, or a mixture of two or more tautomers thereof; wherein E^{a} is (i) C; or (ii) tetravalent C₁₋₆ alkenyl, tetravalent C₃₋₁₀ cycloalkyl, tetravalent C₆₋₁₄ aryl, tetravalent heteroaryl, or tetravalent heterocyclyl, each optionally substituted with one or more substituents Q; and each L^{a} is independently a linker.

In certain embodiments, in Formula (A-IV), E^{a} is C. In certain embodiments, in Formula (A-IV), E^{a} is tetravalent C₁₋₆ alkenyl, optionally substituted with one or more substituents Q. In certain embodiments, in Formula (A-IV), E^{a} is tetravalent C₃₋₁₀ cycloalkyl, optionally substituted with one or more substituents Q. In certain embodiments, in Formula (A-IV), E^{a} is tetravalent C₆₋₁₄ aryl, optionally substituted with one or more substituents Q. In certain embodiments, in Formula (A-IV), E^{a} is tetravalent heteroaryl, optionally substituted with one or more substituents Q. In certain embodiments, in Formula (A-IV), E^{a} is tetravalent heterocyclyl, optionally substituted with one or more substituents Q.

In certain embodiments, in a carbohydrate-oligonucleotide conjugate provided herein, the ASGPR binding moiety is:

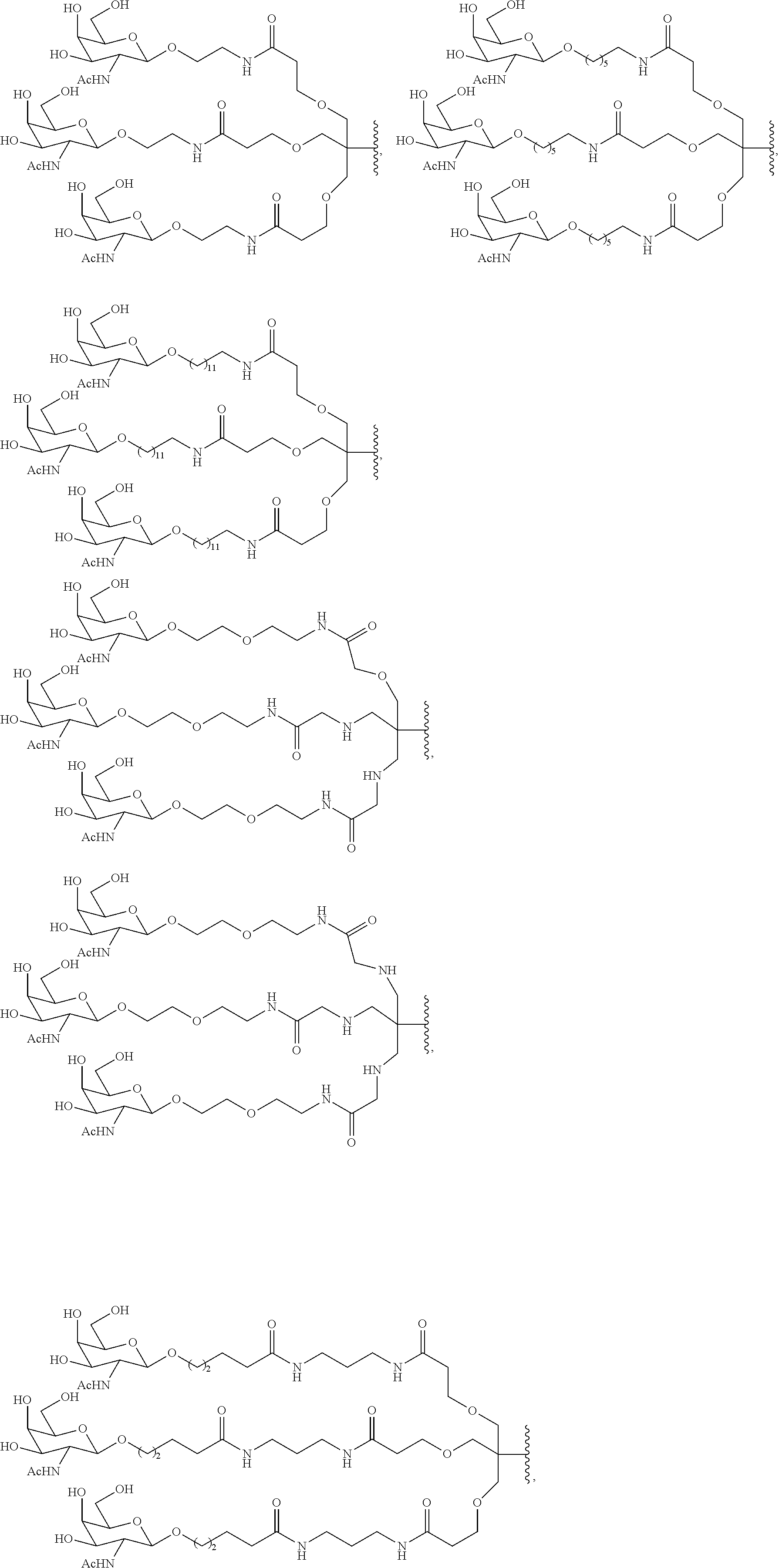

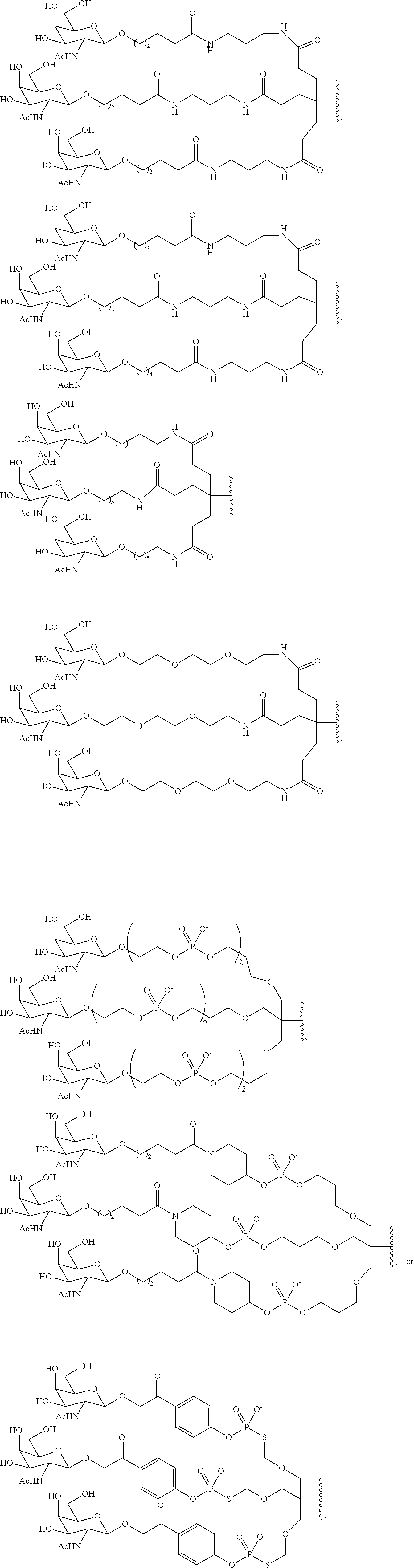

In certain embodiments, in a carbohydrate-oligonucleotide conjugate provided herein, the ASGPR binding moiety is:

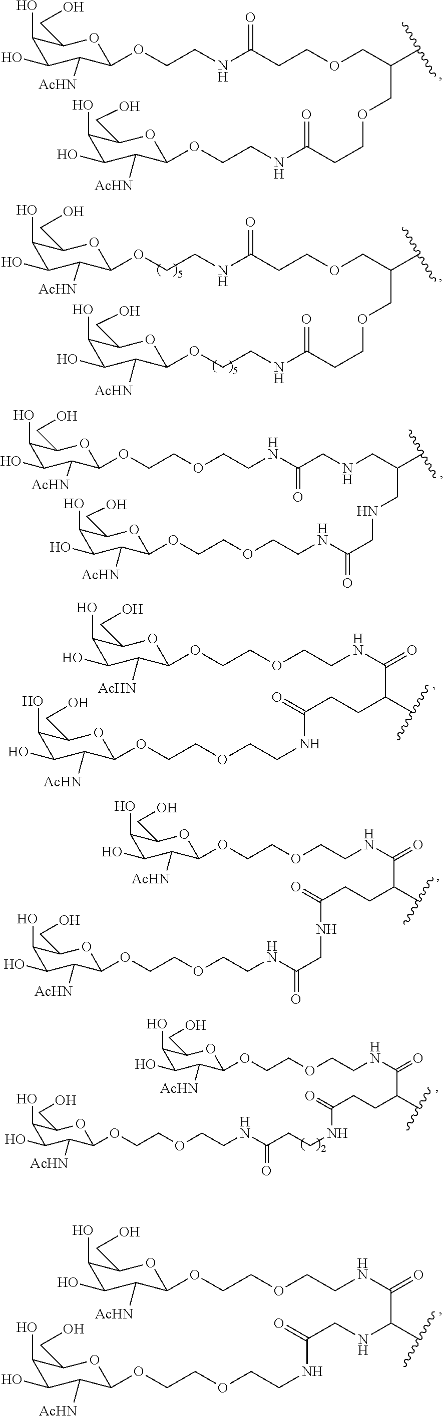

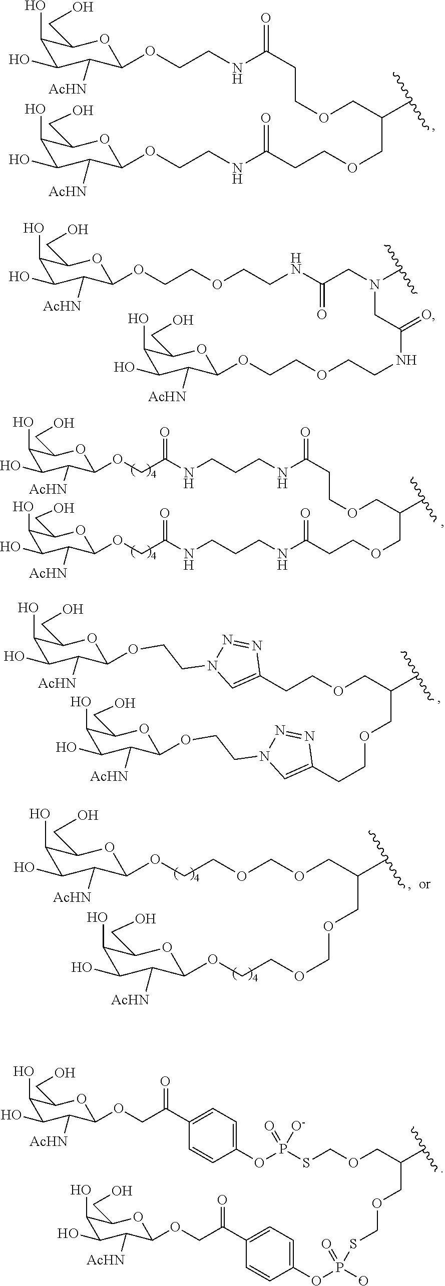

In certain embodiments, in a carbohydrate-oligonucleotide conjugate provided herein, the ASGPR binding moiety is

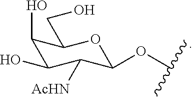 [SMILES: *OC1OC(CO)C(O)C(O)C1NC(C)=O]

In certain embodiments, in a carbohydrate-oligonucleotide conjugate provided herein, the ASGPR binding moiety has the structure of Formula (A-V):

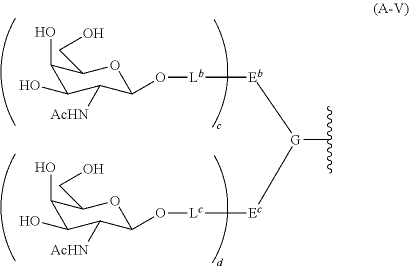

or an enantiomer, a mixture of enantiomers, a diastereomer, a mixture of two or more diastereomers, a tautomer, or a mixture of two or more tautomers thereof; wherein:
   E^{b} and c are:
   (i) c is an integer of 1; and E^{b} is a bond;
   (ii) c is an integer of 2; and E^{b} is (i) CH or N; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q; or
   (iii) c is an integer of 3; and E^{b} is (i) C; or (ii) tetravalent C₁₋₆ heteroalkyl, tetravalent C₁₋₆ alkenyl, tetravalent C₃₋₁₀ cycloalkyl, tetravalent C₆₋₁₄ aryl, tetravalent heteroaryl, or tetravalent heterocyclyl, each optionally substituted with one or more substituents Q;
   E^{c} and d are:
   (i) d is an integer of 1; and E^{c} is bond;
   (ii) d is an integer of 2; and E^{c} is (i) CH or N; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q; or
   (iii) d is an integer of 3; and E^{c} is (i) C; or (ii) tetravalent C₁₋₆ heteroalkyl, tetravalent C₁₋₆ alkenyl, tetravalent C₃₋₁₀ cycloalkyl, tetravalent C₆₋₁₄ aryl, tetravalent heteroaryl, or tetravalent heterocyclyl, each optionally substituted with one or more substituents Q;
G is (i) CH or N; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q; and
   each L^{b} and L^{c} is independently a linker.

In one embodiment, in a carbohydrate-oligonucleotide conjugate provided herein, the ASGPR binding moiety has the structure of Formula (A-VI):

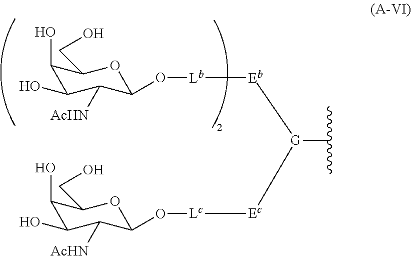

or an enantiomer, a mixture of enantiomers, a diastereomer, a mixture of two or more diastereomers, a tautomer, or a mixture of two or more tautomers thereof; wherein:
   E^{b} is (i) CH or N; or (ii) trivalent C₁₋₆ alkyl, trivalent C₁₋₆ heteroalkyl, trivalent C₁₋₆ alkenyl, trivalent C₃₋₁₀ Cycloalkyl, trivalent C₆₋₁₄ aryl, trivalent heteroaryl, or trivalent heterocyclyl, each optionally substituted with one or more substituents Q;
   E^{c} is a bond;
   G is (i) CH or N; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q; and each L^{b} and L^{c} is independently a linker.

In certain embodiments, in Formula (A-VI), E^{b} is CH or N. In certain embodiments, in Formula (A-VI), E^{b} is CH. In certain embodiments, in Formula (A-VI), E^{b} is N.

In certain embodiments, in Formula (A-VI), E^{b} is trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, in Formula (A-VI), E^{b} is trivalent C₁₋₆ alkyl, optionally substituted with one or more substituents Q. In certain embodiments, in Formula (A-VI), E^{b} is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, in Formula (A-VI), E^{b} is CR^{m}, wherein R^{m} is C₁₋₆ alkyl, optionally substituted with one or more Q. In certain embodiments, in Formula (A-VI), E^{b} is C(CH₃). In certain embodiments, in Formula (A-VI), E^{b} is trivalent C₁₋₆ heteroalkyl, optionally substituted with one or more substituents Q. In certain embodiments, in Formula (A-VI), E^{b} is trivalent C₁₋₆ alkenyl, optionally substituted with one or more substituents Q. In certain embodiments, in Formula (A-VI), E^{b} is trivalent C₃₋₁₀ cycloalkyl, optionally substituted with one or more substituents Q. In certain embodiments, in Formula (A-VI), E^{b} is trivalent C₆₋₁₄ aryl, optionally substituted with one or more substituents Q. In certain embodiments, in Formula (A-VI), E^{b} is trivalent heteroaryl, optionally substituted with one or more substituents Q. In certain embodiments, in Formula (A-VI), E^{b} is trivalent heterocyclyl, optionally substituted with one or more substituents Q.

In certain embodiments, in Formula (A-VI), G is CH or N. In certain embodiments, in Formula (A-VI), G is CH. In certain embodiments, in Formula (A-VI), G is N.

In certain embodiments, in Formula (A-VI), G is trivalent C₁₋₆ alkyl, C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, in Formula (A-VI), G is trivalent C₁₋₆ alkyl, optionally substituted with one or more substituents Q. In certain embodiments, in Formula (A-VI), G is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, in Formula (A-VI), G is CR^{m}, wherein R^{m} is C₁₋₆ alkyl, optionally substituted with one or more Q. In certain embodiments, in Formula (A-VI), G is C(CH₃). In certain embodiments, in Formula (A-VI), G is trivalent C₁₋₆ heteroalkyl, optionally substituted with one or more substituents Q. In certain embodiments, in Formula (A-VI), G is trivalent C₁₋₆ alkenyl, optionally substituted with one or more substituents Q. In certain embodiments, in Formula (A-VI), G is trivalent C₃₋₁₀ cycloalkyl, optionally substituted with one or more substituents Q. In certain embodiments, in Formula (A-VI), G is trivalent C₆₋₁₄ aryl, optionally substituted with one or more substituents Q. In certain embodiments, in Formula (A-VI), G is trivalent heteroaryl, optionally substituted with one or more substituents Q. In certain embodiments, in Formula (A-VI), G is trivalent heterocyclyl, optionally substituted with one or more substituents Q.

In certain embodiments, in Formula (A-VI), E^{b} is CH or N; and G is CH or N.

In another embodiment, in a carbohydrate-oligonucleotide conjugate provided herein, the ASGPR binding moiety has the structure of Formula (A-VII):

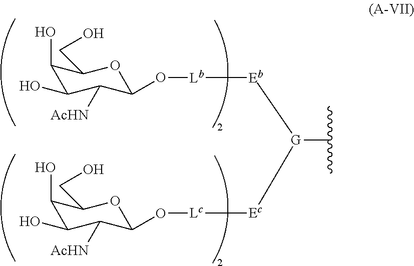

or an enantiomer, a mixture of enantiomers, a diastereomer, a mixture of two or more diastereomers, a tautomer, or a mixture of two or more tautomers thereof; wherein:
   E^{b} and E^{c} are each independently (i) CH or N; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q;
   G is (i) CH or N; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q; and
   each L^{b} and L^{c} is independently a linker.

In certain embodiments, in Formula (A-VII), E^{b} is CH or N. In certain embodiments, in Formula (A-VII), E^{b} is CH. In certain embodiments, in Formula (A-VII), E^{b} is N.

In certain embodiments, in Formula (A-VII), E^{b} is trivalent C₁₋₆ alkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, in Formula (A-VII), E^{b} is trivalent C₁₋₆ alkyl, optionally substituted with one or more substituents Q. In certain embodiments, in Formula (A-VII), E^{b} is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, in Formula (A-VII), E^{b} is CR^{m}, wherein R^{m} is C₁₋₆ alkyl, optionally substituted with one or more Q. In certain embodiments, in Formula (A-VII), E^{b} is C(CH₃). In certain embodiments, in Formula (A-VII), E^{b} is trivalent C₁₋₆ heteroalkyl, optionally substituted with one or more substituents Q. In certain embodiments, in Formula (A-VII), E^{b} is trivalent C₁₋₆ alkenyl, optionally substituted with one or more substituents Q. In certain embodiments, in Formula (A-VII), E^{b} is trivalent C₃₋₁₀ cycloalkyl, optionally substituted with one or more substituents Q. In certain embodiments, in Formula (A-VII), E^{b} is trivalent C₆₋₁₄ aryl, optionally substituted with one or more substituents Q. In certain embodiments, in Formula (A-VII), E^{b} is trivalent heteroaryl, optionally substituted with one or more substituents Q. In certain embodiments, in Formula (A-VII), E^{b} is trivalent heterocyclyl, optionally substituted with one or more substituents Q.

In certain embodiments, in Formula (A-VII), E^{c} is CH or N. In certain embodiments, in Formula (A-VII), E^{c} is CH. In certain embodiments, in Formula (A-VII), E^{c} is N.

In certain embodiments, in Formula (A-VII), E^{c} is trivalent C₁₋₆ alkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, in Formula (A-VII), E^{c} is trivalent C₁₋₆ alkyl, optionally substituted with one or more substituents Q. In certain embodiments, in Formula (A-VII), E^{c} is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, in Formula (A-VII), E^{c} is CR^{m}, wherein R^{m} is C₁₋₆ alkyl, optionally substituted with one or more Q. In certain embodiments, in Formula (A-VII), E^{c} is C(CH₃). In certain embodiments, in Formula (A-VII), E^{c} is trivalent C₁₋₆ heteroalkyl, optionally substituted with one or more substituents Q. In certain embodiments, in Formula (A-VII), E^{c} is trivalent C₁₋₆ alkenyl, optionally substituted with one or more substituents Q. In certain embodiments, in Formula (A-VII), E is trivalent C₃₋₁₀ cycloalkyl, optionally substituted with one or more substituents Q. In certain embodiments, in Formula (A-VII), E^{c} is trivalent C₆₋₁₄ aryl, optionally substituted with one or more substituents Q. In certain embodiments, in Formula (A-VII), E^{c} is trivalent heteroaryl, optionally substituted with one or more substituents Q. In certain embodiments, in Formula (A-VII), E^{c} is trivalent heterocyclyl, optionally substituted with one or more substituents Q.

In certain embodiments, in Formula (A-VII), G is CH or N. In certain embodiments, in Formula (A-VII), G is CH. In certain embodiments, in Formula (A-VII), G is N.

In certain embodiments, in Formula (A-VII), G is trivalent C₁₋₆ alkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, in Formula (A-VII), G is trivalent C₁₋₆ alkyl, optionally substituted with one or more substituents Q. In certain embodiments, in Formula (A-VII), G is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, in Formula (A-VII), G is CR^{m}, wherein R^{m} is C₁₋₆ alkyl, optionally substituted with one or more Q. In certain embodiments, in Formula (A-VII), G is C(CH₃). In certain embodiments, in Formula (A-VII), G is trivalent C₁₋₆ heteroalkyl, optionally substituted with one or more substituents Q. In certain embodiments, in Formula (A-VII), G is trivalent C₁₋₆ alkenyl, optionally substituted with one or more substituents Q. In certain embodiments, in Formula (A-VII), G is trivalent C₃₋₁₀ cycloalkyl, optionally substituted with one or more substituents Q. In certain embodiments, in Formula (A-VII), G is trivalent C₆₋₁₄ aryl, optionally substituted with one or more substituents Q. In certain embodiments, in Formula (A-VII), G is trivalent heteroaryl, optionally substituted with one or more substituents Q. In certain embodiments, in Formula (A-VII), G is trivalent heterocyclyl, optionally substituted with one or more substituents Q.

In certain embodiments, in Formula (A-VII), E^{b} is CH or N; E^{c} is CH or N; and G is CH or N.

In certain embodiments, in any one of Formula (A-V) to (A-VII), E^{b} is

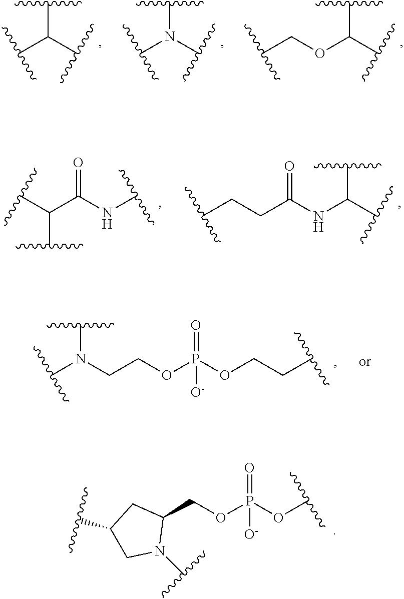

In certain embodiments, in any one of Formula (A-V) to (A-VII), E^{b} is

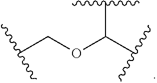 [SMILES: *COC(*)*]

In certain embodiments, in any one of Formula (A-V) to (A-VII), E^{c} is

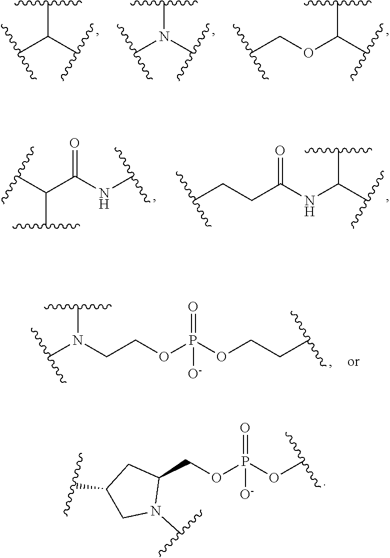

In certain embodiments, in Formula (A-V) or (A-VII), E^{c} is N,

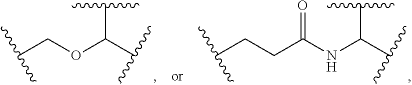 [SMILES: *COC(*)* ; *CCC(=O)NC(*)*]

In certain embodiments, in a carbohydrate-oligonucleotide conjugate provided herein, the ASGPR binding moiety is:

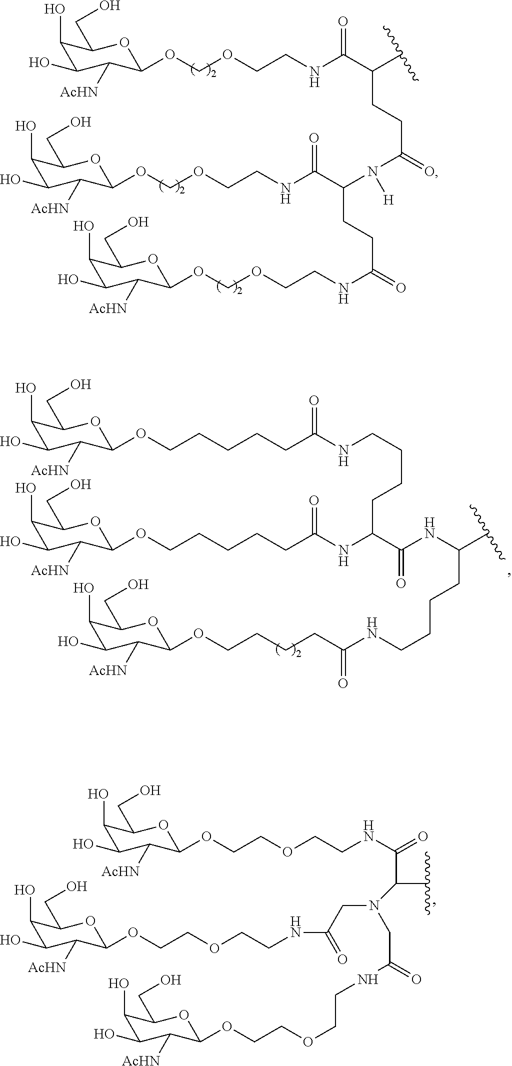

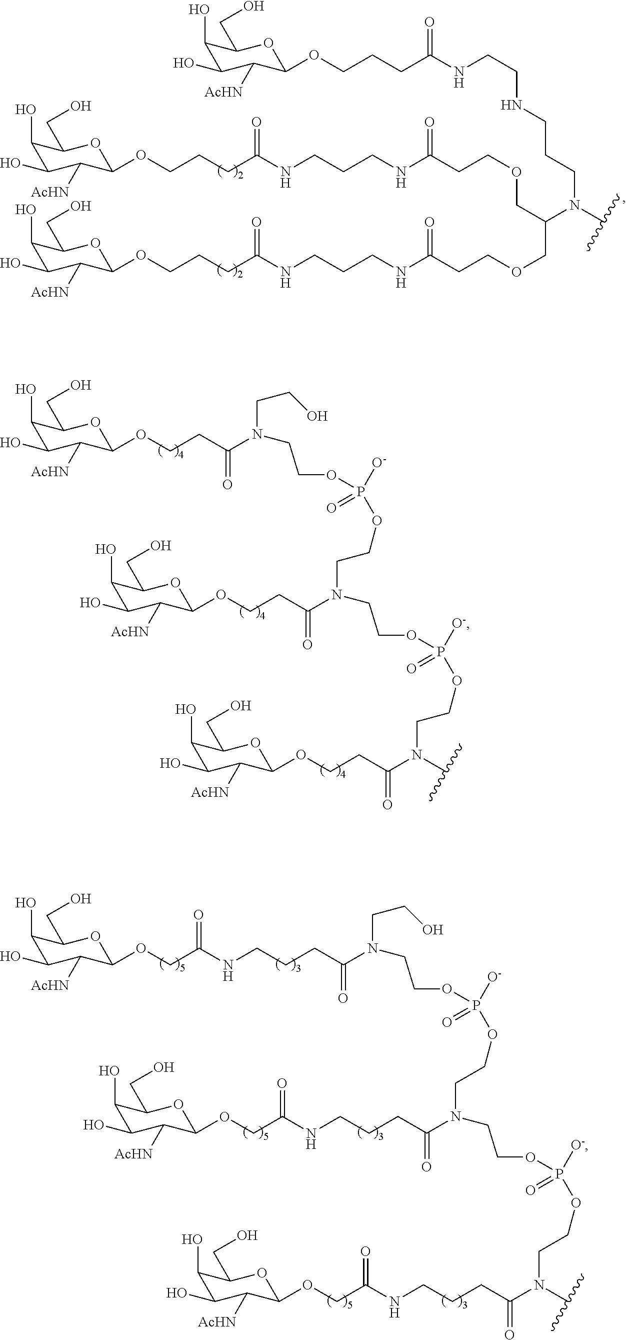

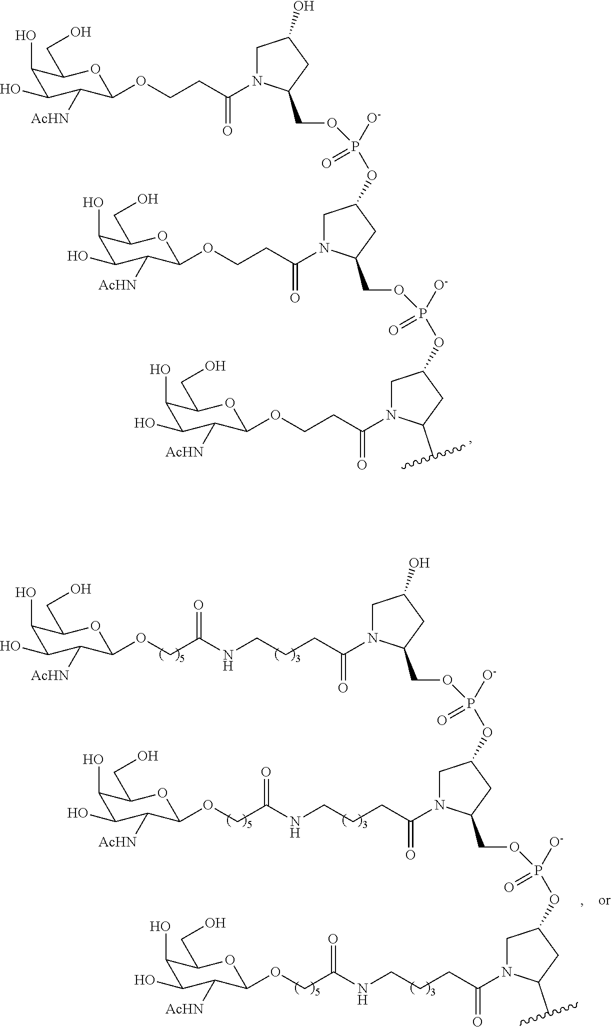

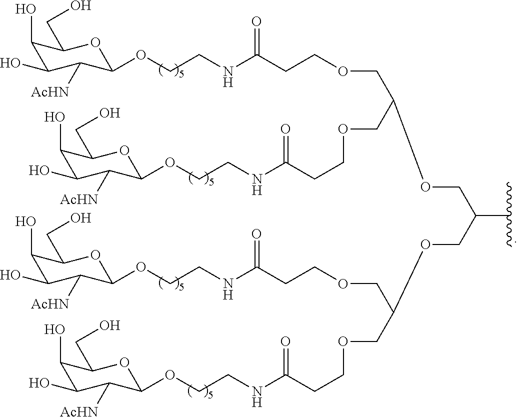

In certain embodiments, in any one of Formulae (A-I) to (A-IV), each L^{a} is independently a cleavable or non-cleavable linker. In certain embodiments, in any one of Formulae (A-I) to (A-IV), each L^{a} is independently a cleavable linker. In certain embodiments, in any one of Formulae (A-I) to (A-IV), each L^{a} is independently a non-cleavable linker.

In certain embodiments, in any one of Formulae (A-I) to (A-IV), each L^{a} is independently a cleavable linker that is sensitive to an acidic pH. In certain embodiments, in any one of Formulae (A-I) to (A-IV), each L^{a} is independently a cleavable linker comprising a reducible disulfide. In certain embodiments, in any one of Formulae (A-I) to (A-IV), each L^{a} is independently a linker cleavable by glutathione. In certain embodiments, in any one of Formulae (A-I) to (A-IV), each L^{a} is independently a linker cleavable by an enzyme. In certain embodiments, in any one of Formulae (A-I) to (A-IV), each L^{a} is independently a linker cleavable by a protease. In certain embodiments, in any one of Formulae (A-I) to (A-IV), each L^{a} is independently a linker cleavable by a lysosomal protease. In certain embodiments, in any one of Formulae (A-I) to (A-IV), each L^{a} is independently a linker cleavable by cathepsin B. In certain embodiments, in any one of Formulae (A-I) to (A-IV), each L^{a} is independently a linker cleavable by a glycosidase. In certain embodiments, in any one of Formulae (A-I) to (A-IV), each L^{a} is independently a linker cleavable by a β-glycosidase. In certain embodiments, in any one of Formulae (A-I) to (A-IV), each L^{a} is independently a linker cleavable by a galactosidase. In certain embodiments, in any one of Formulae (A-I) to (A-IV), each L^{a} is independently a linker cleavable by a β-galactosidase. In certain embodiments, in any one of Formulae (A-I) to (A-IV), each L^{a} is independently a linker cleavable by a glucuronidase. In certain embodiments, in any one of Formulae (A-I) to (A-IV), each L^{a} is independently a linker cleavable by a β-glucuronidase. In certain embodiments, in any one of Formulae (A-I) to (A-IV), each L^{a} is independently a linker cleavable by a phosphatase.

In certain embodiments, in any one of Formulae (A-V) to (A-VII), each L^{b} and L^{c} is independently a cleavable linker that is sensitive to an acidic pH. In certain embodiments, in any one of Formulae (A-V) to (A-VII), each L^{b} and L^{c} is independently a cleavable linker comprising a reducible disulfide. In certain embodiments, in any one of Formulae (A-V) to (A-VII), each L^{b} and L^{c} is independently a linker cleavable by glutathione. In certain embodiments, in any one of (A-V) to (A-VII), each L^{b} and L^{c} is independently a linker cleavable by an enzyme. In certain embodiments, in any one of Formulae (A-V) to (A-VII), each L^{b} and L^{c} is independently a linker cleavable by a protease. In certain embodiments, in any one of Formulae (A-V) to (A-VII), each L^{b} and L^{c} is independently a linker cleavable by a lysosomal protease. In certain embodiments, in any one of Formulae (A-V) to (A-VII), each L^{b} and L^{c} is independently a linker cleavable by cathepsin B. In certain embodiments, in any one of Formulae (A-V) to (A-VII), each L^{b} and L^{c} is independently a linker cleavable by a glycosidase. In certain embodiments, in any one of Formulae (A-V) to (A-VII), each L^{b} and L^{c} is independently a linker cleavable by a β-glycosidase. In certain embodiments, in any one of Formulae (A-V) to (A-VII), each L^{b} and L^{c} is independently a linker cleavable by a galactosidase. In certain embodiments, in any one of Formulae (A-V) to (A-VII), each L^{b} and L^{c} is independently a linker cleavable by a β-galactosidase. In certain embodiments, in any one of Formulae (A-V) to (A-VII), each L^{b} and L^{c} is independently a linker cleavable by a glucuronidase. In certain embodiments, in any one of Formulae (A-V) to (A-VII), each L^{b} and L^{c} is independently a linker cleavable by a β-glucuronidase. In certain embodiments, in any one of Formulae (A-V) to (A-VII), each L^{b} and L^{c} is independently a linker cleavable by a phosphatase.

In certain embodiments, each L^{a} in any one of Formulae (A-I) to (A-IV) is independently a linker having the structure of —Zⁿ—(Rⁿ Zⁿ)_{z}—, wherein:
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene, each of which is optionally substituted with one or more substituents Q;
each Zⁿ is independently a bond, —C(O)—, —C(O)O—, —C(O)NR^{1b}, —C(O)S—, —C(NR^{1a})NR^{1b}—, —C(S)—, —C(S)O—, —C(S)NR^{1b}—, —C(R^{1a})—NO—, —O—, —OC(O)O—, —OC(O)NR^{1b}—, —OC(O)S—, —OC(NR^{1a})NR^{1b}—, —OC(S)O—, —OC(S)NR^{1b}—, —OP(O₂)—, —OP(O)(S)—, —OP(O₂)O—, —OP(O)(S)O—, —OP(O₂)S—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —P(O₂)O—, —P(O)(S)O—, —S—, —S(O)—, —S(O)₂—, —S(O)NR^{1b}—, or —S(O)₂NR^{1b}—;
each R^{1a}, R^{1b}, R^{1c}, and R^{1d} is independently (i) hydrogen or deuterium; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; and
z is an integer of 0, 1, 2, 3, 4, 5, 6, 7, 8, 9, or 10.

In certain embodiments, each L^{b} in any one of Formulae (A-V) to (A-VII) is independently a linker having the structure of —Zⁿ—(Rⁿ Zⁿ)_{z}—, wherein each Rⁿ, Zⁿ, and z is as defined herein.

In certain embodiments, each L^{c} in any one of Formulae (A-V) to (A-VII) is independently a linker having the structure of —Zⁿ—(Rⁿ—Zⁿ)_{z}—, wherein each Rⁿ, Zⁿ, and z is as defined herein.

In certain embodiments, each Rⁿ is independently C₁₋₁₀ alkylene, C₂₋₁₀ alkynylene, C₃₋₁₀ cycloalkylene, C₆₋₁₄ arylene, heteroarylene, or heterocyclylene, each optionally substituted with one or more substituents Q; each Zⁿ is independently a bond, —C(O)—, —C(O)NR^{1b}—, —OC(NR^{1a})NR^{1b}—, —O—, —OC(O)NR^{1b}, —OP(O₂)—, —OP(O)(S)—, —NR^{1b}—, —P(O₂)O—, —P(O)(S)O—, —S—, —S(O)—, —S(O)₂—, or —S(O)₂NR^{1b}—; and z is an integer of 0, 1, 2, 3, 4, 5, or 6; where each R^{1a}, R^{1b}, and R^{1d} is as defined herein.

In certain embodiments, each Rⁿ is independently C₁₋₁₀ alkylene, C₂₋₁₀ alkynylene, C₃₋₁₀ cycloalkylene, C₆₋₁₄ arylene, heteroarylene, or heterocyclylene, each optionally substituted with one or more substituents Q; each Zⁿ is independently a bond, —C(O)—, —C(O)NR^{1b}—, —O—, —OC(O)NR^{1b}—, —OP(O₂)—, —OP(O)(S)—, —NR^{1b}—, —P(O₂)O—, —P(O)(S)O—, —S(O)₂—, or —S(O)₂NR^{1b}—; and z is an integer of 0, 1, 2, 3, 4, 5, 6, 7, or 8; where each R^{1a}, R^{1b}, and R^{1d} is as defined herein.

In certain embodiments, each Rⁿ is independently methanediyl, ethanediyl, propanediyl, butanediyl, pentanediyl, hexanediyl, heptanediyl, octanediyl, nonanediyl, decanediyl, undecanediyl, dodecanediyl, tridecanediyl, cthynediyl, propynediyl, pentynediyl, cyclobutanediyl, cyclopentanediyl, cyclohexanediyl, cycloheptanediyl, bicyclo[2.2.2]octanediyl, phendiyl, pyrazoldiyl, imidazoldiyl, tetrazoldiyl, pyrimidindiyl, 5,6,7,8,9,10-hexahydro-cycloocta[d]pyridazindiyl, azetidindiyl, 1,3-dioxandiyl, pyrrolidindiyl, piperazindiyl, piperidindiyl, or 3,9-diazaspiro[5.5]undecanediyl, each optionally substituted with one or more substituents Q. In certain embodiments, each Rⁿ is independently methanediyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, ethyne-1,2-diyl, propyne-1,3-diyl, 1-pentyne-1,5-diyl, cyclobutanc-1,3-diyl, cyclopentane-1,3-diyl, cyclohexane-1,3-diyl, cyclohexane-1,4-diyl, cycloheptane-1,3-diyl, cycloheptane-1,4-diyl, bicyclo[2.2.2]octane-1,4-diyl, phen-1,3-diyl, phen-1,4-diyl, pyrazol-1,3-diyl, pyrazol-1,4-diyl, imidazol-1,4-diyl, 1,2,3-triazol-1,4-diyl, pyrimidin-2,4-diyl, pyrimidin-2,5-diyl, 5,6,7,8,9,10-hexahydrocycloocta[d]-pyridazin-1,7-diyl, pyrazolidin-1,3-diyl, pyrazolidin-1,4-diyl, azetidin-1,3-diyl, 1,3-dioxan-2,5-diyl, pyrrolidin-1,3-diyl, piperazin-1,4-diyl, piperidin-1,3-diyl, piperidin-1,4-diyl, or 3,9-diazaspiro[5.5]-undecane-3,9-diyl, each optionally substituted with one or more substituents Q.

In certain embodiments, each Rⁿ is independently methanediyl, cyclopropyl-methanediyl, ethanediyl, pentanediyl, hexanediyl, heptanediyl, octanediyl, ethynediyl, propynediyl, pentynediyl, azetidindiyl, pyrrolidindiyl, piperazindiyl, or piperidindiyl. In certain embodiments, each Rⁿ is independently methanediyl, cyclopropylmethanediyl, ethane-1,2-diyl, pentane-1,5-diyl, hexane-1,6-diyl, heptane-1,7-diyl, octane-1,8-diyl, ethyne-1,2-diyl, propyne-1,3-diyl, 1-pentyne-1,5-diyl, azetidin-1,3-diyl, pyrrolidin-1,3-diyl, piperazin-1,4-diyl, or piperidin-1,4-diyl.

In certain embodiments, each Rⁿ is independently C₁₋₁₀ alkylene, C₆₋₁₄ arylene, or heteroarylene, each of which is optionally substituted with one or more substituents Q. In certain embodiments, each Rⁿ is independently methanediyl, ethanediyl, propanediyl, butanediyl, pentanediyl, hexanediyl, phendiyl, or 1,2,3-triazoldiyl, each optionally substituted with one, two, or three substituents Q. In certain embodiments, each Rⁿ is independently methanediyl, ethane-1,2-diyl, propane-1,3-diyl, butane-1,4-diyl, pentane-1,5-diyl, hexane-1,6-diyl, phen-1,4-diyl, or 1,2,3-triazol-1,4-diyl, each optionally substituted with one or more substituents Q.

In certain embodiments, each Zⁿ is independently a bond, —C(O)—, —C(O)O—, —C(O)NH—, —OC(O)NH—, —C(CH₃)═NO—, —O—, —OP(O₂)—, —OP(O)(S)—, —NH—, —N(CH₃)—, —P(O₂)O—, —P(O)(S)O—, —S—, or —S(O)₂—. In certain embodiments, each Zⁿ is independently a bond, —C(O)NH—, —O—, —OP(O₂)—, —OP(O)(S)—, —NH—, —P(O₂)O—, or —P(O)(S)O—.

In certain embodiments, z is an integer of 0. In certain embodiments, z is an integer of 1. In certain embodiments, z is an integer of 2. In certain embodiments, z is an integer of 3. In certain embodiments, z is an integer of 4. In certain embodiments, z is an integer of 5. In certain embodiments, z is an integer of 6. In certain embodiments, z is an integer of 7. In certain embodiments, z is an integer of 8. In certain embodiments, z is an integer of 0, 1, 2, 3, 4, or 5.

In certain embodiments, each Rⁿ is independently methanediyl, ethanediyl, propanediyl, butanediyl, pentanediyl, hexanediyl, heptanediyl, octanediyl, nonanediyl, decanediyl, undecanediyl, dodecanediyl, tridecanediyl, ethynediyl, propynediyl, pentynediyl, cyclobutanediyl, cyclopentanediyl, cyclohexanediyl, cycloheptanediyl, bicyclo[2.2.2]octanediyl, phendiyl, pyrazoldiyl, imidazoldiyl, tetrazoldiyl, pyrimidindiyl, 5,6,7,8,9,10-hexahydro-cycloocta[d]pyridazindiyl, azetidindiyl, 1,3-dioxandiyl, pyrrolidindiyl, piperazindiyl, piperidindiyl, or 3,9-diazaspiro[5.5]undecanediyl, each optionally substituted with one or more substituents Q; each Zⁿ is independently a bond, —C(O)—, —C(O)O—, —C(O)NH—, —OC(O)NH—, —C(CH₃)═NO—, —O—, —OP(O₂)—, —OP(O)(S)—, —NH—, —N(CH₃)—, —P(O₂)O—, —P(O)(S)O—, —S—, or —S(O)₂—; and z is an integer of 0, 1, 2, 3, 4, 5, 6, 7, or 8.

In certain embodiments, each Rⁿ is independently methanediyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, ethyne-1,2-diyl, propyne-1,3-diyl, 1-pentyne-1,5-diyl, cyclobutane-1,3-diyl, cyclopentane-1,3-diyl, cyclohexane-1,3-diyl, cyclohexane-1,4-diyl, cycloheptane-1,3-diyl, cycloheptane-1,4-diyl, bicyclo[2.2.2]octane-1,4-diyl, phen-1,3-diyl, phen-1,4-diyl, pyrazol-1,3-diyl, pyrazol-1,4-diyl, imidazol-1,4-diyl, 1,2,3-triazol-1,4-diyl, pyrimidin-2,4-diyl, pyrimidin-2,5-diyl, 5,6,7,8,9,10-hexahydrocycloocta[d]-pyridazin-1,7-diyl, pyrazolidin-1,3-diyl, pyrazolidin-1,4-diyl, azetidin-1,3-diyl, 1,3-dioxan-2,5-diyl, pyrrolidin-1,3-diyl, piperazin-1,4-diyl, piperidin-1,3-diyl, piperidin-1,4-diyl, or 3,9-diazaspiro[5.5]-undecane-3,9-diyl, each optionally substituted with one or more substituents Q; each Zⁿ is independently a bond, —C(O)—, —C(O)O—, —C(O)NH—, —OC(O)NH—, —C(CH₃)═NO—, —O—, —OP(O₂)—, —OP(O)(S)—, —NH—, —N(CH₃)—, —P(O₂)O—, —P(O)(S)O—, —S—, or —S(O)₂—; and z is an integer of 0, 1, 2, 3, 4, or 5.

In certain embodiments, each Rⁿ is independently methanediyl, cyclopropylmethanediyl, ethanediyl, pentanediyl, hexanediyl, heptanediyl, octanediyl, ethynediyl, propynediyl, pentynediyl, azetidindiyl, pyrrolidindiyl, piperazindiyl, or piperidindiyl; each Zⁿ is independently a bond, —C(O)—, —C(O)NH—, —C(CH₃)—NO—, —O—, —OP(O₂)—, —OP(O)(S)—, —NH—, —N(CH₃), —S—, or —S(O)₂—; and z is an integer of 0, 1, 2, 3, 4, or 5.

In certain embodiments, each Rⁿ is independently methanediyl, cyclopropylmethanediyl, ethane-1,2-diyl, pentane-1,5-diyl, hexane-1,6-diyl, heptane-1,7-diyl, octane-1,8-diyl, ethyne-1,2-diyl, propyne-1,3-diyl, 1-pentyne-1,5-diyl, azetidin-1,3-diyl, pyrrolidin-1,3-diyl, piperazin-1,4-diyl, or piperidin-1,4-diyl; each Zⁿ is independently a bond, —C(O)—, —C(O)NH—, —C(CH₃)═NO—, —O—, —OP(O₂)—, —OP(O)(S)—, —NH—, —N(CH₃)—, —P(O₂)O—, —P(O)(S)O—, —S—, or —S(O)₂—; and z is an integer of 0, 1, 2, 3, 4, or 5.

In certain embodiments, in any one of Formulae (A-I) to (A-IV), each L^{a} is independently:

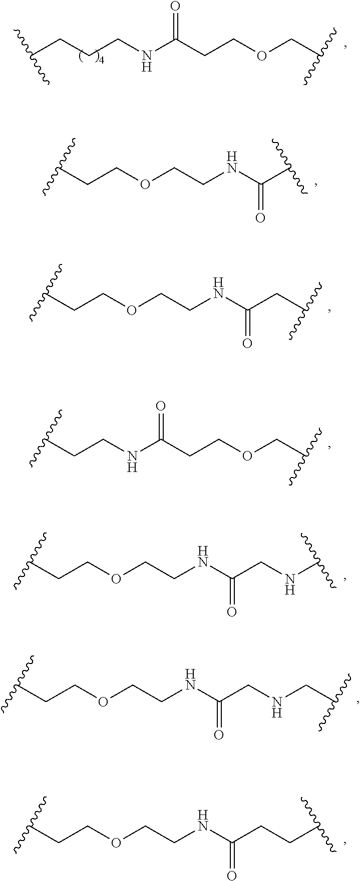

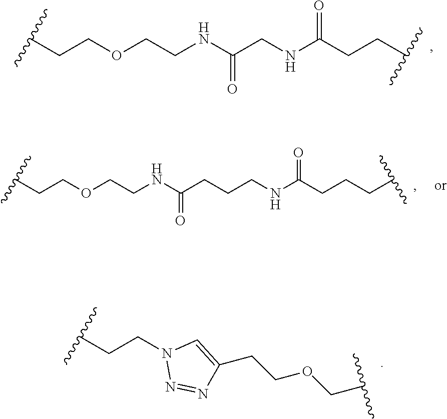 [SMILES: *CCOCCNC(=O)CNC(=O)CC* ; *CCCC(=O)NCCCC(=O)NCCOCC* ; *CCn1cc(CCOC*)nn1]

In certain embodiments, in any one of Formulae (A-I) to (A-IV), each L^{a} is independently:

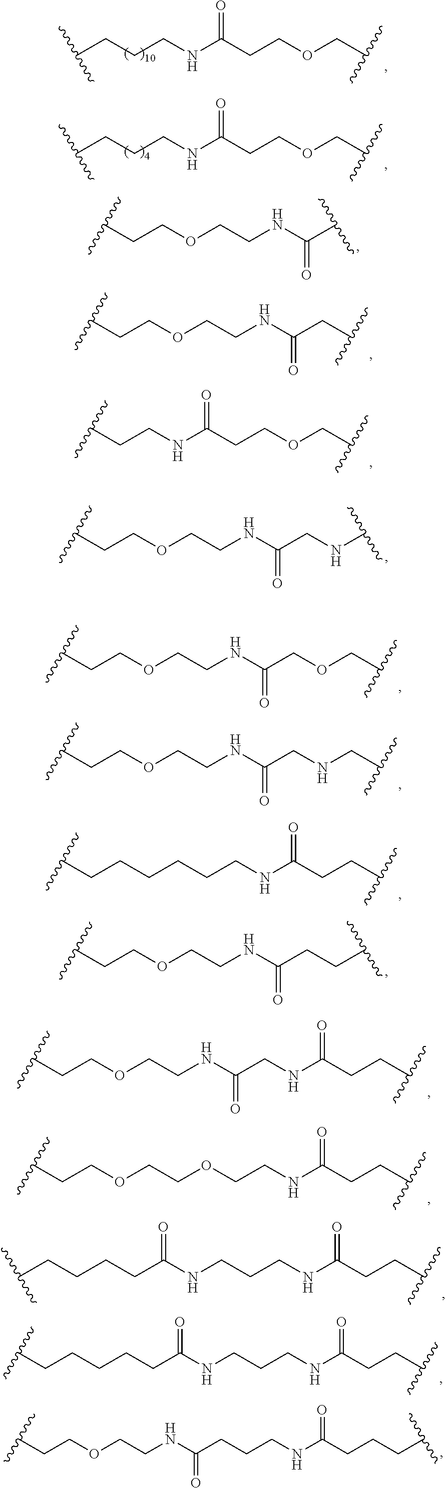

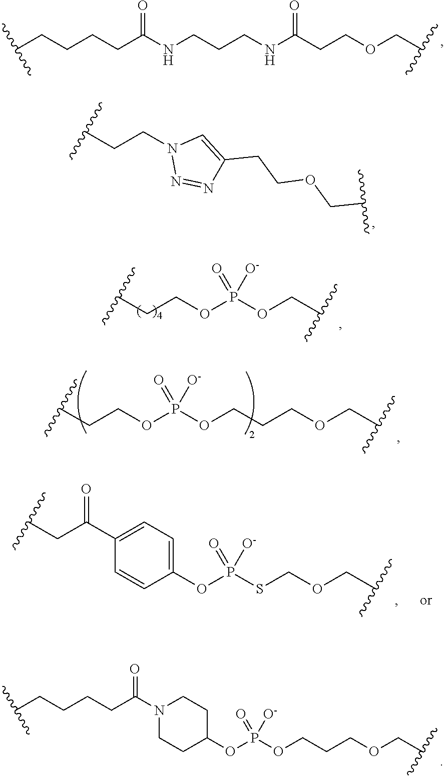

In certain embodiments, in any one of Formulae (A-V) to (A-VII), each L^{b} and L^{c} is independently:

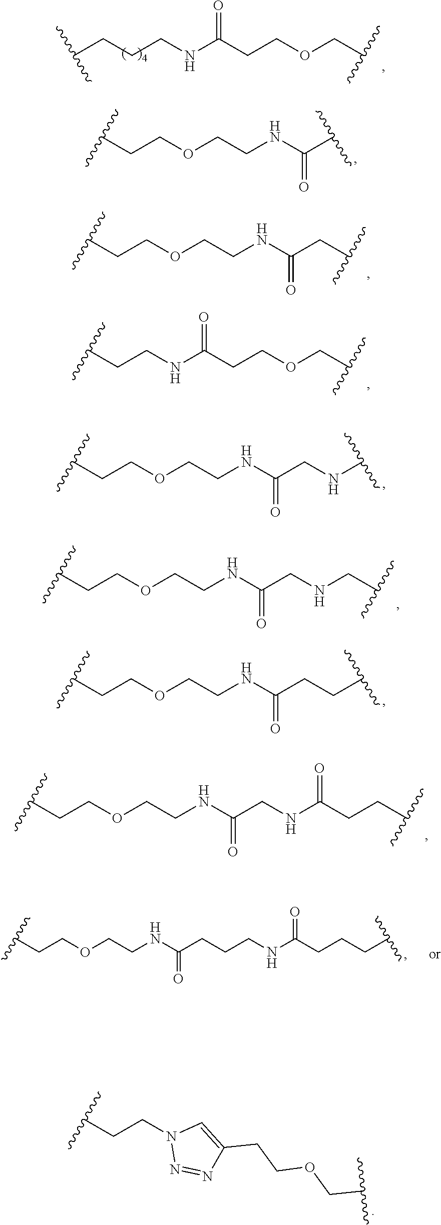

In certain embodiments, in any one of Formulae (A-V) to (A-VII), each L^{b} and L^{c} is independently:

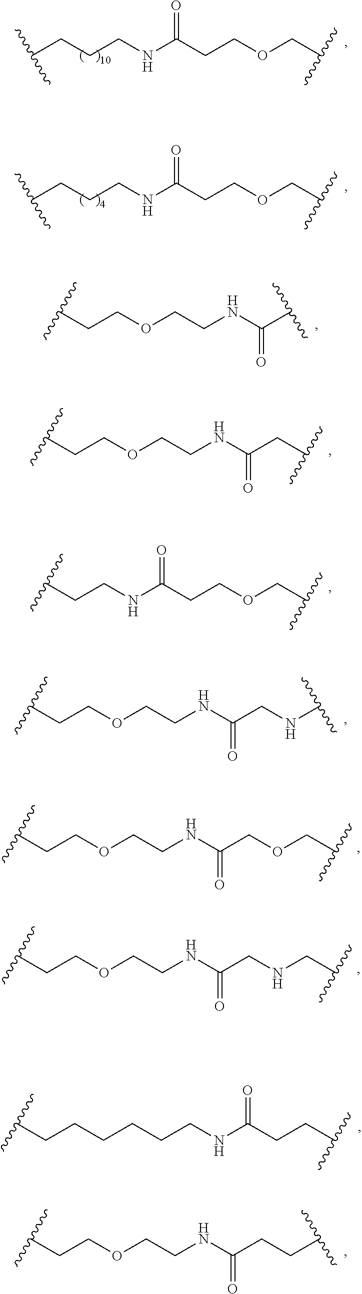

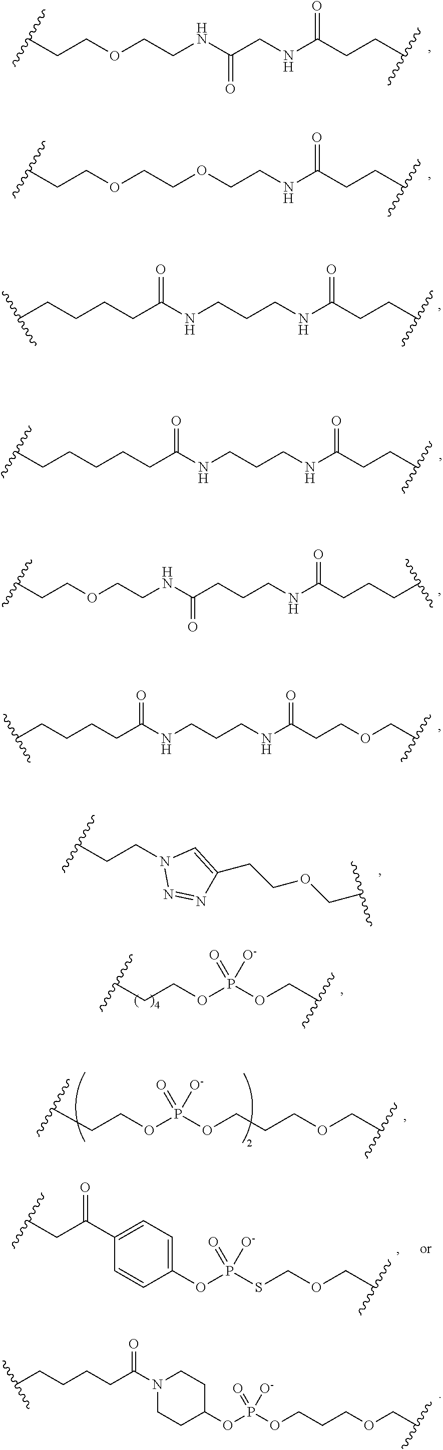

In certain embodiments, the trivalent linker in a carbohydrate-oligonucleotide conjugate provided herein is independently a linker having the structure of:

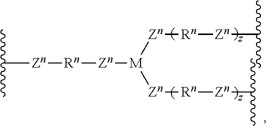

wherein:
   M is (i) N or CH; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q; and
   each Rⁿ, Zⁿ, and z is as defined herein.

In certain embodiments, M is N or CH. In certain embodiments, M is N. In certain embodiments, M is CH.

In certain embodiments, M is trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, M is trivalent C₁₋₆ alkyl, optionally substituted with one or more substituents Q. In certain embodiments, M is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, M is CR^{m}, wherein R^{m} is C₁₋₆ alkyl, optionally substituted with one or more Q. In certain embodiments, M is C(CH₃). In certain embodiments, M is trivalent C₁₋₆ heteroalkyl, optionally substituted with one or more substituents Q. In certain embodiments, M is trivalent C₁₋₆ alkenyl, optionally substituted with one or more substituents Q. In certain embodiments, M is trivalent C₃₋₁₀ cycloalkyl, optionally substituted with one or more substituents Q. In certain embodiments, M is trivalent C₆₋₁₄ aryl, optionally substituted with one or more substituents Q. In certain embodiments, M is trivalent heteroaryl, optionally substituted with one or more substituents Q. In certain embodiments, M is trivalent heterocyclyl, optionally substituted with one or more substituents Q.

In certain embodiments, each Rⁿ is independently C₁₋₁₀ alkylene, C₆₋₁₄ arylene, heteroarylene, or heterocyclyl, each of which is optionally substituted with one or more substituents Q. In certain embodiments, each Rⁿ is independently methanediyl, ethanediyl, propanediyl, butanediyl, pentanediyl, hexanediyl, phendiyl, 1,2,3-triazoldiyl, pyrrolidindiyl, tetrahydrothiendiyl, or tetrahydropyrandiyl, each optionally substituted with one, two, or three substituents Q. In certain embodiments, each Rⁿ is independently methanediyl, ethane-1,2-diyl, acetamidoethane-1,2-diyl, propane-1,3-diyl, 2-hydroxylpropane-1,3-diyl, butane-1,4-diyl, pentane-1,5-diyl, hexane-1,6-diyl, phen-1,4-diyl, 1,2,3-triazol-1,4-diyl, pyrrolidin-1,2-diyl, 2,5-dioxopyrrolidin-1,3-diyl, tetrahydrothien-2,5-diyl, or tetrahydropyran-1,3-diyl. In certain embodiments, each Rⁿ is independently methanediyl, ethane-1,2-diyl, acetamidoethane-1,2-diyl, propane-1,3-diyl, 2-hydroxylpropane-1,3-diyl, butane-1,4-diyl, pentane-1,5-diyl, hexane-1,6-diyl, phen-1,4-diyl, 1,2,3-triazol-1,4-diyl, or 2,5-dioxopyrrolidin-1,3-diyl.

In certain embodiments, each Zⁿ is independently a bond, —C(O)—, —C(O)O—, —C(O)NH—, —C(CH₃)═NO—, —O—, —OC(O)NH—, —NH—, —N(CH₃)—, —OP(O₂)—, —OP(O)(S)—, —P(O₂)O—, —P(O)(S)O—, —S—, or —S(O)₂—. In certain embodiments, each Zⁿ is independently a bond, —C(O)—, —C(O)NH—, —C(CH₃)═NO—, —O—, —NH—, —OP(O₂)—, —P(O₂)O—, —P(O)(S)O—, or —S—.

In certain embodiments, each z is independently an integer of 0, 1, 2, 3, 4, or 5. In certain embodiments, each z is independently an integer of 0, 1, 2, 3, or 4. In certain embodiments, each z is independently an integer of 0, 1, 2, or 3.

In certain embodiments, each Rⁿ is independently methanediyl, ethanediyl, propanediyl, butanediyl, pentanediyl, hexanediyl, phendiyl, 1,2,3-triazoldiyl, pyrrolidindiyl, tetrahydrothiendiyl, or tetrahydropyrandiyl, each optionally substituted with one, two, or three substituents Q; each Zⁿ is independently a bond, —C(O)—, —C(O)O—, —C(O)NH—, —C(CH₃)═NO—, —O—, —OC(O)NH—, —NH—, —N(CH₃)—, —OP(O₂)—, —OP(O)(S)—, —P(O₂)O—, —P(O)(S)O—, —S—, or —S(O)₂; each z is independently an integer of 0, 1, 2, 3, 4, or 5.

In certain embodiments, each Rⁿ is independently methanediyl, ethane-1,2-diyl, acetamidoethane-1,2-diyl, propane-1,3-diyl, 2-hydroxylpropane-1,3-diyl, butane-1,4-diyl, pentane-1,5-diyl, hexane-1,6-diyl, phen-1,4-diyl, 1,2,3-triazol-1,4-diyl, pyrrolidin-1,2-diyl, 2,5-dioxopyrrolidin-1,3-diyl, tetrahydrothien-2,5-diyl, or tetrahydropyran-1,3-diyl; each Zⁿ is independently a bond, —C(O)—, —C(O)O—, —C(O)NH—, —C(CH₃)═NO—, —O—, —OC(O)NH—, —NH—, —N(CH₃)—, —OP(O₂)—, —OP(O)(S)—, —P(O₂)O—, —P(O)(S)O—, —S—, or —S(O)₂—; and each z is independently an integer of 0, 1, 2, 3, or 4.

In certain embodiments, each Rⁿ is independently methanediyl, ethane-1,2-diyl, acetamidoethane-1,2-diyl, propane-1,3-diyl, 2-hydroxylpropane-1,3-diyl, butane-1,4-diyl, pentane-1,5-diyl, hexane-1,6-diyl, phen-1,4-diyl, 1,2,3-triazol-1,4-diyl, or 2,5-dioxopyrrolidin-1,3-diyl; each Zⁿ is independently a bond, —C(O)—, —C(O)NH—, —C(CH₃)—NO—, —O—, —NH—, —OP(O₂)—, —P(O₂)O—, —P(O)(S)O—, or —S—; and each z is independently an integer of 0, 1, 2, or 3.

In certain embodiments, the trivalent linker in a carbohydrate-oligonucleotide conjugate provided herein has the structure of:

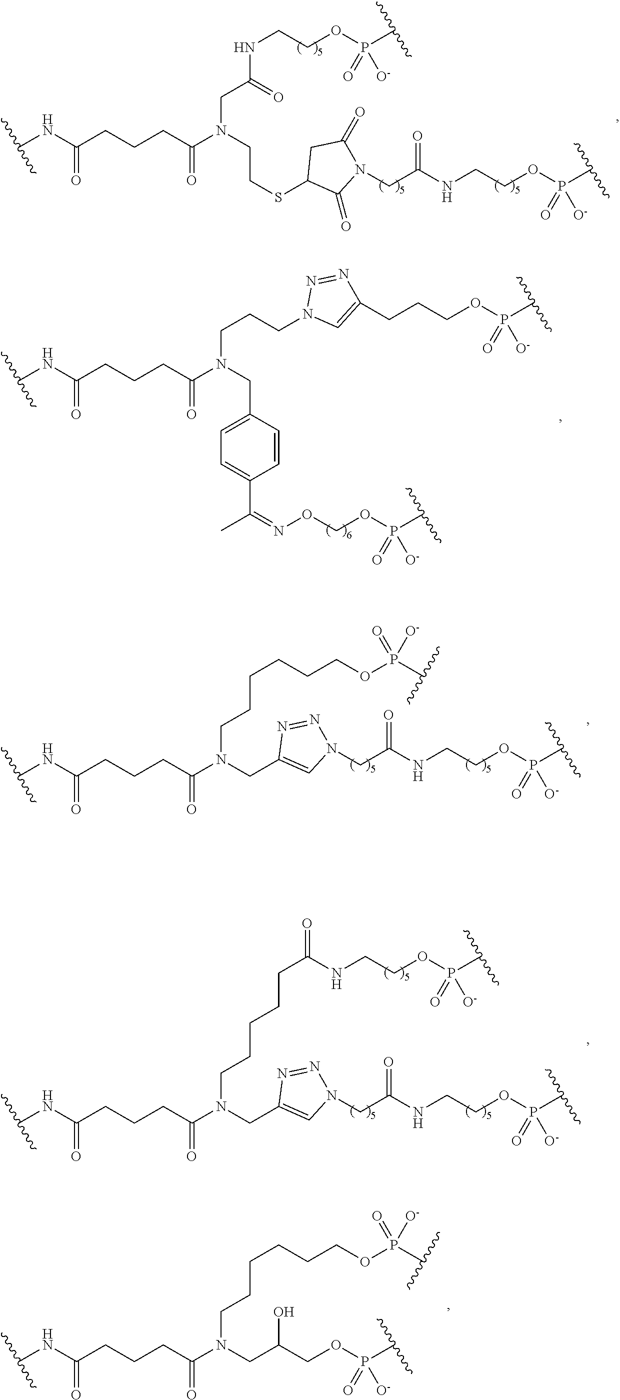

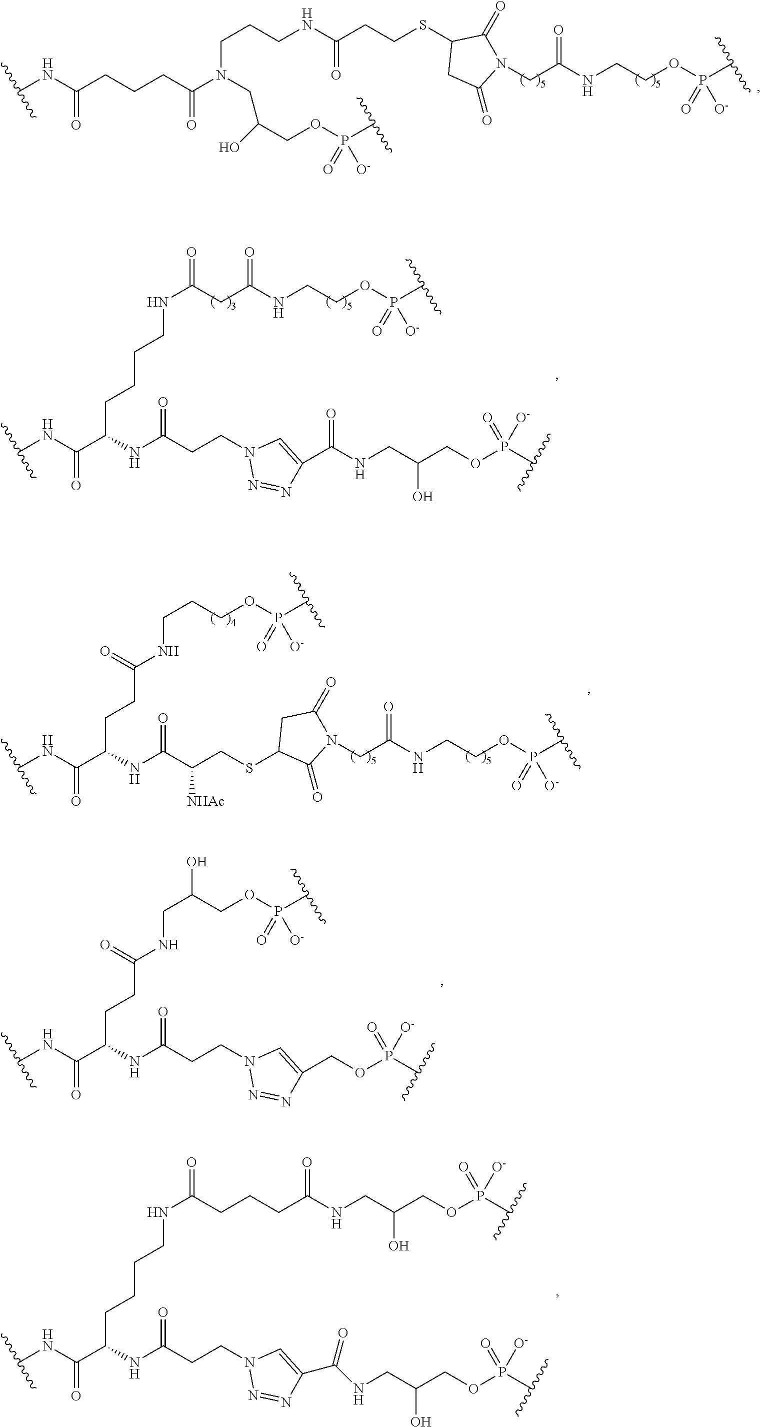

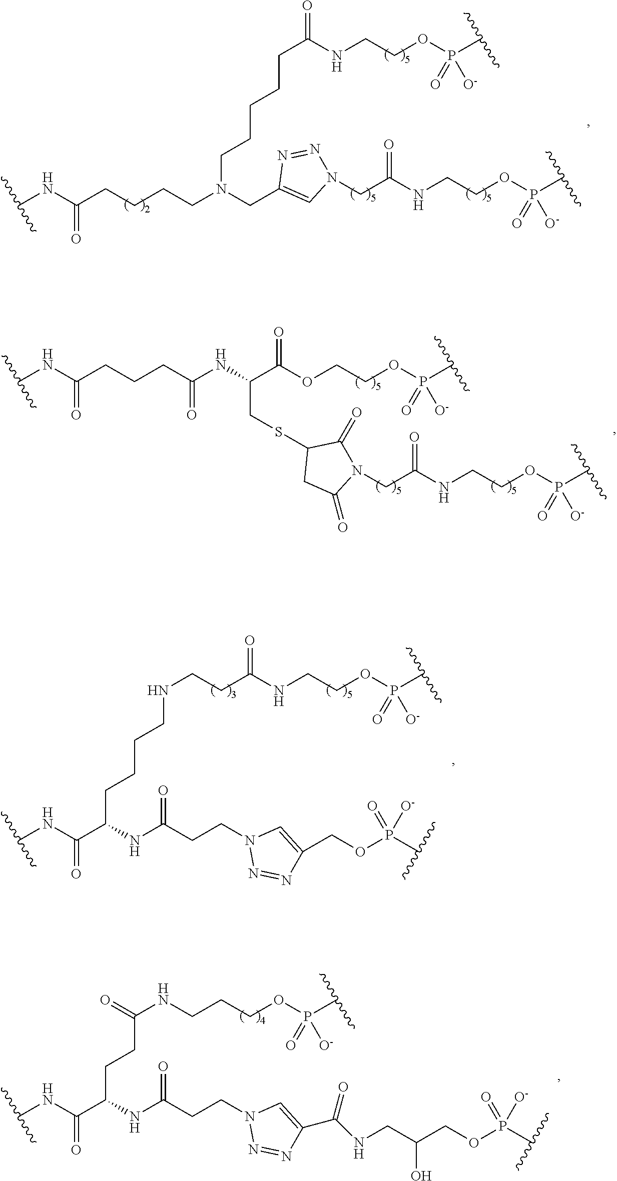

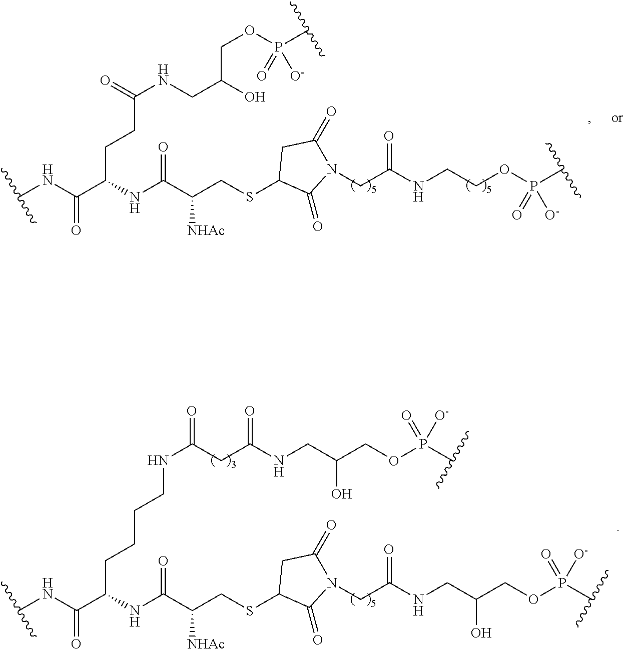

In certain embodiments, the trivalent linker in a carbohydrate-oligonucleotide conjugate provided herein has the structure of:

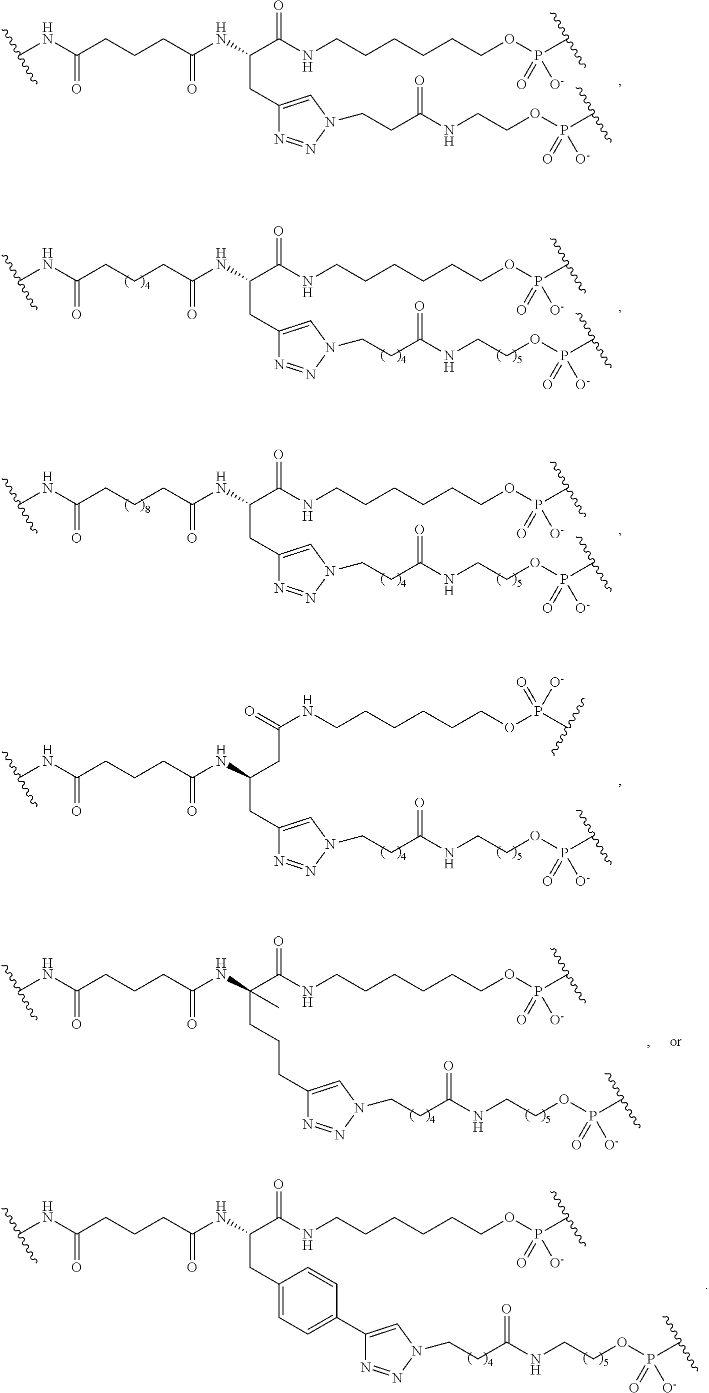

In certain embodiments, the trivalent linker in a carbohydrate-oligonucleotide conjugate provided herein has the structure of:

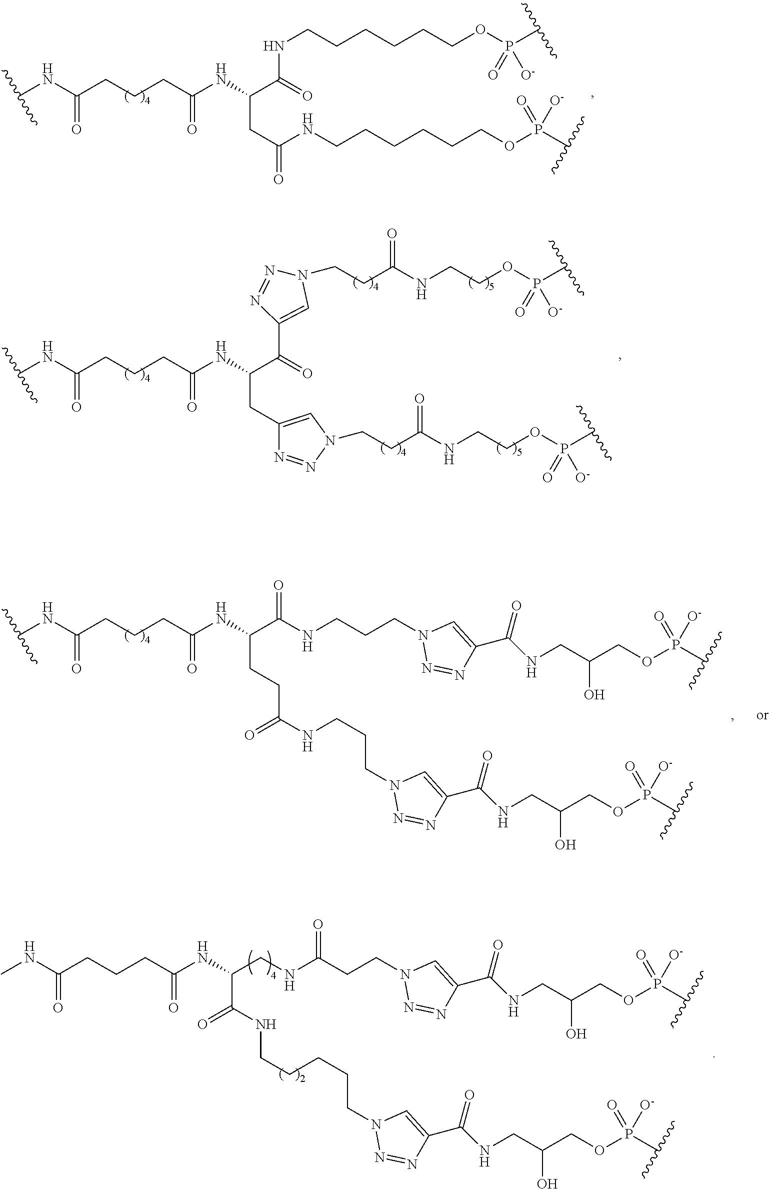

In certain embodiments, the trivalent linker in a carbohydrate-oligonucleotide conjugate provided herein is a cleavable or non-cleavable linker. In certain embodiments, the trivalent linker in a carbohydrate-oligonucleotide conjugate provided herein is a cleavable linker. In certain embodiments, the trivalent linker in a carbohydrate-oligonucleotide conjugate provided herein is a non-cleavable linker.

In certain embodiments, the trivalent linker in a carbohydrate-oligonucleotide conjugate provided herein is a cleavable linker that is sensitive to an acidic pH. In certain embodiments, the trivalent linker in a carbohydrate-oligonucleotide conjugate provided herein is a cleavable linker comprising a reducible disulfide. In certain embodiments, the trivalent linker in a carbohydrate-oligonucleotide conjugate provided herein is a linker cleavable by glutathione. In certain embodiments, the trivalent linker in a carbohydrate-oligonucleotide conjugate provided herein is a linker cleavable by an enzyme. In certain embodiments, the trivalent linker in a carbohydrate-oligonucleotide conjugate provided herein is a linker cleavable by a protease. In certain embodiments, the trivalent linker in a carbohydrate-oligonucleotide conjugate provided herein is a linker cleavable by a lysosomal protease. In certain embodiments, the trivalent linker in a carbohydrate-oligonucleotide conjugate provided herein is a linker cleavable by cathepsin B. In certain embodiments, the trivalent linker in a carbohydrate-oligonucleotide conjugate provided herein is a linker cleavable by a glycosidase. In certain embodiments, the trivalent linker in a carbohydrate-oligonucleotide conjugate provided herein is a linker cleavable by a β-glycosidase. In certain embodiments, the trivalent linker in a carbohydrate-oligonucleotide conjugate provided herein is a linker cleavable by a galactosidase. In certain embodiments, the trivalent linker in a carbohydrate-oligonucleotide conjugate provided herein is a linker cleavable by a β-galactosidase. In certain embodiments, the trivalent linker in a carbohydrate-oligonucleotide conjugate provided herein is a linker cleavable by a glucuronidase. In certain embodiments, the trivalent linker in a carbohydrate-oligonucleotide conjugate provided herein is a linker cleavable by a β-glucuronidase. In certain embodiments, the trivalent linker in a carbohydrate-oligonucleotide conjugate provided herein is a linker cleavable by a phosphatase. Exemplary linkers suitable for carbohydrate-oligonucleotide conjugate provided herein include, but are not limited to, those disclosed in Beck et al., Nat. Rev. Drug Discov. 2017, 16, 317-37; Bargh et al., Chem. Soc. Rev. 2019, 48, 4361-74; the disclosure of each of which is incorporated herein by reference in its entirety.

In certain embodiments, each divalent linker in a carbohydrate-oligonucleotide conjugate provided herein is independently a linker having the structure of —Zⁿ—(Rⁿ—Zⁿ)_{z}—, wherein each Rⁿ, Zⁿ, and z is as defined herein.

In certain embodiments, each divalent linker in a carbohydrate-oligonucleotide conjugate provided herein independently has the structure of:

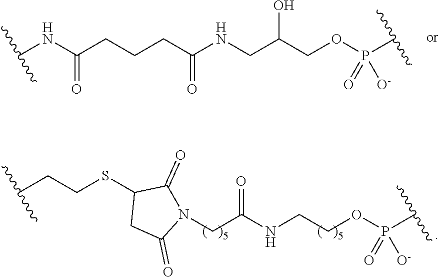

In certain embodiments, each divalent linker in a carbohydrate-oligonucleotide conjugate provided herein is independently a cleavable or non-cleavable linker. In certain embodiments, each divalent linker in a carbohydrate-oligonucleotide conjugate provided herein is independently a cleavable linker. In certain embodiments, each divalent linker in a carbohydrate-oligonucleotide conjugate provided herein is independently a non-cleavable linker.

In certain embodiments, each divalent linker in a carbohydrate-oligonucleotide conjugate provided herein is independently a cleavable linker that is sensitive to an acidic pH. In certain embodiments, each divalent linker in a carbohydrate-oligonucleotide conjugate provided herein is independently a cleavable linker comprising a reducible disulfide. In certain embodiments, each divalent linker in a carbohydrate-oligonucleotide conjugate provided herein is independently a linker cleavable by glutathione. In certain embodiments, each divalent linker in a carbohydrate-oligonucleotide conjugate provided herein is independently a linker cleavable by an enzyme. In certain embodiments, each divalent linker in a carbohydrate-oligonucleotide conjugate provided herein is independently a linker cleavable by a protease. In certain embodiments, each divalent linker in a carbohydrate-oligonucleotide conjugate provided herein is independently a linker cleavable by a lysosomal protease. In certain embodiments, each divalent linker in a carbohydrate-oligonucleotide conjugate provided herein is independently a linker cleavable by cathepsin B. In certain embodiments, each divalent linker in a carbohydrate-oligonucleotide conjugate provided herein is independently a linker cleavable by a glycosidase. In certain embodiments, each divalent linker in a carbohydrate-oligonucleotide conjugate provided herein is independently a linker cleavable by a β-glycosidase. In certain embodiments, each divalent linker in a carbohydrate-oligonucleotide conjugate provided herein is independently a linker cleavable by a galactosidase. In certain embodiments, each divalent linker in a carbohydrate-oligonucleotide conjugate provided herein is independently a linker cleavable by a β-galactosidase. In certain embodiments, each divalent linker in a carbohydrate-oligonucleotide conjugate provided herein is independently a linker cleavable by a glucuronidase. In certain embodiments, each divalent linker in a carbohydrate-oligonucleotide conjugate provided herein is independently a linker cleavable by a β-glucuronidase. In certain embodiments, each divalent linker in a carbohydrate-oligonucleotide conjugate provided herein is independently a linker cleavable by a phosphatase. Exemplary linkers suitable for carbohydrate-oligonucleotide conjugate provided herein include, but are not limited to, those disclosed in Beck et al., Nat. Rev. Drug Discov. 2017, 16, 317-37; Bargh et al., Chem. Soc. Rev. 2019, 48, 4361-74; the disclosure of each of which is incorporated herein by reference in its entirety.

In one embodiment, provided herein is a carbohydrate-oligonucleotide conjugate having the structure of Formula (I):

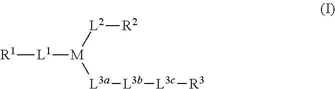

wherein:
   R¹ is an ASGPR binding moiety;
   R² and R³ are each independently an oligonucleotide;
   L¹, L², L^{3a}, and L^{3c} are each independently a linker;
   L^{3b} is (i) a bond; or (ii) heteroarylene or heterocyclylene, each optionally substituted with one or more substituents Q; and
   M is (i) N or CH; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q.

In certain embodiments, in Formula (I), L^{3b} is a bond. In certain embodiments, in Formula (I), L^{3b} is heteroarylene or heterocyclylene, each optionally substituted with one or more substituents Q. In certain embodiments, in Formula (I), L^{3b} is heteroarylene, optionally substituted with one or more substituents Q. In certain embodiments, in Formula (I), L^{3b} is monocyclic heteroarylene, optionally substituted with one or more substituents Q. In certain embodiments, in Formula (I), L^{3b} is 5- or 6-membered heteroarylene, each optionally substituted with one or more substituents Q. In certain embodiments, in Formula (I), L^{3b} is 5-membered heteroarylene, each optionally substituted with one or more substituents Q. In certain embodiments, in Formula (I), L^{3b} is [1,2,3]triazoldiyl, optionally substituted with one or more substituents Q. In certain embodiments, in Formula (I), L^{3b} is [1,2,3]triazol-1,4-diyl, optionally substituted with one or more substituents Q. In certain embodiments, in Formula (I), L^{3b} is 6-membered heteroarylene, each optionally substituted with one or more substituents Q. In certain embodiments, in Formula (I), L^{3b} is bicyclic heteroarylene, optionally substituted with one or more substituents Q. In certain embodiments, in Formula (I), L^{3b} is tricyclic heteroarylene, optionally substituted with one or more substituents Q.

In certain embodiments, in Formula (I), L^{3b} is heterocyclylene, optionally substituted with one or more substituents Q. In certain embodiments, in Formula (I), L^{3b} is monocyclic heterocyclylene, optionally substituted with one or more substituents Q. In certain embodiments, in Formula (I), L^{3b} is bicyclic heterocyclylene, optionally substituted with one or more substituents Q. In certain embodiments, in Formula (I), L^{3b} is 4,5,6,7,8,9-hexahydrocyclo-octa[d][1,2,3]triazoldiyl or 5,6,7,8,9,10-hexahydrocycloocta[d]pyridazindiyl, each optionally substituted with one or more substituents Q. In certain embodiments, in Formula (I), L^{3b} is 4,5,6,7,8,9-hexahydrocycloocta[d][1,2,3]triazol-1,6-diyl, 4,5,6,7,8,9-hexahydrocycloocta[d]-[1,2,3]triazol-1,7-diyl, or 5,6,7,8,9,10-hexahydrocycloocta[d]pyridazin-1,7-diyl, each optionally substituted with one or more substituents Q.

In certain embodiments, in Formula (I), L^{3b} is tricyclic heterocyclylene, optionally substituted with one or more substituents Q. In certain embodiments, in Formula (I), L^{3b} is 1,5a,6,6a-tetrahydrocyclopropa[5,6]cycloocta[1,2-d][1,2,3]triazoldiyl, optionally substituted with one or more substituents Q. In certain embodiments, in Formula (I), L^{3b} is 1,5a,6,6a-tetrahydro-cyclopropa[5,6]cycloocta[1,2-d][1,2,3]triazol-1,6-diyl, optionally substituted with one or more substituents Q. In certain embodiments, in Formula (I), L^{3b} is tetracyclic heterocyclylene, optionally substituted with one or more substituents Q. In certain embodiments, in Formula (I), L^{3b} is 8,9-dihydrodibenzo[b,f][1,2,3]triazolo[4,5-d]azocindiyl, optionally substituted with one or more substituents Q. In certain embodiments, in Formula (I), L^{3b} is 8,9-dihydrodibenzo[b,f]-[1,2,3]triazolo[4,5-d]azocin-1,8-diyl or 8,9-dihydrodibenzo[b,f][1,2,3]triazolo-[4,5-d]azocin-3,8-diyl, each optionally substituted with one or more substituents Q.

In certain embodiments, in Formula (I), L^{3b} is

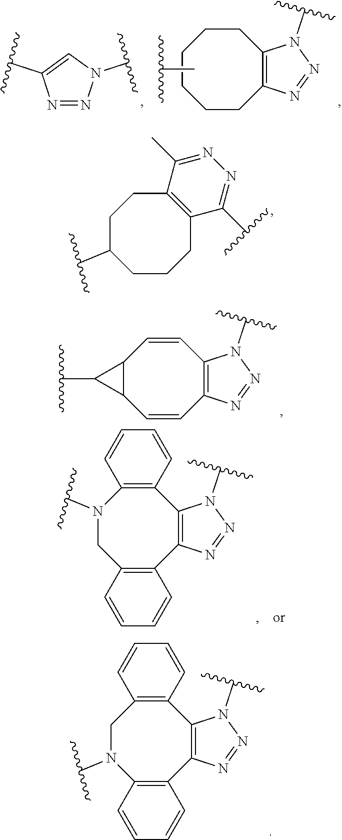

In certain embodiments, in Formula (I), L^{3b} is

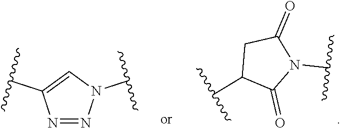 [SMILES: *c1cn(*)nn1 ; *C1CC(=O)N(*)C1=O]

In another embodiment, provided herein is a carbohydrate-oligonucleotide conjugate having the structure of Formula (II):

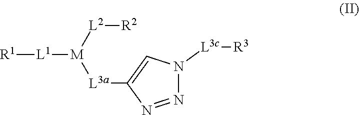

wherein R¹, R², R³, L¹, L², L^{3a}, L^{3c}, and M are each as defined herein.

In certain embodiments, in Formula (I) or (II), R¹ is an ASGPR binding moiety as described herein. In certain embodiments, in Formula (I) or (II), R¹ is an ASGPR binding moiety of Formula (A-I). In certain embodiments, in Formula (I) or (II), R¹ is an ASGPR binding moiety of Formula (A-II). In certain embodiments, in Formula (I) or (II), R¹ is an ASGPR binding moiety of Formula (A-III). In certain embodiments, in Formula (I) or (II), R¹ is an ASGPR binding moiety of Formula (A-IV). In certain embodiments, in Formula (I) or (II), R¹ is an ASGPR binding moiety of Formula (A-V). In certain embodiments, in Formula (I) or (II), R¹ is an ASGPR binding moiety of Formula (A-VI). In certain embodiments, in Formula (I) or (II), R¹ is an ASGPR binding moiety of Formula (A-VII).

In certain embodiments, in Formula (I) or (II), R¹ is

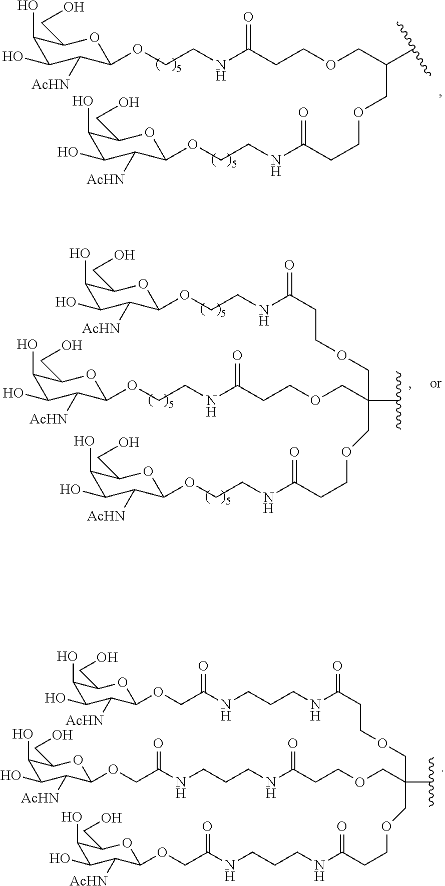

In certain embodiments, in Formula (I) or (II), R² is an oligonucleotide as described herein. In certain embodiments, in Formula (I) or (II), R² is a single-stranded oligonucleotide as described herein. In certain embodiments, in Formula (I) or (II), R² is a single-stranded oligodeoxyribonucleotide or oligoribonucleotide. In certain embodiments, in Formula (I) or (II), R² is a single-stranded oligodeoxyribonucleotide (ssDNA). In certain embodiments, in Formula (I) or (II), R² is a single-stranded oligoribonucleotide (ssRNA). In certain embodiments, in Formula (I) or (II), R² is an ASO, miRNA, mRNA, or tRNA. In certain embodiments, in Formula (I) or (II), R² is a single-stranded oligoribonucleotide.

In certain embodiments, in Formula (I) or (II), R² is a single-stranded oligonucleotide comprising the nucleotide sequence of SEQ ID NO: 1, 3, 5, 7, 9, or 11. In certain embodiments, in Formula (I) or (II), R² is a single-stranded oligonucleotide comprising the nucleotide sequence of SEQ ID NO: 1, 5, or 9.

In certain embodiments, in Formula (I) or (II), R² is a single-stranded oligonucleotide comprising the nucleotide sequence of any one of SEQ ID NOs: 1 to 25. In certain embodiments, in Formula (I) or (II), R² is a single-stranded oligonucleotide comprising the nucleotide sequence of SEQ ID NO: 1, 3, 5, 7, 9, 11, 13, 15, 17, 19, 21, 23, or 25. In certain embodiments, in Formula (I) or (II), R² is a single-stranded oligonucleotide comprising the nucleotide sequence of SEQ ID NO: 1, 3, 5, 7, 9, 11, 13, 15, 17, 19, 21, or 23. In certain embodiments, in Formula (I) or (II), R² is a single-stranded oligonucleotide comprising the nucleotide sequence of SEQ ID NO: 1, 3, 5, 9, 13, 15, 19, or 23.

In certain embodiments, in Formula (I) or (II), R² is a double-stranded oligonucleotide as described herein. In certain embodiments, in Formula (I) or (II), R² is a double-stranded oligodeoxyribonucleotide or oligoribonucleotide. In certain embodiments, in Formula (I) or (II), R² is a double-stranded oligodeoxyribonucleotide. In certain embodiments, in Formula (I) or (II), R² is a double-stranded oligoribonucleotide.

In certain embodiments, in Formula (I) or (II), R² is a siRNA. In certain embodiments, in Formula (I) or (II), R² is a siRNA comprising a pair of nucleotide sequences of SEQ ID NOs: 1 and 2, 3 and 4, 5 and 6, 7 and 8, 9 and 10, or 11 and 12. In certain embodiments, in Formula (I) or (II), R² is a siRNA comprising a pair of nucleotide sequences of SEQ ID NOs: 1 and 2, 5 and 6, or 9 and 10.

In certain embodiments, in Formula (I) or (II), R² is a siRNA. In certain embodiments, in Formula (I) or (II), R² is a siRNA comprising a pair of nucleotide sequences of SEQ ID NOs: 1 and 2, 3 and 4, 5 and 6, 7 and 8, 9 and 10, 11 and 12, 13 and 14, 15 and 16, 17 and 18, 19 and 20, 21 and 22, or 23 and 24. In certain embodiments, in Formula (I) or (II), R² is a siRNA comprising a pair of nucleotide sequences of SEQ ID NOs: 1 and 2, 3 and 4, 5 and 6, 9 and 10, 13 and 14, 15 and 16, 19 and 20, or 23 and 24.

In certain embodiments, in Formula (I) or (II), R³ is an oligonucleotide as described herein. In certain embodiments, in Formula (I) or (II), R³ is a single-stranded oligonucleotide as described herein. In certain embodiments, in Formula (I) or (II), R³ is a single-stranded oligodeoxyribonucleotide or oligoribonucleotide. In certain embodiments, in Formula (I) or (II), R³ is a single-stranded oligodeoxyribonucleotide. In certain embodiments, in Formula (I) or (II), R³ is an ASO, miRNA, mRNA, or tRNA. In certain embodiments, in Formula (I) or (II), R³ is a single-stranded oligonucleotide comprising the nucleotide sequence of SEQ ID NO: 1, 3, 5, 7, 9, or 11. In certain embodiments, in Formula (I) or (II), R³ is a single-stranded oligonucleotide comprising the nucleotide sequence of SEQ ID NO: 3, 7, or 11.

In certain embodiments, in Formula (I) or (II), R³ is a single-stranded oligonucleotide comprising the nucleotide sequence of any one of SEQ ID NOs: 1 to 25. In certain embodiments, in Formula (I) or (II), R³ is a single-stranded oligonucleotide comprising the nucleotide sequence of SEQ ID NO: 1, 3, 5, 7, 9, 11, 13, 15, 17, 19, 21, 23, or 25. In certain embodiments, in Formula (I) or (II), R³ is a single-stranded oligonucleotide comprising the nucleotide sequence of SEQ ID NO: 1, 3, 5, 7, 9, 11, 13, 15, 17, 19, 21, or 23. In certain embodiments, in Formula (I) or (II), R³ is a single-stranded oligonucleotide comprising the nucleotide sequence of SEQ ID NO: 1, 3, 7, 11, 17, 19, 21, or 25. In certain embodiments, in Formula (I) or (II), R³ is a single-stranded oligonucleotide comprising the nucleotide sequence of SEQ ID NO: 1, 3, 7, 11, 17, 19, or 21.

In certain embodiments, in Formula (I) or (II), R³ is a double-stranded oligonucleotide as described herein. In certain embodiments, in Formula (I) or (II), R³ is a double-stranded oligodeoxyribonucleotide or oligoribonucleotide. In certain embodiments, in Formula (I) or (II), R³ is a double-stranded oligodeoxyribonucleotide. In certain embodiments, in Formula (I) or (II), R³ is a double-stranded oligoribonucleotide.

In certain embodiments, in Formula (I) or (II), R³ is a siRNA. In certain embodiments, in Formula (I) or (II), R³ is a siRNA comprising a pair of nucleotide sequences of SEQ ID NOs: 1 and 2, 3 and 4, 5 and 6, 7 and 8, 9 and 10, or 11 and 12. In certain embodiments, in Formula (I) or (II), R³ is a siRNA comprising a pair of nucleotide sequences of SEQ ID NOS: 3 and 4, 7 and 8, or 11 and 12.

In certain embodiments, in Formula (I) or (II), R³ is a siRNA. In certain embodiments, in Formula (I) or (II), R³ is a siRNA comprising a pair of nucleotide sequences of SEQ ID NOs: 1 and 2, 3 and 4, 5 and 6, 7 and 8, 9 and 10, 11 and 12, 13 and 14, 15 and 16, 17 and 18, 19 and 20, 21 and 22, or 23 and 24. In certain embodiments, in Formula (I) or (II), R³ is a siRNA comprising a pair of nucleotide sequences of SEQ ID NOs: 1 and 2, 3 and 4, 7 and 8, 11 and 12, 17 and 18, 19 and 20, or 21 and 22.

In certain embodiments, in Formula (I) or (II), L¹ is a linker as defined herein. In certain embodiments, in Formula (I) or (II), L¹ is a linker having the structure of —Zⁿ—(Rⁿ—Zⁿ)_{z}—, wherein each Rⁿ, Zⁿ, and z is as defined herein. In certain embodiments, in Formula (I) or (II), L¹ is —NHC(O)(CH₂)ₑC(O)NH—, wherein e is an integer of 1, 2, 3, 4, 5, 6, 7, 8, 9, 10, 11, 12, 13, 14, or 15. In certain embodiments, in Formula (I) or (II), L¹ is —NHC(O)(CH₂)ₑC(O)NH—, wherein e is an integer of 3, 6, or 10. In certain embodiments, in Formula (I) or (II), L¹ is —NHC(O)(CH₂)₃C(O)NH—. In certain embodiments, in Formula (I) or (II), L¹ is —NHC(O)(CH₂)₆C(O)NH—. In certain embodiments, in Formula (I) or (II), L¹ is —NHC(O)(CH₂)₁₀C(O)NH—. In certain embodiments, in Formula (I) or (II), L¹ is

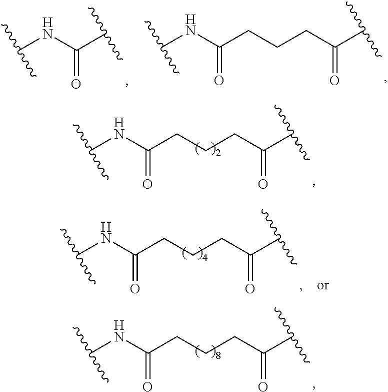

In certain embodiments, in Formula (I) or (II), L² is a linker as defined herein. In certain embodiments, in Formula (I) or (II), L² is a linker having the structure of —Zⁿ—(Rⁿ Zⁿ)_{z}—, wherein each Rⁿ, Zⁿ, and z is as defined herein. In certain embodiments, in Formula (I) or (II), L² is —(CH₂)_{f}C(O)NH(CH₂)_{g}OP(O₂), wherein f is an integer of 1, 2, 3, 4, 5, or 6; and g is an integer of 1, 2, 3, 4, 5, 6, 7, 8, 9, or 10. In certain embodiments, in Formula (I) or (II), L² is —(CH₂)NHC(O)(CH₂)₆OP(O₂)— or —NHC(O)(CH₂)₆OP(O₂)—. In certain embodiments, in Formula (I) or (II), L² is —(CH₂)NHC(O)(CH₂)₆OP(O₂)—. In certain embodiments, in Formula (I) or (II), L² is —NHC(O)(CH₂)₆OP(O₂)—.

In certain embodiments, in Formula (I) or (II), L² is a linker comprising —O—, —S—, or —N(H)—. In certain embodiments, in Formula (I) or (II), L² is a linker containing —O—, —S—, or —N(H)—. In certain embodiments, in Formula (I) or (II), L² is a linker comprising C₆₋₁₄ arylene or heterocyclylene, each optionally substituted with one or more substituents Q. In certain embodiments, in Formula (I) or (II), L² is a linker comprising phendiyl or monocyclic heterocyclylene, each optionally substituted with one or more substituents Q. In certain embodiments, in Formula (I) or (II), L² is a linker comprising phendiyl, or 5- or 6-membered heterocyclylene, each optionally substituted with one or more substituents Q. In certain embodiments, in Formula (I) or (II), L² is a linker comprising phendiyl, pyrrolidindiyl, tetrahydrothiophendiyl, tetrahydropyrandiyl, or piperidindiyl, each optionally substituted with one or more substituents Q.

In certain embodiments, in Formula (I) or (II), L² is —X(CH₂)ₕX—, wherein each X is independently —O—, —S—, or —N(H)—; and h is an integer of 2, 3, 4, 5, 6, 7, 8, 9, 10, 11, or 12. In certain embodiments, in Formula (I) or (II), L² is —X(CH₂)ᵢCH(OH)(CH₂)ⱼX—, wherein each X is as defined herein; and i and i are each independently an integer of 2, 3, 4, 5, 6, 7, 8, 9, or 10.

In certain embodiments, in Formula (I) or (II), L² is

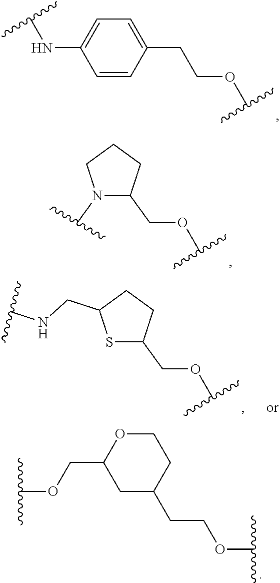 [SMILES: *Nc1ccc(CCO*)cc1 ; *OCC1CCCN1* ; *NCC1CCC(CO*)S1 ; *OCCC1CCOC(CO*)C1]

In certain embodiments, in Formula (I) or (II), L² is

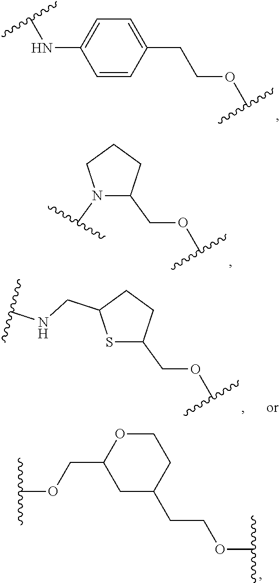

wherein L² is attached to a 5′-terminus of an oligonucleotide. In certain embodiments, in Formula (I) or (II), L² is

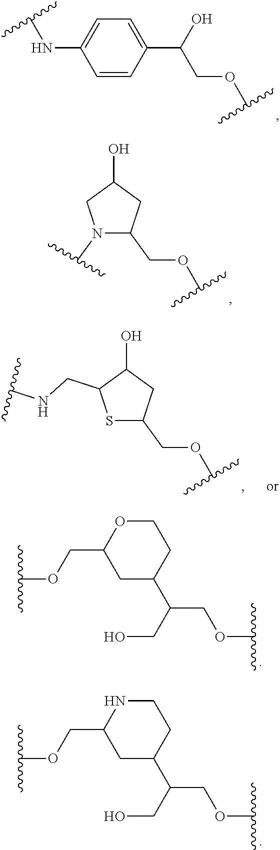 [SMILES: *Nc1ccc(C(O)CO*)cc1 ; *OCC1CC(O)CN1* ; *NCC1SC(CO*)CC1O ; *OCC1CC(C(CO)CO*)CCO1 ; *OCC1CC(C(CO)CO*)CCN1]

In certain embodiments, in Formula (I) or (II), L² is

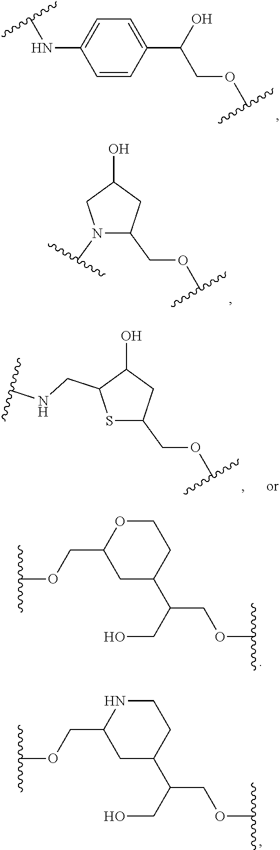

wherein L² is attached to a 3′-terminus of an oligonucleotide.

In certain embodiments, in Formula (I) or (II), L² is

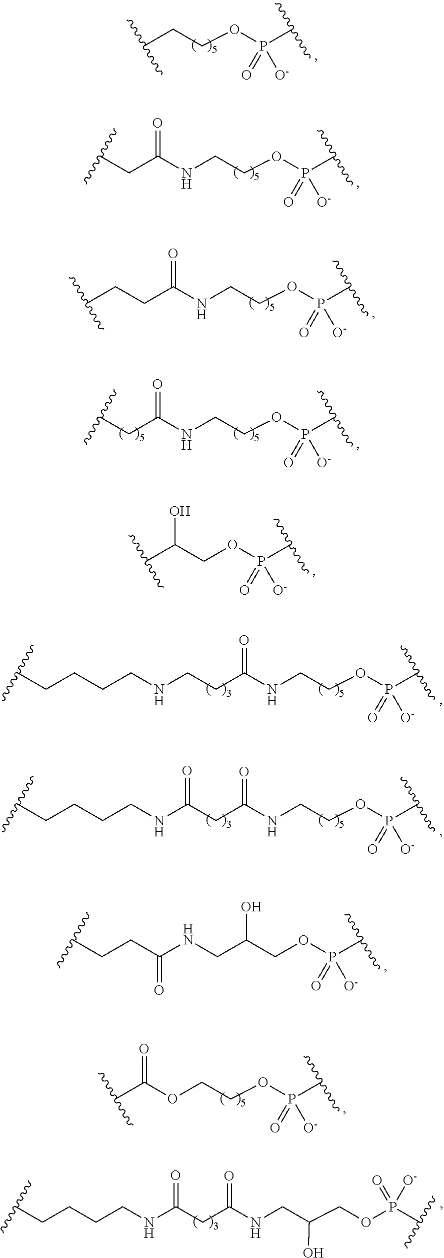

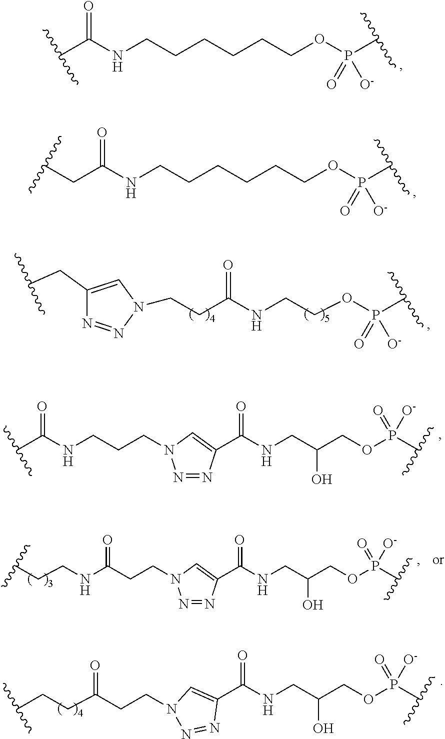

In certain embodiments, in Formula (I) or (II), L^{3a} is a linker as defined herein. In certain embodiments, in Formula (I) or (II), L^{3a} is a linker having the structure of —Zⁿ—(Rⁿ—Zⁿ)_{z}—, wherein each Rⁿ, Zⁿ, and z is as defined herein. In certain embodiments, in Formula (I) or (II), L^{3a} is C₁₋₁₀ alkylene or C₇₋₁₅ aralkylene, each optionally substituted with one or more substituents Q. In certain embodiments, in Formula (I) or (II), L^{3a} is methylene or benzdiyl, each optionally substituted with one or more substituents Q. In certain embodiments, in Formula (I) or (II), L^{3a} is —(CH₂)— or —(CH₂)-phen-1,4-diyl, each optionally substituted with one or more substituents Q.

In certain embodiments, in Formula (I) or (II), L^{3a} is —CH₂—, —CH₂CH₂—, —CH₂CH₂CH₂—, —CH₂CH₂CH₂CH₂—,

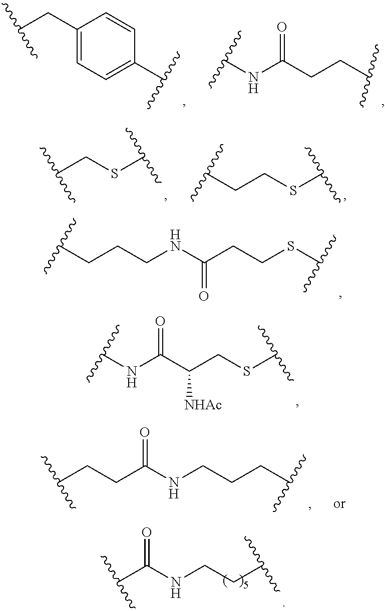

In certain embodiments, in Formula (I) or (II), L^{3c} is a linker as defined herein. In certain embodiments, in Formula (I) or (II), L^{3c} is a linker having the structure of —Zⁿ—(Rⁿ—Zⁿ)_{z}—, wherein each Rⁿ, Zⁿ, and z is as defined herein. In certain embodiments, in Formula (I) or (II), L^{3c} is —(CH₂)ₘC(O)NH(CH₂)ₙOP(O₂)—, wherein m and n are each independently an integer of 1, 2, 3, 4, 5, 6, 7, 8, 9, or 10. In certain embodiments, in Formula (I) or (II), L^{3c} is —(CH₂)₅C(O)NH(CH₂)₆OP(O₂)—.

In certain embodiments, in Formula (I) or (II), L^{3c} is a linker comprising —O—, —S—, or —N(H)—. In certain embodiments, in Formula (I) or (II), L^{3c} is a linker containing —O—, —S—, or —N(H)—. In certain embodiments, in Formula (I) or (II), L^{3c} is a linker comprising C₆₋₁₄ arylene or heterocyclylene, each optionally substituted with one or more substituents Q. In certain embodiments, in Formula (I) or (II), L^{3c} is a linker comprising phendiyl or monocyclic heterocyclylene, each optionally substituted with one or more substituents Q. In certain embodiments, in Formula (I) or (II), L^{3c} is a linker comprising phendiyl, or 5- or 6-membered heterocyclylene, each optionally substituted with one or more substituents Q. In certain embodiments, in Formula (I) or (II), L^{3c} is a linker comprising phendiyl, pyrrolidindiyl, tetrahydrothiophendiyl, tetrahydropyrandiyl, or piperidindiyl, each optionally substituted with one or more substituents Q.

In certain embodiments, in Formula (I) or (II), L^{3c} is —X(CH₂)ₚX—, wherein each X is independently —O—, —S—, or —N(H)—; and p is an integer of 2, 3, 4, 5, 6, 7, 8, 9, 10, 11, or 12. In certain embodiments, in Formula (I) or (II), L^{3c} is —X(CH₂)_{q}CH(OH)(CH₂)ᵣX—, wherein each X is as defined herein; and q and r are each independently an integer of 2, 3, 4, 5, 6, 7, 8, 9, or 10. In certain embodiments, in Formula (I) or (II), L^{3c} is

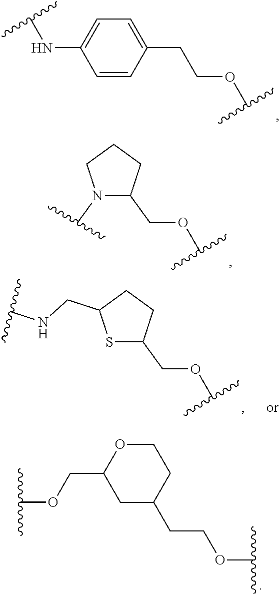 [SMILES: *Nc1ccc(CCO*)cc1 ; *OCC1CCCN1* ; *NCC1CCC(CO*)S1 ; *OCCC1CCOC(CO*)C1]

In certain embodiments, in Formula (I) or (II),

L^{3c} is

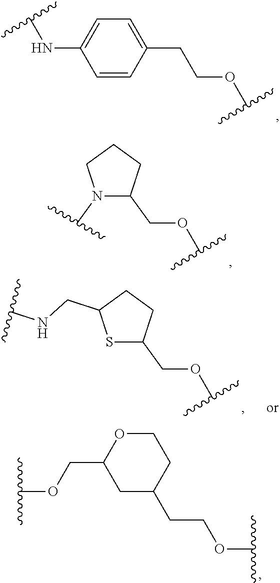

wherein L^{3c} is attached to a 5′-terminus of an oligonucleotide. In certain embodiments, in Formula (I) or (II), L^{3c} is

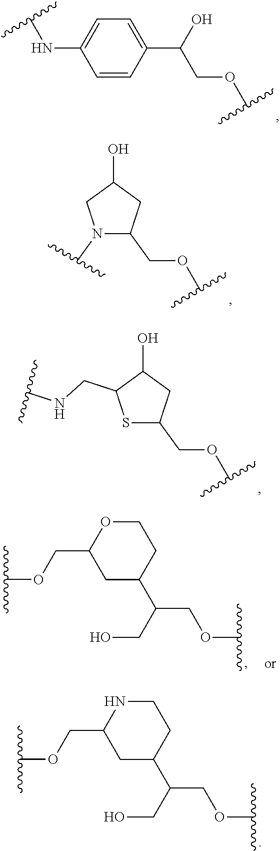 [SMILES: *Nc1ccc(C(O)CO*)cc1 ; *OCC1CC(O)CN1* ; *NCC1SC(CO*)CC1O ; *OCC1CC(C(CO)CO*)CCO1 ; *OCC1CC(C(CO)CO*)CCN1]

In certain embodiments, in Formula (I) or (II), L^{3c} is

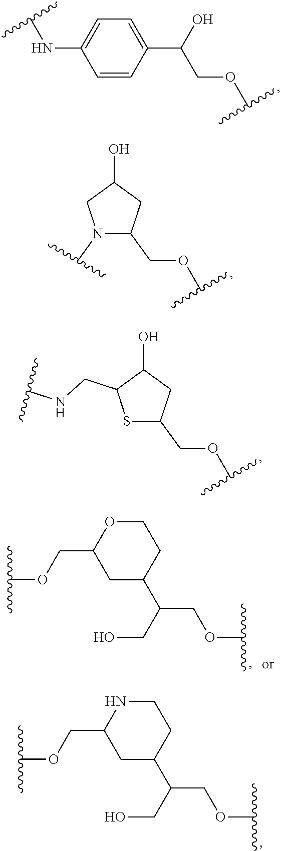

wherein L^{3c} is attached to a 3′-terminus of an oligonucleotide.

In certain embodiments, in Formula (I) or (II), L^{3c} is

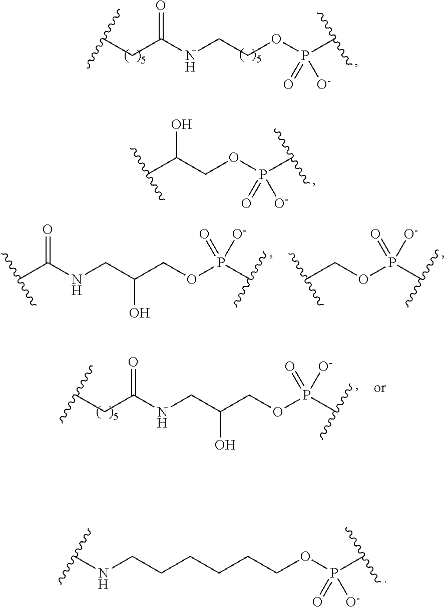

In certain embodiments, in Formula (I) or (II), M is N or CH. In certain embodiments, in Formula (I) or (II), M is N. In certain embodiments, in Formula (I) or (II), M is CH.

In certain embodiments, in Formula (I) or (II), M is trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl, each optionally substituted with one or more substituents Q. In certain embodiments, in Formula (I) or (II), M is trivalent C₁₋₆ alkyl, optionally substituted with one or more substituents Q. In certain embodiments, in Formula (I) or (II), M is CR^{m}, wherein R^{m} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more Q. In certain embodiments, in Formula (I) or (II), M is CR^{m}, wherein R^{m} is C₁₋₆ alkyl, optionally substituted with one or more Q. In certain embodiments, in Formula (I) or (II), M is C(CH₃). In certain embodiments, in Formula (I) or (II), M is trivalent C₁₋₆ heteroalkyl, optionally substituted with one or more substituents Q. In certain embodiments, in Formula (I) or (II), M is trivalent C₁₋₆ alkenyl, optionally substituted with one or more substituents Q. In certain embodiments, in Formula (I) or (II), M is trivalent C₃₋₁₀ cycloalkyl, optionally substituted with one or more substituents Q. In certain embodiments, in Formula (I) or (II), M is trivalent C₆₋₁₄ aryl, optionally substituted with one or more substituents Q. In certain embodiments, in Formula (I) or (II), M is trivalent heteroaryl, optionally substituted with one or more substituents Q. In certain embodiments, in Formula (I) or (II), M is trivalent heterocyclyl, optionally substituted with one or more substituents Q.

In certain embodiments, in Formula (I) or (II), L¹ is —NHC(O)(CH₂)ₑC(O)NH—, wherein e is an integer of 1, 2, 3, 4, 5, 6, 7, 8, 9, 10, 11, 12, 13, 14, or 15; L² is —(CH₂)_{f}(C(O)NH(CH₂)_{g}OP(O₂)—, wherein f is an integer of 1, 2, 3, 4, 5, or 6; and g is an integer of 1, 2, 3, 4, 5, 6, 7, 8, 9, or 10; L^{3a} is methylene or benzdiyl, each optionally substituted with one or more substituents Q; L^{3c} is —(CH₂)ₘC(O)NH(CH₂)ₙOP(O₂)—, wherein m and n are each independently an integer of 1, 2, 3, 4, 5, 6, 7, 8, 9, or 10; and M is trivalent C₁₋₆ alkyl,

In one embodiment, provided herein is a carbohydrate-oligonucleotide conjugate of:

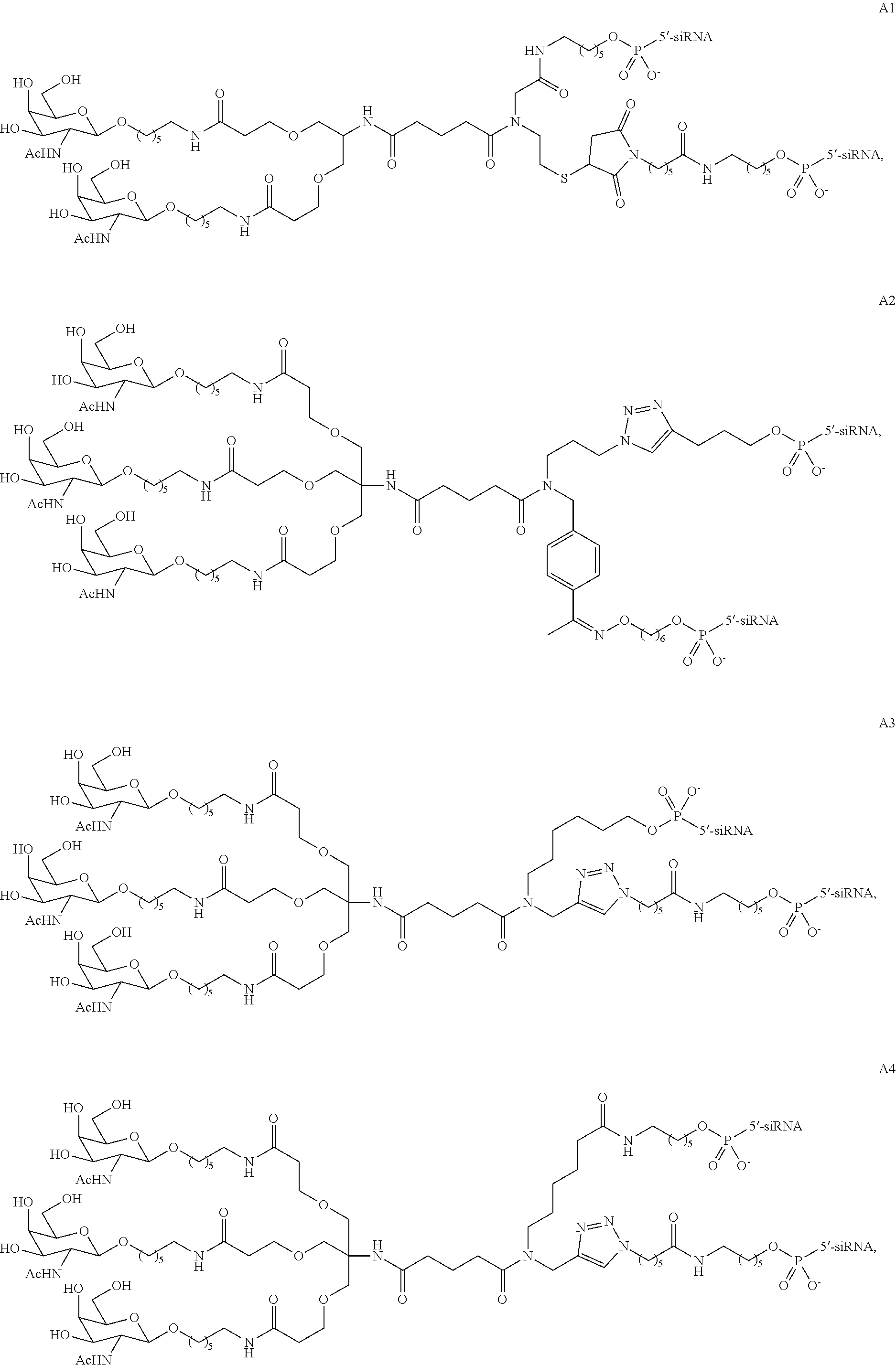

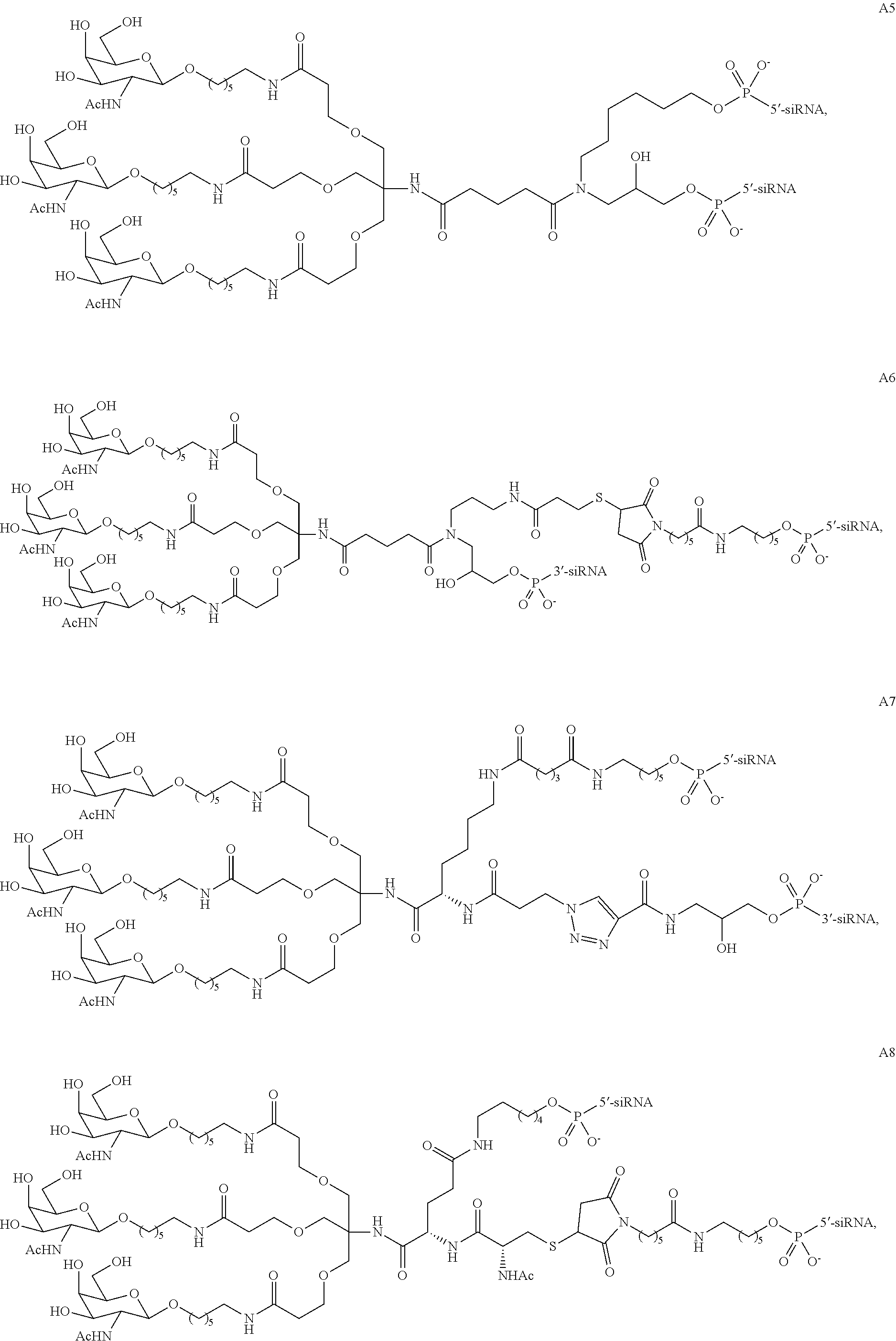

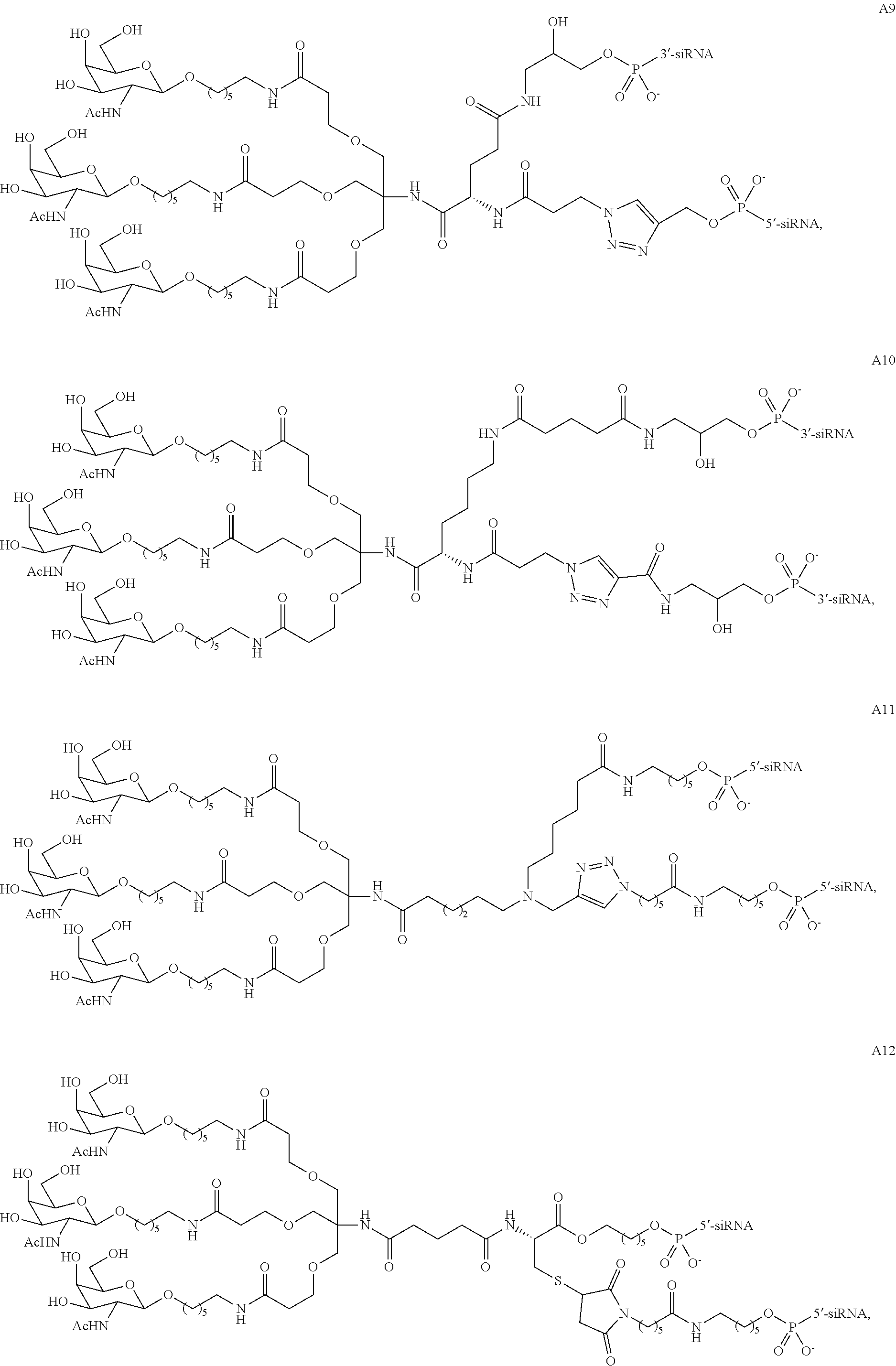

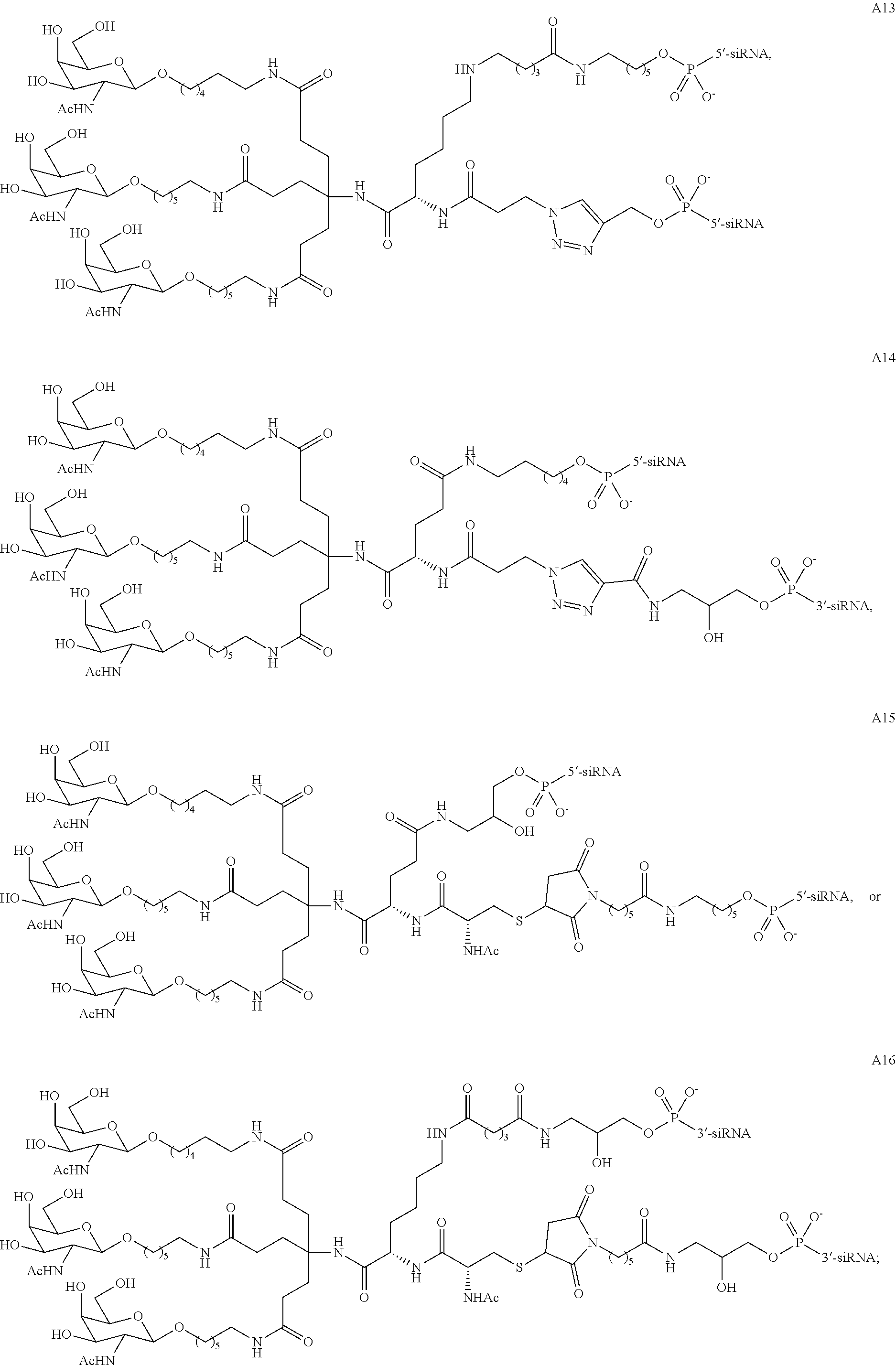

wherein each 3′-siRNA independently represents a siRNA with one of its 3′-termini connected to a trivalent linker; and each 5′-siRNA independently represents a siRNA with one of its 5′-termini connected to a trivalent linker.

In another embodiment, provided herein is a carbohydrate-oligonucleotide of:

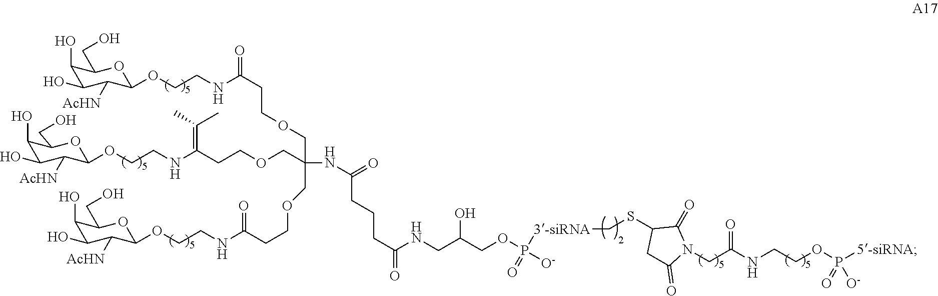

wherein 3′-siRNA represents a siRNA with one of its 3′-termini connected to a first divalent linker and one of its 5′-termini connected to a second divalent linker; and 5′-siRNA represents a siRNA with one of its 5′-termini connected to the second divalent linker.

In yet another embodiment, provided herein is a carbohydrate-oligonucleotide of:

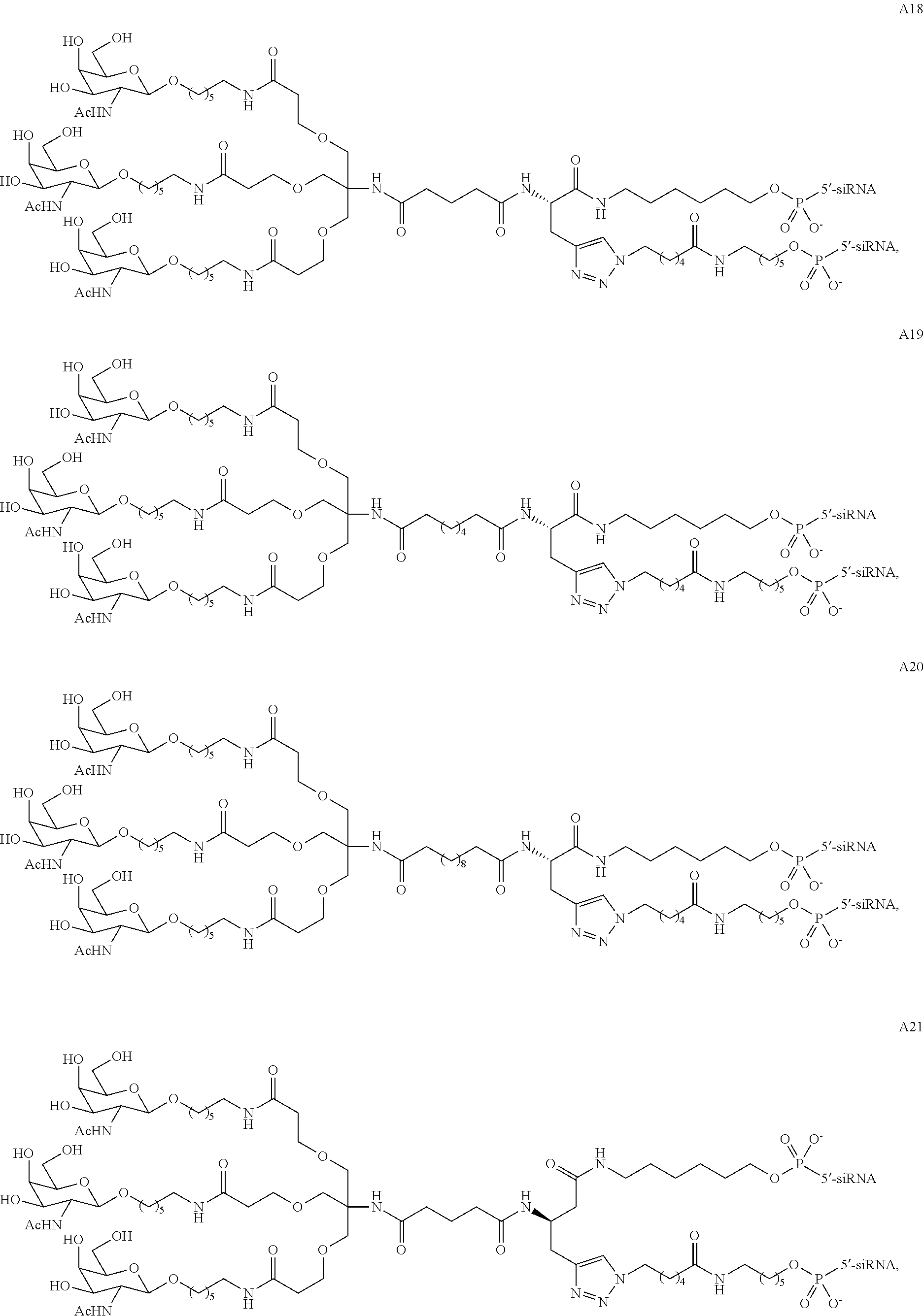

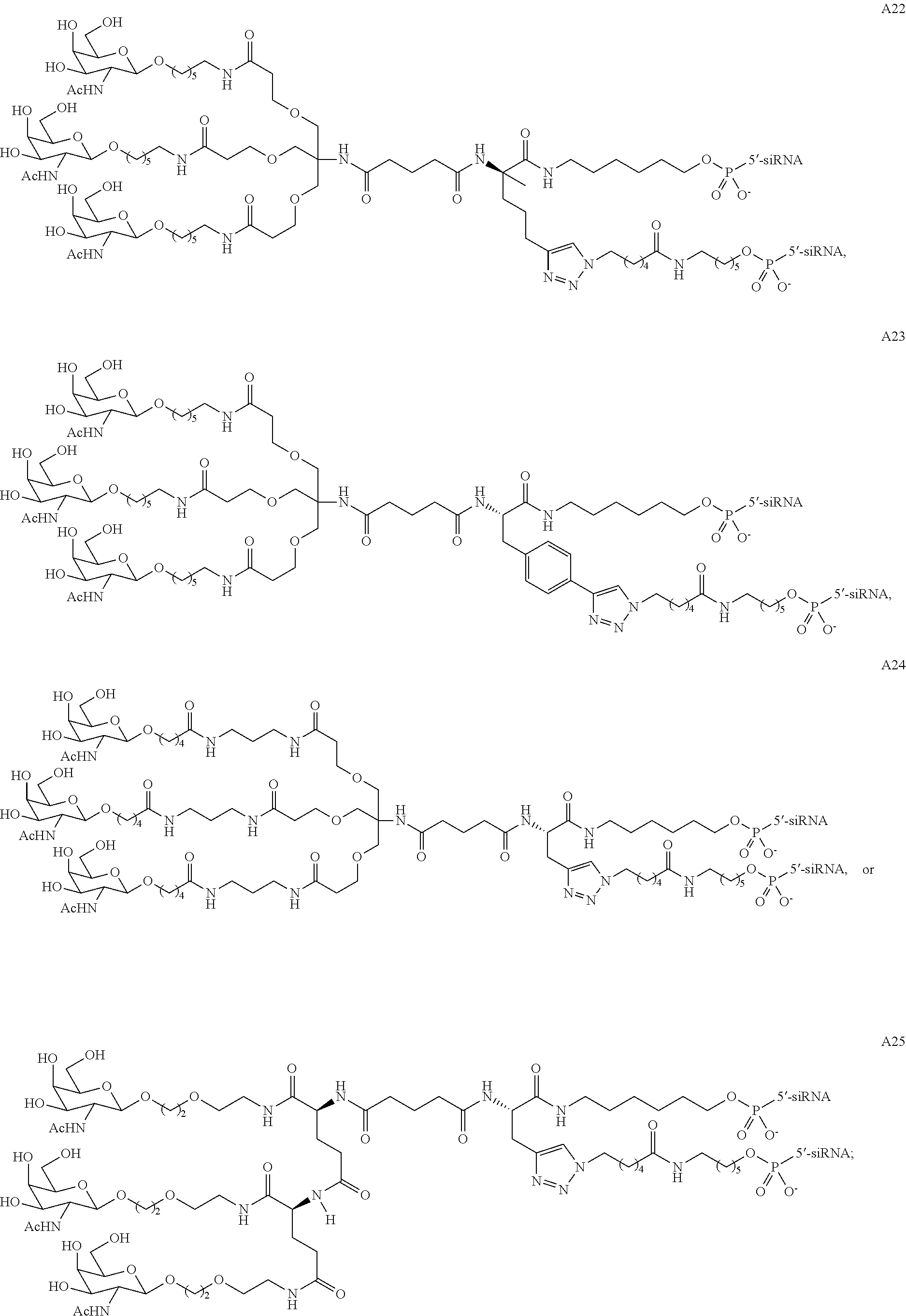

wherein each 5′-siRNA independently represents a siRNA with one of its 5′-termini connected to a trivalent linker.

In yet another embodiment, provided herein is a carbohydrate-oligonucleotide conjugate of:

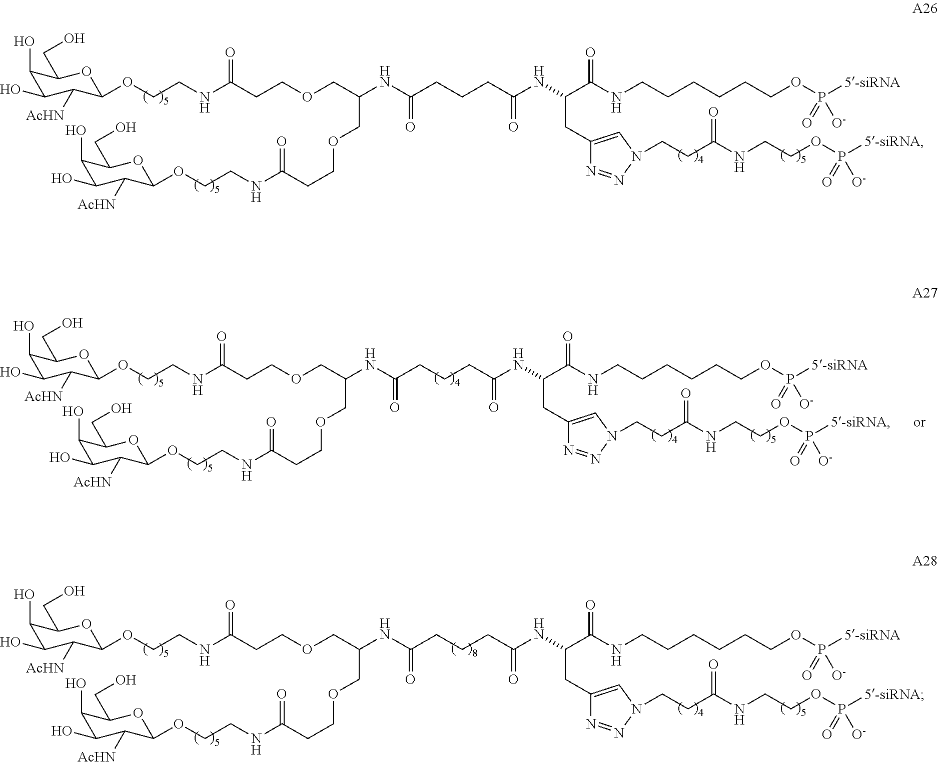

wherein each 5′-siRNA independently represents a siRNA with one of its 5′-termini connected to a trivalent linker.

In yet another embodiment, provided herein is a carbohydrate-oligonucleotide conjugate of:

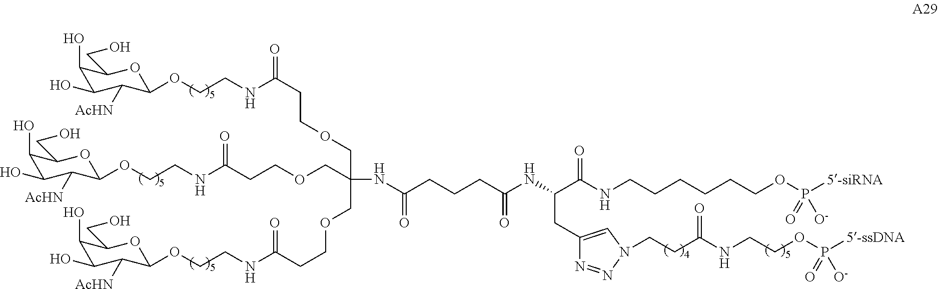

wherein 5′-siRNA represents a siRNA with one of its 5′-termini connected to a trivalent linker; and 5′-ssDNA represents a single stranded DNA with its 5′-terminus connected to the trivalent linker.

In yet another embodiment, provided herein is a carbohydrate-oligonucleotide conjugate of:

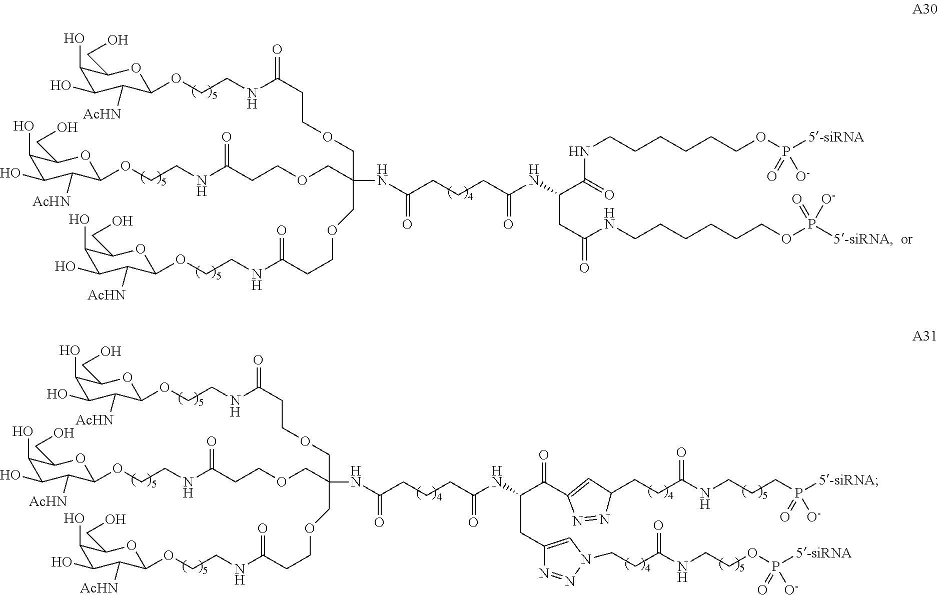

wherein each 5′-siRNA independently represents a siRNA with one of its 5′-termini connected to a trivalent linker.

In still another embodiment, provided herein is a carbohydrate-oligonucleotide conjugate of:

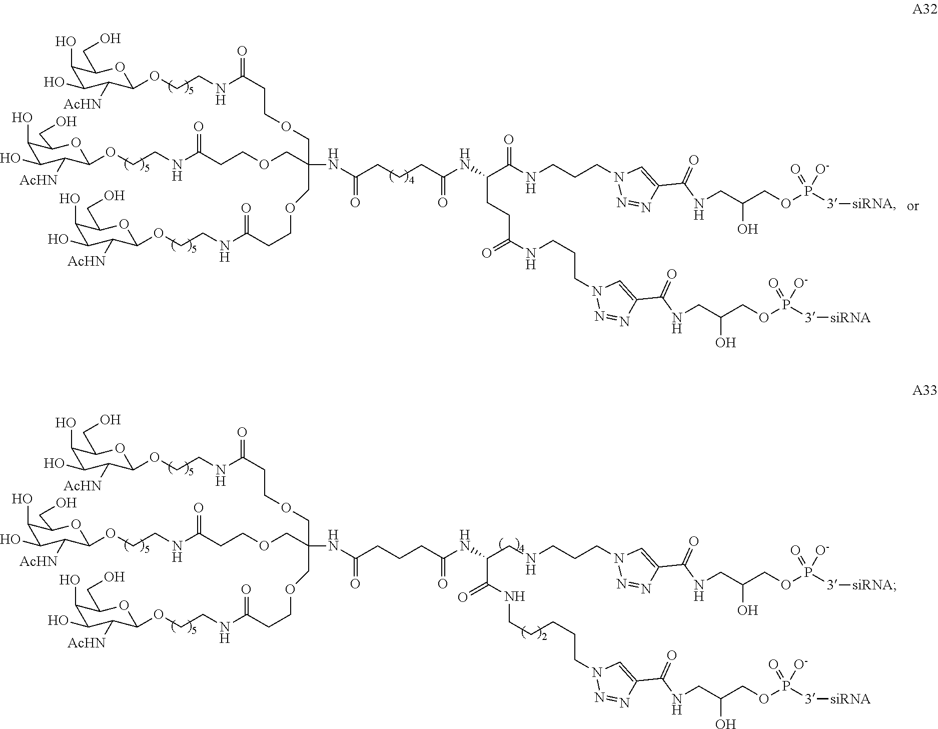

wherein each 3′-siRNA independently represents a siRNA with one of its 3′-termini connected to a trivalent linker.

In one embodiment, provided herein is a carbohydrate-oligonucleotide conjugate of:

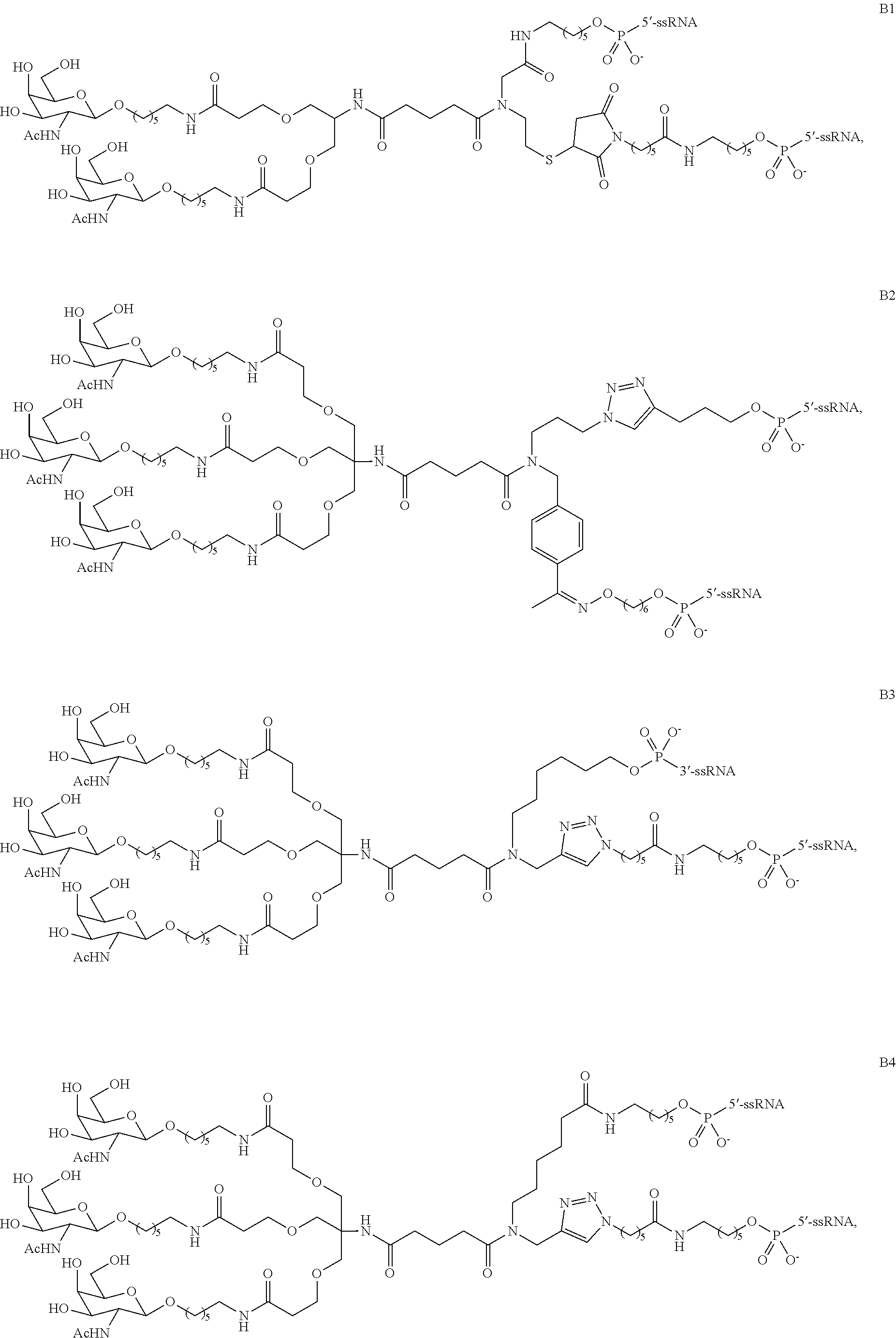

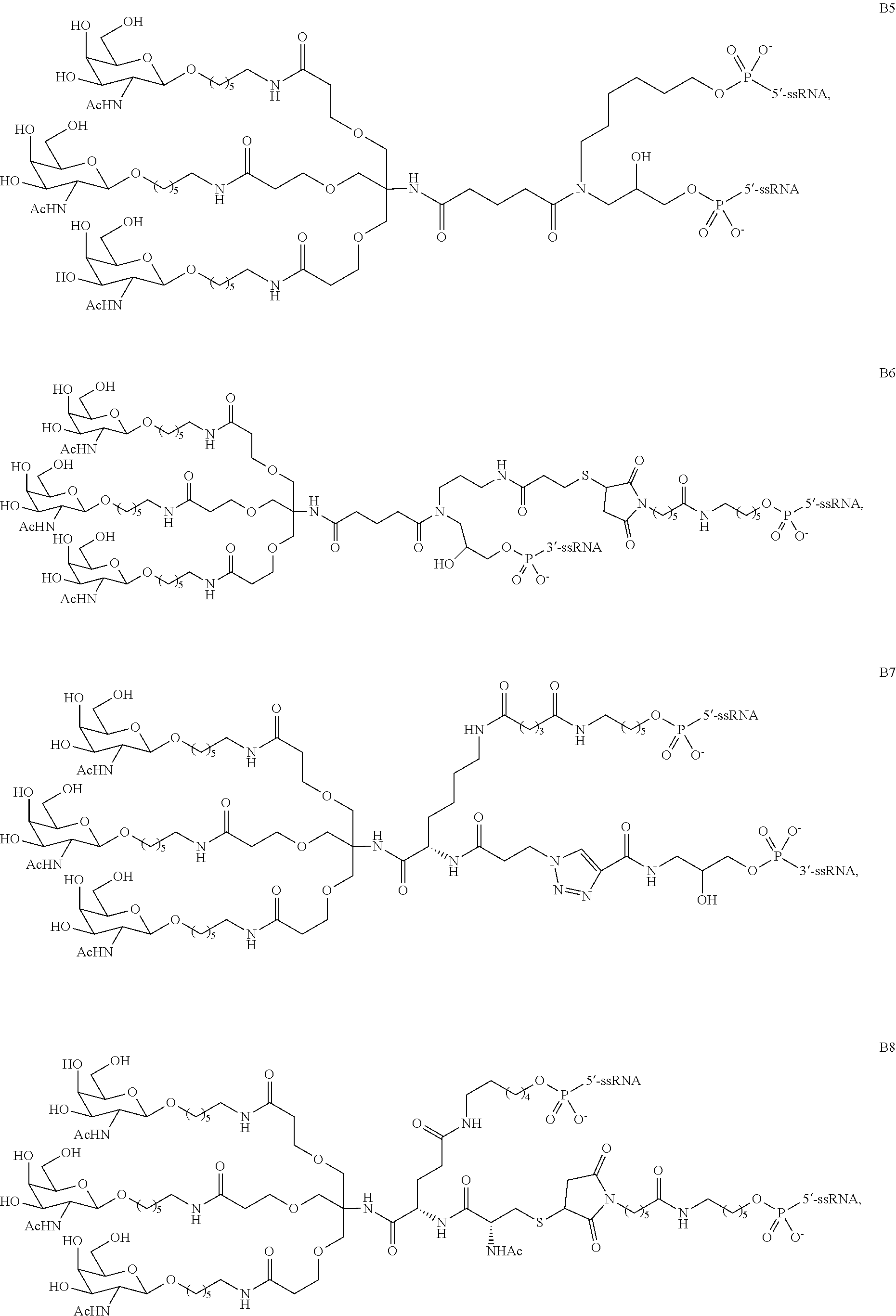

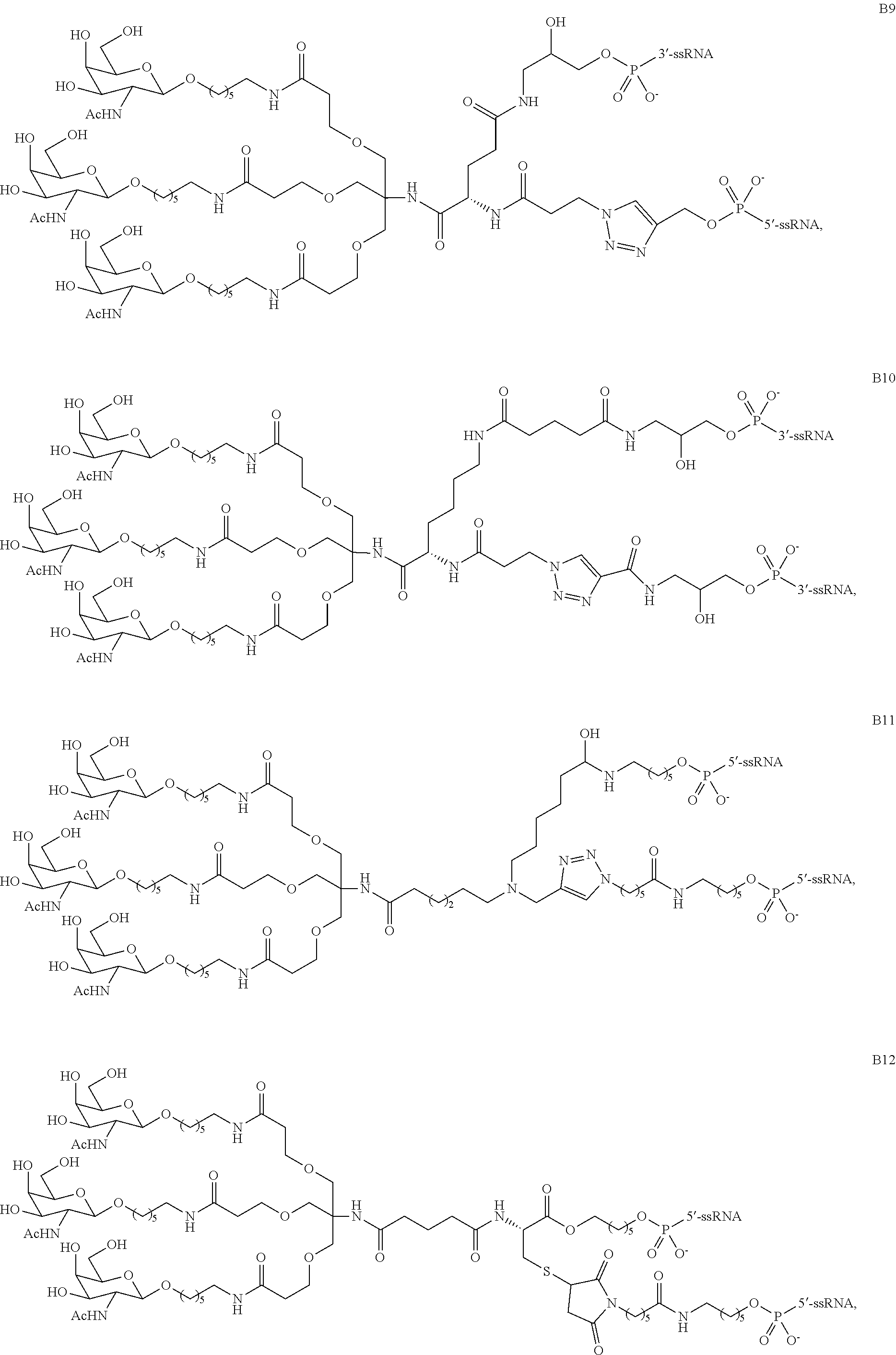

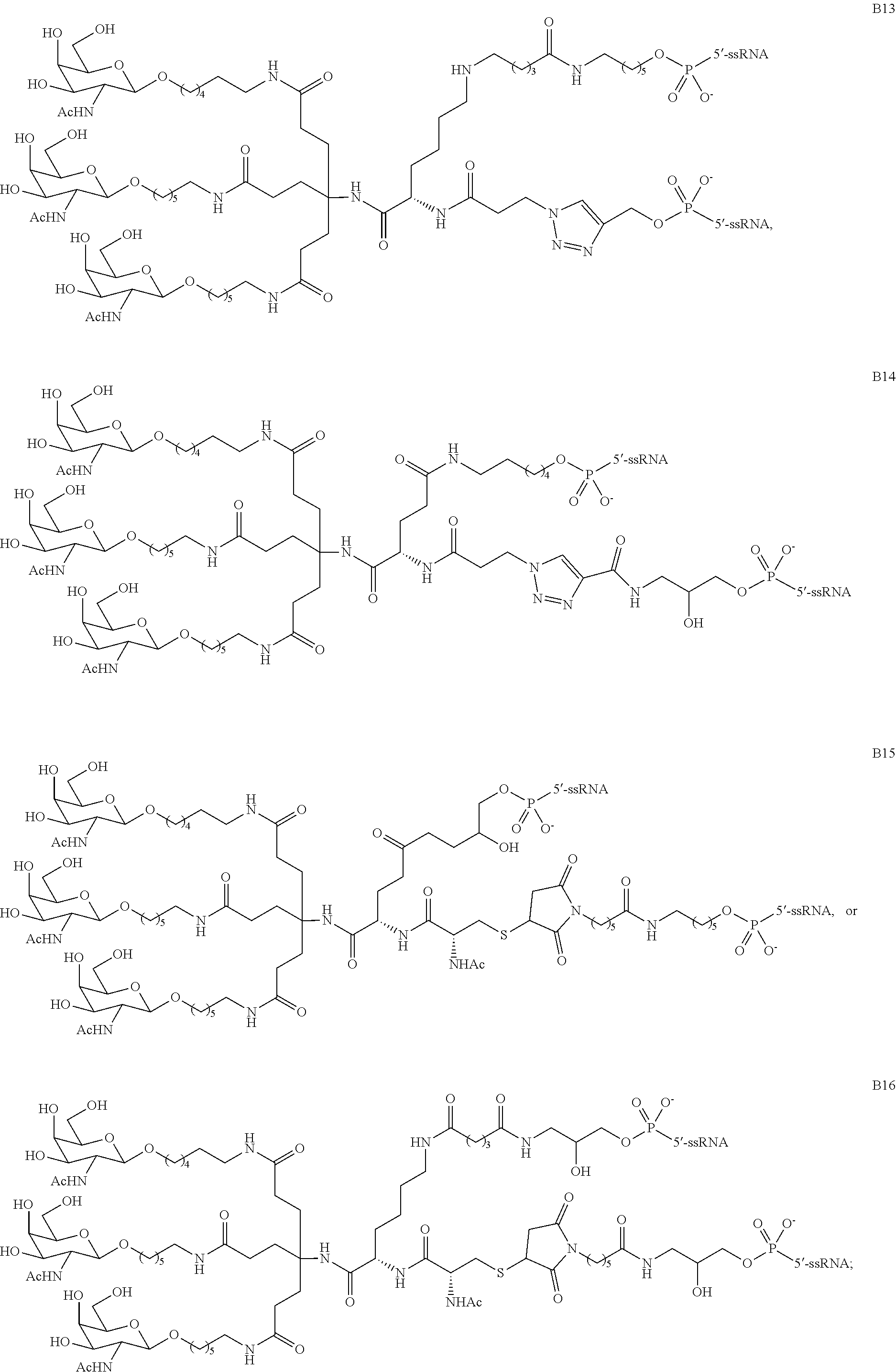

wherein each 3′-ssRNA represents independently a single-stranded RNA with its 3′-terminus connected to a trivalent linker; and each 5′-ssRNA independently represents a single-stranded RNA with its 5′-terminus connected to a trivalent linker.

In another embodiment, provided herein is a carbohydrate-oligonucleotide conjugate of:

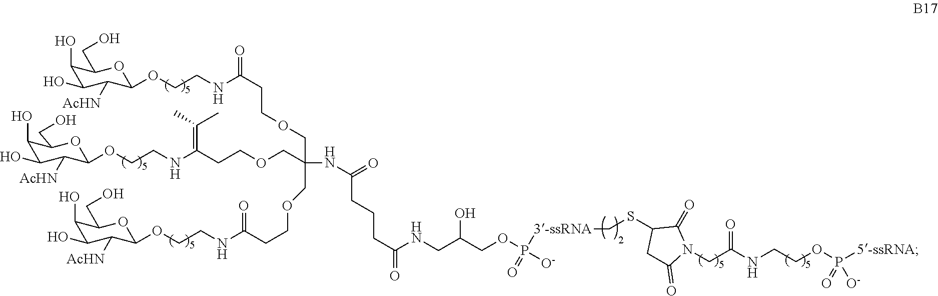

wherein 3′-ssRNA represents a single-stranded RNA with its 3′-terminus connected to a first divalent linker and its 5′-terminus connected to a second divalent linker; and 5′-ssRNA represents a single-stranded RNA with its 5′-terminnus connected to the second divalent linker.

In yet another embodiment, provided herein is a carbohydrate-oligonucleotide conjugate of:

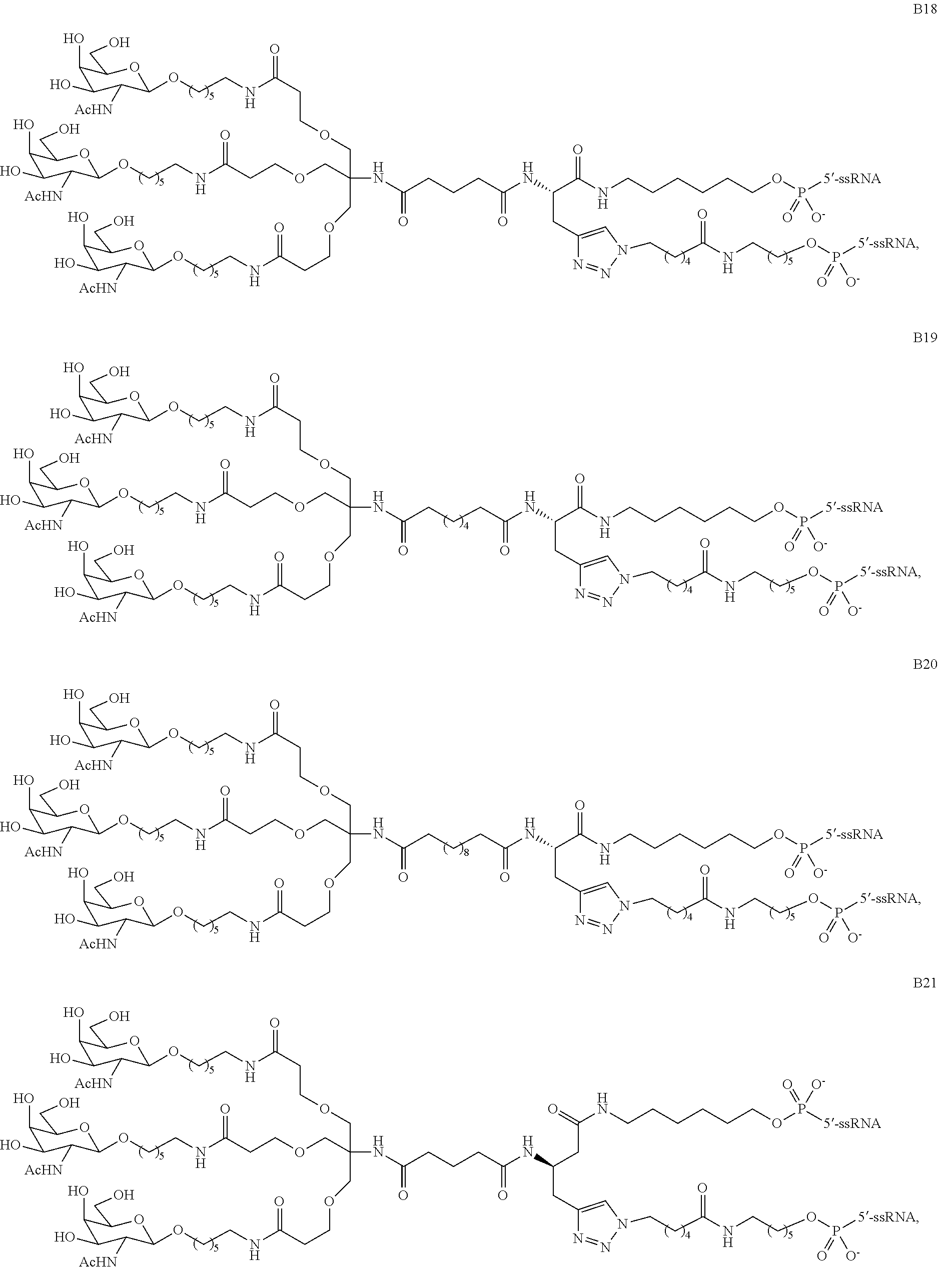

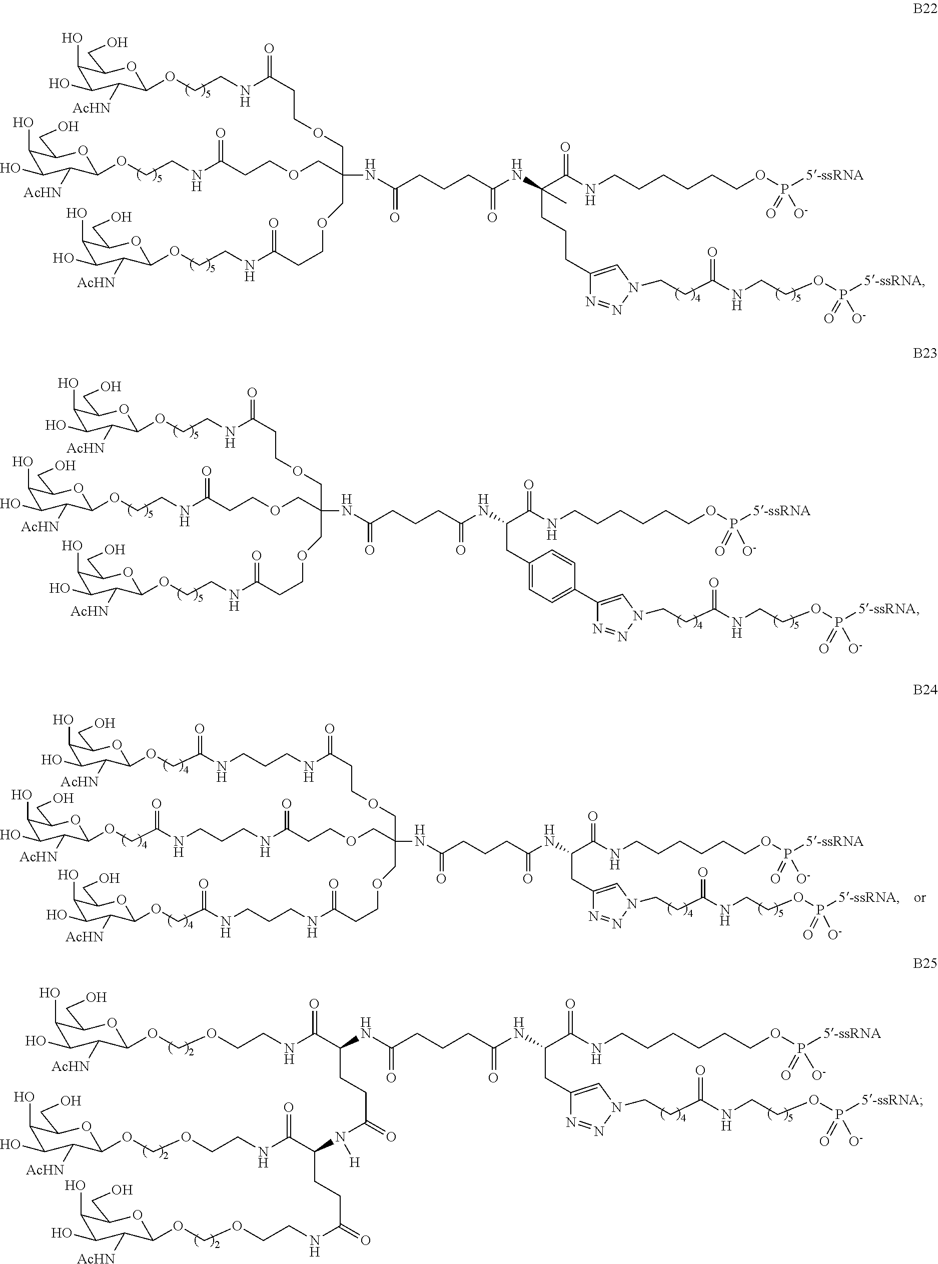

wherein each 5′-ssRNA independently represents a single-stranded RNA with its 5′-terminus connected to a trivalent linker.

In yet another embodiment, provided herein is a carbohydrate-oligonucleotide conjugate of:

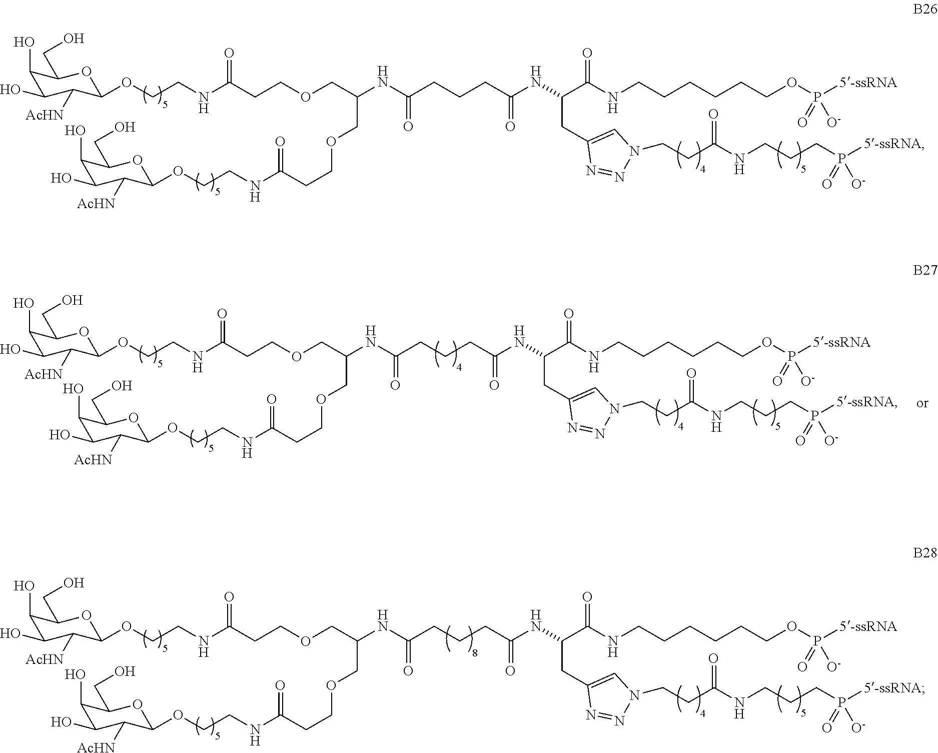

wherein each 5′-ssRNA independently represents a single-stranded RNA with its 5′-terminus connected to a trivalent linker.

In yet another embodiment, provided herein is a carbohydrate-oligonucleotide conjugate of:

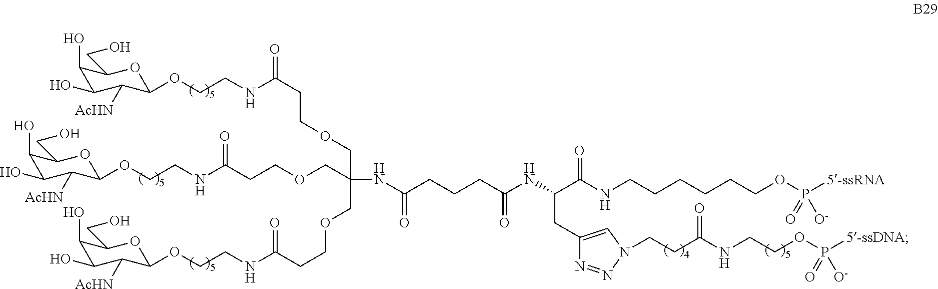

wherein 5′-siRNA represents a single-stranded RNA with its 5′-terminus connected to a trivalent linker; and 5′-ssDNA represents a single stranded DNA with its 5′-terminus connected to the trivalent linker.

In yet another embodiment, provided herein is a carbohydrate-oligonucleotide conjugate of:

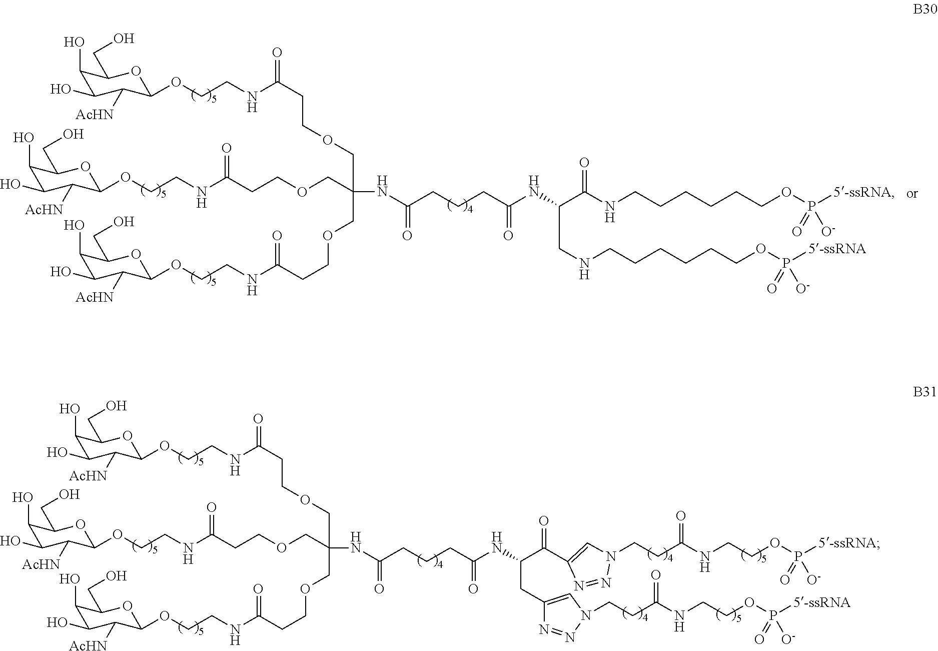

wherein each 5′-ssRNA independently represents a single-stranded RNA with its 5′-terminus connected to a trivalent linker.

In still another embodiment, provided herein is a carbohydrate-oligonucleotide conjugate of:

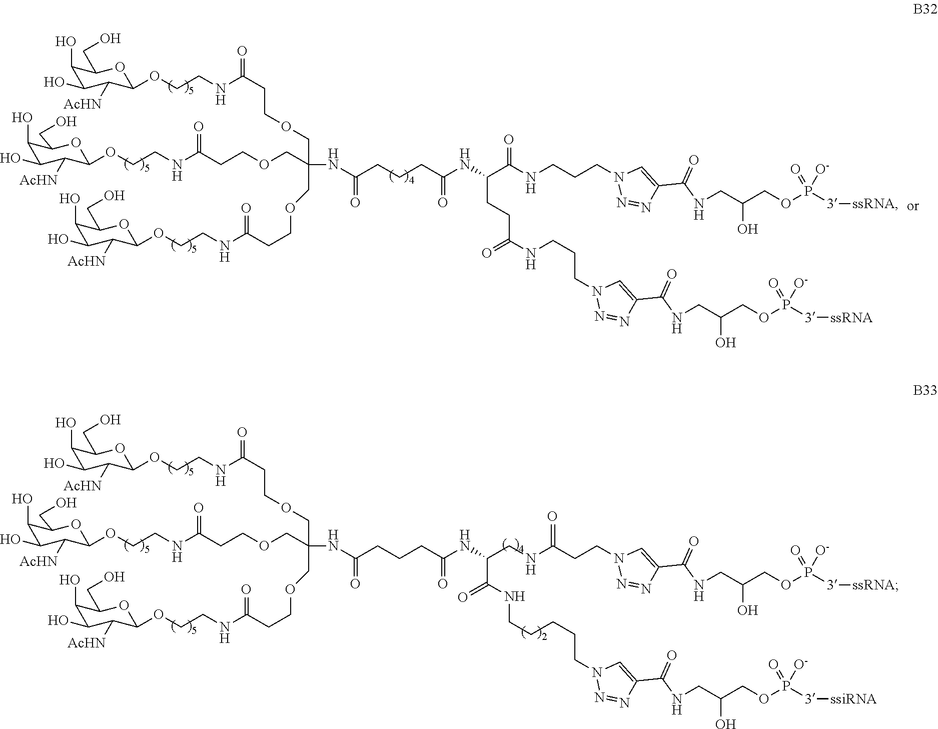

wherein each 3′-ssRNA independently represents a single-stranded RNA with its 3′-terminus connected to a trivalent linker.

In one embodiment, provided herein is a carbohydrate-oligonucleotide conjugate that is any one of carbohydrate-oligonucleotide conjugates A18a to A18c.

In another embodiment, provided herein is a carbohydrate-oligonucleotide conjugate that is any one of carbohydrate-oligonucleotide conjugates A4a, A18a to A18h, and A19a to A29a.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 1 and 2; and the second oligonucleotide duplex comprises the nucleotide sequences of SEQ ID NOs: 3 and 4.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 5 and 6; and the second oligonucleotide duplex comprises the nucleotide sequences of SEQ ID NOs: 7 and 8.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 9 and 10; and the second oligonucleotide duplex comprises the nucleotide sequences of SEQ ID NOs: 11 and 12.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 13 and 14; and the second oligonucleotide duplex comprises the nucleotide sequences of SEQ ID NOs: 7 and 8.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 15 and 16; and the second oligonucleotide duplex comprises the nucleotide sequences of SEQ ID NOs: 17 and 18.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 9 and 10; and the second oligonucleotide duplex comprises the nucleotide sequences of SEQ ID NOs: 19 and 20.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 19 and 20; and the second oligonucleotide duplex comprises the nucleotide sequences of SEQ ID NOs: 21 and 22.

In certain embodiments, a carbohydrate-oligonucleotide conjugate provided herein comprises first and second oligonucleotide duplexes, wherein the first oligonucleotide duplex comprises the nucleotide sequences of SEQ ID NOs: 3 and 4; and the second oligonucleotide duplex comprises the nucleotide sequences of SEQ ID NOs: 1 and 2.

In certain embodiments, a carbohydrate-oligonucleotide conjugate provided herein comprises an oligonucleotide duplex and a single-stranded oligonucleotide, wherein the oligonucleotide duplex comprises the nucleotide sequences of SEQ ID NOs: 23 and 24; and the single-stranded oligonucleotide comprises the nucleotide sequence of SEQ ID NO: 25.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 1; and the second single-stranded oligonucleotide comprises the nucleotide sequence of SEQ ID NO: 3.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 5; and the second single-stranded oligonucleotide comprises the nucleotide sequence of SEQ ID NO: 7.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 9; and the second single-stranded oligonucleotide comprises the nucleotide sequence of SEQ ID NO: 11.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 13; and the second single-stranded oligonucleotide comprises the nucleotide sequence of SEQ ID NO: 7.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 15; and the second single-stranded oligonucleotide comprises the nucleotide sequence of SEQ ID NO: 17.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 9; and the second single-stranded oligonucleotide comprises the nucleotide sequence of SEQ ID NO: 19.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 19; and the second single-stranded oligonucleotide comprises the nucleotide sequence of SEQ ID NO: 21.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 3; and the second single-stranded oligonucleotide comprises the nucleotide sequence of SEQ ID NO: 1.

In certain embodiments, a carbohydrate-oligonucleotide conjugate provided herein comprises first and second single-stranded oligonucleotides, wherein the first single-stranded oligonucleotide comprises the nucleotide sequence of SEQ ID NO: 23; and the second single-stranded oligonucleotide comprises the nucleotide sequence of SEQ ID NO: 25.

### Pharmaceutical Compositions

In one embodiment, provided herein is a pharmaceutical composition, comprising a carbohydrate-oligonucleotide conjugate provided herein and a pharmaceutically acceptable excipient.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intramuscular administration. In still another embodiment, the pharmaceutical composition provided herein is formulated in a dosage form for subcutaneous administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a carbohydrate-oligonucleotide conjugate provided herein) sufficient to produce the desired therapeutic effect, in association with the required pharmaceutical excipient(s). Examples of a unit-dosage form include, but are not limited to, an ampoule and syringe. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); min (minute or minutes); h (hour or hours); AcOH (acetic acid); DEA (diethylamine); DIPEA (N,N-diisopropylethyl-amine); DMSO (dimethyl sulfoxide); DMTr (4,4-dimethoxytrityl); EDCI (1-ethyl-3-(3-dimethyl-aminopropyl) carbodiimide); ETT (5-(ethylthio)tetrazole); HBTU (2-(1H-benzotriazole-1-yl)-1,1,3,3-tetramethyluronium hexafluorophosphate); HOSu (N-hydroxy-succinimide); NaOAc (sodium acetate); iPr (isopropyl); Pfp (pentafluorophenyl); SDPD (succinimidyl 3-(2-pyridyl-dithio) propionate); TBAF (tetrabutylammonium fluoride); TBDPS (tert-butyldiphenylsilyl); TCEP (tris(2-carboxyethyl) phosphine); TFAPFP (pentafluorophenyl trifluoroacetate); THF (tetrahydrofuran); MS (mass spectrometry); and prep-HPLC (preparative high performance liquid chromatography).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of GalNAc-siRNA Conjugate A1

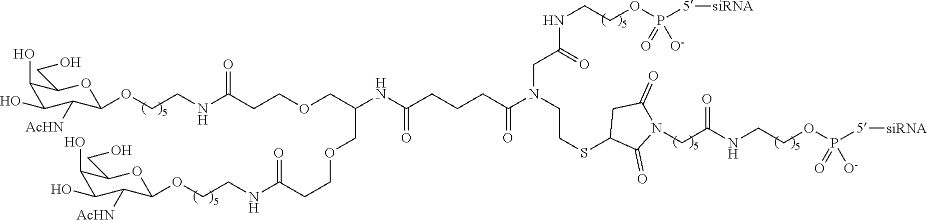

GalNAc-siRNA conjugate A1 is prepared as shown in Scheme 1, wherein R^{p} is a moiety having the structure of

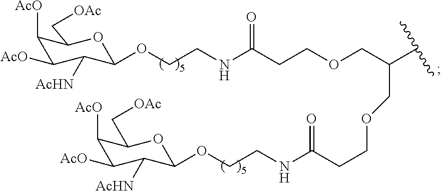

R^{q} is an ASGPR binding moiety having the structure of

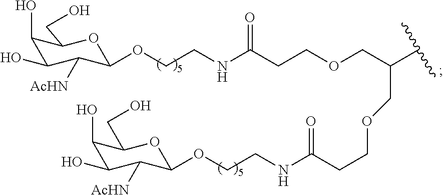

each 5′-ssRNA independently represents a single-stranded RNA (e.g., a sense or antisense sequence of a siRNA, or an ASO sequence) with its 5′-terminus connected to a trivalent linker; and each 5′-siRNA independently represents a siRNA with one of its 5′-termini connected to a trivalent linker. In Scheme 1, compound 1.2 is prepared similarly as described in WO 2020/259497 A1, the disclosure of which is incorporated herein by reference in its entirety.

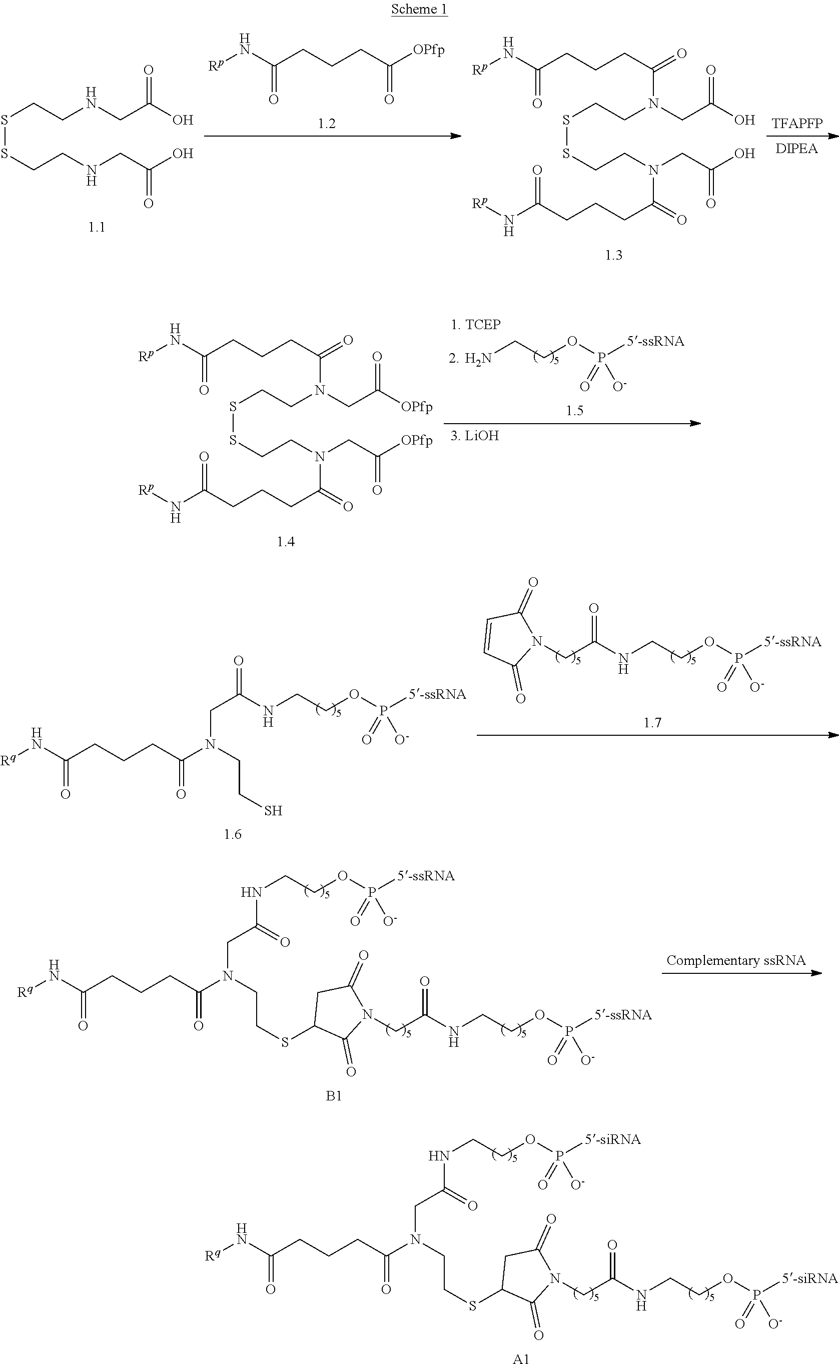

### Example 2

### Preparation of GalNAc-siRNA Conjugate A2

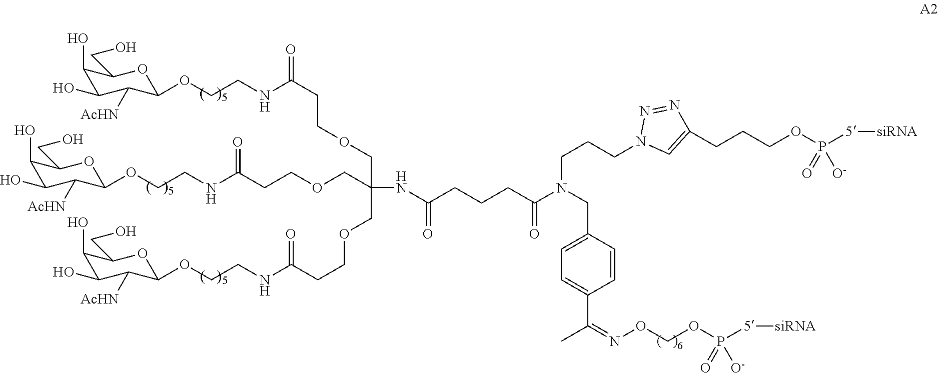

GalNAc-siRNA conjugate A2 is prepared as shown in Scheme 2, wherein R^{g} is a moiety having the structure of

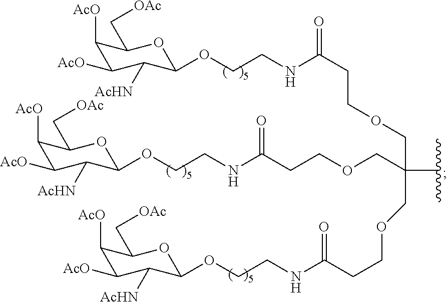

R^{t} is an ASGPR binding moiety having the structure of

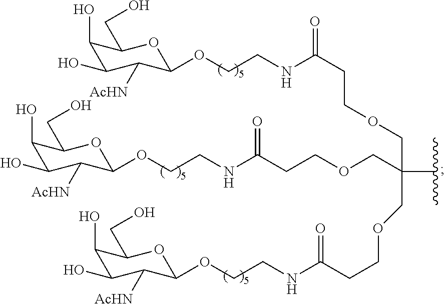

each 5′-ssRNA independently represents a single-stranded RNA (e.g., a sense or antisense sequence of a siRNA, or an ASO sequence) with its 5′-terminus connected to a trivalent linker; and each 5′-siRNA independently represents a siRNA with one of its 5′-termini connected to a trivalent linker. In Scheme 2, compound 2.2 is prepared similarly as described in WO 2020/259497 A1, the disclosure of which is incorporated herein by reference in its entirety.

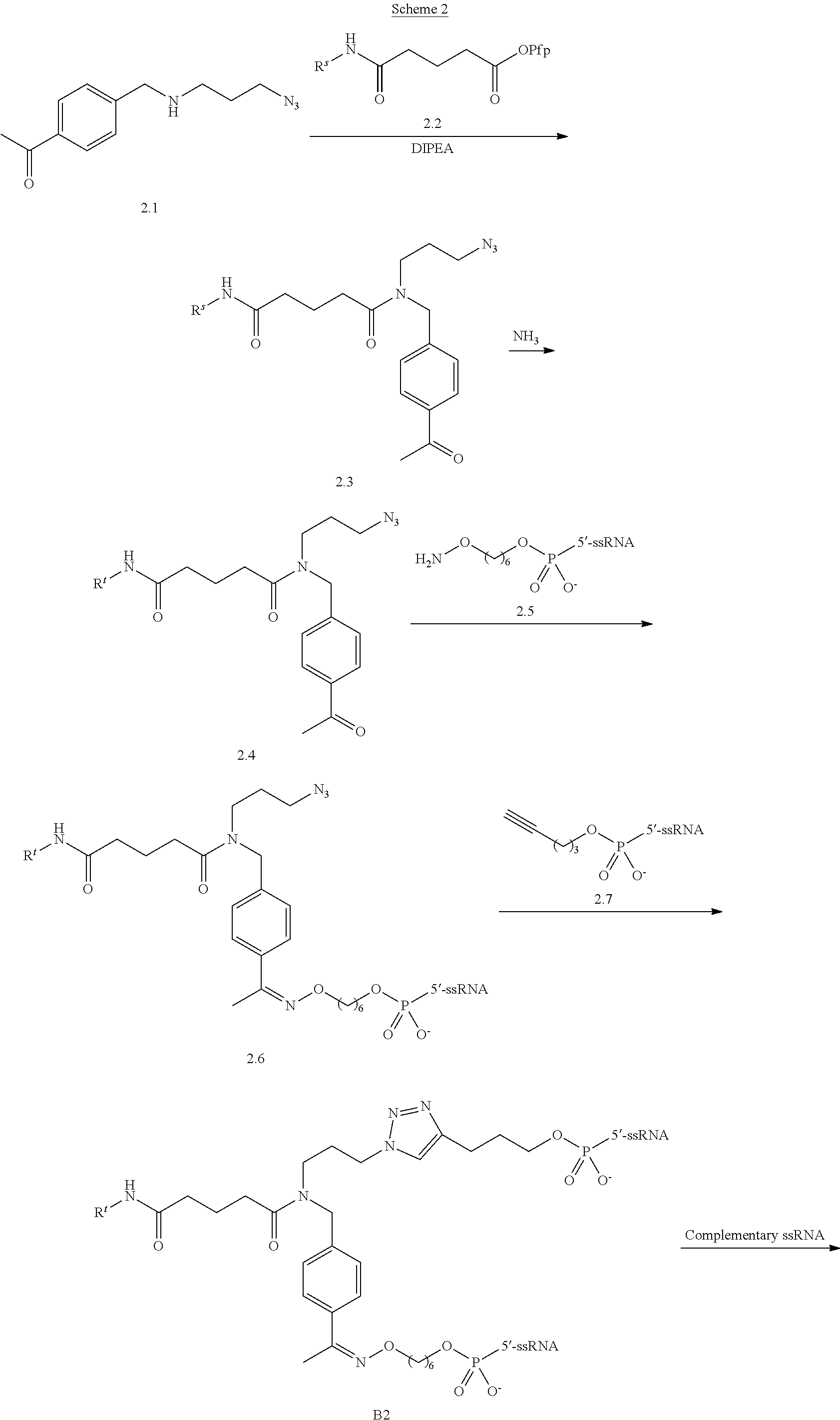

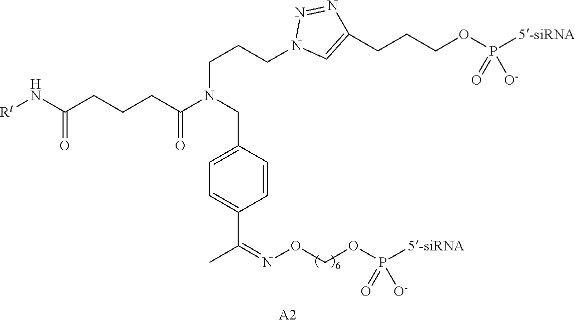

### Example 3

### Preparation of GalNAc-siRNA Conjugate A3

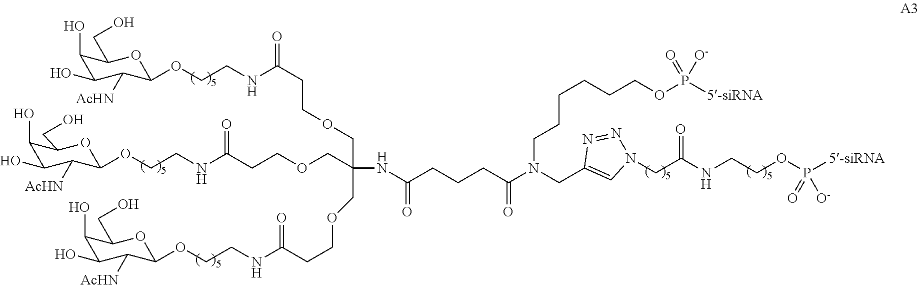

GalNAc-siRNA conjugate A3 is prepared as shown in Scheme 3, wherein R^{s} and R^{t} are as described in Example 2; each 5′-ssRNA independently represents a single-stranded RNA (e.g., a sense or antisense sequence of a siRNA, or an ASO sequence) with its 5′-terminus connected to a trivalent linker; and each 5′-siRNA independently represents a siRNA with one of its 5′-termini connected to a trivalent linker.

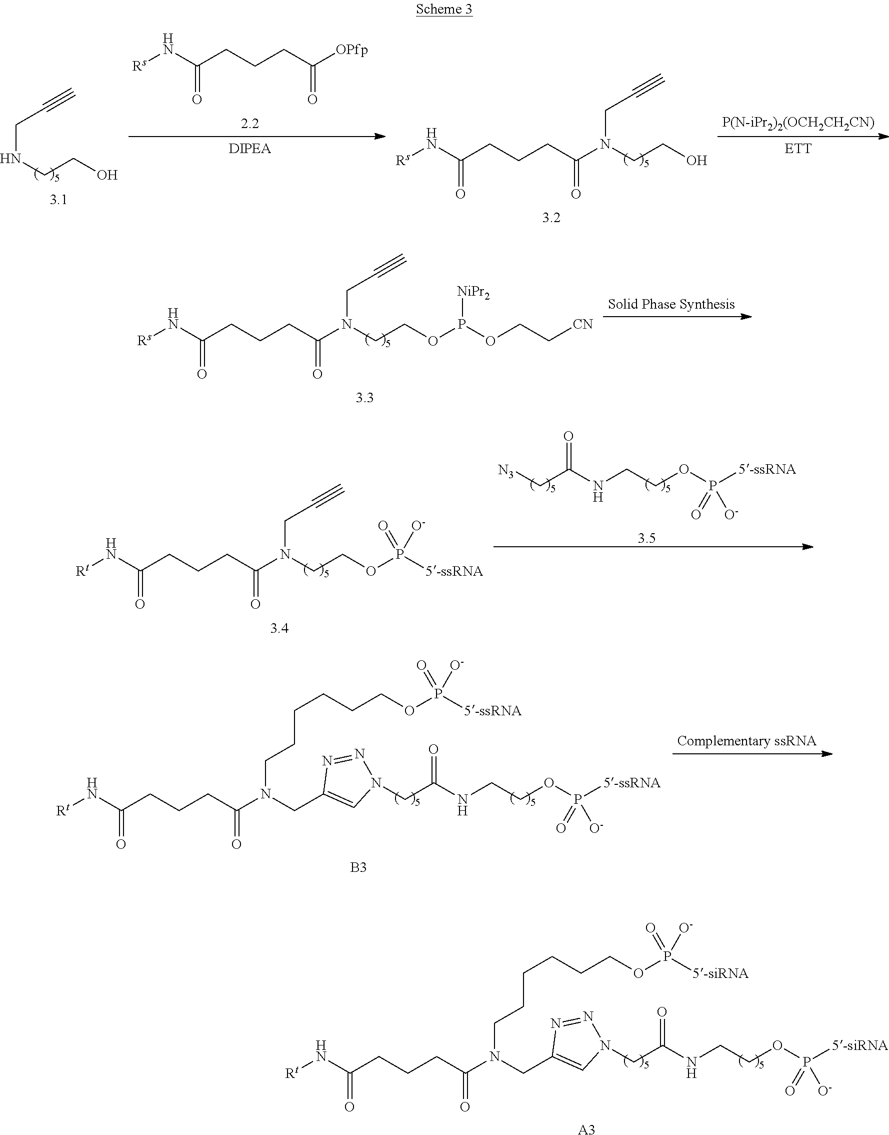

### Example 4

### Preparation of GalNAc-siRNA Conjugate A4a

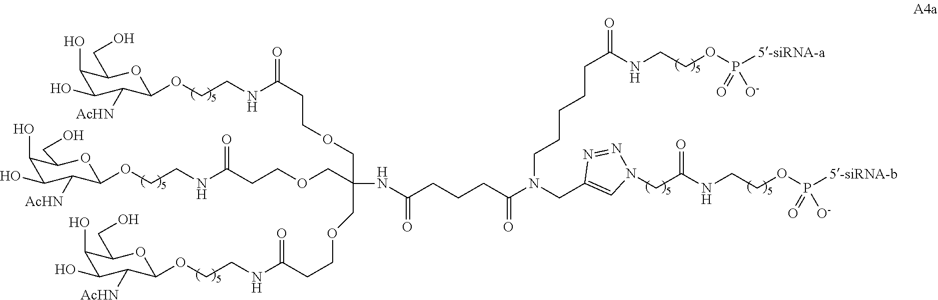

GalNAc-siRNA conjugate A4a was prepared as shown in Schemes 4A and 4B, wherein R^{s} and R^{t} are as described in Example 2; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to a trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to a trivalent linker and an antisense strand having the nucleotide sequence of the nucleotide sequence of SEQ ID NO: 4.

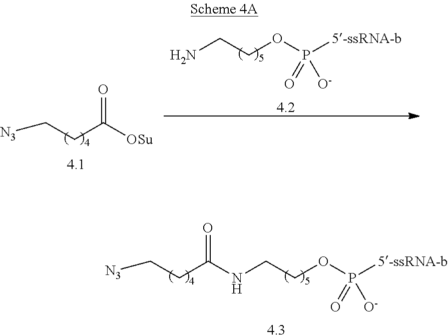

In Schemes 4A and 4B, the oligonucleotides of compounds 4.2 (SEQ ID NO: 1), 4.5 (SEQ ID NO: 3), 4.9 (SEQ ID NO: 2), and 4.10 (SEQ ID NO: 4) were prepared by solid-phase oligonucleotide synthesis.

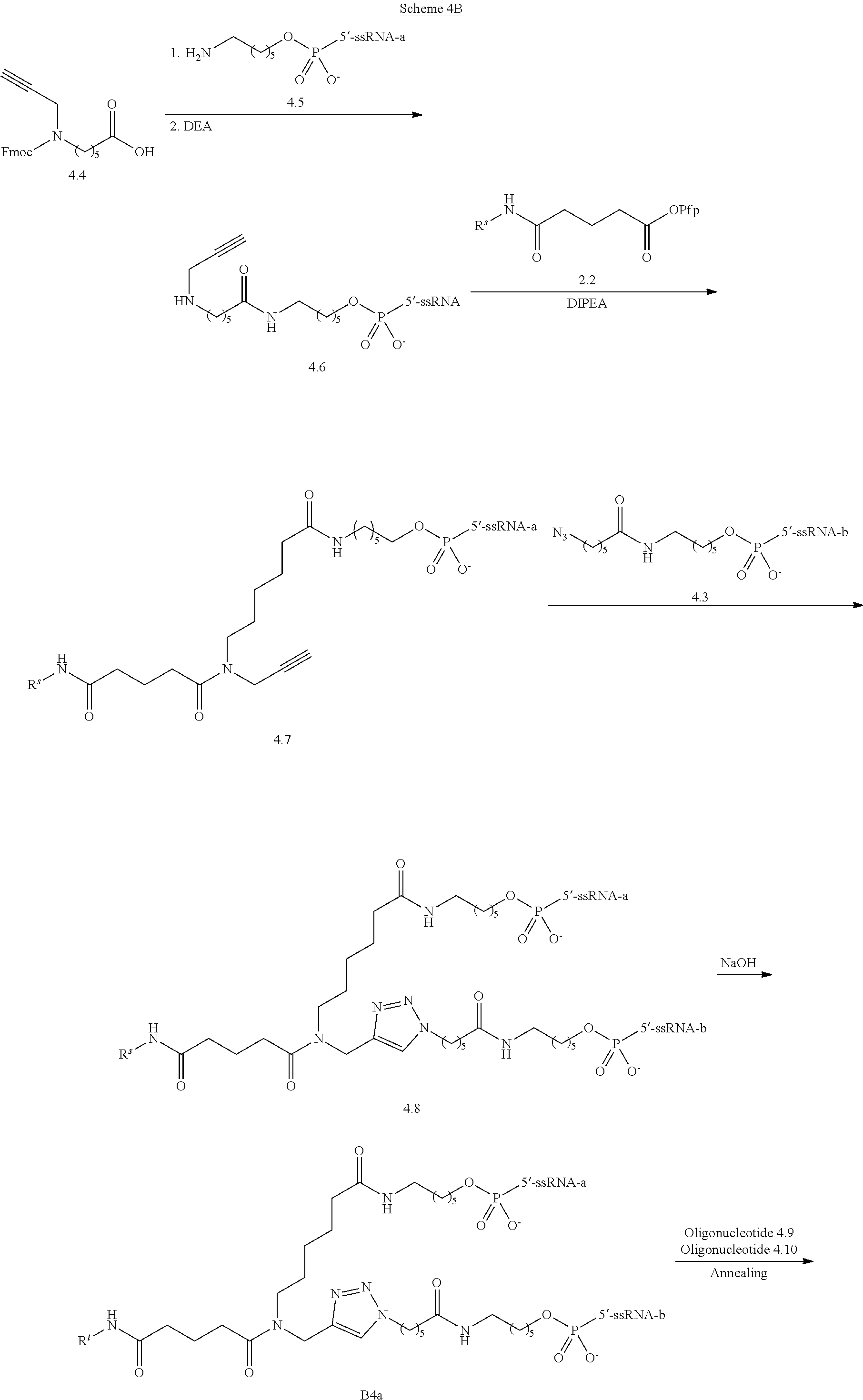

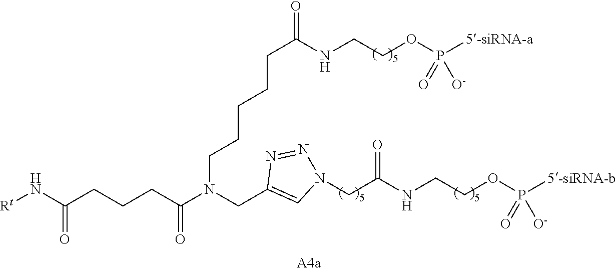

Preparation of compound 4.3. To a solution of compound 4.2 (210 mg, 29 μmol) in 0.1 M aqueous Na₂B₃O₇ solution (1 mL) was added compound 4.1 (37 mg, 147 μmol) in DMSO (1 mL). After the reaction mixture was sonicated for 2 h, NaOAc (164 mg, 2 mmol) was added. The reaction mixture was then centrifuged, and the pellets were collected and dried to afford compound 4.3 (150 mg) in 70% yield. MS m/z: [M+H]⁺ Calcd for C₂₂₉H₃₀₂F₃N₈₀O₁₄₂P₂₁S₄ 7283.04; Found 7283.21.

Preparation of compound 4.6. To a solution of compound 4.5 (280 mg, 38 μmol) in 0.1 M aqueous Na₂B₃O₇ solution (2.6 mL) were added DIPEA (0.5 mL) and compound 4.4 (88 mg, 159 μmol) in DMSO (2.6 mL). After the reaction mixture was sonicated for 2 h, a DEA solution (2 mL) was added. The reaction mixture was sonicated for another 2 h. NaOAc (467 mg, 5.7 mmol) was then added. The reaction mixture was then centrifuged, and the pellets were collected and dried to afford compound 4.6 (210 mg) in 73% yield. MS m/z: [M+H]⁺ Calcd for C₂₂₉H₃₀₅F₂N₆₆O₁₅₁P₂₁S₄ 7214.96; Found 7215.17.

Preparation of compound 4.7. To a solution of compound 4.6 (210 mg, 28 μmol) in 0.1 M aqueous Na₂B₃O₇ solution (3.5 mL) were added DIPEA (0.5 mL) and compound 2.2 (332 mg, 175 μmol) in DMSO (4 mL). After the reaction mixture was sonicated for 2 h, NaOAc (656 mg, 8 mmol) was added. The reaction mixture was then centrifuged, and the pellets were collected and dried to afford compound 4.7 (160 mg) in 62% yield. MS m/z: [M+H]⁺ Calcd for C₃₀₇H₄₂₈F₂N₇₃O₁₈₆P₂₁S₄ 8933.81; Found 8933.98.

Preparation of compound 4.8. To a solution of compound 4.3 (55 mg, 8 μmol) and compound 4.7 (45 mg, 6 μmol) in water (3 mL) was copper(I) bromide dimethyl sulfide (3 mg, 15 μmol). After stirring at 60° C. for 2 h, NaOAc (256 mg, 3 mmol) was added. The reaction mixture was then centrifuged, and the pellets were collected and dried to afford compound 4.8 (53 mg). MS m/z: [M+H]⁺ Calcd for C₅₃₆H₇₃₀F₅N₁₅₃O₃₂₈P₄₂S₈ 16216.85; Found 16217.16.

Preparation of compound B4a. To a solution of compound 4.8 (53 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (29 mg), which was purified by C18 reverse-phase prep-HPLC and concentrated to about 1 mL to afford compound B4a (13.7 mg) in 17% yield. MS m/z: [M+H]⁺ C₅₁₈H₇₁₂F₅N₁₅₃O₃₁₉P₄₂S₈ 15838.52; Found 15838.75.

Preparation of compound A4a. A solution of compound B4a (1 eq.) and oligonucleotides 4.9 (1 eq.) and 4.10 (1 eq.) in water was heated at 70-95° C. for about 1 to 5 min and then cooled to room temperature to afford compound A4a.

### Example 5

### Preparation of GalNAc-siRNA Conjugate A5

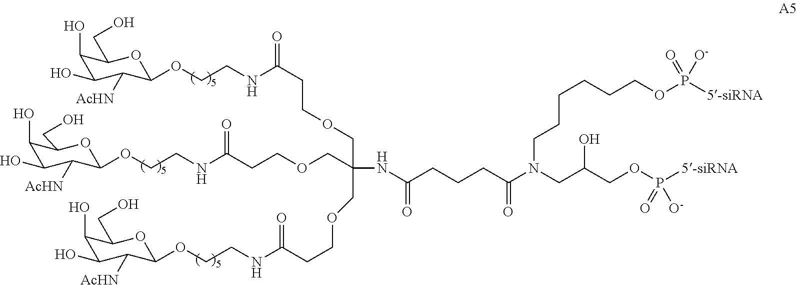

GalNAc-siRNA conjugate A5 is prepared as shown in Scheme 5, wherein R^{s} and R^{t} are as described in Example 2; each 5′-ssRNA independently represents a single-stranded RNA (e.g., a sense or antisense sequence of a siRNA, or an ASO sequence) with its 5′-terminus connected to a trivalent linker; and each 5′-siRNA independently represents a siRNA with one of its 5′-termini connected to a trivalent linker.

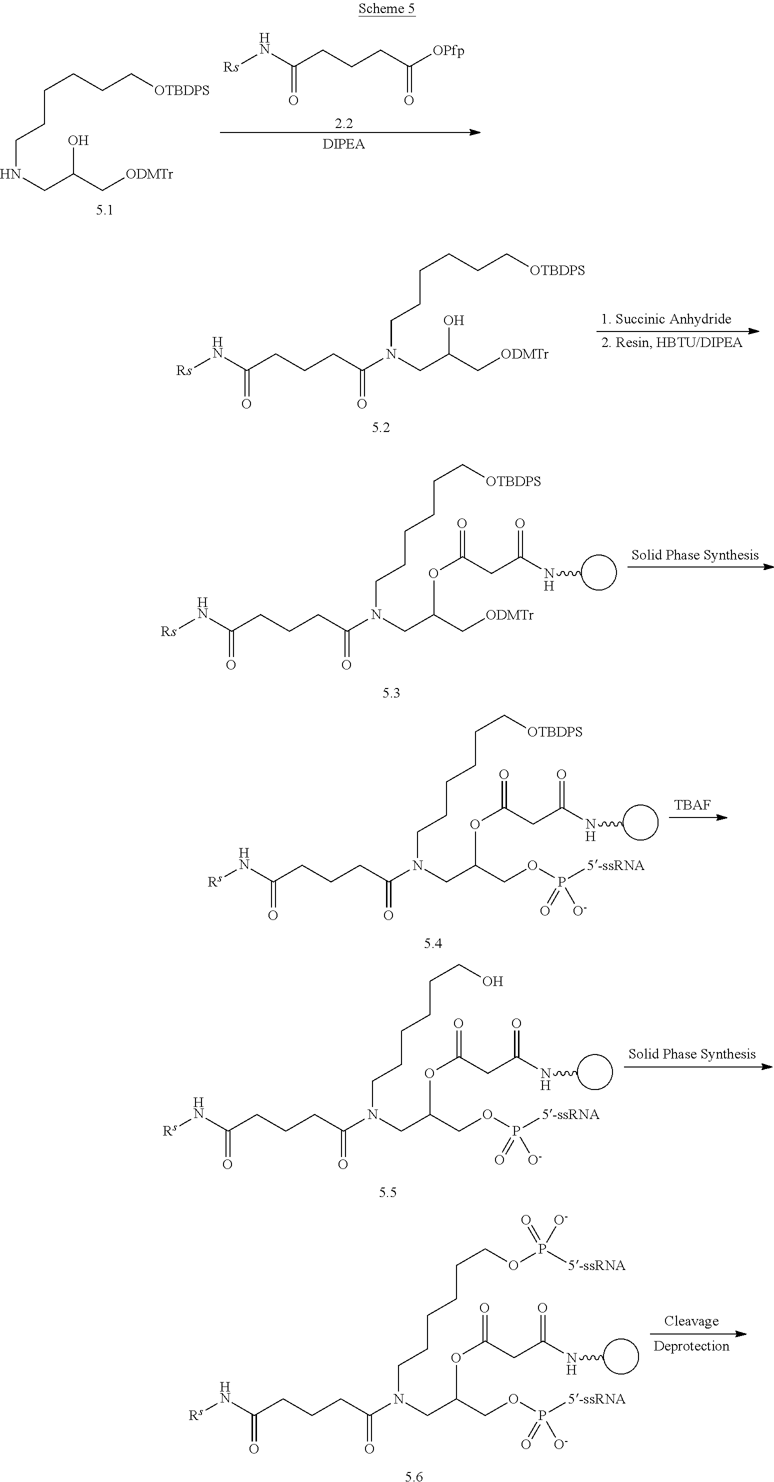

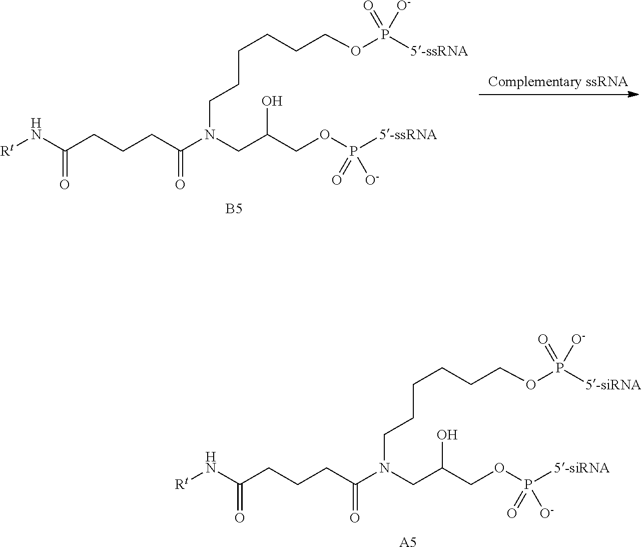

### Example 6

### Preparation of GalNAc-siRNA Conjugate A6

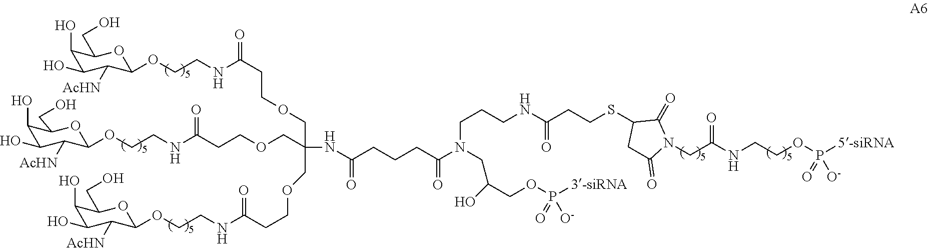

GalNAc-siRNA conjugate A6 is prepared as shown in Scheme 6, wherein R^{s} and R^{t} are as described in Example 2; 3′-ssRNA represents a single-stranded RNA (e.g., a sense or antisense sequence of a siRNA, or an ASO sequence) with its 3′-terminus connected to a trivalent linker; 5′-ssRNA represents a single-stranded RNA (e.g., a sense or antisense sequence of a siRNA, or an ASO sequence) with its 5′-terminus connected to a trivalent linker; 3′-siRNA represents a siRNA with one of its 3′-termini connected to a trivalent linker; and 5′-siRNA represents a siRNA with one of its 5′-termini connected to a trivalent linker.

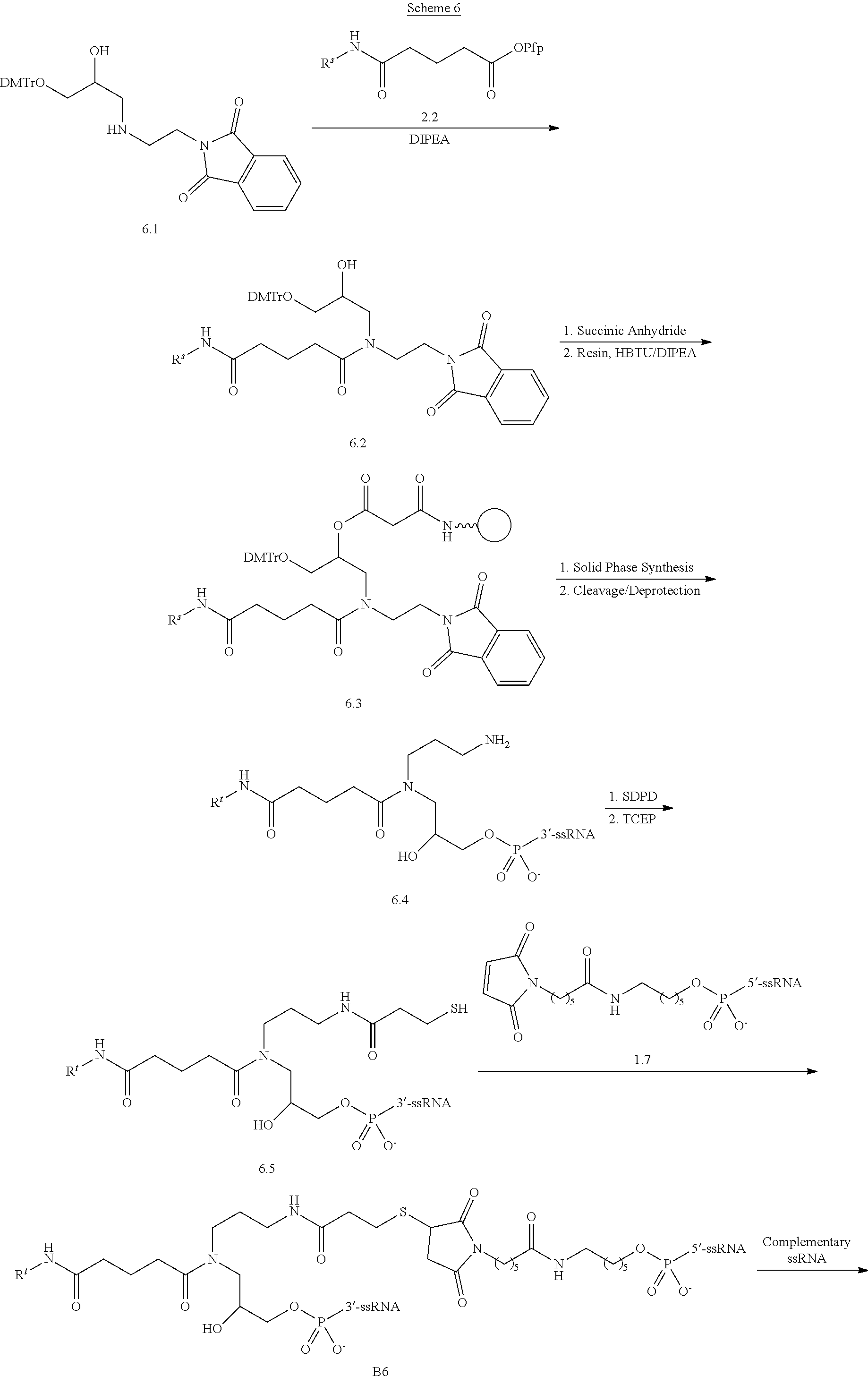

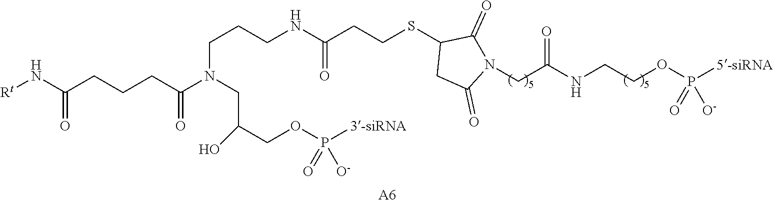

### Example 7

### Preparation of GalNAc-siRNA Conjugate A7

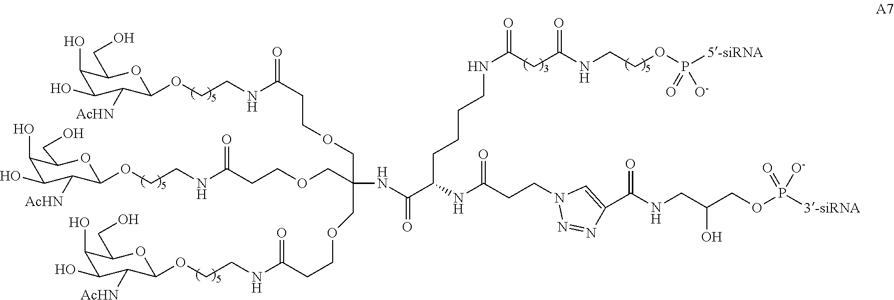

GalNAc-siRNA conjugate A7 is prepared as shown in Scheme 7, wherein R^{s} and R^{t} are as described in Example 2; 3′-ssRNA represents a single-stranded RNA (e.g., a sense or antisense sequence of a siRNA, or an ASO sequence) with its 3′-terminus connected to a trivalent linker; 5′-ssRNA represents a single-stranded RNA (e.g., a sense or antisense sequence of a siRNA, or an ASO sequence) with its 5′-terminus connected to a trivalent linker; 3′-siRNA represents a siRNA with one of its 3′-termini connected to a trivalent linker; and 5′-siRNA represents a siRNA with one of its 5′-termini connected to a trivalent linker.

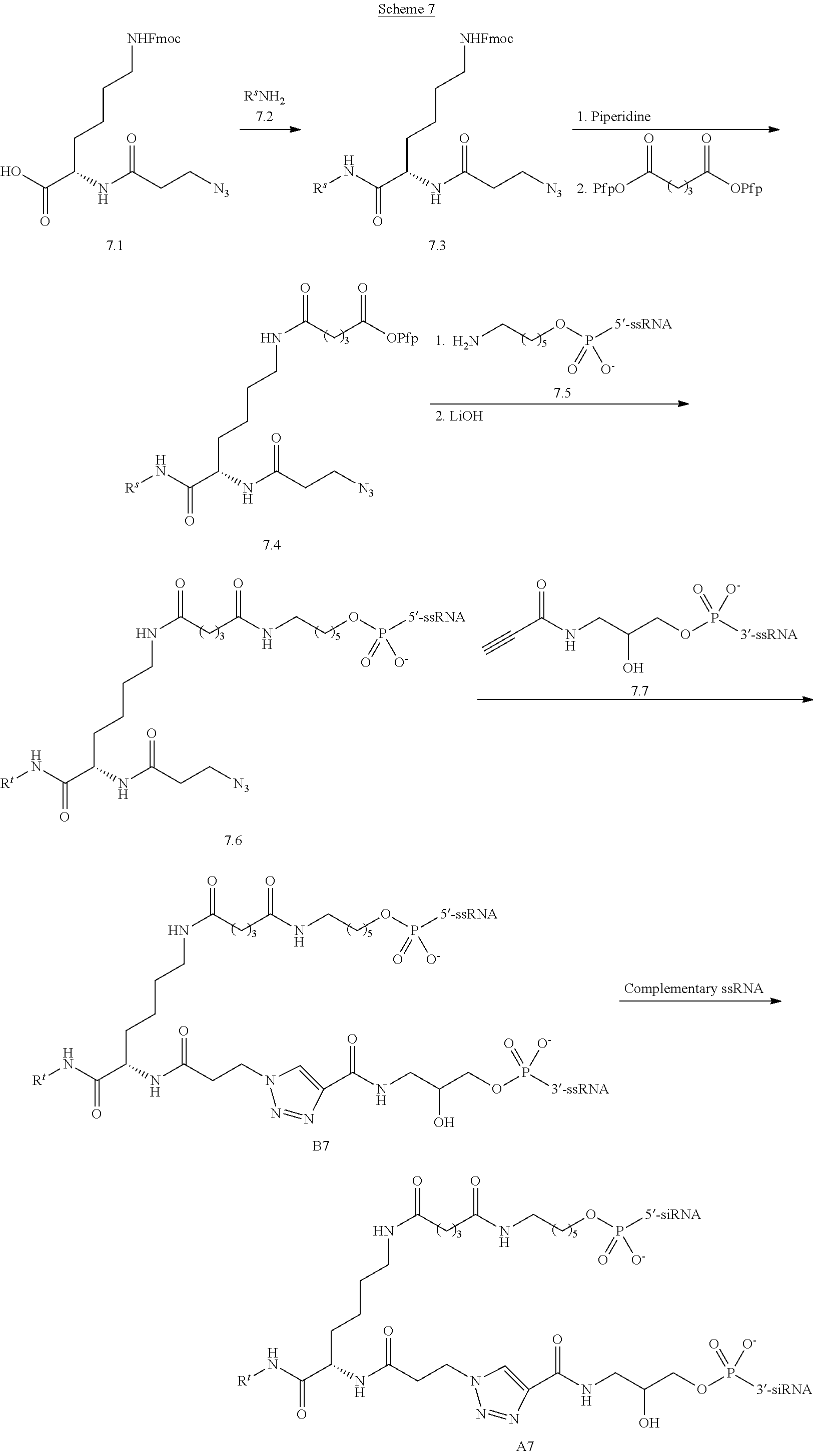

### Example 8

### Preparation of GalNAc-siRNA Conjugate A8

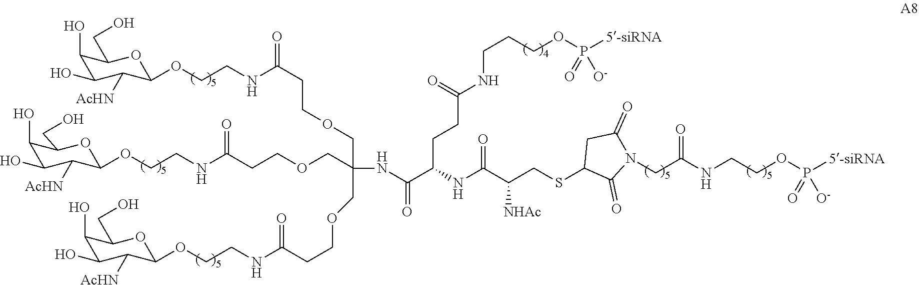

GalNAc-siRNA conjugate A8 is prepared as shown in Scheme 8, wherein R^{s} and R^{t} are as described in Example 2; each 5′-ssRNA independently represents a single-stranded RNA (e.g., a sense or antisense sequence of a siRNA, or an ASO sequence) with its 5′-terminus connected to a trivalent linker; and each 5′-siRNA independently represents a siRNA with one of its 5′-termini connected to a trivalent linker.

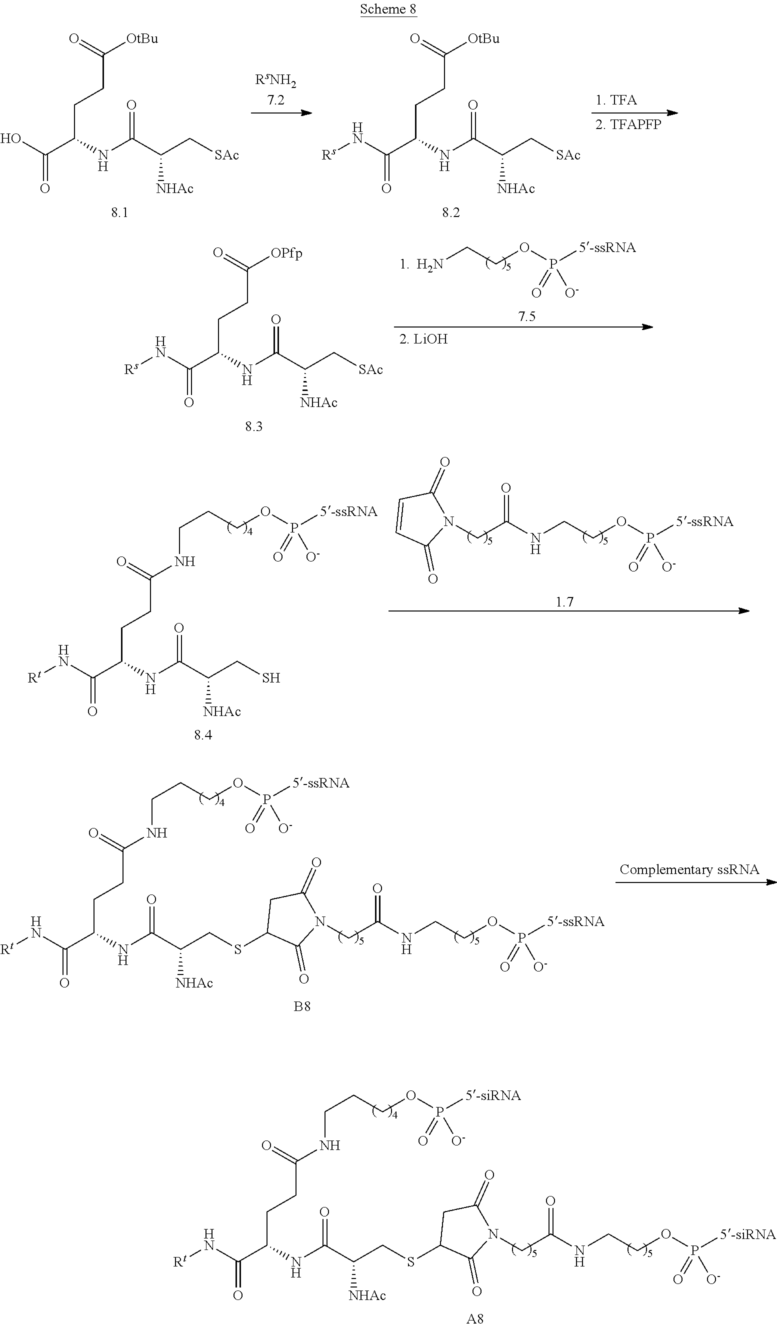

### Example 9

### Preparation of GalNAc-siRNA Conjugate A9

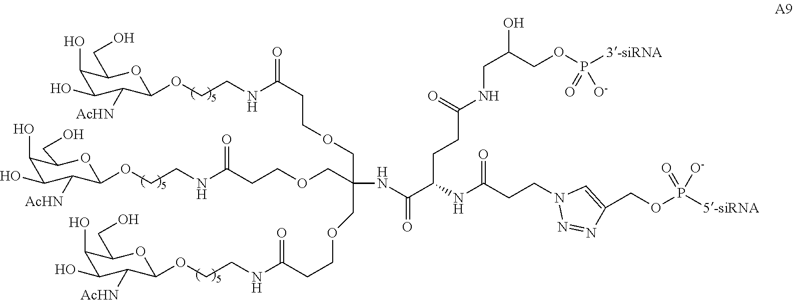

GalNAc-siRNA conjugate A9 is prepared as shown in Scheme 9, wherein R^{s} and R^{t} are as described in Example 2; 3′-ssRNA represents a single-stranded RNA (e.g., a sense or antisense sequence of a siRNA, or an ASO sequence) with its 3′-terminus connected to a trivalent linker; 5′-ssRNA represents a single-stranded RNA (e.g., a sense or antisense sequence of a siRNA, or an ASO sequence) with its 5′-terminus connected to a trivalent linker; 3′-siRNA represents a siRNA with one of its 3′-termini connected to a trivalent linker; and 5′-siRNA represents a siRNA with one of its 5′-termini connected to a trivalent linker.

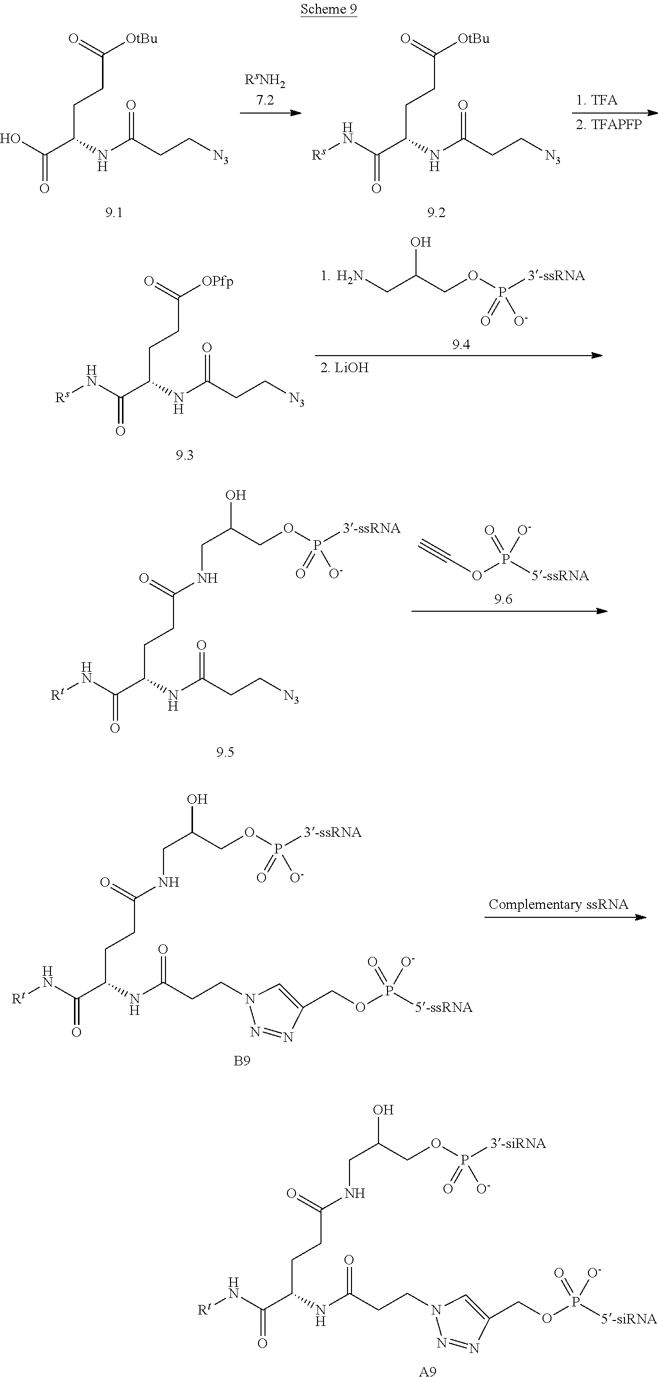

### Example 10

### Preparation of GalNAc-siRNA Conjugate A10

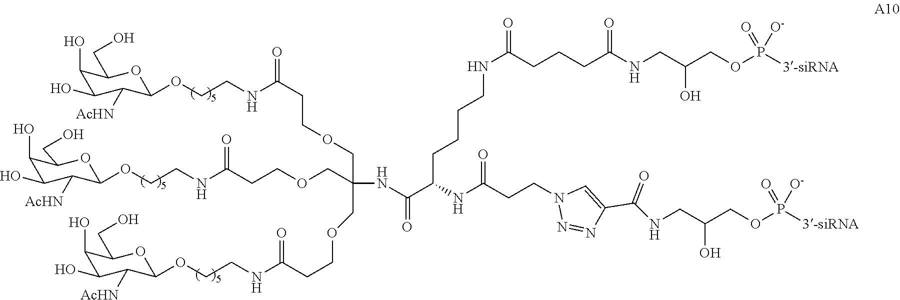

GalNAc-siRNA conjugate A10 is prepared as shown in Scheme 9, wherein R^{s} and R^{t} are as described in Example 2; each 3′-ssRNA independently represents a single-stranded RNA (e.g., a sense or antisense sequence of a siRNA, or an ASO sequence) with its 3′-terminus connected to a trivalent linker; and each 3′-siRNA independently represents a siRNA with one of its 3′-termini connected to a trivalent linker.

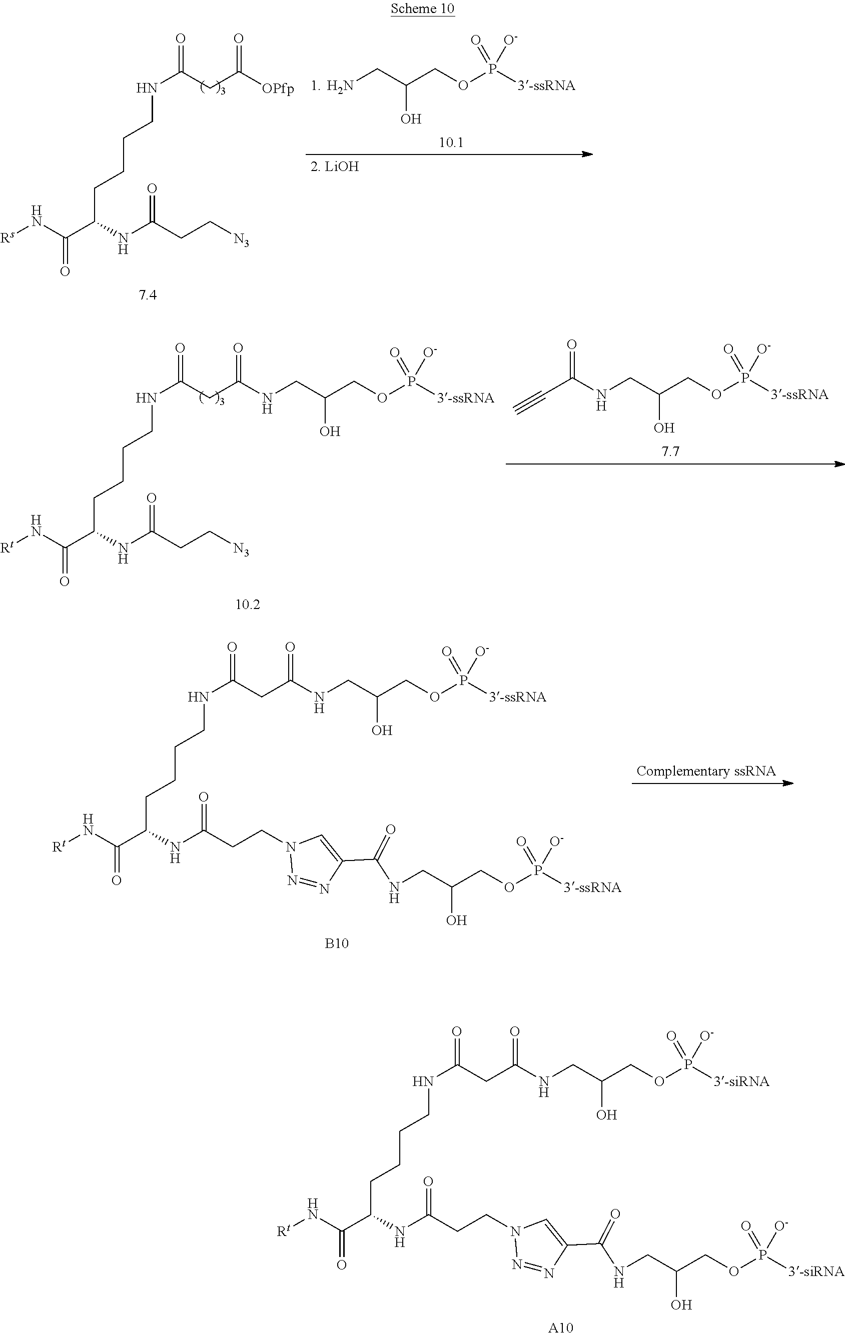

### Example 11

### Preparation of GalNAc-siRNA Conjugate A11

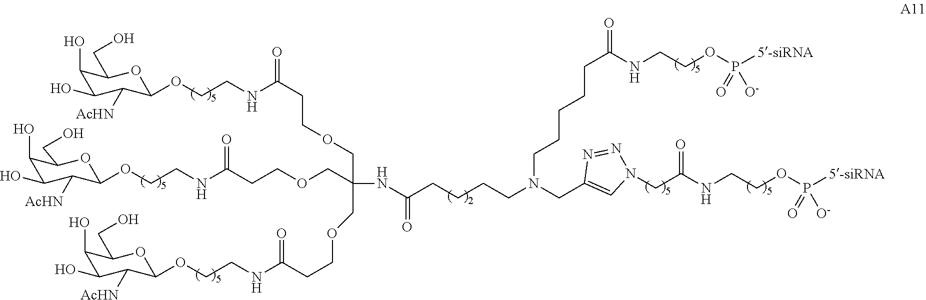

GalNAc-siRNA conjugate A11 is prepared as shown in Scheme 11, wherein R^{s} and R^{t} are as described in Example 2; each 5′-ssRNA independently represents a single-stranded RNA (e.g., a sense or antisense sequence of a siRNA, or an ASO sequence) with its 5′-terminus connected to a trivalent linker; and each 5′-siRNA independently represents a siRNA with one of its 5′-termini connected to a trivalent linker.

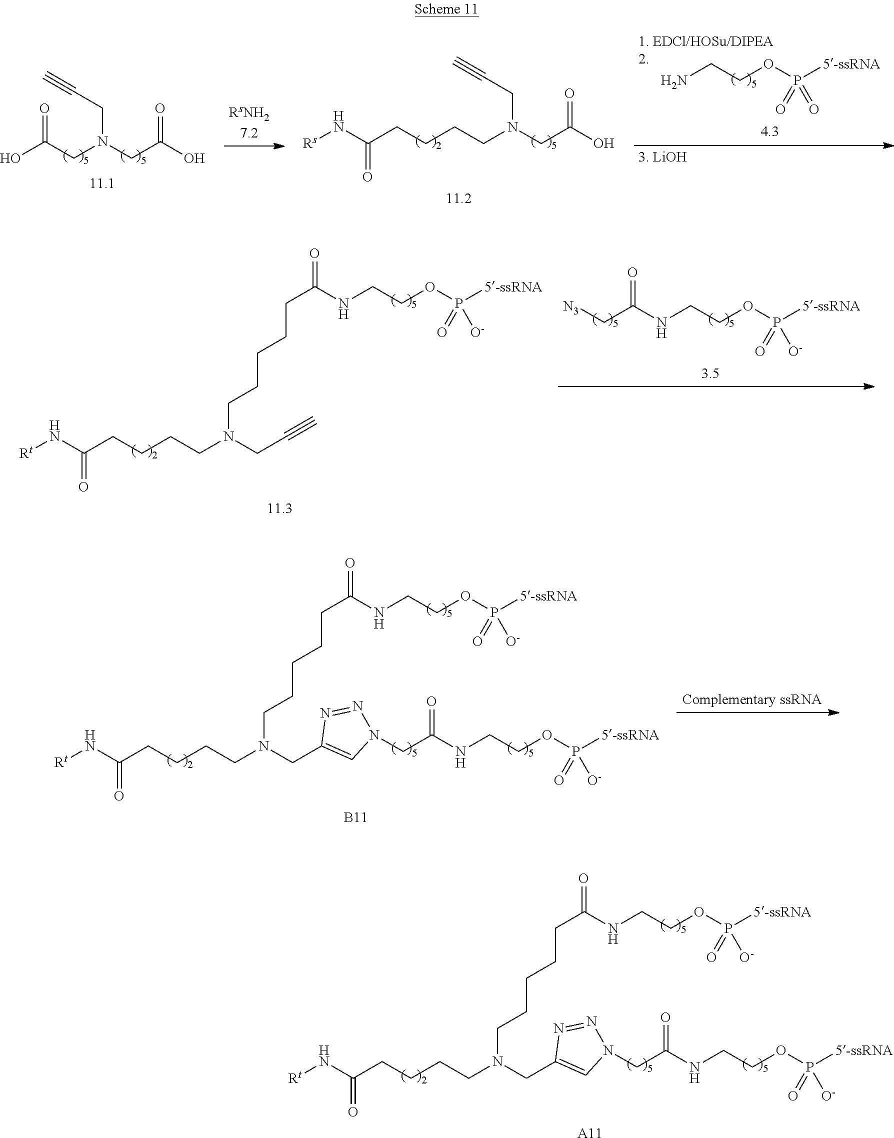

### Example 12

### Preparation of GalNAc-siRNA Conjugate A13

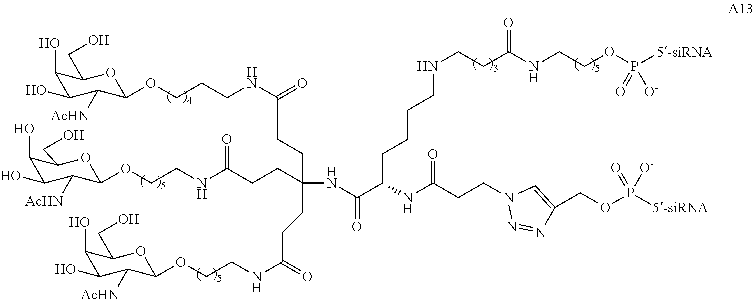

GalNAc-siRNA conjugate A13 is prepared as shown in Scheme 10, wherein each 5′-ssRNA independently represents a single-stranded RNA (e.g., a sense or antisense sequence of a siRNA, or an ASO sequence) with its 5′-terminus connected to a trivalent linker; and each 5′-siRNA independently represents a siRNA with one of its 5′-termini connected to a trivalent linker.

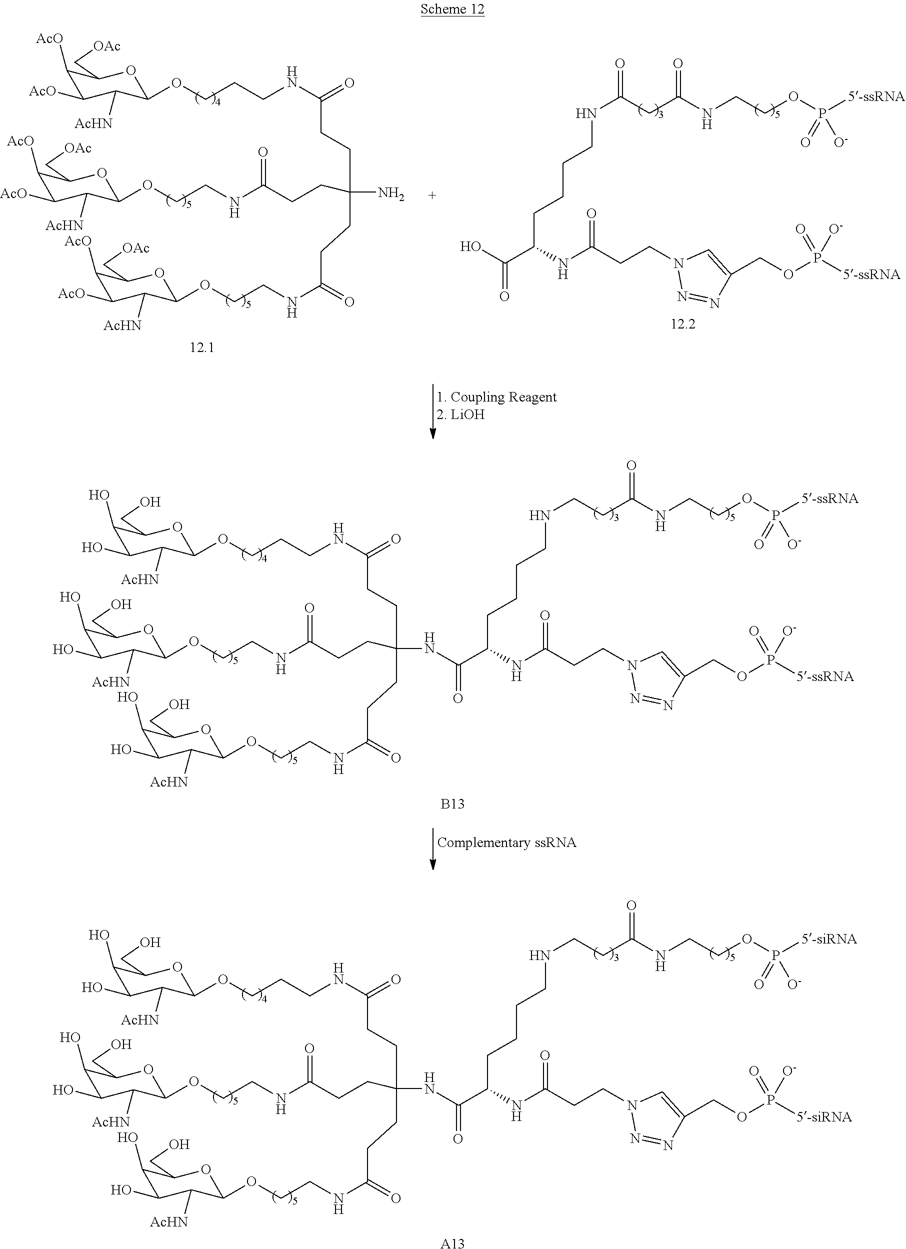

### Example 13

### Preparation of GalNAc-siRNA Conjugate A17

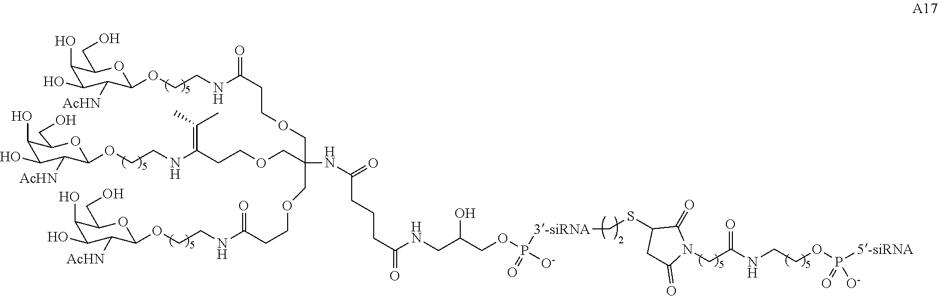

GalNAc-siRNA conjugate A17 is prepared as shown in Scheme 13, wherein R^{s} and R^{t} are as described in Example 2; 3′-ssRNA represents a single-stranded RNA (e.g., a sense or antisense sequence of a siRNA, or an ASO sequence) with its 3′-terminus connected to a first divalent linker and its 5′-terminus to a second divalent linker; 5′-ssRNA represents a single-stranded RNA (e.g., a sense or antisense sequence of a siRNA, or an ASO sequence) with its 5′-terminus to the second divalent linker; 3′-siRNA represents a siRNA with one of its 3′-termini connected to a first divalent linker and one of its 5′-termini connected to a second divalent linker; and 5′-siRNA represents a siRNA with one of its 5′-termini connected to the second divalent linker.

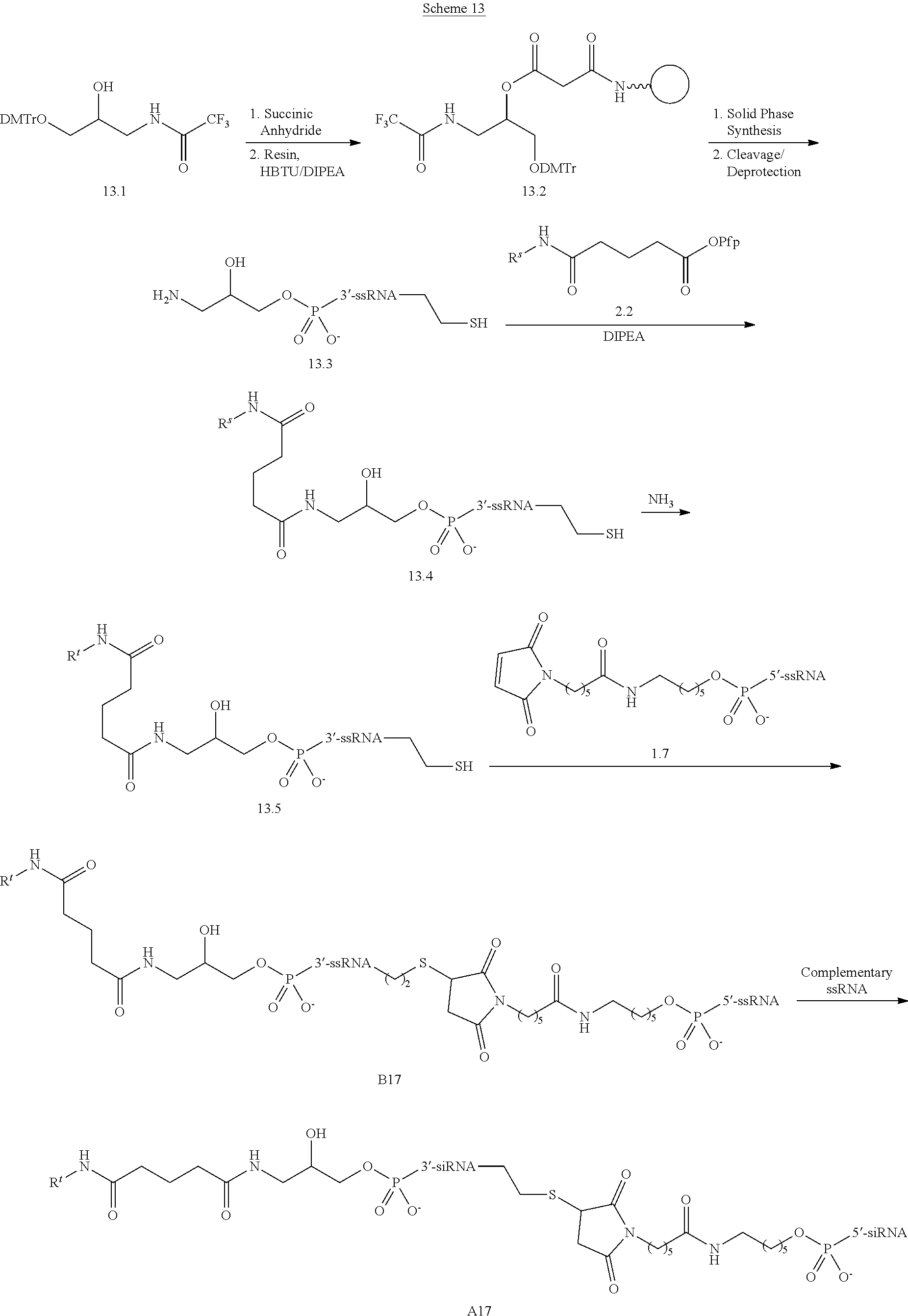

### Example 14

### Preparation of GalNAc-siRNA Conjugate A18a

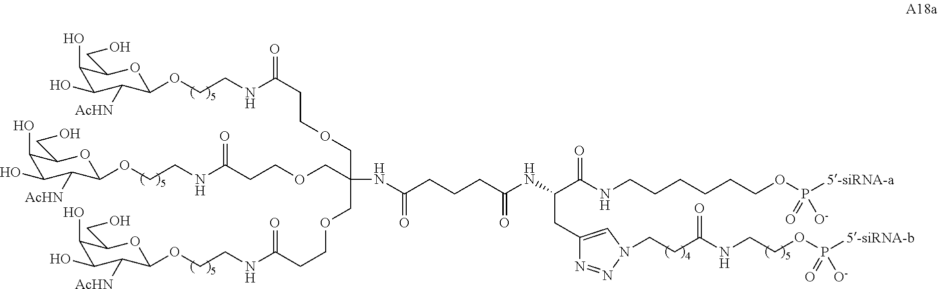

GalNAc-siRNA conjugate A18a was prepared as shown in Scheme 14, wherein R^{s} and R^{t} are as described in Example 2; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

Preparation of compound 14.3. To a solution of compound 14.2 (280 mg, 38 μmol) in 0.1 M aqueous Na₂B₃O₇ solution (2.6 mL) were added DIPEA (0.5 mL) and compound 14.1 (79 mg, 159 μmol) in DMSO (2.6 mL). After the reaction mixture was sonicated for 2 h, a DEA solution (2 mL) was added. The reaction mixture was sonicated for another 2 h. NaOAc (467 mg, 5.7 mmol) was then added. The reaction mixture was centrifuged, and the pellets were collected and dried to afford compound 14.3 (250 mg) in 88% yield. MS m/z: [M+H]⁺ Calcd for C₂₂₅H₂₉₇F₂N₆₆O₁₅₁P₂₁S₄ 7158.85; Found 7158.92.

Preparation of compound 14.4. To a solution of compound 14.3 (250 mg, 34 μmol) in 0.1 M aqueous Na₂B₃O₇ solution (3.5 mL) were added DIPEA (0.5 mL) and compound 2.2 (399 mg, 210 μmol) in DMSO (4 mL). After the reaction mixture was sonicated for 2 h, NaOAc (656 mg, 8 mmol) was added. The reaction mixture was then centrifuged, and the pellets were collected and dried to afford compound 14.4 (270 mg) in 87% yield. MS m/z: [M+H]⁺ Calcd for C₃₀₃H₄₂₀F₂N₇₃O₁₈₆P₂₁S₄ 8877.7; Found 8877.78.

Preparation of compound 14.5. To a solution of compound 4.3 (74 mg, 10 μmol) and compound 14.4 (60 mg, 8 μmol) in water (3 mL) was copper(I) bromide dimethyl sulfide (4 mg, 19 μmol). After stirring at 60° C. for 2 h, NaOAc (246 mg, 3 mmol) was added. The reaction mixture was then centrifuged, and the pellets were collected and dried to afford compound 14.5 (65 mg). MS m/z: [M+H]⁺ Calcd for C₅₃₂H₇₂₂F₅N₁₅₃O₃₂₈P₄₂S₈ 16160.74; Found 16161.09.

Preparation of compound B18a. To a solution of compound 14.5 (65 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (51 mg), which was purified by C18 reverse-phase prep-HPLC and concentrated to about 1 mL to afford compound B18a (20.5 mg) in 19% yield. MS m/z: [M+H]⁺ Calcd for C₅₁₄H₇₀₄F₅N₁₅₃O₃₁₉P₄₂S₈ 15782.41; Found 15782.53.

Preparation of compound A18a. A solution of compound B18a (1 eq.) and oligonucleotides 4.9 (1 eq.) and 4.10 (1 eq.) in water was heated at 70-95° C. for about 1 to 5 min and then cooled to room temperature to afford compound A18a.

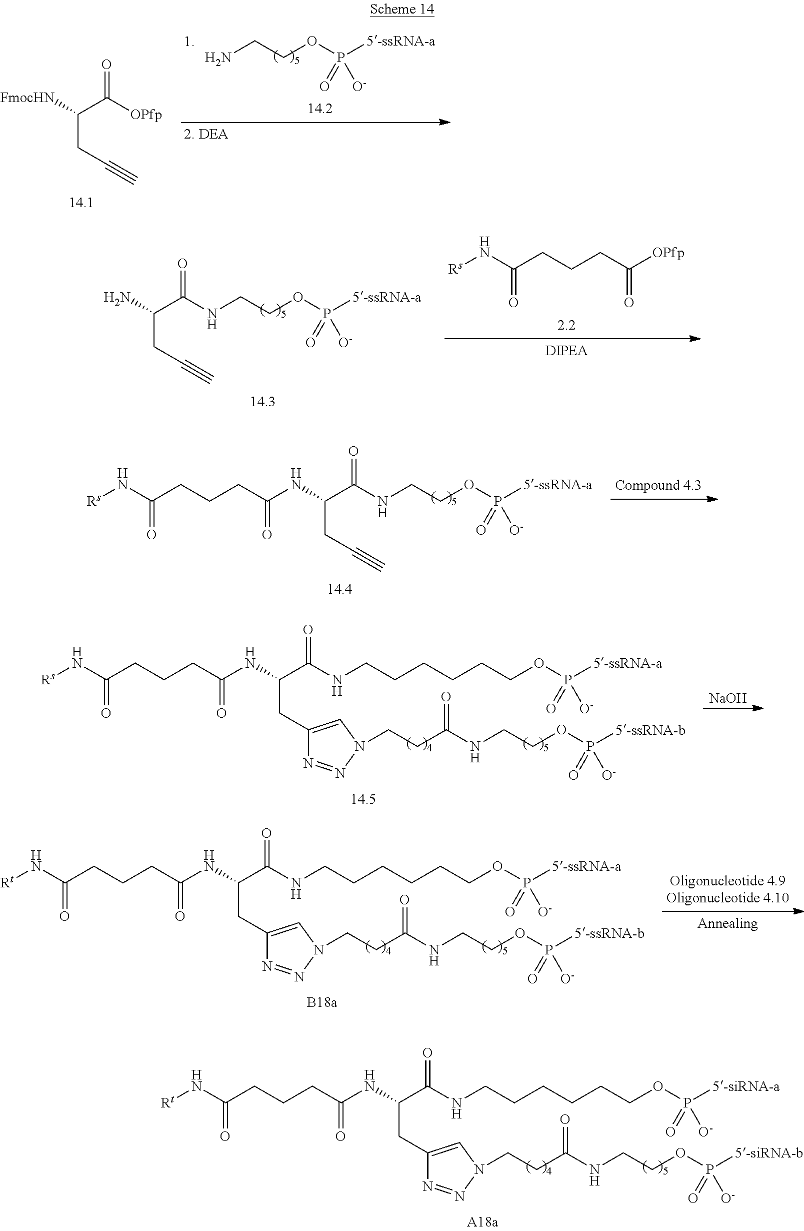

GalNAc-siRNA conjugates A18b to A18h were prepared similarly as shown in Example 14.

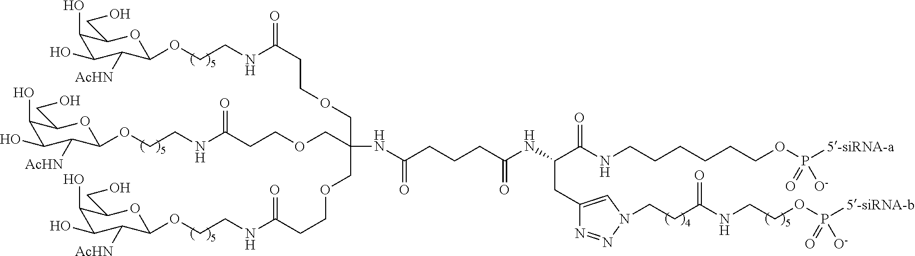

| Conjugate | siRNA-a Sense | siRNA-a Antisense | siRNA-b Sense | siRNA-b Antisense |
|---|---|---|---|---|
| A18b | SEQ ID NO: 5 | SEQ ID NO: 6 | SEQ ID NO: 7 | SEQ ID NO: 8 |
| A18c | SEQ ID NO: 9 | SEQ ID NO: 10 | SEQ ID NO: 11 | SEQ ID NO: 12 |
| A18d | SEQ ID NO: 13 | SEQ ID NO: 14 | SEQ ID NO: 7 | SEQ ID NO: 8 |
| A18e | SEQ ID NO: 15 | SEQ ID NO: 16 | SEQ ID NO: 17 | SEQ ID NO: 18 |
| A18f | SEQ ID NO: 9 | SEQ ID NO: 10 | SEQ ID NO: 19 | SEQ ID NO: 20 |
| A18g | SEQ ID NO: 19 | SEQ ID NO: 20 | SEQ ID NO: 21 | SEQ ID NO: 22 |
| A18h | SEQ ID NO: 3 | SEQ ID NO: 4 | SEQ ID NO: 1 | SEQ ID NO: 2 |

GalNAc-siRNA conjugates B18b to B18g were prepared similarly as shown in Example 14.

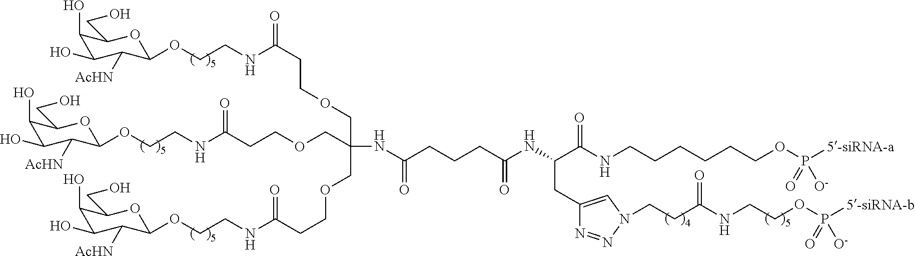

| Conjugate | siRNA-a Sense | siRNA-b Sense | MS m/z: [M + H]⁺ |
|---|---|---|---|
| B18b | SEQ ID NO: 5 | SEQ ID NO: 7 | Calcd for C₅₁₁H₆₉₄F₈N₁₅₂O₃₁₇P₄₂S₈ |
| | | | 15738.95; Found 15739.41 |
| B18c | SEQ ID NO: 9 | SEQ ID NO: 11 | Calcd for C₅₀₆H₆₅₉F₂₁N₁₇₂O₂₉₃P₄₂S₈ |
| | | | 15786.84; Found 15787.41 |
| B18d | SEQ ID NO: 13 | SEQ ID NO: 7 | Calcd for C₅₁₂H₆₉₉F₈N₁₅₉O₃₀₉P₄₂S₈ |
| | | | 15734.39; Found 15734.78 |
| B18e | SEQ ID NO: 15 | SEQ ID NO: 27 | Calcd for C₅₁₆H₆₉₉F₈N₁₆₇O₃₁₀P₄₂S₈ |
| | | | 15910.49; Found 15910.75 |
| B18f | SEQ ID NO: 9 | SEQ ID NO: 19 | Calcd for C₅₁₇H₆₈₉F₁₃N₁₈₄O₂₈₉P₄₂S₈ |
| | | | 15909.56; Found 15909.81 |
| B18g | SEQ ID NO: 28 | SEQ ID NO: 30 | Calcd for C₅₁₄H₆₈₁F₁₆N₁₈₅O₂₈₈P₄₂S₈ |
| | | | 15920.46; Found 15920.66 |

### Example 15

### Preparation of GalNAc-siRNA Conjugate A19a

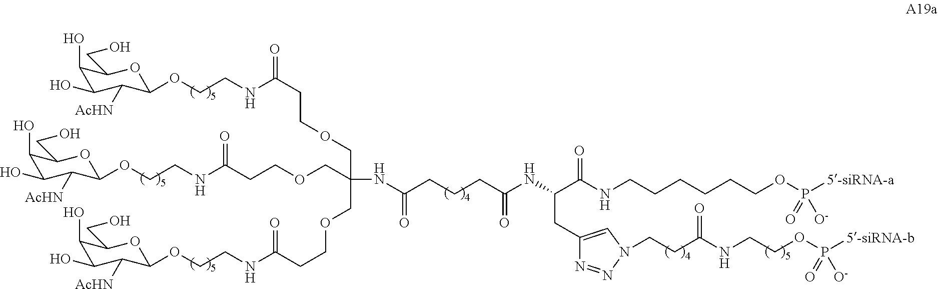

GalNAc-siRNA conjugate A19a was prepared as shown in Scheme 15, wherein R^{s} and R^{t} are as described in Example 2; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

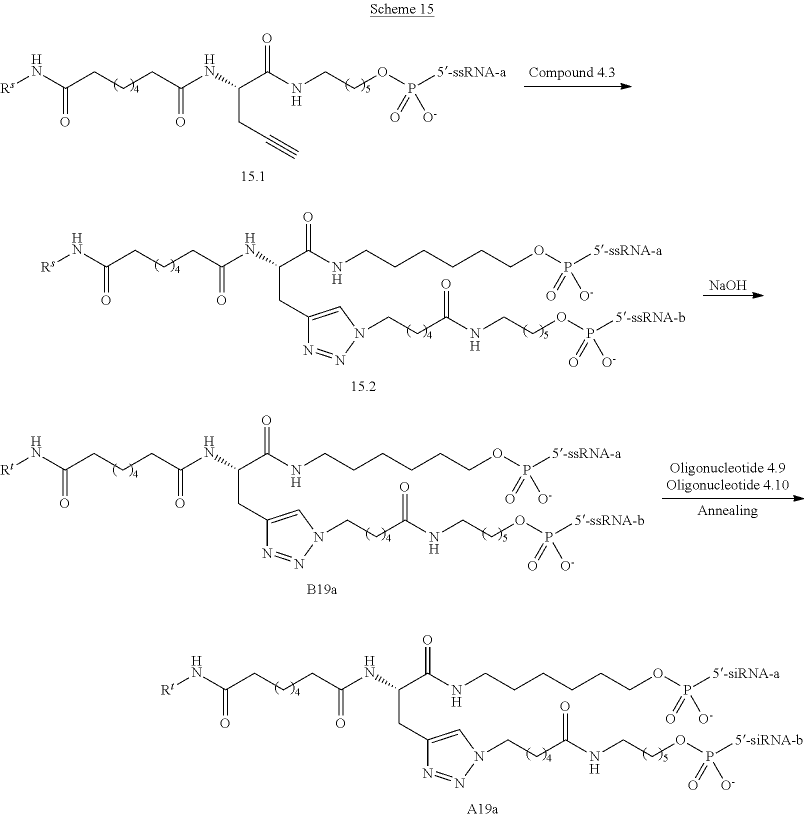

Preparation of compound 15.2. To a solution of compound 15.1 (62 mg, 9 μmol) and compound 4.3 (76 mg, 10 μmol) in water (3 mL) was copper(I) bromide dimethyl sulfide (4 mg, 19 μmol). After stirring at 60° C. for 2 h, NaOAc (246 mg, 3 mmol) was added. The reaction mixture was then centrifuged, and the pellets were collected and dried to afford compound 15.2 (78 mg). MS m/z: [M+H]⁺ Calcd for C₅₃₅H₇₂₈F₅N₁₅₃O₃₂₈P₄₂S₈ 16202.82; Found 16202.93.

Preparation of compound B19a. To a solution of compound 15.2 (78 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (52 mg), which was purified by C18 reverse-phase prep-HPLC and concentrated to about 1 mL to afford compound B19a (26.3 mg) in 24% yield. MS m/z: [M+H]⁺ C₅₁₇H₇₁₀F₅N₁₅₃O₃₁₉P₄₂S₈ 15824.49; Found 15824.72.

Preparation of compound A19a. A solution of compound B19a (1 eq.) and oligonucleotides 4.9 (1 eq.) and 4.10 (1 eq.) in water was heated at 70-95° C. for about 1 to 5 min and then cooled to room temperature to afford compound A19a.

### Example 16

### Preparation of GalNAc-siRNA Conjugate A20a

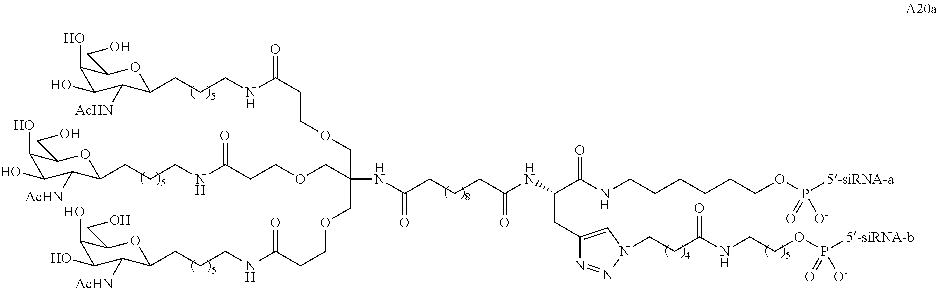

GalNAc-siRNA conjugate A20a was prepared as shown in Scheme 16, wherein R^{s} and R^{t} are as described in Example 2; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

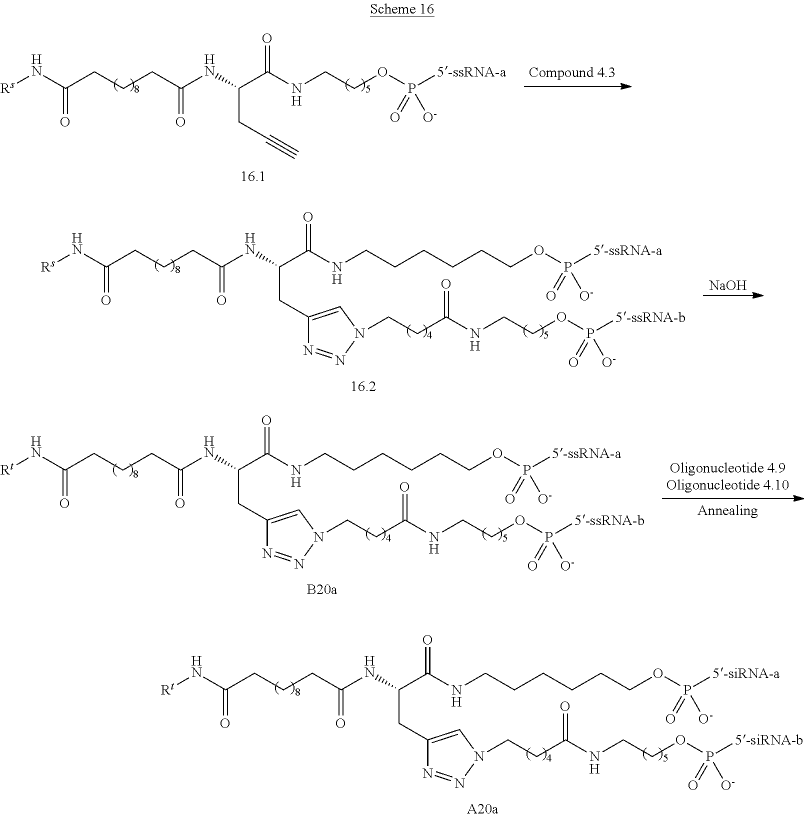

Preparation of compound 16.2. To a solution of compound 16.1 (75 mg, 10 μmol) and compound 4.3 (92 mg, 13 μmol) in water (3 mL) was copper(I) bromide dimethyl sulfide (5 mg, 24 μmol). After stirring at 60° C. for 2 h, NaOAc (246 mg, 3 mmol) was added. The reaction mixture was then centrifuged, and the pellets were collected and dried to afford compound 16.2 (67 mg). MS m/z: [M+H]⁺ Calcd for C₅₃₉H₇₃₆F₅N₁₅₃O₃₂₈P₄₂S₈ 16258.93; Found 16258.94.

Preparation of compound B20a. To a solution of compound 16.2 (67 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (51 mg), which was purified by C18 reverse-phase prep-HPLC and concentrated to about 1 mL to afford compound B20a (23.9 mg) in 18% yield. MS m/z: [M+H]⁺ C₅₂₁H₇₁₈F₅N₁₅₃O₃₁₉P₄₂S₈ 15880.6; Found 15880.86.

Preparation of compound A20a. A solution of compound B20a (1 eq.) and oligonucleotides 4.9 (1 eq.) and 4.10 (1 eq.) in water was heated at 70-95° C. for about 1 to 5 min and then cooled to room temperature to room temperature to afford compound A20a.

### Example 17

### Preparation of GalNAc-siRNA Conjugate A21a

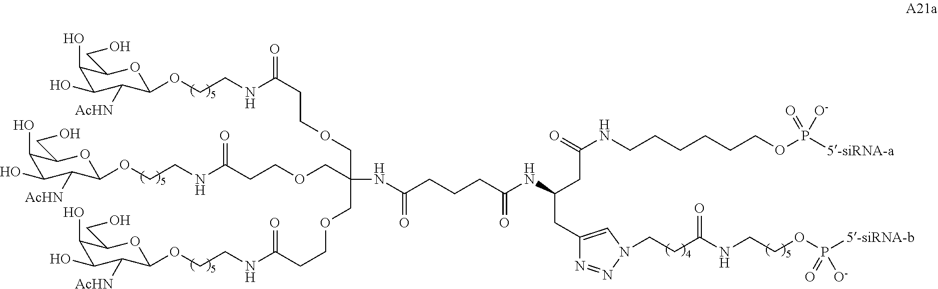

GalNAc-siRNA conjugate A21a was prepared as shown in Scheme 17, wherein R^{s} and R^{t} are as described in Example 2; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

Preparation of compound 17.2. To a solution of compound 17.1 (80 mg, 11 μmol) and compound 4.3 (98 mg, 14 μmol) in water (3 mL) was copper(I) bromide dimethyl sulfide (6 mg, 29 μmol). After stirring at 60° C. for 2 h, NaOAc (246 mg, 3 mmol) was added. The reaction mixture was then centrifuged, and the pellets were collected and dried to afford compound 16.2 (73 mg). MS m/z: [M+H]⁺ Calcd for C₅₃₃H₇₂₄F₅N₁₅₃O₃₂₈P₄₂S₈ 16174.77; Found 16175.1.

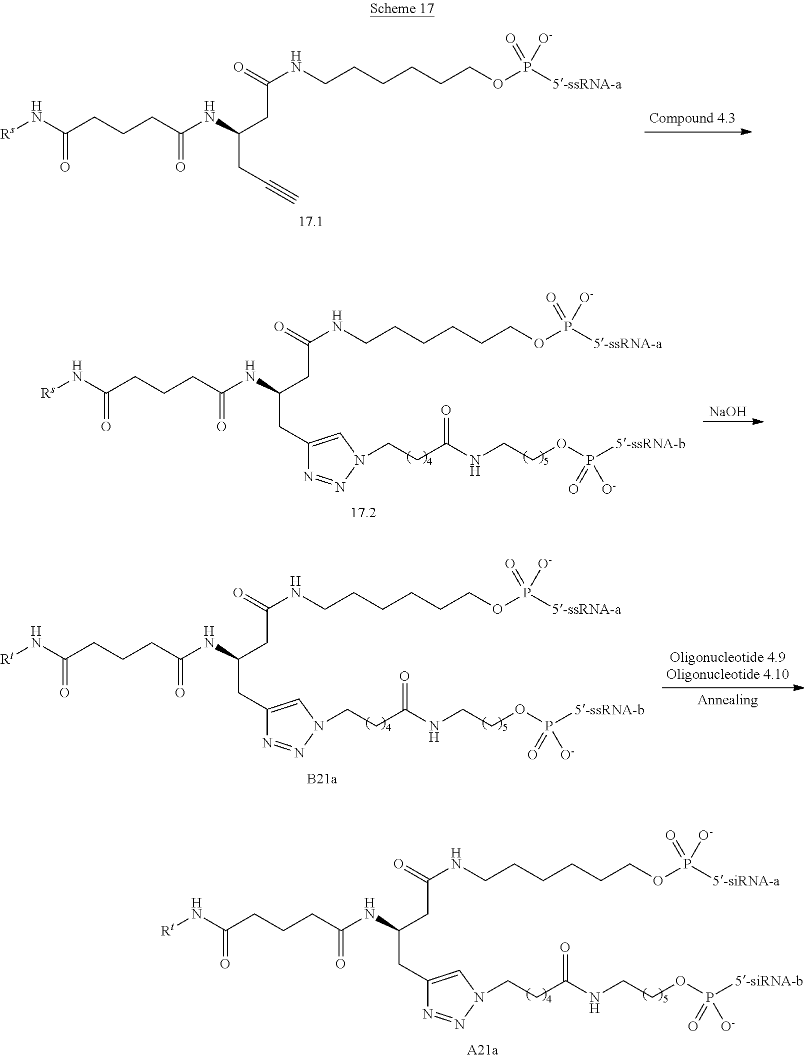

Preparation of compound B21a. To a solution of compound 17.2 (73 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (57 mg), which was purified by C18 reverse-phase prep-HPLC and concentrated to about 1 mL to afford compound B21a (25.7 mg) in 18% yield. MS m/z: [M+H]⁺ C₅₁₅H₇₀₆F₅N₁₅₃O₃₁₉P₄₂S₈ 15796.44; Found 15797.07.

Preparation of compound A21a. A solution of compound B21a (1 eq.) and oligonucleotides 4.9 (1 eq.) and 4.10 (1 eq.) in water was heated at 70-95° C. for about 1 to 5 min and then cooled to room temperature to room temperature to afford compound A21a.

### Example 18

### Preparation of GalNAc-siRNA Conjugate A22a

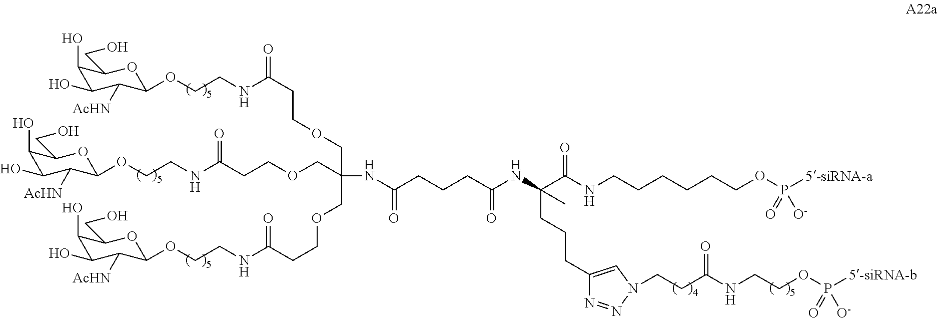

GalNAc-siRNA conjugate A22a was prepared as shown in Scheme 18, wherein R^{s} and R^{t} are as described in Example 2; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

Preparation of compound 18.2. To a solution of compound 18.1 (56 mg, 8 μmol) and compound 4.3 (69 mg, 9 μmol) in water (3 mL) was copper(I) bromide dimethyl sulfide (4 mg, 19 μmol). After stirring at 60° C. for 2 h, NaOAc (246 mg, 3 mmol) was added. The reaction mixture was then centrifuged, and the pellets were collected and dried to afford compound 18.2 (76 mg). MS m/z: [M+H]⁺ Calcd for C₅₃₅H₇₂₈F₅N₁₅₃O₃₂₈P₄₂S₈ 16202.82; Found 16202.95.

Preparation of compound B22a. To a solution of compound 18.2 (76 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (66 mg), which was purified by C18 reverse-phase prep-HPLC and concentrated to about 1 mL to afford compound B22a (19.8 mg) in 20% yield. MS m/z: [M+H]⁺ C₅₁₇H₇₁₀F₅N₁₅₃O₃₁₉P₄₂S₈ 15824.49; Found 15824.67.

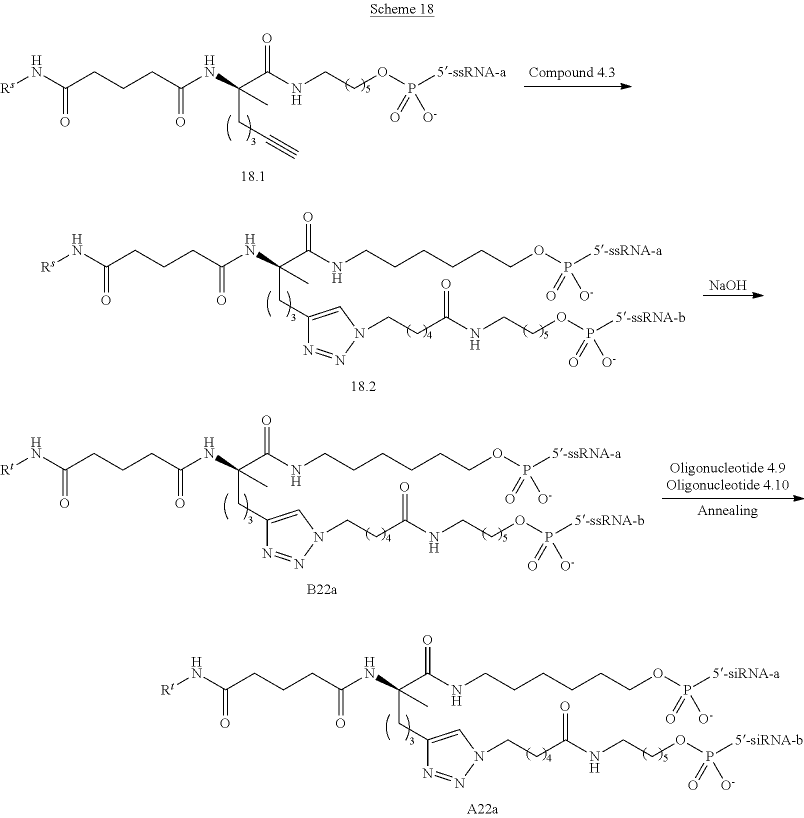

Preparation of compound A22a. A solution of compound B22a (1 eq.) and oligonucleotides 4.9 (1 eq.) and 4.10 (1 eq.) in water was heated at 70-95° C. for about 1 to 5 min and then cooled to room temperature to room temperature to afford compound A22a.

### Example 19

### Preparation of GalNAc-siRNA Conjugate A23a

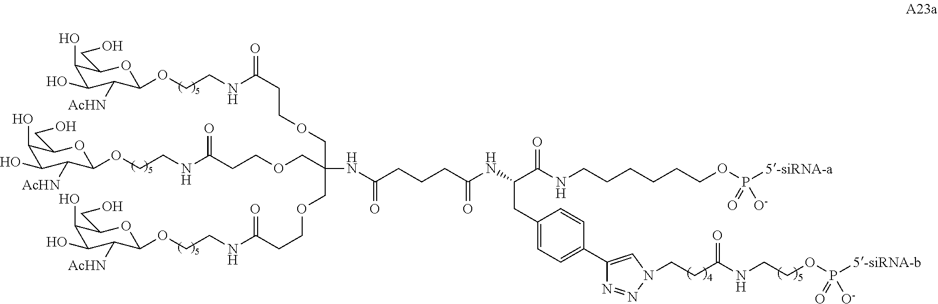

GalNAc-siRNA conjugate A23a was prepared as shown in Scheme 19, wherein R^{s} and R^{t} are as described in Example 2; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

Preparation of compound 19.2. To a solution of compound 19.1 (60 mg, 8 μmol) and compound 4.3 (74 mg, 10 μmol) in water (3 mL) was copper(I) bromide dimethyl sulfide (4 mg, 19 μmol). After stirring at 60° C. for 2 h, NaOAc (246 mg, 3 mmol) was added. The reaction mixture was then centrifuged, and the pellets were collected and dried to afford compound 19.2 (82 mg). MS m/z: [M+H]⁺ Calcd for C₅₃₈H₇₂₆F₅N₁₅₃O₃₂₈P₄₂S₈ 16236.84; Found 16236.98.

Preparation of compound B22a. To a solution of compound 18.2 (82 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (56 mg), which was purified by C18 reverse-phase prep-HPLC and concentrated to about 1 mL to afford compound B23a (21.7 mg) in 20% yield. MS m/z: [M+H]⁺ C₅₂₀H₇₀₈F₅N₁₅₃O₃₁₉P₄₂S₈ 15858.51; Found 15858.84.

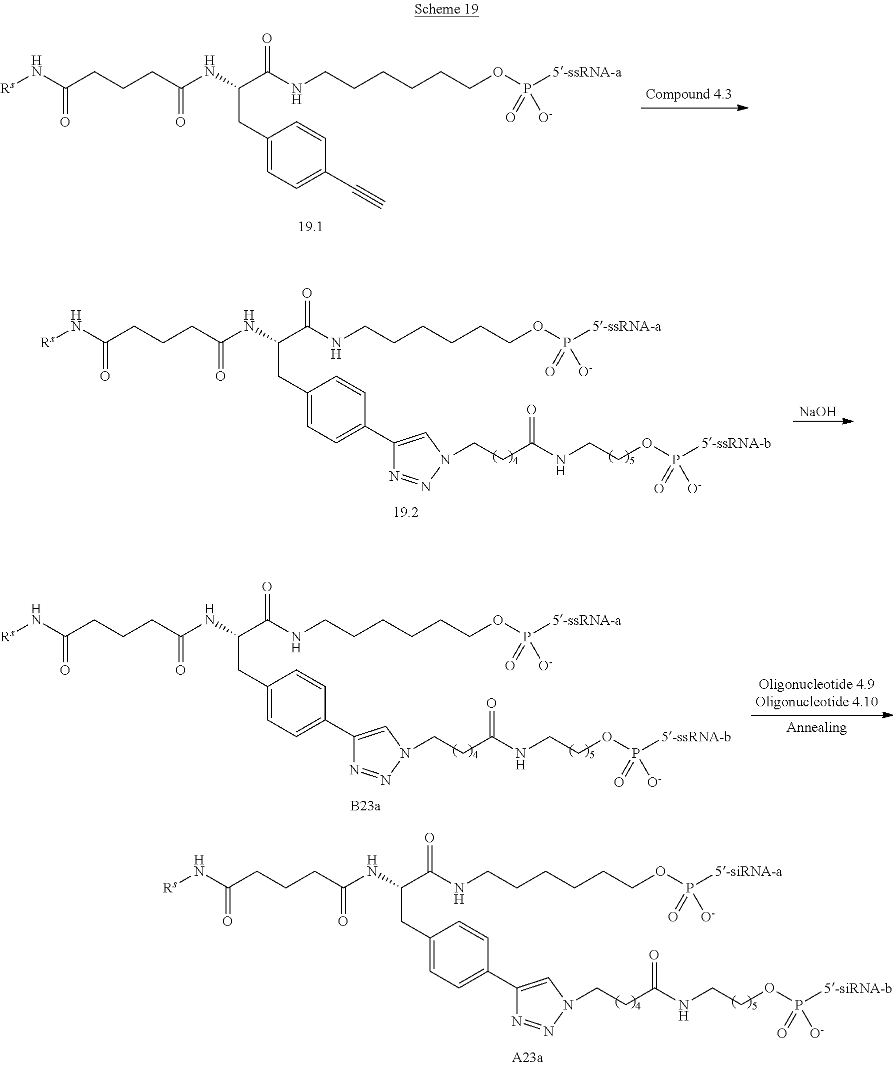

Preparation of compound A23a. A solution of compound B23a (1 eq.) and oligonucleotides 4.9 (1 eq.) and 4.10 (1 eq.) in water was heated at 70-95° C. for about 1 to 5 min and then cooled to room temperature to room temperature to afford compound A23a.

### Example 20

### Preparation of GalNAc-siRNA Conjugate A24a

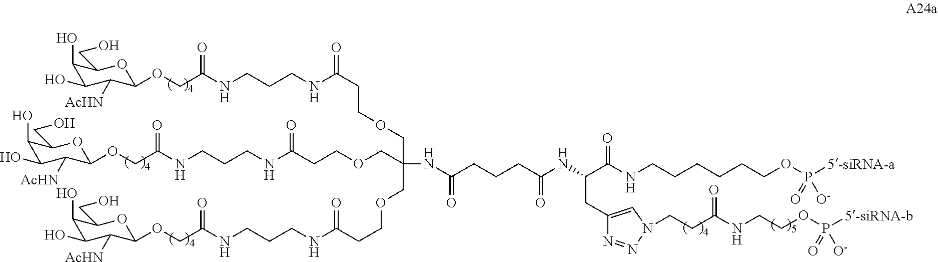

GalNAc-siRNA conjugate A24a was prepared as shown in Scheme 20, wherein R^{u} is a moiety having the structure of

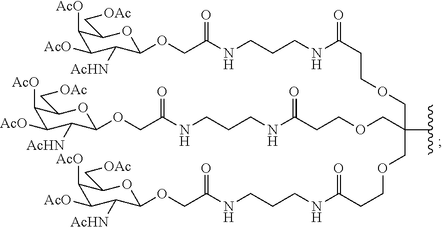 [SMILES: *C(COCCC(=O)NCCCNC(=O)COC1OC(COC(C)=O)C(OC(C)=O)C(OC(C)=O)C1NC(C)=O)(COCCC(=O)NCCCNC(=O)COC1OC(COC(C)=O)C(OC(C)=O)C(OC(C)=O)C1NC(C)=O)COCCC(=O)NCCCNC(=O)COC1OC(COC(C)=O)C(OC(C)=O)C(OC(C)=O)C1NC(C)=O]

R^{v} is an ASGPR binding moiety having the structure of

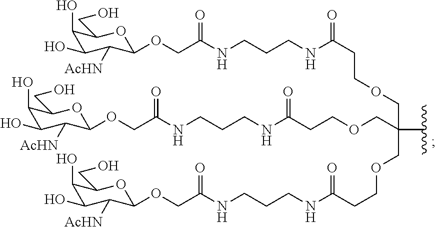 [SMILES: *C(COCCC(=O)NCCCNC(=O)COC1OC(CO)C(O)C(O)C1NC(C)=O)(COCCC(=O)NCCCNC(=O)COC1OC(CO)C(O)C(O)C1NC(C)=O)COCCC(=O)NCCCNC(=O)COC1OC(CO)C(O)C(O)C1NC(C)=O]

5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

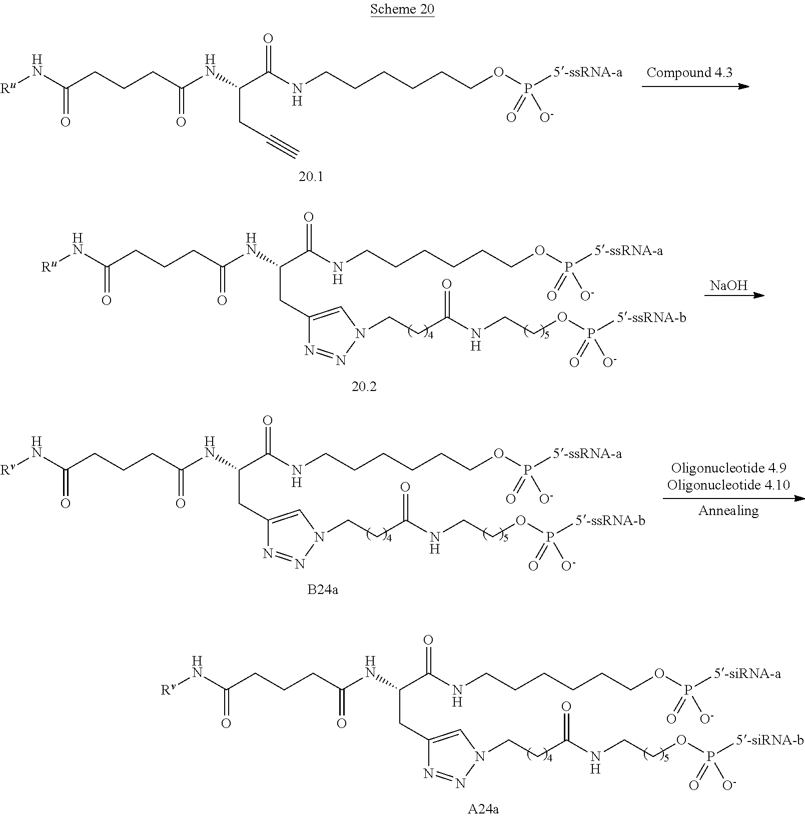

Preparation of compound 20.2. To a solution of compound 20.1 (72 mg, 10 μmol) and compound 4.3 (87 mg, 12 μmol) in water (3 mL) was copper(I) bromide dimethyl sulfide (5 mg, 24 μmol). After stirring at 60° C. for 2 h, NaOAc (246 mg, 3 mmol) was added. The reaction mixture was then centrifuged, and the pellets were collected and dried to afford compound 20.2 (61 mg). MS m/z: [M+H]⁺ Calcd for C₅₃₈H₇₃₁F₅N₁₅₆O₃₃₁P₄₂S₈ 16331.9; Found 16332.0.

Preparation of compound B24a. To a solution of compound 20.2 (61 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (38 mg), which was purified by C18 reverse-phase prep-HPLC and concentrated to about 1 mL to afford compound B24a (18.8 mg) in 15% yield. MS m/z: [M+H]⁺ C₅₂₀H₇₁₃F₅N₁₅₆O₃₂₂P₄₂S₈ 15953.56; Found 15953.67.

Preparation of compound A24a. A solution of compound B24a (1 eq.) and oligonucleotides 4.9 (1 eq.) and 4.10 (1 eq.) in water was heated at 70-95° C. for about 1 to 5 min and then cooled to room temperature to room temperature to afford compound A24a.

### Example 21

### Preparation of GalNAc-siRNA Conjugate A25a

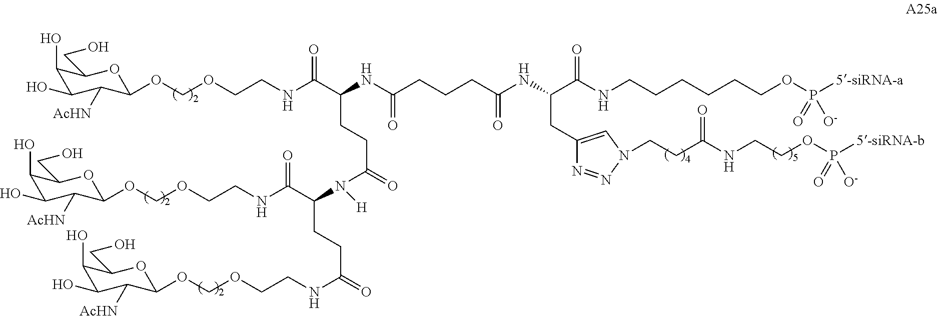

GalNAc-siRNA conjugate A25a was prepared as shown in Scheme 21, wherein R^{x} is a moiety having the structure of

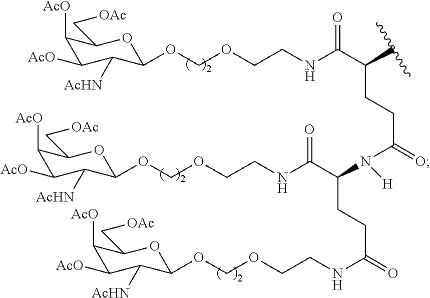

R^{y} is an ASGPR binding moiety having the structure of

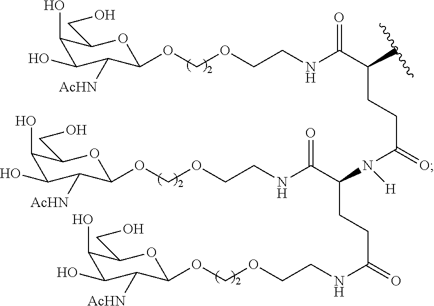

5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

Preparation of compound 21.2. To a solution of compound 21.1 (72 mg, 10 μmol) and compound 4.3 (89 mg, 12 μmol) in water (3 mL) was copper(I) bromide dimethyl sulfide (5 mg, 24 μmol). After stirring at 60° C. for 2 h, NaOAc (246 mg, 3 mmol) was added. The reaction mixture was then centrifuged, and the pellets were collected and dried to afford compound 21.2 (68 mg). MS m/z: [M+H]⁺ Calcd for C₅₂₃H₇₀₃F₅N₁₅₄O₃₂₉P₄₂S₈ 16063.5; Found 16063.97.

Preparation of compound B25a. To a solution of compound 21.2 (68 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (46 mg), which was purified by C18 reverse-phase prep-HPLC and concentrated to about 1 mL to afford compound B25a (19.8 mg) in 15% yield. MS m/z: [M+H]⁺ C₅₀₅H₆₈₅F₅N₁₅₄O₃₂₀P₄₂S₈ 15685.16; Found 15684.94.

Preparation of compound A25a. A solution of compound B25a (1 eq.) and oligonucleotides 4.9 (1 eq.) and 4.10 (1 eq.) in water was heated at 70-95° C. for about 1 to 5 min and then cooled to room temperature to room temperature to afford compound A25a.

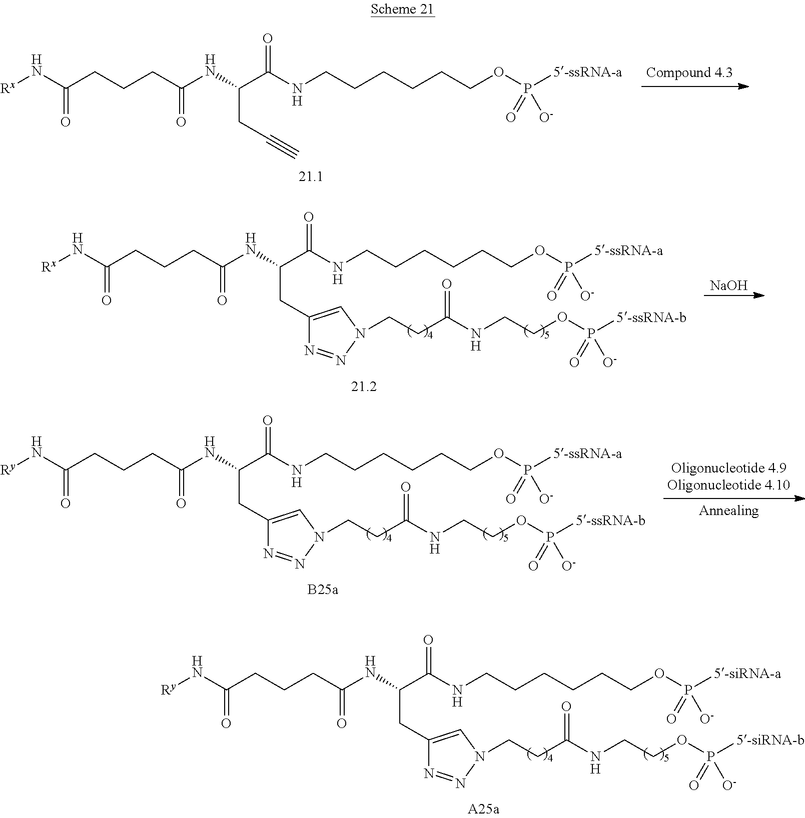

### Example 22

### Preparation of GalNAc-siRNA Conjugate A26a

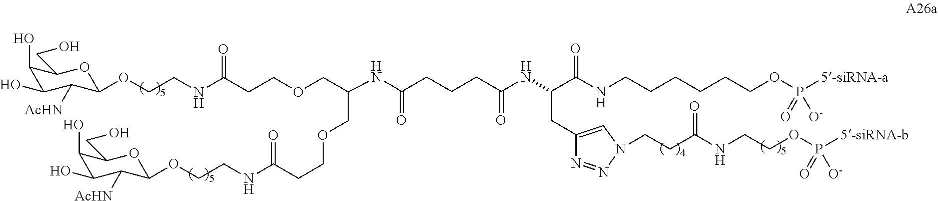

GalNAc-siRNA conjugate A26a was prepared as shown in Scheme 22, wherein R^{p} and R^{q} are as described in Example 1; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

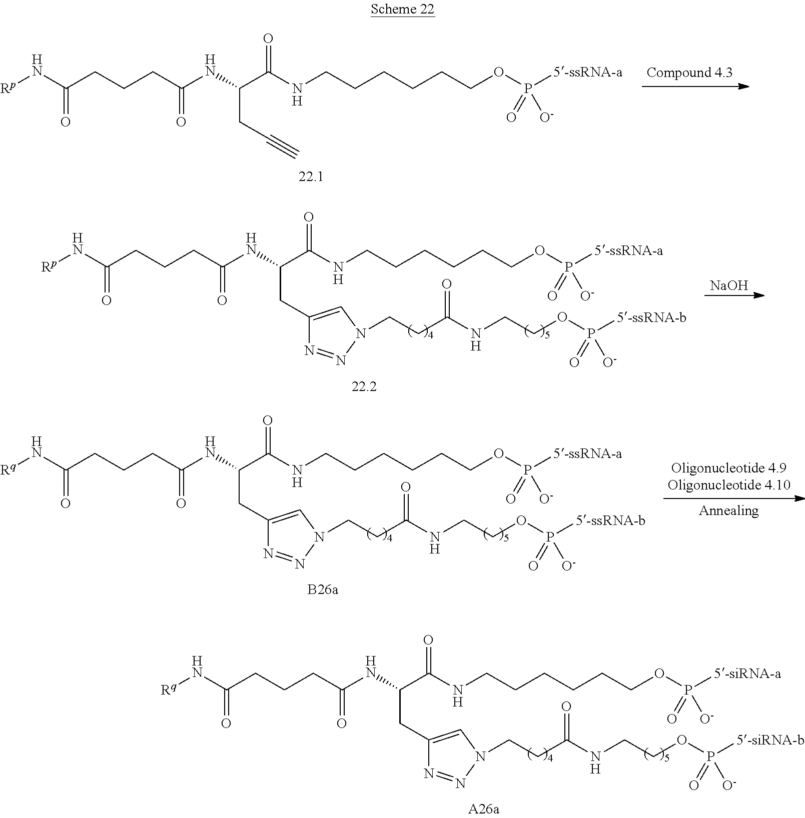

Preparation of compound 22.2. To a solution of compound 22.1 (72 mg, 10 μmol) and compound 4.3 (94 mg, 13 μmol) in water (3 mL) was copper(I) bromide dimethyl sulfide (5 mg, 24 μmol). After stirring at 60° C. for 2 h, NaOAc (246 mg, 3 mmol) was added. The reaction mixture was then centrifuged, and the pellets were collected and dried to afford compound 22.2 (68 mg). MS m/z: [M+H]⁺ Calcd for C₅₀₈H₆₈₄F₅N₁₅₁O₃₁₇P₄₂S₈ 15630.17; Found 15630.67.

Preparation of compound B26a. To a solution of compound 22.2 (68 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (49 mg), which was purified by C18 reverse-phase prep-HPLC and concentrated to about 1 mL to afford compound B26a (26.7 mg) in 20% yield. MS m/z: [M+H]⁺ C₄₉₆H₆₇₂F₅N₁₅₁O₃₁₁P₄₂S₈ 15377.95; Found 15378.25.

Preparation of compound A26a. A solution of compound B26a (1 eq.) and oligonucleotides 4.9 (1 eq.) and 4.10 (1 eq.) in water was heated at 70-95° C. for about 1 to 5 min and then cooled to room temperature to room temperature to afford compound A26a.

### Example 23

### Preparation of GalNAc-siRNA Conjugate A27a

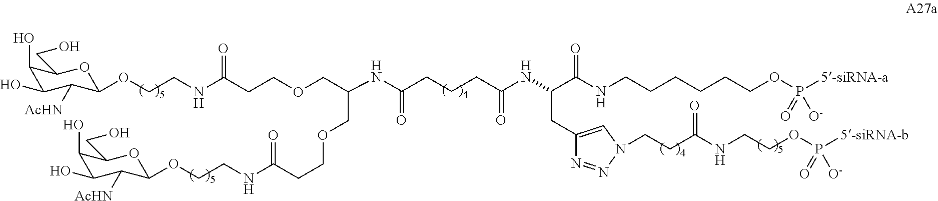

GalNAc-siRNA conjugate A27a was prepared as shown in Scheme 23, wherein R^{p} and R^{q} are as described in Example 1; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

Preparation of compound 23.2. To a solution of compound 23.1 (68 mg, 9 μmol) and compound 4.3 (89 mg, 12 μmol) in water (3 mL) was copper(I) bromide dimethyl sulfide (5 mg, 24 μmol). After stirring at 60° C. for 2 h, NaOAc (246 mg, 3 mmol) was added. The reaction mixture was then centrifuged, and the pellets were collected and dried to afford compound 23.2 (72 mg). MS m/z: [M+H]⁺ Calcd for C₅₁₁H₆₉₀F₅N₁₅₁O₃₁₇P₄₂S₈ 15672.25; Found 15672.31.

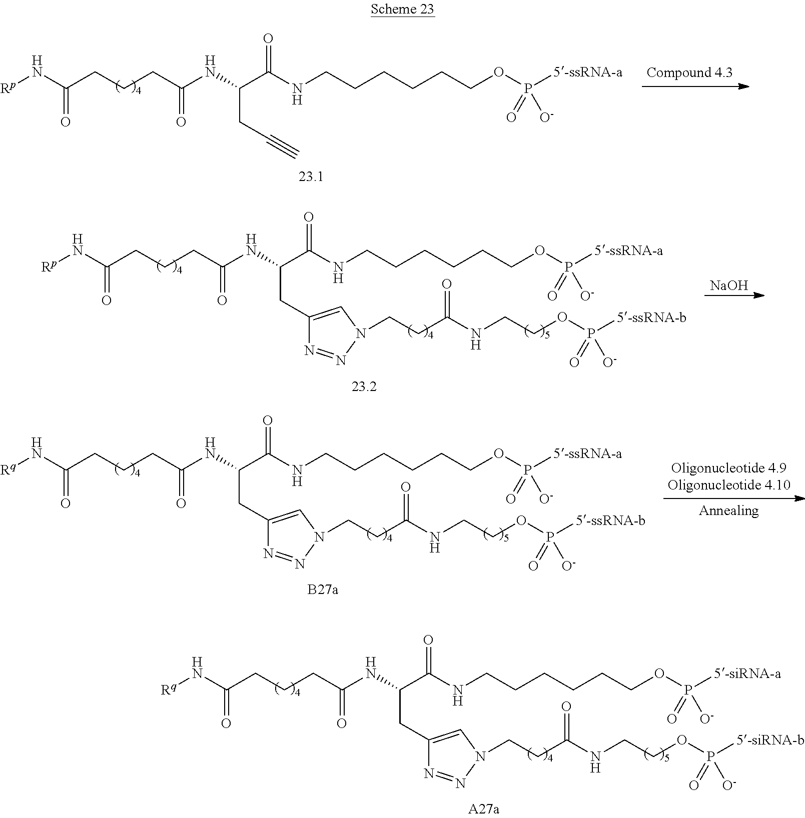

Preparation of compound B27a. To a solution of compound 23.2 (72 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (56 mg), which was purified by C18 reverse-phase prep-HPLC and concentrated to about 1 mL to afford compound B27a (25.4 mg) in 20% yield. MS m/z: [M+H]⁺ C₄₉₉H₆₇₈F₅N₁₅₁O₃₁₁P₄₂S₈ 15420.03; Found 15420.46.

Preparation of compound A27a. A solution of compound B27a (1 eq.) and oligonucleotides 4.9 (1 eq.) and 4.10 (1 eq.) in water was heated at 70-95° C. for about 1 to 5 min and then cooled to room temperature to room temperature to afford compound A27a.

### Example 24

### Preparation of GalNAc-siRNA Conjugate A28a

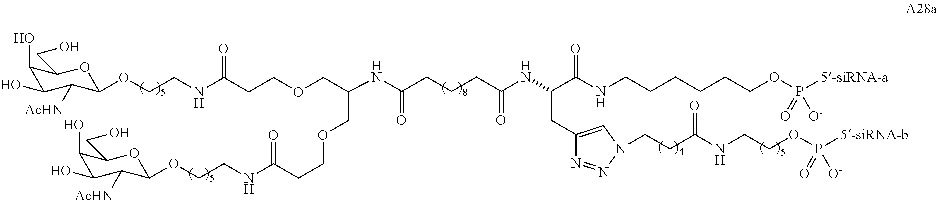

GalNAc-siRNA conjugate A28a was prepared as shown in Scheme 24, wherein R^{p} and R^{q} are as described in Example 1; and wherein 5′-ssRNA-a has the nucleotide sequence of SEQ ID NO: 1 with its 5′-terminus connected to a trivalent linker; 5′-ssRNA-b has the nucleotide sequence of SEQ ID NO: 3 with its 5′-terminus connected to the trivalent linker; 5′-siRNA-a comprises a sense strand having the nucleotide sequence of SEQ ID NO: 1 with its 5′-terminus connected to a trivalent linker and an antisense strand having the nucleotide sequence of SEQ ID NO: 2; and 5′-siRNA-b comprises a sense strand having the nucleotide sequence of SEQ ID NO: 3 with its 5′-terminus connected to the trivalent linker and an antisense strand having the nucleotide sequence of the nucleotide sequence of SEQ ID NO: 4.

Preparation of compound 24.2. To a solution of compound 24.1 (75 mg, 9 μmol) and compound 4.3 (97 mg, 13 μmol) in water (3 mL) was copper(I) bromide dimethyl sulfide (5 mg, 24 μmol). After stirring at 60° C. for 2 h, NaOAc (246 mg, 3 mmol) was added. The reaction mixture was then centrifuged, and the pellets were collected and dried to afford compound 24.2 (81 mg). MS m/z: [M+H]⁺ Calcd for C₅₁₅H₆₉₈F₅N₁₅₁O₃₁₇P₄₂S₈ 15728.36; Found 15730.0.

Preparation of compound B28a. To a solution of compound 24.2 (81 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (64 mg), which was purified by C18 reverse-phase prep-HPLC and concentrated to about 1 mL to afford compound B28a (29.3 mg) in 21% yield. MS m/z: [M+H]⁺ C₅₀₃H₆₈₆F₅N₁₅₁O₃₁₁P₄₂S₈ 15476.14; Found 15475.88.

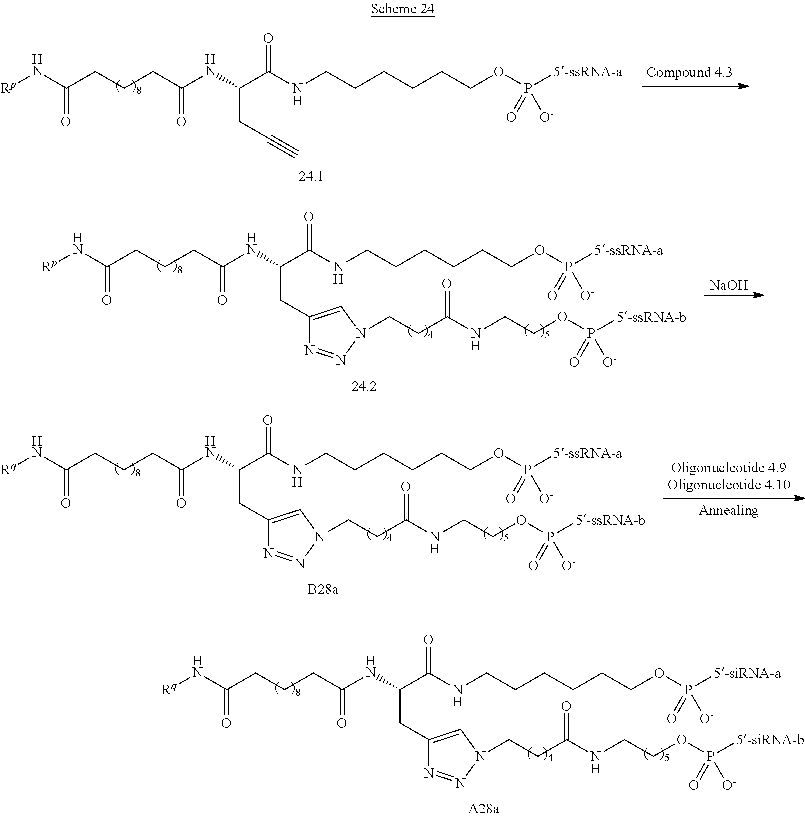

Preparation of compound A28a. A solution of compound B28a (1 eq.) and oligonucleotides 4.9 (1 eq.) and 4.10 (1 eq.) in water was heated at 70-95° C. for about 1 to 5 min and then cooled to room temperature to afford compound A28a.

### Example 25

### Preparation of GalNAc-siRNA Conjugate A29a

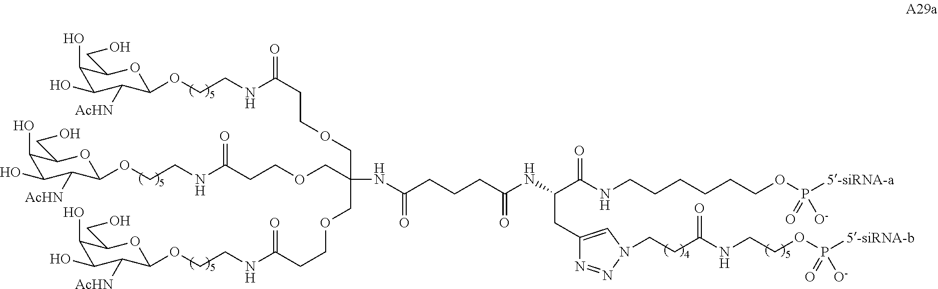

GalNAc-siRNA conjugate A25a was prepared as shown in Scheme 25, wherein R^{s} and R^{t} are as described in Example 2; and wherein 5′-ssRNA-a has the nucleotide sequence of SEQ ID NO: 23 with its 5′-terminus connected to a trivalent linker; 5′-ssDNA has the nucleotide sequence of SEQ ID NO: 25 with its 5′-terminus connected to the trivalent linker; 5′-siRNA-a comprises a sense strand having the nucleotide sequence of SEQ ID NO: 23 with its 5′-terminus connected to a trivalent linker and an antisense strand having the nucleotide sequence of SEQ ID NO: 24.

In Scheme 25, the oligonucleotides of compounds 25.1 (SEQ ID NO: 23), 25.2 (SEQ ID NO: 25), and 25.4 (SEQ ID NO: 24) were prepared by solid-phase oligonucleotide synthesis.

Preparation of compound 25.3. To a solution of compound 25.1 (60 mg, 9 μmol) and compound 25.2 (81 mg, 11 μmol) in water (3 mL) was copper(I) bromide dimethyl sulfide (4 mg, 19 μmol). After stirring at 60° C. for 2 h, NaOAc (246 mg, 3 mmol) was added. The reaction mixture was then centrifuged, and the pellets were collected and dried to afford compound 25.3 (68 mg). MS m/z: [M+H]⁺ Calcd for C₅₁₂H₆₈₂F₄N₁₆₃O₂₈₁P₄₁S₂₅ 15763.37; Found 15763.01.

Preparation of compound B29a. To a solution of compound 25.3 (68 mg) in water (1 mL) was an aqueous 30% NaOH solution (0.12 mL). After shaking at 35° C. for 30 min, the reaction mixture was neutralized with HOAc. The resulting precipitates were collected and dried to yield a crude product (41 mg), which was purified by C18 reverse-phase prep-HPLC and concentrated to about 1 mL to afford compound B29a (13.9 mg) in 12% yield. MS m/z: [M+H]⁺ C₄₉₄H₆₆₄F₄N₁₆₃O₂₇₂P₄₁S₂₅ 15385.03; Found 15385.45.

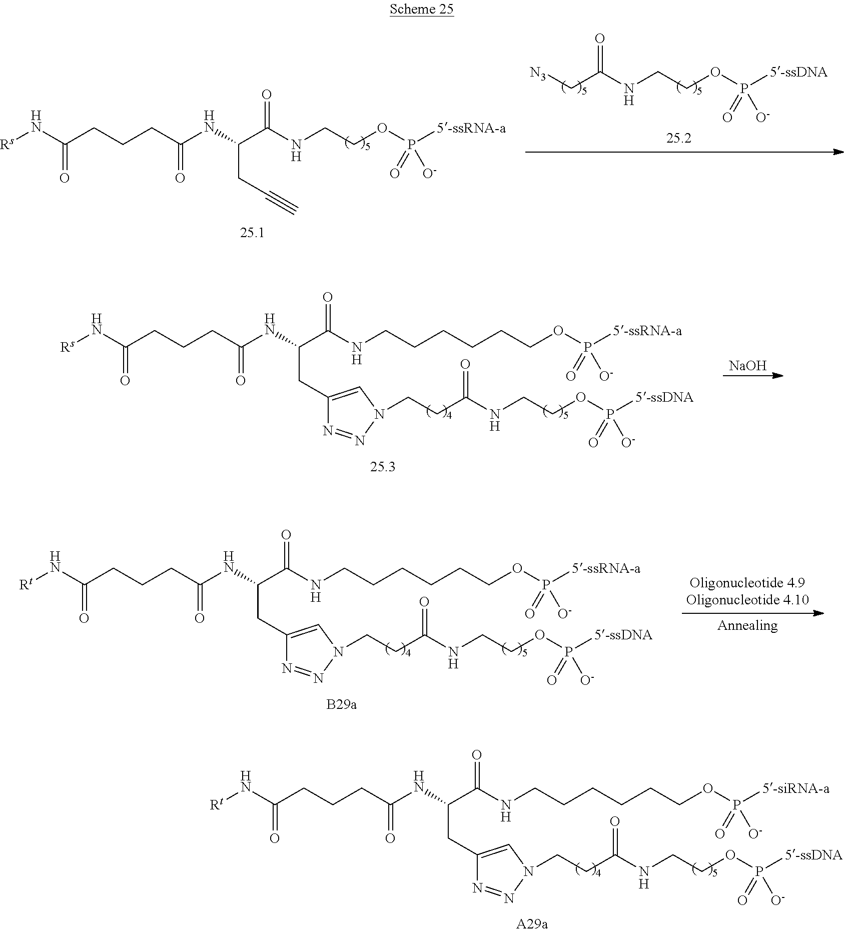

Preparation of compound A29a. A solution of compound B29a (1 eq.) and oligonucleotides 25.4 (1 eq.) in water was heated at 70-95° C. for about 1 to 5 min and then cooled to room temperature to afford compound A29a.

GalNAc-siRNA conjugates A4, A12, A14 to A16, A18 to A33, B4, B12, B14 to B16, and B18 to B33 are prepared similarly according to the procedures described herein.

### Example B1

Suppression of the mRNA Expression of ANGPTL3 and PCSK9

Resuscitated primary human hepatocytes (PPH) were plated in a 96-well plate at 6×10⁵ cells/mL in INVITROGRO. The cells were then treated with GalNAc-siRNA conjugate A18a at 10 nM, 100 nM, and 500 nM for 48 h at 37° C. under 5% CO₂. After the cells were collected by centrifugation, the RNAs of the cells were extracted and reverse-transcribed to cDNAs. The mRNA expression levels of ANGPTL3 and PCSK9 were analyzed by qPRC. The results are summarized in Table 1.

TABLE 1
Inhibition of ANGPTL3 and PCSK9 mRNAs expression levels

| GalNAc- | Inhibition of mRNA Expression Level (%) | | | | | |
|---|---|---|---|---|---|---|
| siRNA | ANGPTL3 | | | PCSK9 | | |
| conjugate | 500 nM | 100 nM | 10 nM | 500 nM | 100 nM | 10 nM |
| A18a | 86.0 ± | 84.3 ± | 75.5 ± | 88.2 ± | 82.1 ± | 62.4 ± |
| | 1.0 | 3.2 | 4.3 | 2.7 | 4.5 | 6.5 |

### Example B2

Suppression of the mRNA Expression of HAO1 and LDHA

Resuscitated primary human hepatocytes (PPH) were plated in a 96-well plate at 6×10⁵ cells/mL in INVITROGRO. The cells were then treated with GalNAc-siRNA conjugate A18b at 10 nM, 100 nM, and 500 nM for 48 h at 37° C. under 5% CO₂. After the cells were collected by centrifugation, the RNAs of the cells were extracted and reverse-transcribed to cDNAs. The mRNA expression levels of HAO1 and LDHA were analyzed by qPRC. The results are summarized in Table 2.

TABLE 2
Inhibition of HAO1 and LDHA expression levels

| GalNAc- | Inhibition of mRNA Expression Level (%) | | | | | |
|---|---|---|---|---|---|---|
| siRNA | HAO1 | | | LDHA | | |
| conjugate | 500 nM | 100 nM | 10 nM | 500 nM | 100 nM | 10 nM |
| A18b | 83.8 ± | 87.3 ± | 81.7 ± | 98.4 ± | 98.7 ± | 87.7 ± |
| | 1.5 | 2.0 | 2.2 | 0.7 | 0.1 | 3.8 |

### Example B3

Suppression of the mRNA Expression of C3 and CFB

Resuscitated primary human hepatocytes (PPH) were plated in a 96-well plate at 6×10⁵ cells/mL in INVITROGRO. The cells were then treated with GalNAc-siRNA conjugate A18c at 10 nM, 100 nM, and 500 nM for 48 h at 37° C. under 5% CO₂. After the cells were collected by centrifugation, the RNAs of the cells were extracted and reverse-transcribed to cDNAs. The mRNA expression levels of C3 and CFB were analyzed by qPRC. The results are summarized in Table 3.

TABLE 3
Inhibition of C3 and CFB expression levels

| GalNAc- | Inhibition of mRNA Expression Level (%) | | | | | |
|---|---|---|---|---|---|---|
| siRNA | C3 | | | CFB | | |
| conjugate | 500 nM | 100 nM | 10 nM | 500 nM | 100 nM | 10 nM |
| A18c | 95.3 ± | 92.3 ± | 80.2 ± | 91.2 ± | 78.8 ± | 61.7 ± |
| | 1.2 | 0.5 | 4.9 | 1.5 | 4.6 | 8.5 |

### Example B4

Suppression of mRNA Expression in Hep3 B Cells

Hep3 B cells were plated in a 96-well plate at 2×10⁵ cells/mL in OPTI-MEM medium. The cells were then treated with a GalNAc-siRNA conjugate at 0.1 and 1 nM for 24 h at 37° C. under 5% CO₂. After the cells were collected by centrifugation, the RNAs of the cells were extracted and reverse-transcribed to cDNAs. The mRNA expression levels of ANGPTL3, C3, C5, CFB, and PCSK9 were analyzed by qPRC. The results are summarized in Tables 4 to 7, wherein A represents an inhibition of no less than 80%; B represents an inhibition of less than 80% but no less than 60%; and C represents an inhibition of less than 60% but no less than 40%.

TABLE 4
Inhibition of ANGPTL3 and PCSK9 mRNAs Expression Levels

| | Inhibition of mRNA Expression Level | |
|---|---|---|
| GalNAc-siRNA | ANGPTL3 | PCSK9 |

| Conjugate | 1 nM | 0.1 nM | 1 nM | 0.1 nM |
|---|---|---|---|---|
| A4a DS0613 | A | C | A | B |
| A18a DS0601 | A | B | A | B |
| A19a DS0602 | A | B | A | B |
| A20a DS0603 | A | C | A | B |
| A21a DS0604 | A | B | A | B |
| A23a DS0606 | A | B | A | B |
| A24a DS0607 | A | B | A | B |
| A25a DS0608 | A | B | A | B |
| A26a DS0610 | A | B | A | B |
| A27a DS0611 | A | C | A | B |
| A28a DS0612 | A | C | A | B |

TABLE 5
Inhibition of C3 and C5 mRNAs Expression Levels

| | Inhibition of mRNA Expression Level | |
|---|---|---|
| GalNAc-siRNA | C3 | C5 |

| Conjugate | 1 nM | 0.1 nM | 1 nM | 0.1 nM |
|---|---|---|---|---|
| A18f | A | B | A | B |

TABLE 6
Inhibition of C3 and CFB mRNAs Expression Levels

| | Inhibition of mRNA Expression Level | |
|---|---|---|
| GalNAc-siRNA | C3 | CFB |

| Conjugate | 1 nM | 0.1 nM | 1 nM | 0.1 nM |
|---|---|---|---|---|
| A18g | A | B | A | B |

TABLE 7
Inhibition of C5 and CFB mRNAs Expression Levels

| | Inhibition of mRNA Expression Level | |
|---|---|---|
| GalNAc-siRNA | C5 | CFB |

| Conjugate | 1 nM | 0.1 nM | 1 nM | 0.1 nM |
|---|---|---|---|---|
| A18c | A | B | A | B |

### Example B5

Suppression of mRNA Expression in NCI-H1944 Cells

NCI-H₁₉₄₄ cells were plated in a 96-well plate at 2×10⁵ cells/mL in OPTI-MEM medium. The cells were then treated with a GalNAc-siRNA conjugate at 0.1 and 1 nM for 24 h at 37° C. under 5% CO₂. After the cells were collected by centrifugation, the RNAs of the cells were extracted and reverse-transcribed to cDNAs. The mRNA expression levels of HAO1 and LDHA were analyzed by qPRC. The results are summarized in Table 8, wherein A represents an inhibition of no less than 80%; B represents an inhibition of less than 80% but no less than 60%; and C represents an inhibition of less than 60% but no less than 40%.

TABLE 8
Inhibition of HAO1 and LDHA mRNAs Expression Levels

| | Inhibition of mRNA Expression Level | |
|---|---|---|
| GalNAc-siRNA | HAO1 | LDHA |

| Conjugate | 1 nM | 0.1 nM | 1 nM | 0.1 nM |
|---|---|---|---|---|
| A18d DS0901 | A | A | A | B |
| A18e DS0902 | A | A | A | A |

### Example B6

Effects of GalNAc-siRNA Conjugates on ANGPTL3, hPCSK9, LDL-C, and TG in hPCSK9-UTR Mice

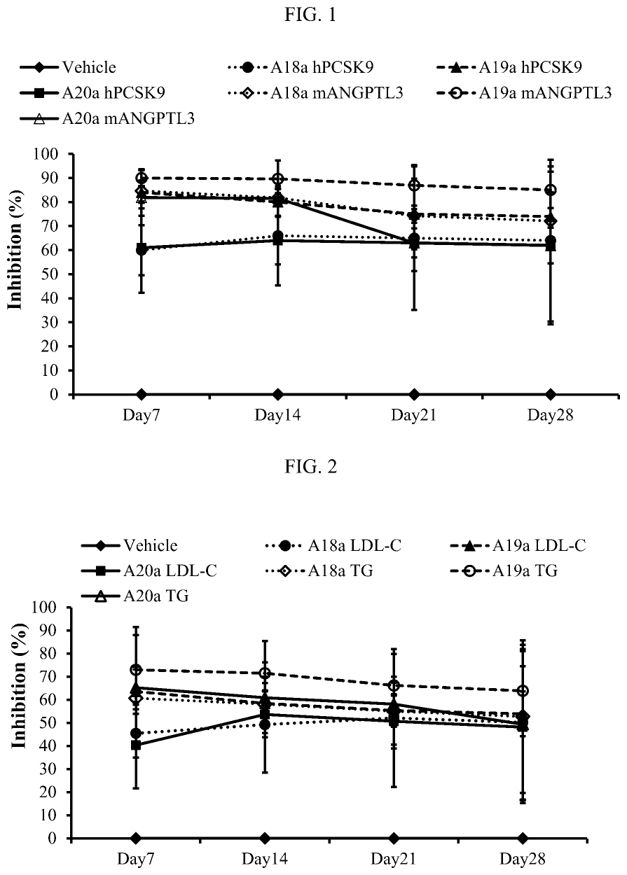

The inhibitory effects of a GalNAc-siRNA conjugate on ANGPTL3, hPCSK9, LDL-C, and TG were evaluated in hPCSK9-UTR mice. The GalNAc-siRNA conjugate (6 mg/kg) was administered subcutaneously to hPCSK9-UTR mice (6-8 weeks of age) on Day 0. Blood samples were collected from each mouse on Day 0, Day 7, Day 14, Day 21, and Day 28. The LDL-C and TG levels in each blood sample were analyzed by blood biochemistry. The hPCSK9 and mANGPTL3 protein levels were analyzed by ELISA. The results are shown in FIGS. 1 and 2.

### Example B7

### Effects of GalNAc-siRNA Conjugates on C3, C5, and CFB in Mice

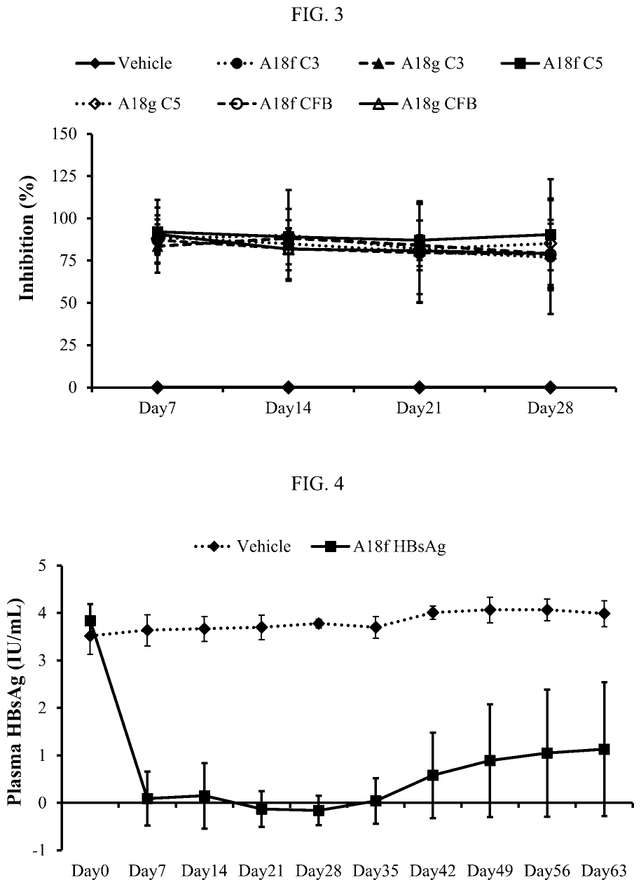

The inhibitory effects of a GalNAc-siRNA conjugate on C3, C5, and CFB were evaluated in mice. The GalNAc-siRNA conjugate (6 mg/kg) was administered subcutaneously to mice (6-8 weeks of age) on Day 0. Blood samples were collected from each mouse on Day 0, Day 7, Day 14, Day 21, and Day 28. The C3, C5, and CFB protein levels were analyzed by ELISA. The results are shown in FIG. 3.

### Example B8

### Antiviral Effect of GalNAc-siRNA Conjugate in HBV Infected Mice

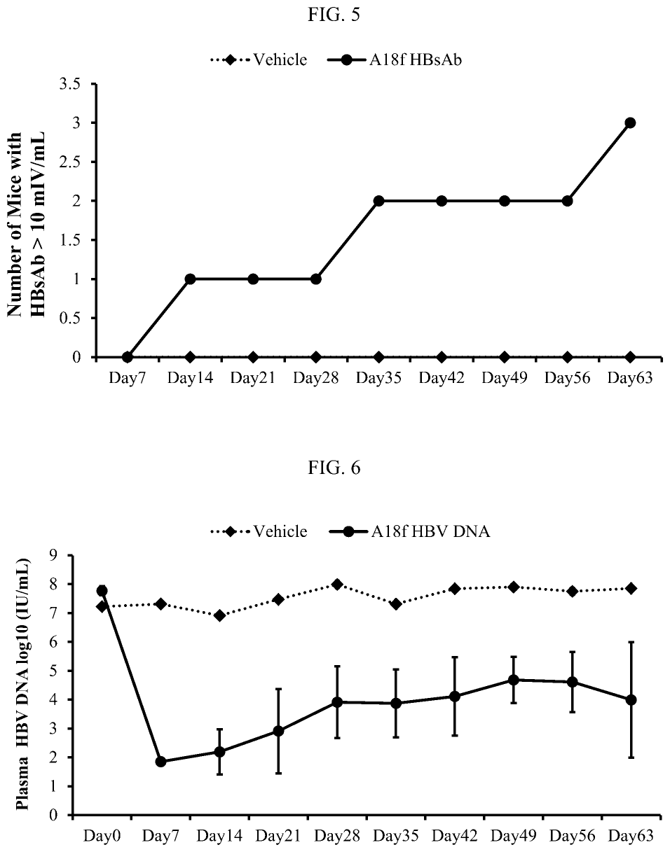

Fifteen specific-pathogen-free (SPF) C57BL/6 male mice were housed under a controlled environment for 7 days. After daily observation, the mice were confirmed to be healthy and normal. Each mouse was injected intravenously with rAAV8-1.3 HBV ayw viruses (1×10¹¹ vg/150 μL). On the 4th week after the intravenous injection, a blood sample was collected from the orbital venous plexus of each mouse to detect its HBsAg level in the plasma. Ten mice were randomly divided into two groups according to the HBsAg detection value with 5 mice per group. Blood samples were collected on Day 0 to detect HBV DNA and HBsAg in the mouse serum. A GalNAc-siRNA conjugate was administered by subcutaneous injection on Day 0. Blood samples were collected from each mouse on Day 7, Day 14, Day 21, Day 28, Day 35, Day 42, Day 49, Day 56, and Day 63. The levels of HBV DNA, HbsAg, and HBsAb in each mouse serum were analyzed. The results are shown in FIGS. 4 to 6.

Sequences described herein are provided in the sequence table below.

SEQUENCE TABLE

| SEQ ID NO: | Description | Amino Acid Sequence |
|---|---|---|
| 1 | PCSK9 Sense RNA | CsUsAGACfCUfGUdTUUGCUUUUsGsU |
| 2 | PCSK9 Antisense RNA | AsfCsAfAfAfAGfCAfAAfACfAGfGUfCUAGsAsA |
| 3 | ANGPTL3 Sense RNA | GsCsUCAACAfUfAfUUUGAUCAGsUsA |
| 4 | ANGPTL3 Antisense RNA | UsfAsCfUGfAUfCAfAAfUAfUGUUfGAsfGsC |
| 5 | LDHA Sense RNA | AsUsGUUGfUCfCfUfUUUUAUCUGsAsU |
| 6 | LDHA Antisense RNA | CsfAsGfAUfAAfAAAGfGAfCAfACAUsGsC |
| 7 | HAO1 Sense RNA | GsAsCUUUfCAfUfCfCUGGAAAUAsUSA |
| 8 | HAO1 Antisense RNA | UsfAsUAUfUUfCfCAGGAfUGfAAAGUCsCsA |
| 9 | C5 Sense RNA | AsAsfGCfAAfGAfUfAfUUfUUUfAUfAAsUSA |
| 10 | C5 Antisense RNA | UsfAsfUUfAUAfAAfAAUAfUCfUUfGCUUsUsUdTdT |
| 11 | CFB Sense RNA | fUsGsfUGfUUfCAfAfAfGUfCAfAGfGAfUsAsfU |
| 12 | CFB Antisense RNA | AsfUsAfUCfCUfUGfACUUfUGfAAfCAfCASUsG |
| 13 | LDHA Sense RNA | CsUsUCAAfGUfUfCfAUCAUUCCCsAsA |
| 14 | LDHA Antisense RNA | UsfUsGGGfAAfUfGAUGAfACfUUGAAGsAsU |
| 15 | LDHA Sense RNA | GsCsAGAUfGAfAfCfUUGCUCUUGsUsU |
| 16 | LDHA Antisense RNA | AsfAsCAAfGAfGfCAAGUfUCfAUCUGCsCsA |
| 17 | HAO1 Sense RNA | GsCsAUGUfAUfUfAfCUUGACAAAsGsA |
| 18 | HAO1 Antisense RNA | UsfCsUUUfGUfCFAAGUAfAUfACAUGCsUsG |
| 19 | C3 Sense RNA | AsAsCAAGfAAfGfAfACAAACUCAsCsA |
| 20 | C3 Antisense RNA | UsfGsUGAfGUUUGUUCfUUfCUUGUUsCsA |
| 21 | CFB Sense RNA | fUsGsfUGfUUfCAfAfAfGUfCAfAGfGAfUsAsfU |
| 22 | CFB Antisense RNA | AsfUsAfUCfCUfUGfACUUfUGfAAfCAfCASUsG |
| 23 | HBV Sense RNA | GsGsGUfUUfUfUfCUUGUUGACsAsA |
| 24 | HBV Antisense RNA | UsUsGUCAfACAAGfAAfAAACCCsUsU |
| 25 | HBV DNA | dTdGdAdCdTdGdTdGdAdAdCdGdTdTdCdGdAdGdA |
| | | dTdGdA |

fA, fC, fG, and fU represent 2′-fluoroadenosine, 2′-fluorocytidine, 2′-fluorogunaosine, and 2′-fluorouridine, respectively; A, C, G, and U represent 2′-O-methyladenosine, 2′-O-methylcytidine, 2′-O-methylguanosine, or 2′-O-methyluridine, respectively; dA, dC, dG, and dT represent 2′-deoxyadenosine, 2′-deoxycytidine, 2′- deoxyguanosine, and 2′-deoxythymidine, respectively; and s represents phosphorothioate.

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A carbohydrate-oligonucleotide conjugate comprising an ASGPR binding moiety and two oligonucleotides.

2. The carbohydrate-oligonucleotide conjugate of claim 1, comprising an ASGPR binding moiety, two oligonucleotides, and a trivalent linker.

3. The carbohydrate-oligonucleotide conjugate of claim 1 or 2, having the structure of Formula (I): 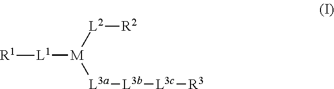
wherein:
R¹ is an ASGPR binding moiety;
R² and R³ are each independently an oligonucleotide;
L¹, L², L^{3a}, and L^{3c} are each independently a linker;
L^{3b} is (i) heteroarylene or heterocyclylene; or (ii) a bond; and
M is (i) N or CH; or (ii) trivalent C₁₋₆ alkyl, trivalent C₁₋₆ heteroalkyl, trivalent C₁₋₆ alkenyl, trivalent C₃₋₁₀ cycloalkyl, trivalent C₆₋₁₄ aryl, trivalent heteroaryl, or trivalent heterocyclyl;
wherein each alkyl, heteroalkyl, alkenyl, cycloalkyl, aryl, heteroaryl, heteroarylene, heterocyclyl, or heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{c}, —OS(O)₂R^{c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

4. The carbohydrate-oligonucleotide conjugate of claim 3, wherein L^{3b} is heteroarylene, optionally substituted with one or more substituents Q.

5. The carbohydrate-oligonucleotide conjugate of claim 3 or 4, wherein L^{3b} is monocyclic heteroarylene, optionally substituted with one or more substituents Q.

6. The carbohydrate-oligonucleotide conjugate of any one of claims 3 to 5, wherein L^{3b} is 5-membered heteroarylene, each optionally substituted with one or more substituents Q.

7. The carbohydrate-oligonucleotide conjugate of any one of claims 3 to 6, wherein L^{3b} is [1,2,3]triazoldiyl, optionally substituted with one or more substituents Q.

8. The carbohydrate-oligonucleotide conjugate of any one of claims 3 to 7, having the structure of Formula (II):

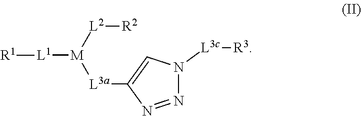

9. The carbohydrate-oligonucleotide conjugate of any one of claims 3 to 8, wherein R¹ is an ASGPR binding moiety comprising from about 1 to about 10 N-acetylgalactosamines.

10. The carbohydrate-oligonucleotide conjugate of any one of claims 3 to 9, wherein R¹ is an ASGPR binding moiety comprising about 1, about 2, about 3, about 4, or about 5 N-acetylgalactosamines.

11. The carbohydrate-oligonucleotide conjugate of any one of claims 3 to 10, wherein R¹ is an ASGPR binding moiety comprising about 2, about 3, or about 4 N-acetylgalactosamines.

12. The carbohydrate-oligonucleotide conjugate of any one of claims 3 to 11, wherein R¹ is an ASGPR binding moiety having the structure of Formula (A-I): 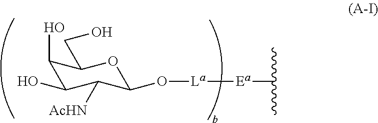
or an enantiomer, a mixture of enantiomers, a diastereomer, a mixture of two or more diastereomers, a tautomer, or a mixture of two or more tautomers thereof; wherein:
E^{a} and b are:
(i) b is an integer of 2; and E^{a} is a trivalent linker;
(ii) b is an integer of 1; and E^{a} is a bond, CH₂, or NH; or
(iii) b is an integer of 3; and E^{a} is a tetravalent linker; and
each L^{a} is independently a linker.

13. The carbohydrate-oligonucleotide conjugate of claim 12, wherein b is an integer of 2; and E^{a} is a trivalent linker.

14. The carbohydrate-oligonucleotide conjugate of claim 12 or 13, wherein E^{a} is CH or N.

15. The carbohydrate-oligonucleotide conjugate of claim 12, wherein b is an integer of 3 and E^{a} is a tetravalent linker.

16. The carbohydrate-oligonucleotide conjugate of claim 15, wherein E^{a} is C.

17. The carbohydrate-oligonucleotide conjugate of any one of claims 12 to 16, wherein each L^{a} is independently a linker having the structure of —Zⁿ—(Rⁿ Zⁿ)_{z}—, wherein:
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene, each optionally substituted with one or more substituents Q;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —C(R^{1a})═NO—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}, —NR^{1a}S(O)NR^{1b}—, —NR^{1a}S(O)₂NR^{1b}—, —P(O₂)O)—, —P(O)(S)O—, —S—, —S(O)—, —S(O)₂—, —S(O)NR^{1b}—, or —S(O)₂NR^{1b}—;
each R^{1a}, R^{1b}, R^{1c}, and R^{1d} is independently (i) hydrogen or deuterium; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more substituents Q; and
z is an integer of 0, 1, 2, 3, 4, 5, 6, 7, 8, 9, or 10.

18. The carbohydrate-oligonucleotide conjugate of claim 17, wherein each Rⁿ is independently C₁₋₁₀ alkylene, C₆₋₁₄ arylene, or heteroarylene, each of which is optionally substituted with one or more substituents Q.

19. The carbohydrate-oligonucleotide conjugate of claim 17 or 18, wherein each Rⁿ is independently methanediyl, ethanediyl, propanediyl, butanediyl, pentanediyl, hexanediyl, phendiyl, 1,2,3-triazoldiyl, pyrrolidindiyl, or piperidindiyl, each optionally substituted with one, two, or three substituents Q.

20. The carbohydrate-oligonucleotide conjugate of any one of claims 17 to 19, wherein each Rⁿ is independently methanediyl, ethane-1,2-diyl, propane-1,3-diyl, butane-1,4-diyl, pentane-1,5-diyl, hexane-1,6-diyl, phen-1,4-diyl, 1,2,3-triazol-1,4-diyl, pyrrolidin-1,3-diyl, or piperidin-1,4-diyl, each optionally substituted with one or more substituents Q.

21. The carbohydrate-oligonucleotide conjugate of any one of claims 17 to 20, wherein each Zⁿ is independently a bond, —C(O)—, —C(O)O—, —C(O)NH—, —OC(O)NH—, —C(CH₃)═NO—, —O—, —OP(O₂)O—, —OP(O₂)S—, —NH—, —N(CH₃)—, —S—, or —S(O)₂—.

22. The carbohydrate-oligonucleotide conjugate of any one of claims 17 to 21, wherein each Zⁿ is independently a bond, —C(O)NH—, —O—, —OP(O₂)O—, —OP(O₂)S—, or —NH—.

23. The carbohydrate-oligonucleotide conjugate of any one of claims 17 to 22, wherein z is an integer of 0, 1, 2, 3, 4, or 5.

24. The carbohydrate-oligonucleotide conjugate of any one of claims 17 to 23, wherein each L^{a} is independently:

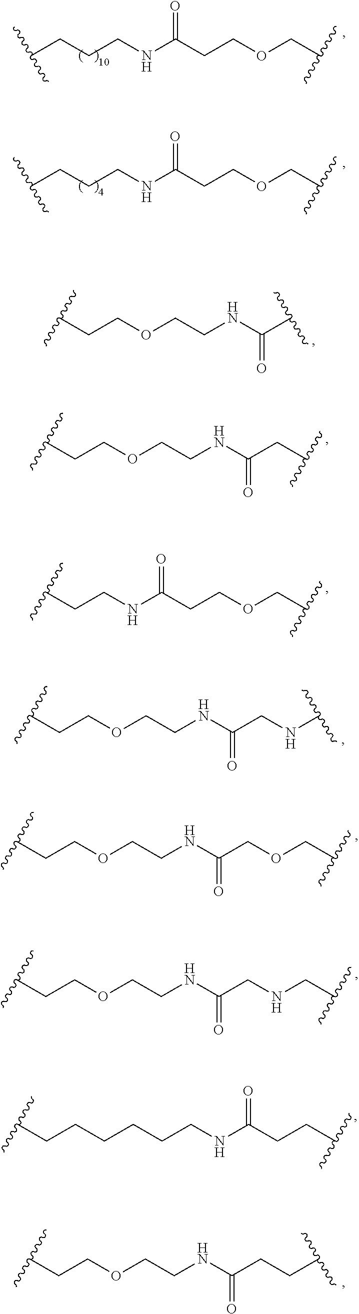

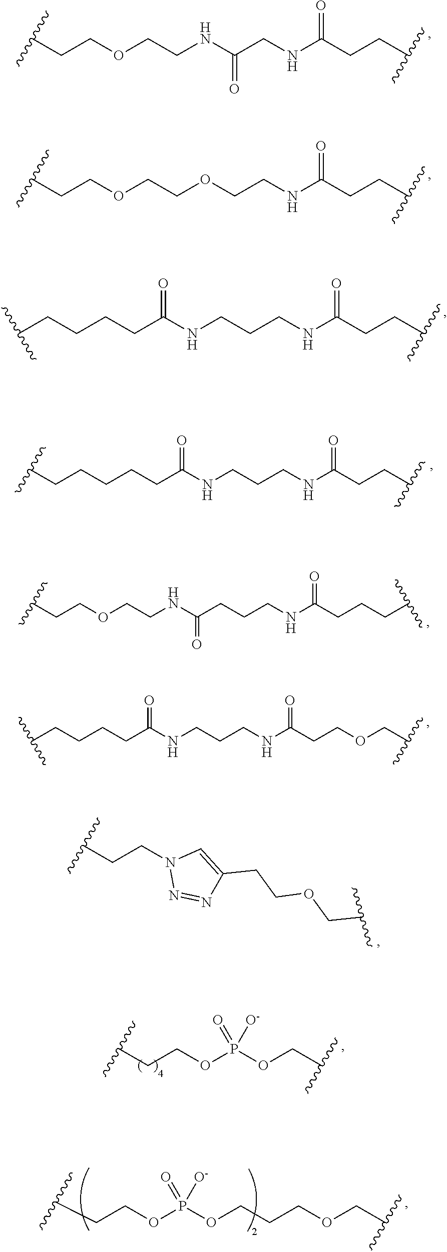

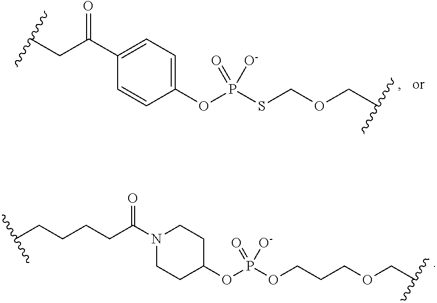 [SMILES: *COCSP(=O)([O-])Oc1ccc(C(=O)C*)cc1 ; *CCCCC(=O)N1CCC(OP(=O)([O-])OCCCOC*)CC1]

25. The carbohydrate-oligonucleotide conjugate of any one of claims 3 to 11, wherein the ASGPR binding moiety has the structure of Formula (A-V): 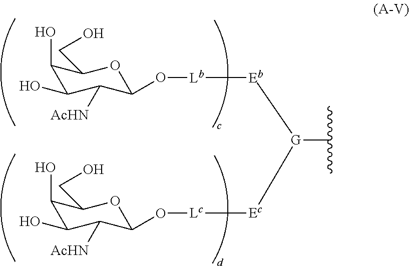
or an enantiomer, a mixture of enantiomers, a diastereomer, a mixture of two or more diastereomers, a tautomer, or a mixture of two or more tautomers thereof; wherein:
E^{b} and c are:
(i) c is an integer of 2; and E^{b} is a trivalent linker;
(ii) c is an integer of 1; and E^{b} is a bond; or
(iii) c is an integer of 3; and E^{b} is a tetravalent linker;
E^{c} and d are:
(i) d is an integer of 1; and E^{c} is bond;
(ii) d is an integer of 2; and E^{c} is a trivalent linker; or
(iii) d is an integer of 3; and E^{c} is a tetravalent linker;
G is a trivalent linker; and
each L^{b} and L^{c} is independently a divalent linker.

26. The carbohydrate-oligonucleotide conjugate of claim 25, wherein c is an integer of 2; and E^{b} is a trivalent linker.

27. The carbohydrate-oligonucleotide conjugate of claim 25, wherein E^{b} is

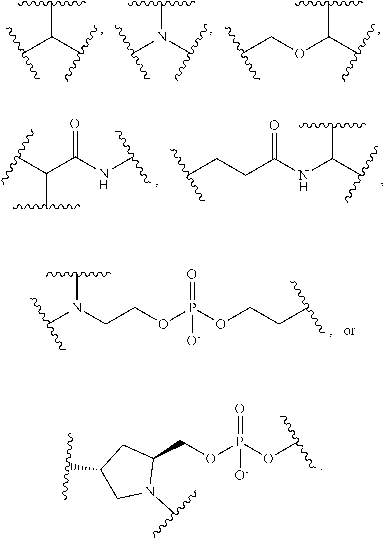

28. The carbohydrate-oligonucleotide conjugate of claim 25, wherein c is an integer of 1; and E^{b} is a bond.

29. The carbohydrate-oligonucleotide conjugate of any one of claims 25 to 28, wherein d is an integer of 2; and E^{c} is a trivalent linker.

30. The carbohydrate-oligonucleotide conjugate of 25 to 29, wherein E^{c} is

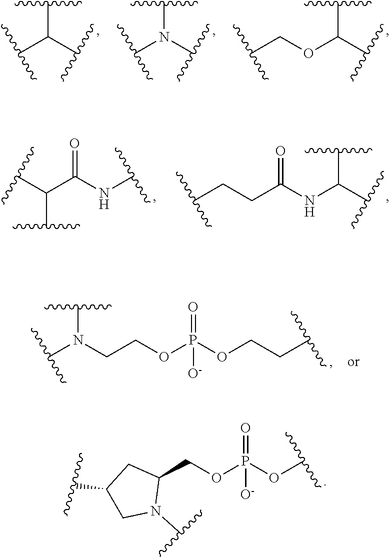

31. The carbohydrate-oligonucleotide conjugate of any one of claims 25 to 30, wherein each L^{b} and L^{c} is independently a linker having the structure of —Zⁿ—(Rⁿ—Zⁿ)_{z}—, wherein:
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene, each optionally substituted with one or more substituents;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —C(R^{1a})═NO—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —P(O₂)O—, —P(O)(S)O—, —S—, —S(O)—, —S(O)₂—, —S(O)NR^{1b}, or —S(O)₂NR^{1b}—;
each R^{1a}, R^{1b}, R^{1c}, and R^{1d} is independently (i) hydrogen or deuterium; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more substituents; and
z is an integer of 0, 1, 2, 3, 4, 5, 6, 7, 8, 9, or 10.

32. The carbohydrate-oligonucleotide conjugate of claim 31, wherein each Rⁿ is independently C₁₋₁₀ alkylene, C₆₋₁₄ arylene, or heteroarylene, each of which is optionally substituted with one or more substituents Q.

33. The carbohydrate-oligonucleotide conjugate of claim 31 or 32, wherein each Rⁿ is independently methanediyl, ethanediyl, propanediyl, butanediyl, pentanediyl, hexanediyl, phendiyl, 1,2,3-triazoldiyl, pyrrolidindiyl, or piperidindiyl, each optionally substituted with one, two, or three substituents Q.

34. The carbohydrate-oligonucleotide conjugate of any one of claims 31 to 33, wherein each Rⁿ is independently methanediyl, ethane-1,2-diyl, propane-1,3-diyl, butane-1,4-diyl, pentane-1,5-diyl, hexane-1,6-diyl, phen-1,4-diyl, 1,2,3-triazol-1,4-diyl, pyrrolidin-1,3-diyl, or piperidin-1,4-diyl, each optionally substituted with one or more substituents Q.

35. The carbohydrate-oligonucleotide conjugate of any one of claims 31 to 34, wherein each Zⁿ is independently a bond, —C(O)—, —C(O)O)—, —C(O)NH—, —OC(O)NH—, —C(CH₃)═NO—, —O—, —OP(O₂)O—, —OP(O₂)S—, —NH—, —N(CH₃)—, —P(O₂)O—, —P(O)(S)O—, —S—, or —S(O)₂—.

36. The carbohydrate-oligonucleotide conjugate of any one of claims 31 to 35, wherein each Zⁿ is independently a bond, —C(O)NH—, —O—, —OP(O₂)O—, —OP(O₂)S—, or —NH—.

37. The carbohydrate-oligonucleotide conjugate of any one of claims 31 to 36, wherein z is an integer of 0, 1, 2, 3, 4, or 5.

38. The carbohydrate-oligonucleotide conjugate of claim 31 or 37, wherein each L^{b} and L^{c} is independently:

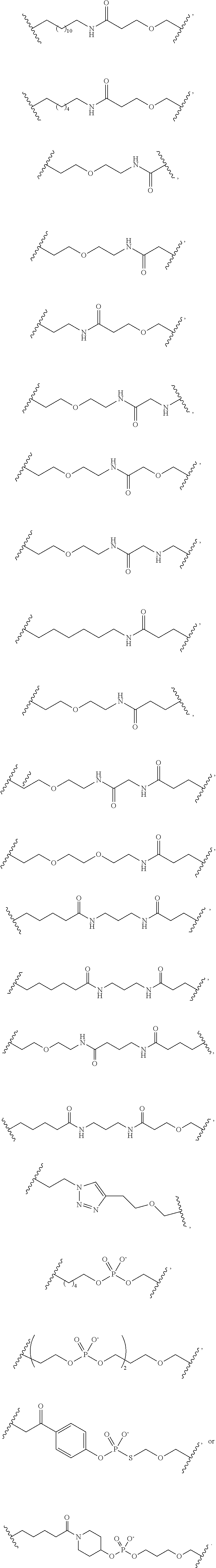

39. The carbohydrate-oligonucleotide conjugate of any one of claims 3 to 38, wherein R¹ is an ASGPR binding moiety having the structure of:

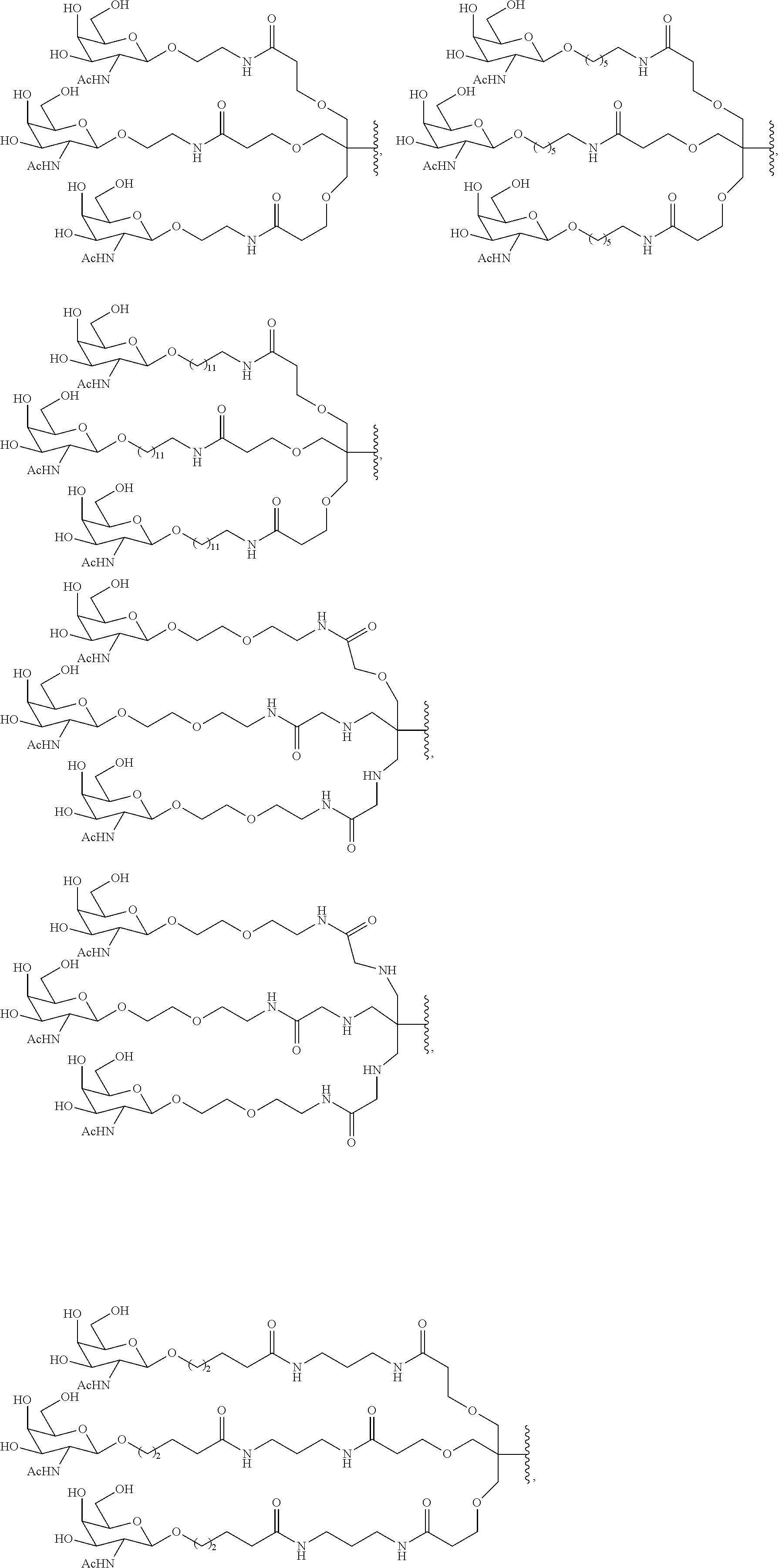

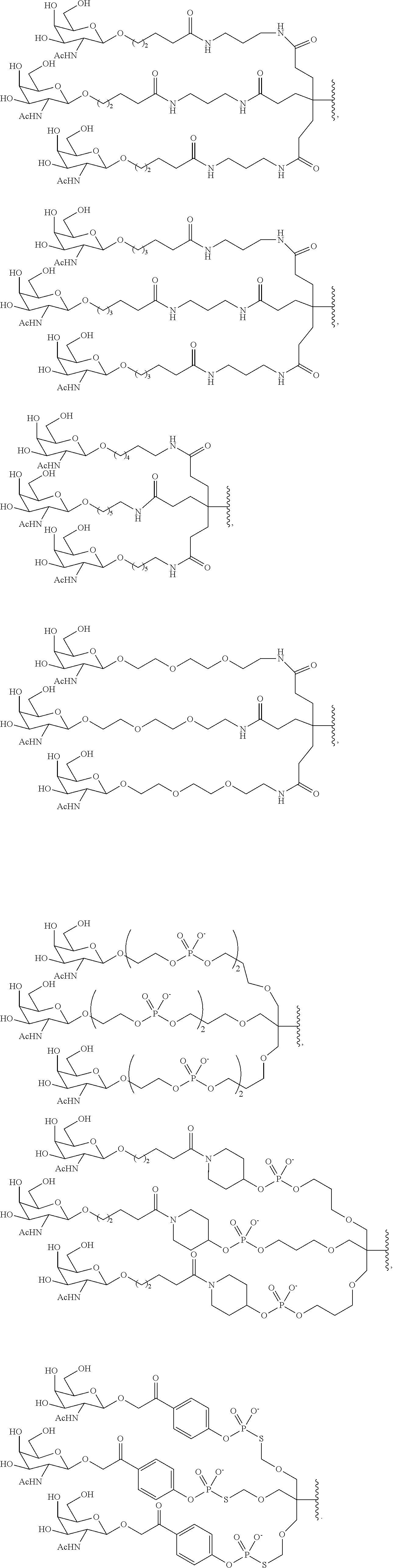

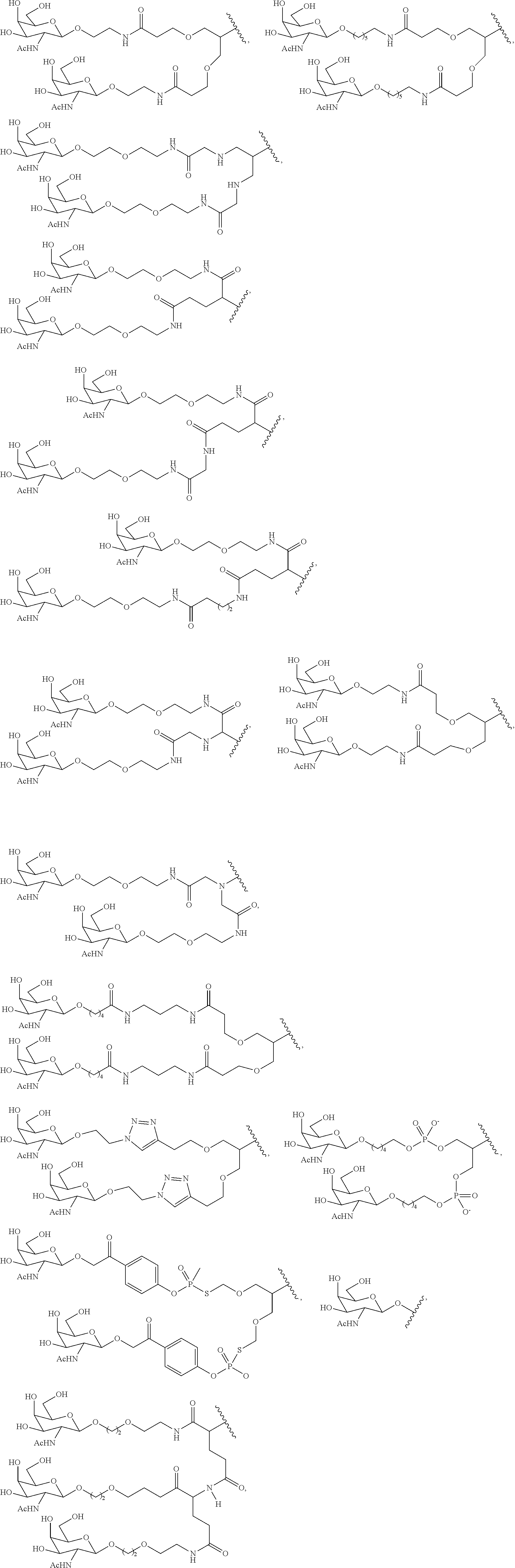

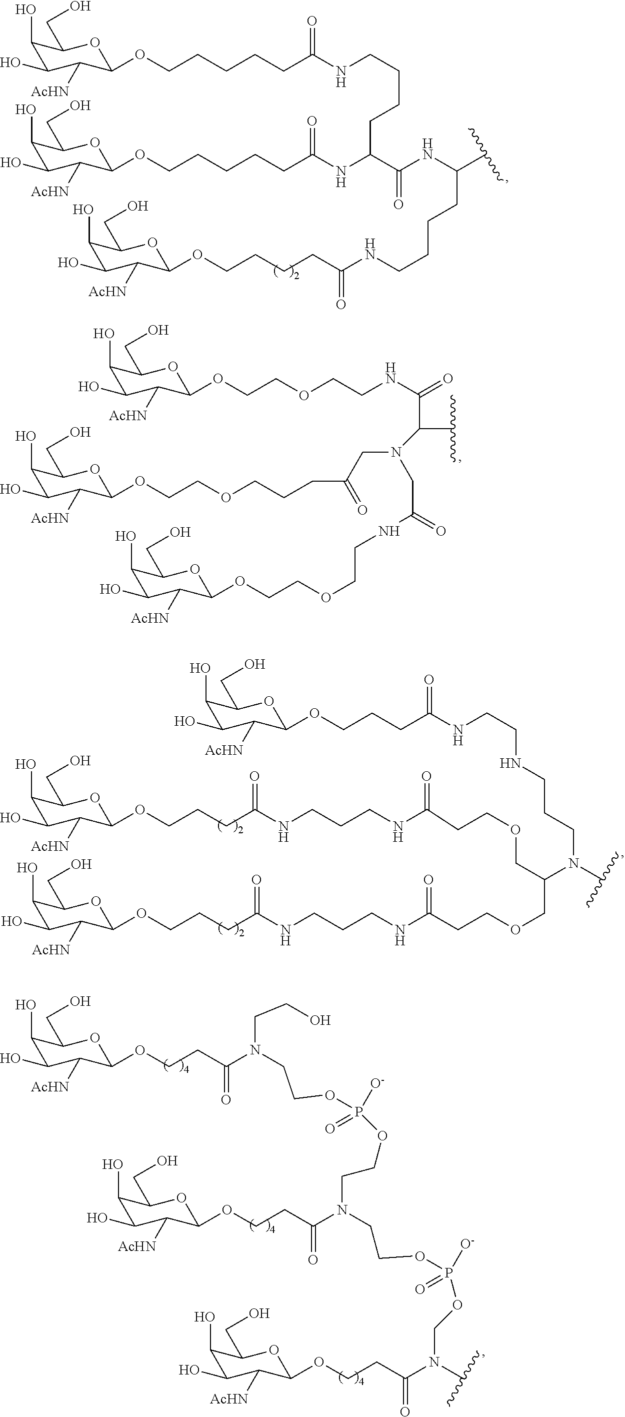

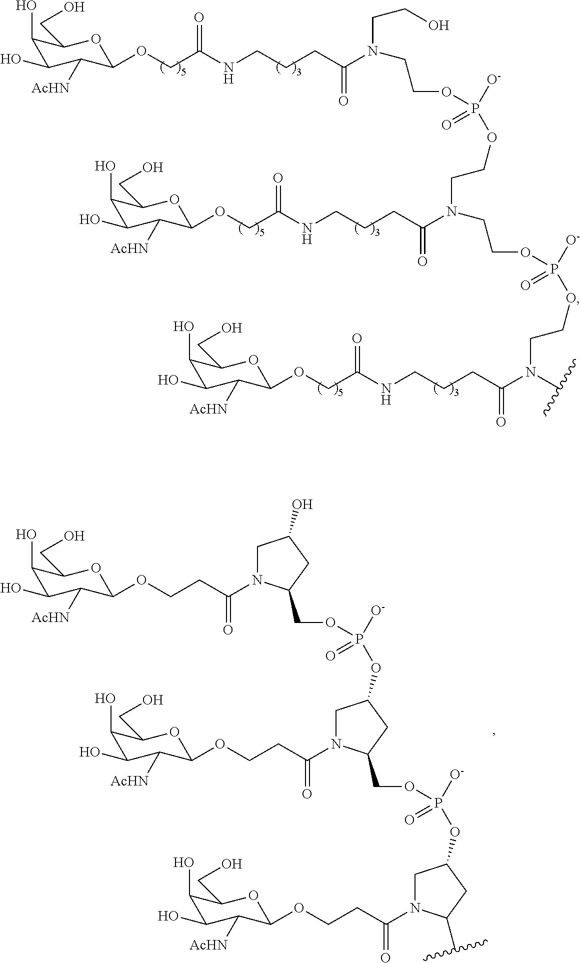

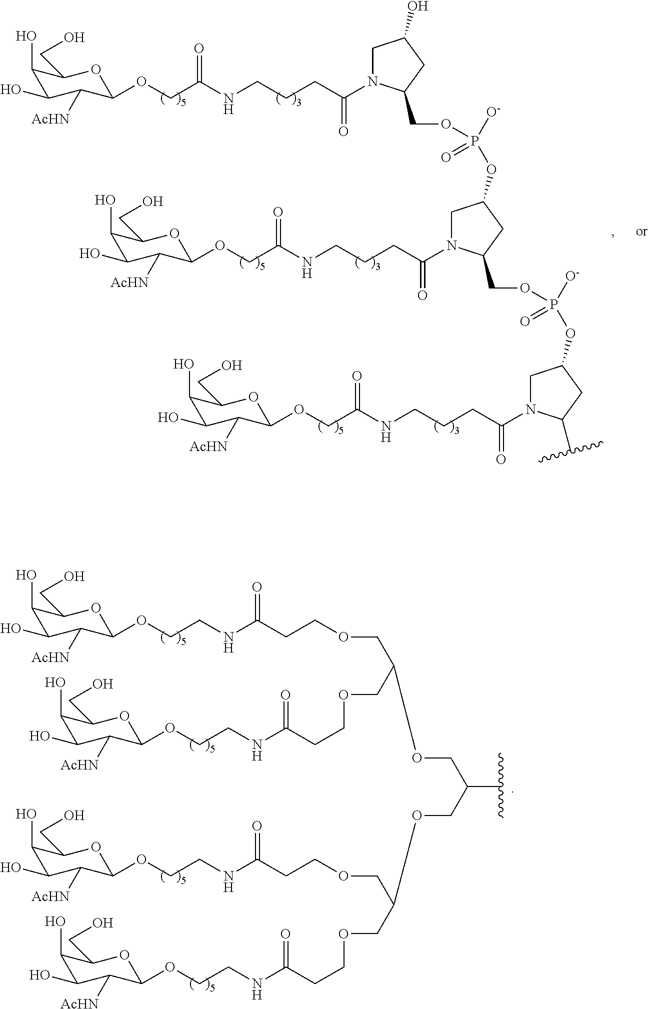

40. The carbohydrate-oligonucleotide conjugate of any one of claims 3 to 39, wherein R² is a double-stranded siRNA.

41. The carbohydrate-oligonucleotide conjugate of claim 40, wherein each strand of the double-stranded siRNA independently comprises from about 10 to about 50, from about 10 to about 30, or from about 15 to about 25 nucleotides.

42. The carbohydrate-oligonucleotide conjugate of any one of claims 3 to 39, wherein R² is a single-stranded oligonucleotide.

43. The carbohydrate-oligonucleotide conjugate of claim 42, wherein R² is a single-stranded oligodeoxyribonucleotide.

44. The carbohydrate-oligonucleotide conjugate of claim 42 or 43, wherein the single-stranded oligonucleotide comprises from about 10 to about 50, from about 10 to about 30, or from about 15 to about 25 nucleotides.

45. The carbohydrate-oligonucleotide conjugate of any one of claims 3 to 44, wherein R³ is a double-stranded siRNA.

46. The carbohydrate-oligonucleotide conjugate of claim 45, wherein each strand of the double-stranded siRNA independently comprises from about 10 to about 50, from about 10 to about 30, or from about 15 to about 25 nucleotides.

47. The carbohydrate-oligonucleotide conjugate of any one of claims 3 to 44, wherein R³ is a single-stranded oligonucleotide.

48. The carbohydrate-oligonucleotide conjugate of claim 47, wherein R³ is a single-stranded oligodeoxyribonucleotide.

49. The carbohydrate-oligonucleotide conjugate of claim 47 or 48, wherein the single-stranded oligonucleotide comprises from about 10 to about 50, from about 10 to about 30, or from about 15 to about 25 nucleotides.

50. The carbohydrate-oligonucleotide conjugate of any one of claims 41, 44, 46, and 49, wherein each nucleotide is independently a natural nucleotide or modified nucleotide.

51. The carbohydrate-oligonucleotide conjugate of any one of claims 41, 44, 46, 49, and 50, wherein each nucleotide is independently adenylate, cytidylate, guanylate, uridylate, 2′-fluoroadenosine, 2′-fluorocytidine, 2′-fluorogunaosine, 2′-fluorouridine, 2′-deoxyadenosine, 2′-deoxycytidine, 2′-deoxygunaosine, 2′-deoxythymidine, 2′-O-methyladenosine, 2′-O-methyl-cytidine, 2′-O-methylguanosine, or 2′-O-methyluridine.

52. The carbohydrate-oligonucleotide conjugate of any one of claims 41 to 51, wherein each strand has one or more phosphate linkage groups each independently replaced with phosphorothioate or phosphorodithiate.

53. The carbohydrate-oligonucleotide conjugate of any one of claims 3 to 41 and 45 to 52, wherein R² is a double-stranded siRNA comprising a pair of nucleotide sequences of SEQ ID NOs: 1 and 2, 3 and 4, 5 and 6, 7 and 8, 9 and 10, 11 and 12, 13 and 14, 15 and 16, 17 and 18, 19 and 20, 21 and 22, or 23 and 24.

54. The carbohydrate-oligonucleotide conjugate of any one of claims 3 to 41 and 45 to 53, wherein R² is a double-stranded siRNA comprising a pair of nucleotide sequences of SEQ ID NOs: 1 and 2, 3 and 4, 5 and 6, 9 and 10, 13 and 14, 15 and 16, 19 and 20, or 23 and 24.

55. The carbohydrate-oligonucleotide conjugate of any one of claims 3 to 46 and 50 to 54, wherein R³ is a double-stranded siRNA comprising a pair of nucleotide sequences of SEQ ID NOs: 1 and 2, 3 and 4, 5 and 6, 7 and 8, 9 and 10, 11 and 12, 13 and 14, 15 and 16, 17 and 18, 19 and 20, 21 and 22, or 23 and 24.

56. The carbohydrate-oligonucleotide conjugate of any one of claims 3 to 46 and 50 to 55, wherein R³ is a double-stranded siRNA comprising a pair of nucleotide sequences of SEQ ID NOs: 1 and 2, 3 and 4, 7 and 8, 11 and 12, 17 and 18, 19 and 20, or 21 and 22.

57. The carbohydrate-oligonucleotide conjugate of any one of claims 3 to 44 and 47 to 56, wherein R³ is a single-stranded oligonucleotide comprising the nucleotide sequence of SEQ ID NO: 1, 3, 5, 7, 9, 11, 13, 15, 17, 19, 21, 23, or 25.

58. The carbohydrate-oligonucleotide conjugate of any one of claims 3 to 57, wherein L¹, L², L^{3a}, and L^{3c} are each independently a linker having the structure of —Zⁿ—(Rⁿ—Zⁿ)_{z}—, wherein:
each Rⁿ is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene, each optionally substituted with one or more substituents;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —C(R^{1a})═NO—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)—NR^{1b}—, —P(O₂)O—, —P(O)(S)O—, —S—, —S(O)—, —S(O)₂—, —S(O)NR^{1b}—, or —S(O)₂NR^{1b}—;
each R^{1a}, R^{1b}, R^{1c}, and R^{1d} is independently (i) hydrogen or deuterium; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each optionally substituted with one or more substituents; and
z is an integer of 0, 1, 2, 3, 4, 5, 6, 7, 8, 9, or 10.

59. The carbohydrate-oligonucleotide conjugate of claim 58, wherein each Rⁿ is independently C₁₋₁₀ alkylene, C₆₋₁₄ arylene, or heteroarylene, each of which is optionally substituted with one or more substituents Q.

60. The carbohydrate-oligonucleotide conjugate of claim 58 or 59, wherein each Rⁿ is independently methanediyl, ethanediyl, propanediyl, butanediyl, pentanediyl, hexanediyl, phendiyl, 1,2,3-triazoldiyl, or pyrrolidindiyl, each optionally substituted with one, two, or three substituents Q.

61. The carbohydrate-oligonucleotide conjugate of any one of claims 58 to 60, wherein each Rⁿ is independently methanediyl, ethane-1,2-diyl, propane-1,3-diyl, butane-1,4-diyl, pentane-1,5-diyl, hexane-1,6-diyl, phen-1,4-diyl, 1,2,3-triazol-1,4-diyl, or pyrrolidin-1,3-diyl, each optionally substituted with one or more substituents Q.

62. The carbohydrate-oligonucleotide conjugate of any one of claims 58 to 61, wherein each Zⁿ is independently a bond, —C(O)—, —C(O)O—, —C(O)NH—, —OC(O)NH—, —C(CH₃)═NO—, —O—, —OP(O₂)O—, —OP(O₂)S—, —NH—, —N(CH₃)—, —P(O₂)O)—, —P(O₂)S—, —S—, or —S(O)₂—.

63. The carbohydrate-oligonucleotide conjugate of any one of claims 58 to 62, wherein each Zⁿ is independently a bond, —C(O)NH—, —O—, —OP(O₂)O—, —OP(O₂)S—, —NH—, —P(O₂)O—, or —P(O₂)S—.

64. The carbohydrate-oligonucleotide conjugate of any one of claims 58 to 63, wherein z is an integer of 0, 1, 2, 3, 4, or 5.

65. The carbohydrate-oligonucleotide conjugate of any one of claims 3 to 64, wherein L¹ is —NHC(O)(CH₂)ₑC(O)NH—, wherein e is an integer of 1, 2, 3, 4, 5, 6, 7, 8, 9, 10, 11, 12, 13, 14, or 15.

66. The carbohydrate-oligonucleotide conjugate of any one of claims 3 to 65, wherein L¹ is

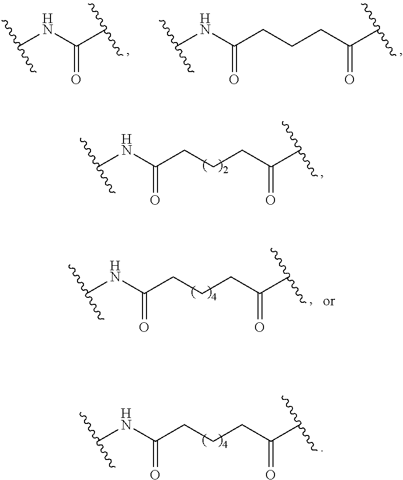

67. The carbohydrate-oligonucleotide conjugate of any one of claims 3 to 66, wherein L² has the structure of —X(CH₂)ₕX—; each X is independently —O—, —S—, or —N(H)—; and h is an integer of 2, 3, 4, 5, 6, 7, 8, 9, 10, 11, or 12.

68. The carbohydrate-oligonucleotide conjugate of any one of claims 3 to 66, wherein L² has the structure of —X(CH₂)ᵢCH(OH)(CH₂)ⱼX—; each X is independently —O—, —S—, or —N(H)—; and i and j are each independently an integer of 2, 3, 4, 5, 6, 7, 8, 9, or 10.

69. The carbohydrate-oligonucleotide conjugate of any one of claims 3 to 66, wherein L² has the structure of 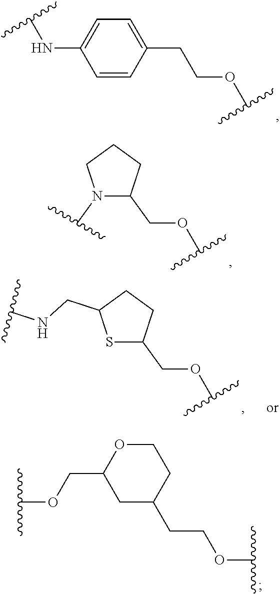 [SMILES: *Nc1ccc(CCO*)cc1 ; *OCC1CCCN1* ; *NCC1CCC(CO*)S1 ; *OCCC1CCOC(CO*)C1]
and L² is attached to a 5′-terminus of an oligonucleotide.

70. The carbohydrate-oligonucleotide conjugate of any one of claims 3 to 66, wherein L² has the structure of 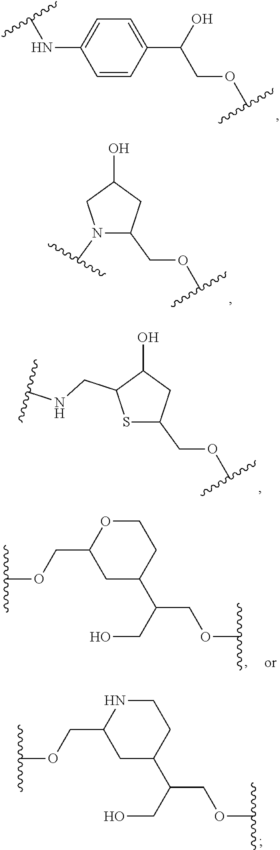 [SMILES: *Nc1ccc(C(O)CO*)cc1 ; *OCC1CC(O)CN1* ; *NCC1SC(CO*)CC1O ; *OCC1CC(C(CO)CO*)CCO1 ; *OCC1CC(C(CO)CO*)CCN1]
and L² is attached to a 3′-terminus of an oligonucleotide.

71. The carbohydrate-oligonucleotide conjugate of any one of claims 3 to 66, wherein L² is:

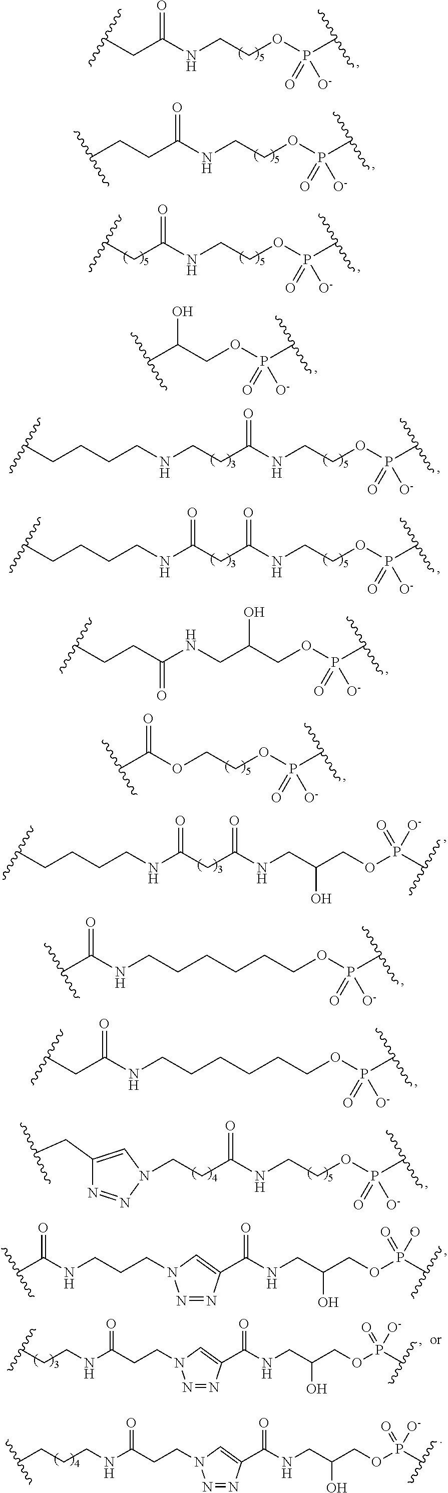

72. The carbohydrate-oligonucleotide conjugate of any one of claims 3 to 71, wherein L^{3a} is —CH₂—, —CH₂CH₂—, —CH₂CH₂CH₂—, —CH₂CH₂CH₂CH₂—,

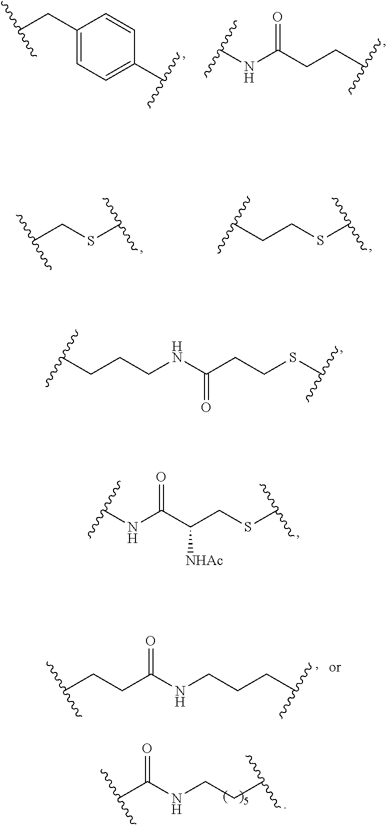

73. The carbohydrate-oligonucleotide conjugate of any one of claims 3 to 72, wherein L^{3c} has the structure of —X(CH₂)ₚX—; each X is independently —O—, —S—, or —N(H)—; and p is an integer of 2, 3, 4, 5, 6, 7, 8, 9, 10, 11, or 12.

74. The carbohydrate-oligonucleotide conjugate of any one of claims 3 to 72, wherein L^{3c} has the structure of —X(CH₂)_{q}CH(OH)(CH₂)ₚX—; each X is independently —O—, —S—, or —N(H)—; and q and r are each independently an integer of 2, 3, 4, 5, 6, 7, 8, 9, or 10.

75. The carbohydrate-oligonucleotide conjugate of any one of claims 3 to 72, wherein L^{3c} has the structure of 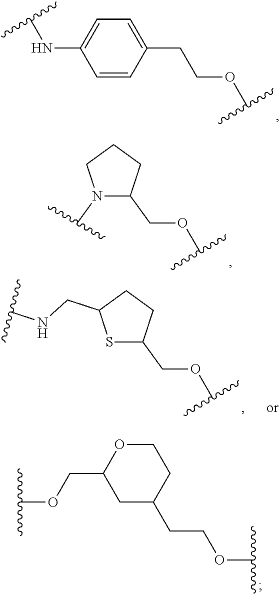 [SMILES: *Nc1ccc(CCO*)cc1 ; *OCC1CCCN1* ; *NCC1CCC(CO*)S1 ; *OCCC1CCOC(CO*)C1]
and L² is attached to a 5′-terminus of an oligonucleotide.

76. The carbohydrate-oligonucleotide conjugate of any one of claims 3 to 72, wherein L^{3c} has the structure of 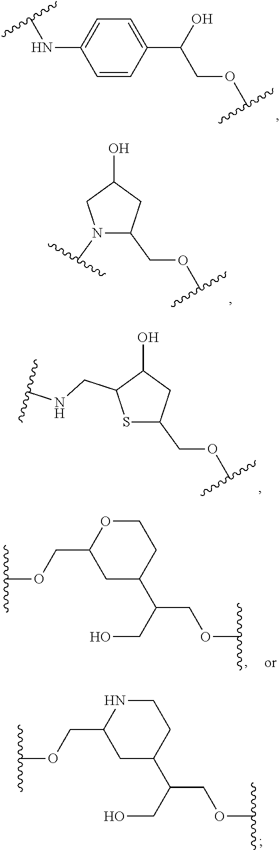 [SMILES: *Nc1ccc(C(O)CO*)cc1 ; *OCC1CC(O)CN1* ; *NCC1SC(CO*)CC1O ; *OCC1CC(C(CO)CO*)CCO1 ; *OCC1CC(C(CO)CO*)CCN1]
and L² is attached to a 3′-terminus of an oligonucleotide.

77. The carbohydrate-oligonucleotide conjugate of any one of claims 3 to 72, wherein L^{3c} is

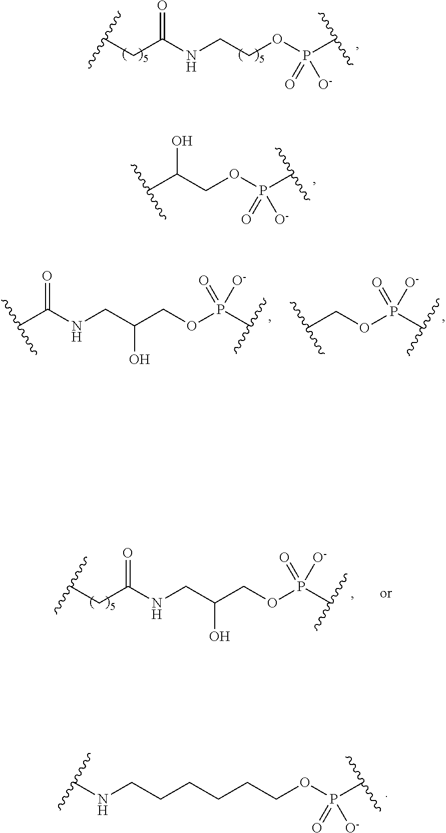

78. The carbohydrate-oligonucleotide conjugate of any one of claims 3 to 77, wherein M is N, CH, or C(CH₃).

79. The carbohydrate-oligonucleotide conjugate of any one of claims 3 to 78, wherein the trivalent moiety 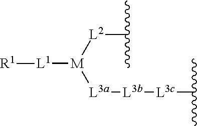
has the structure of:

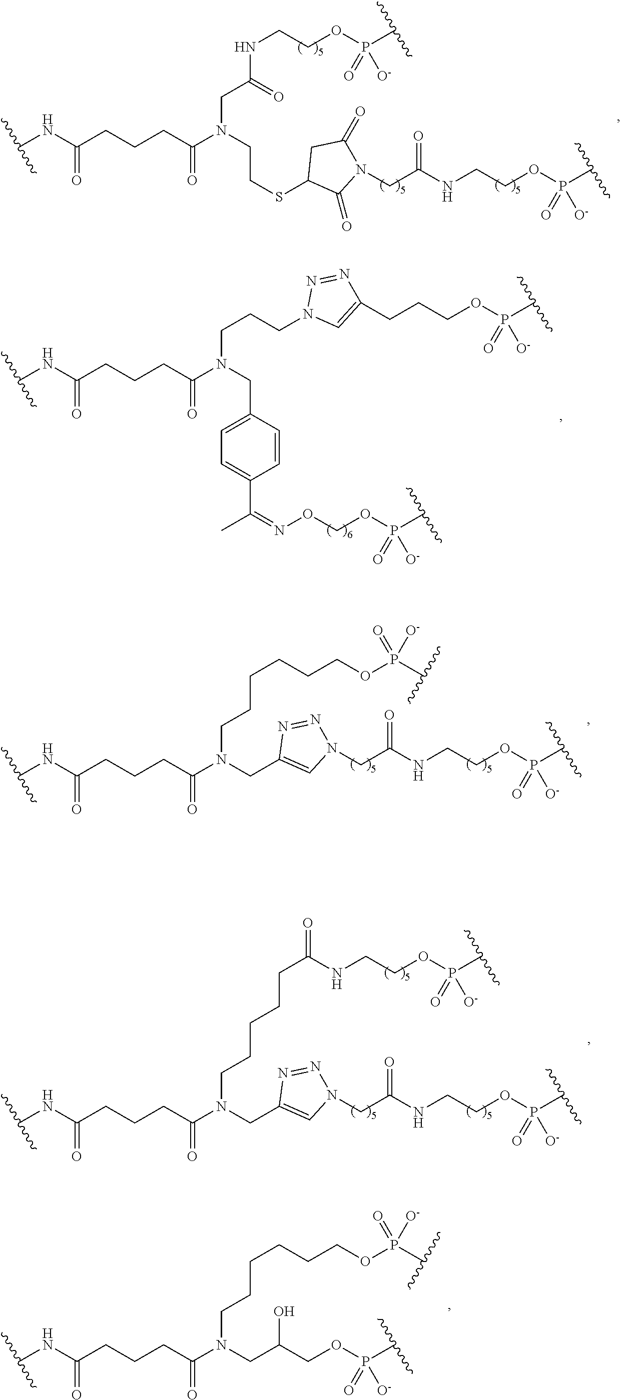

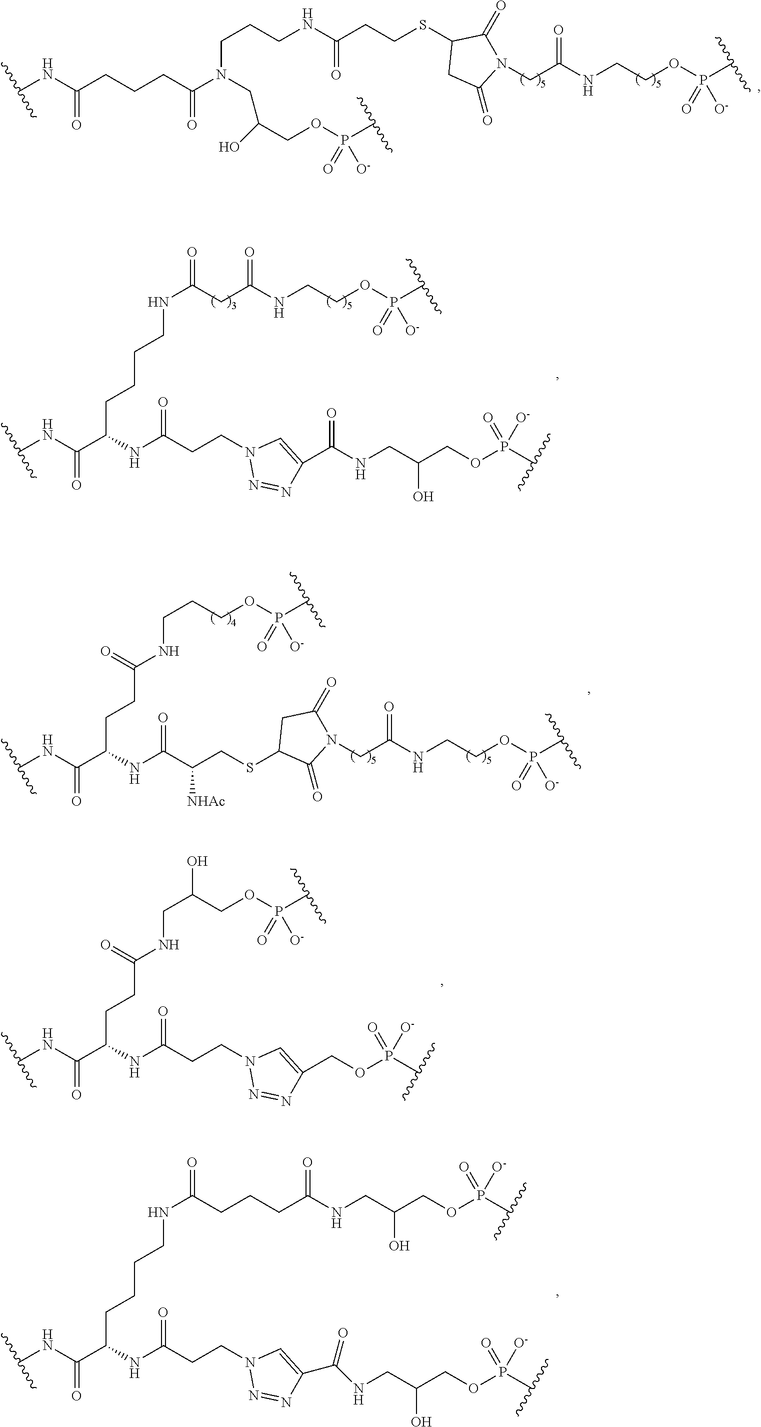

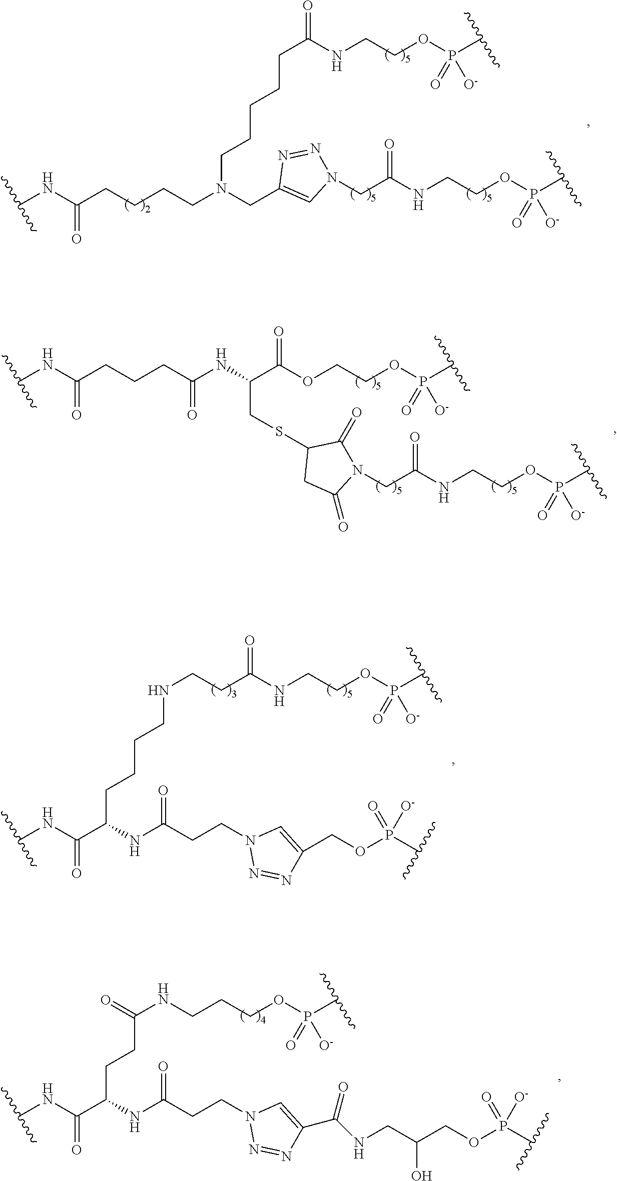

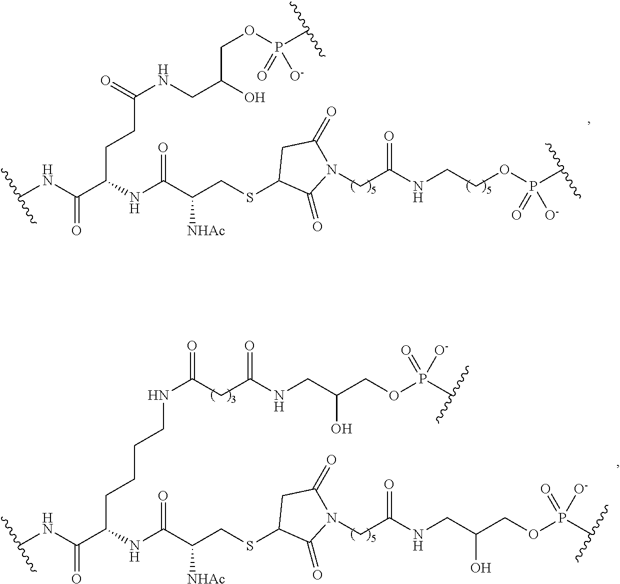

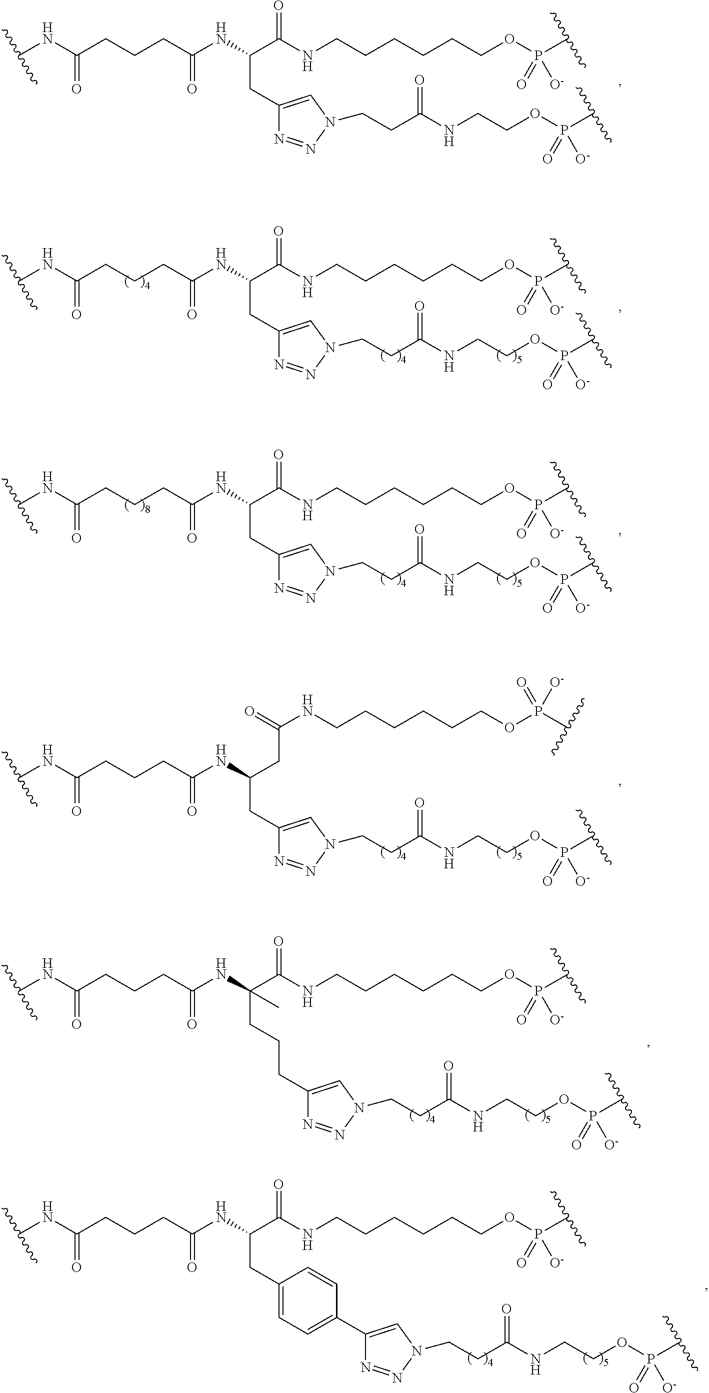

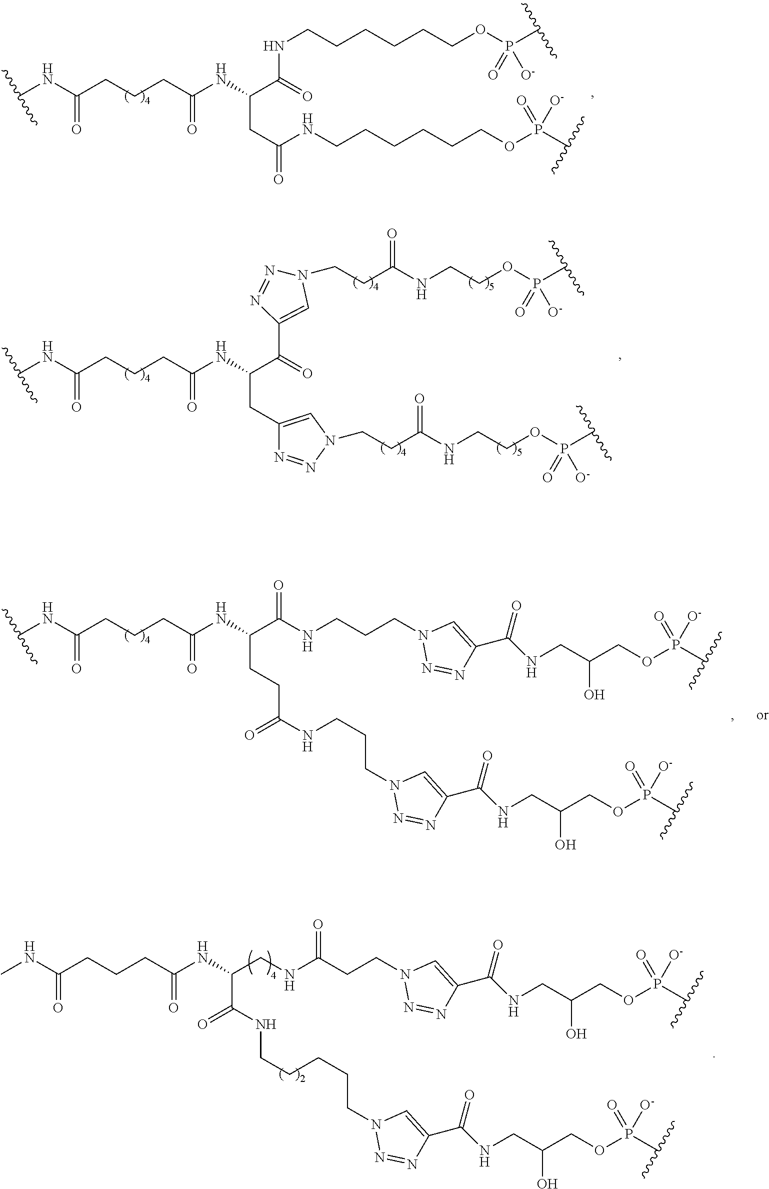

80. The carbohydrate-oligonucleotide conjugate of any one of claims 1 to 3, having the structure of: 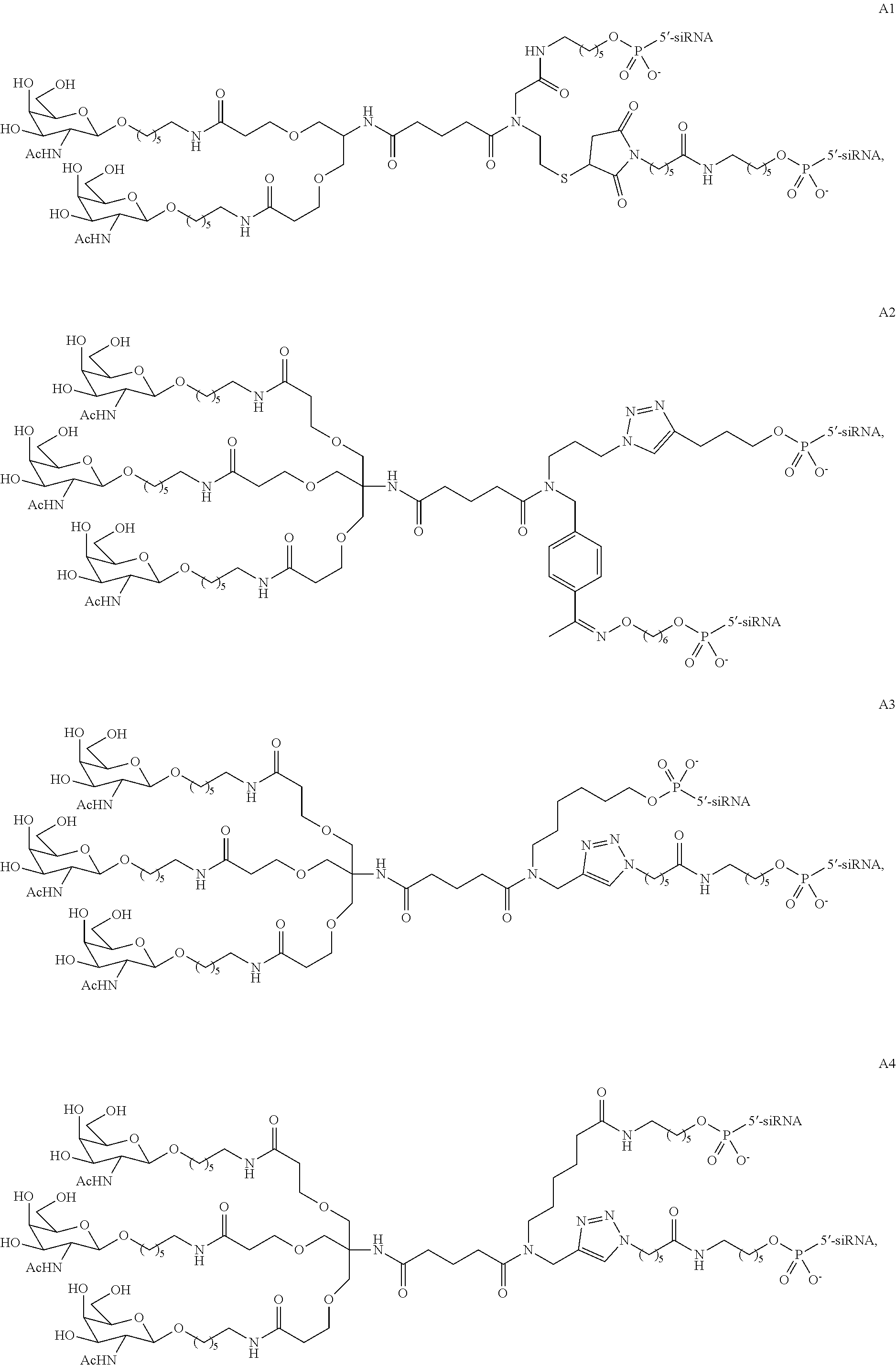 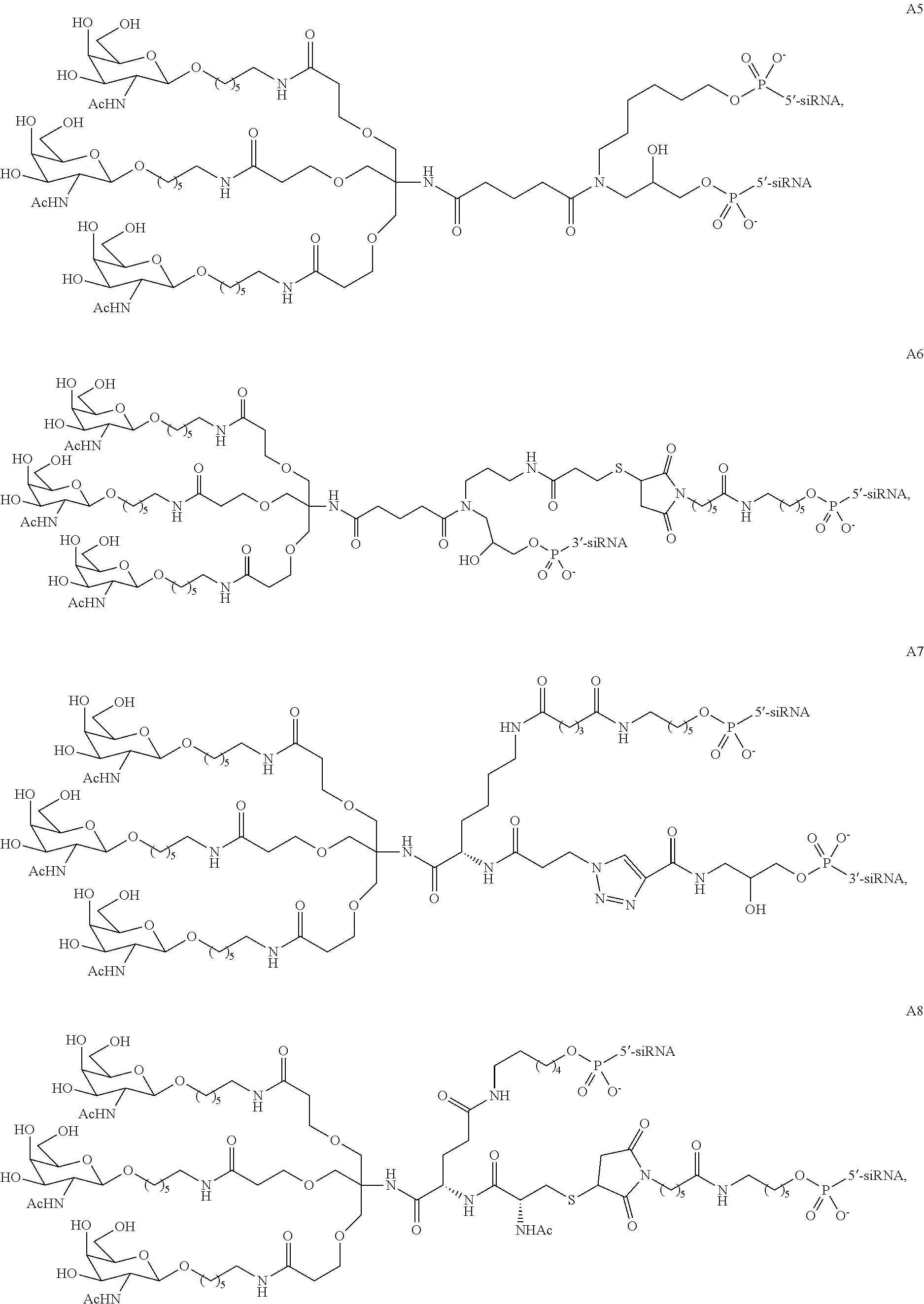 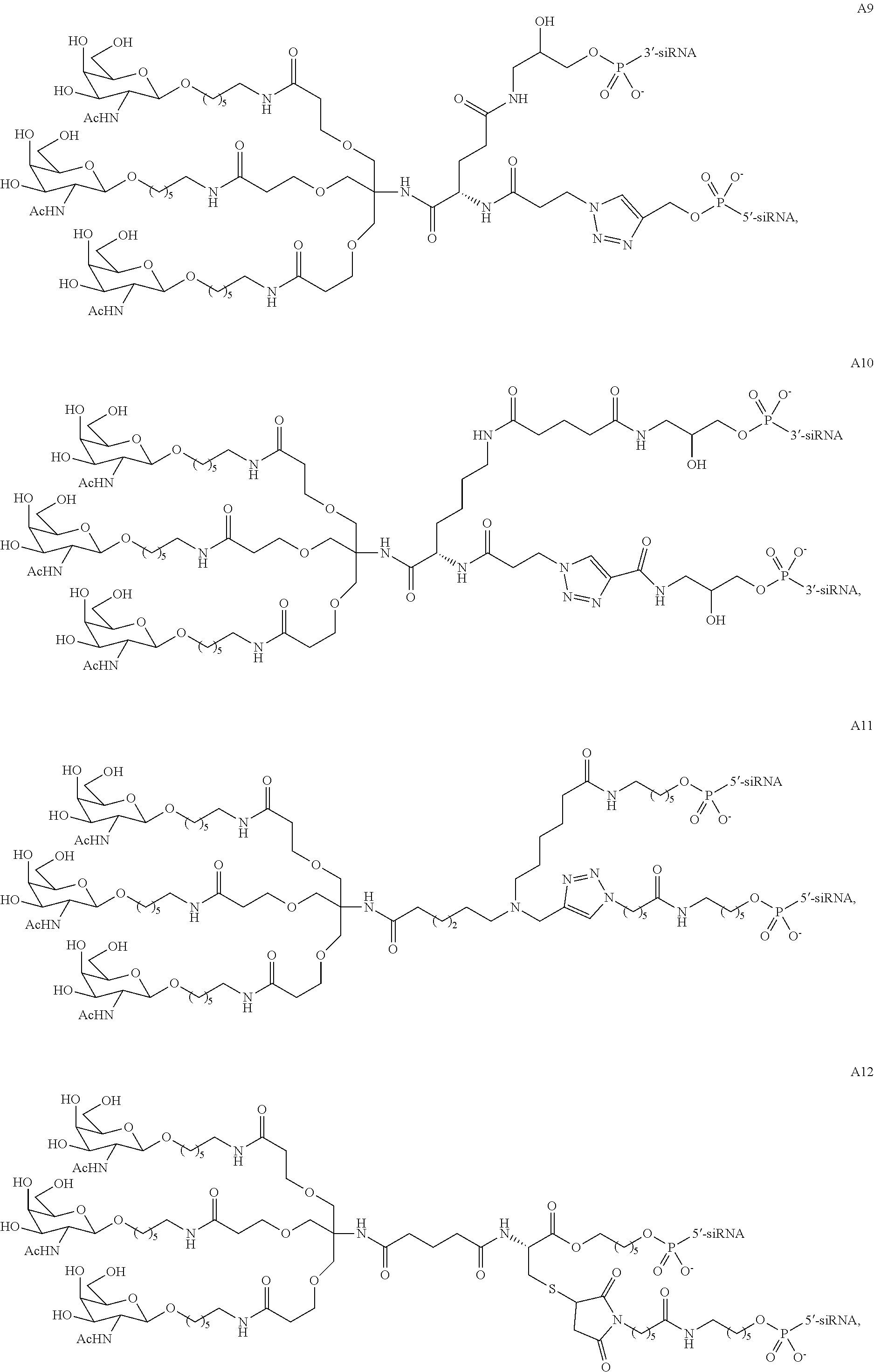 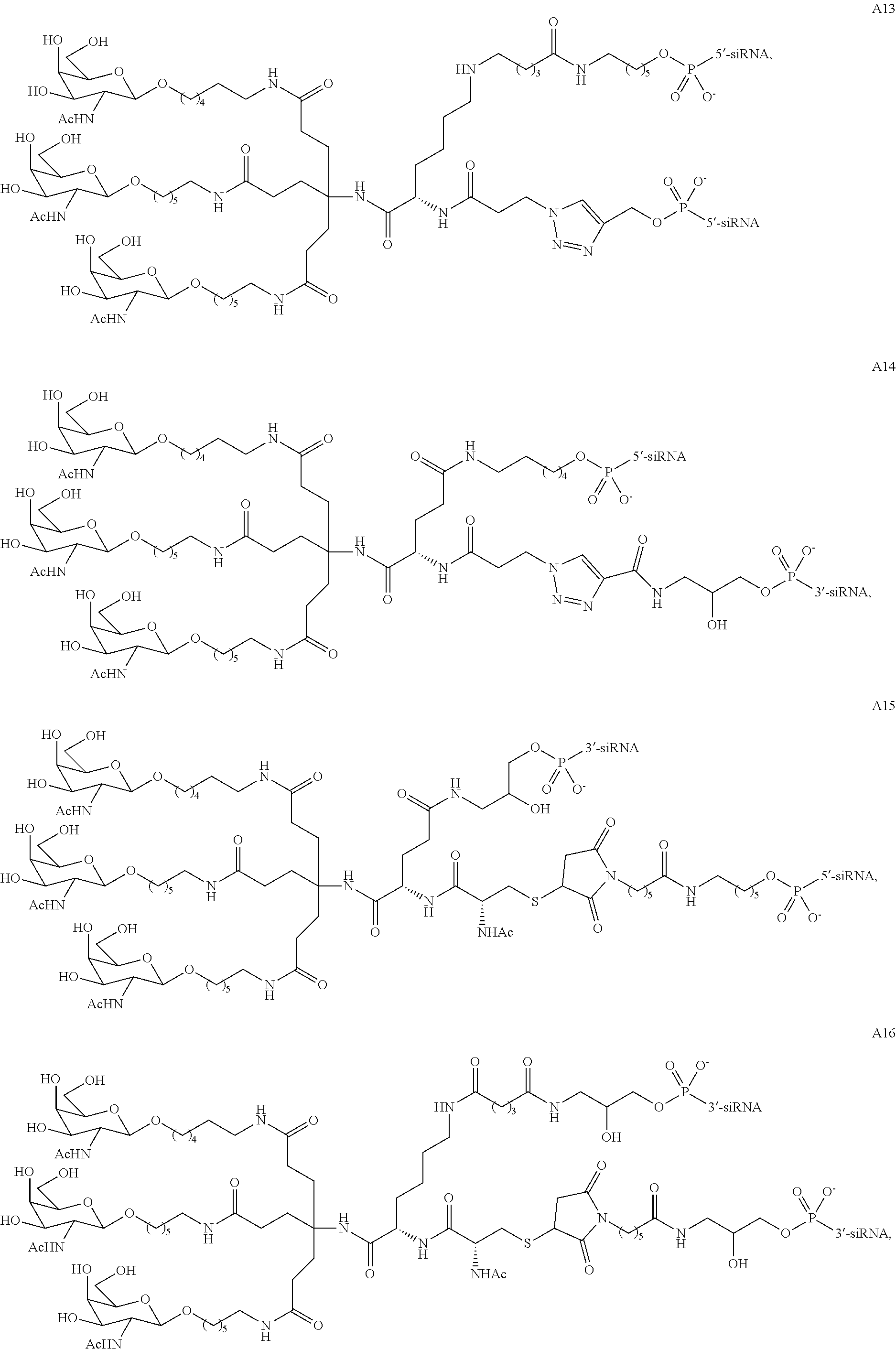 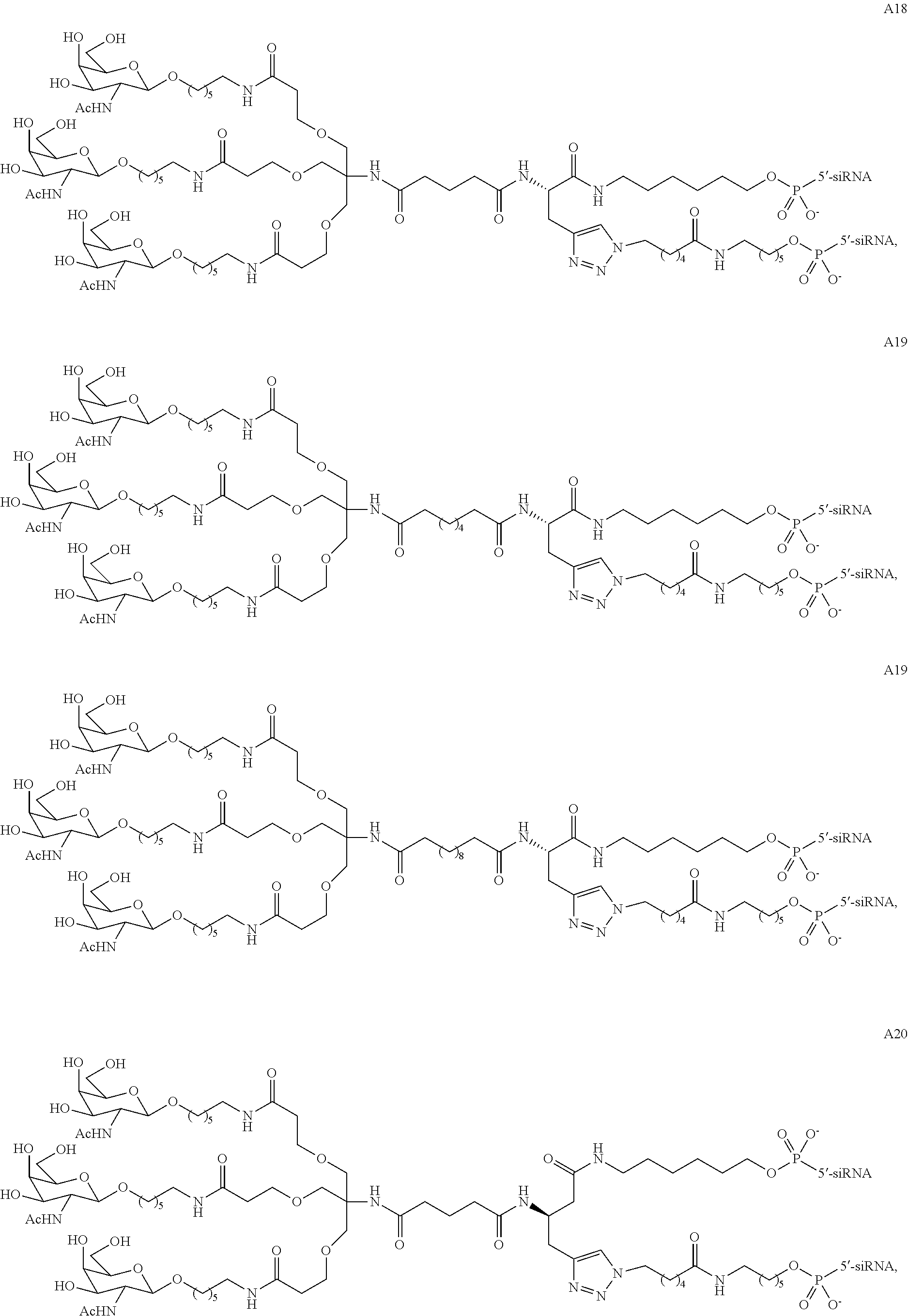 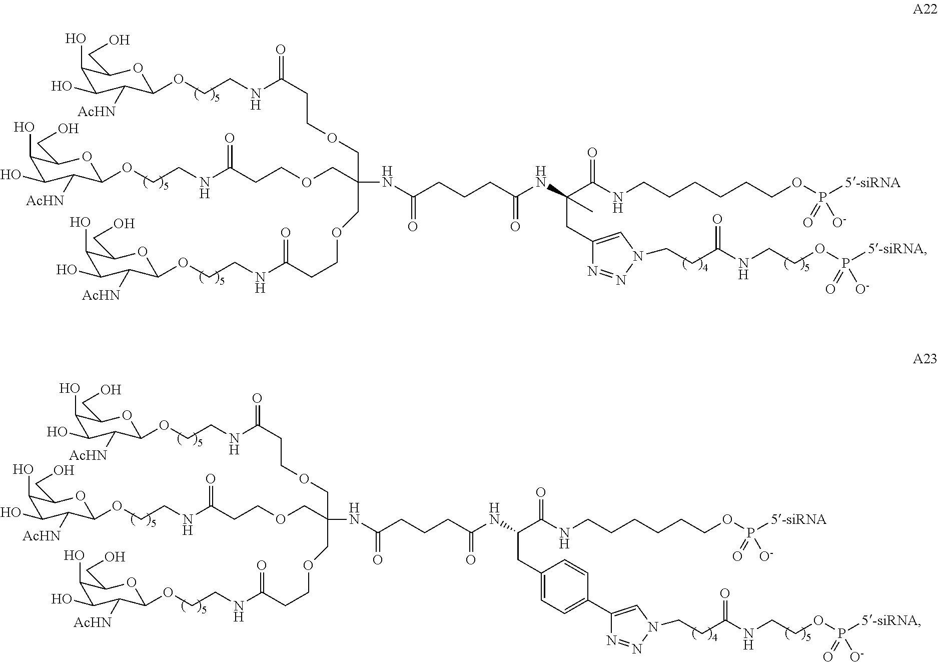 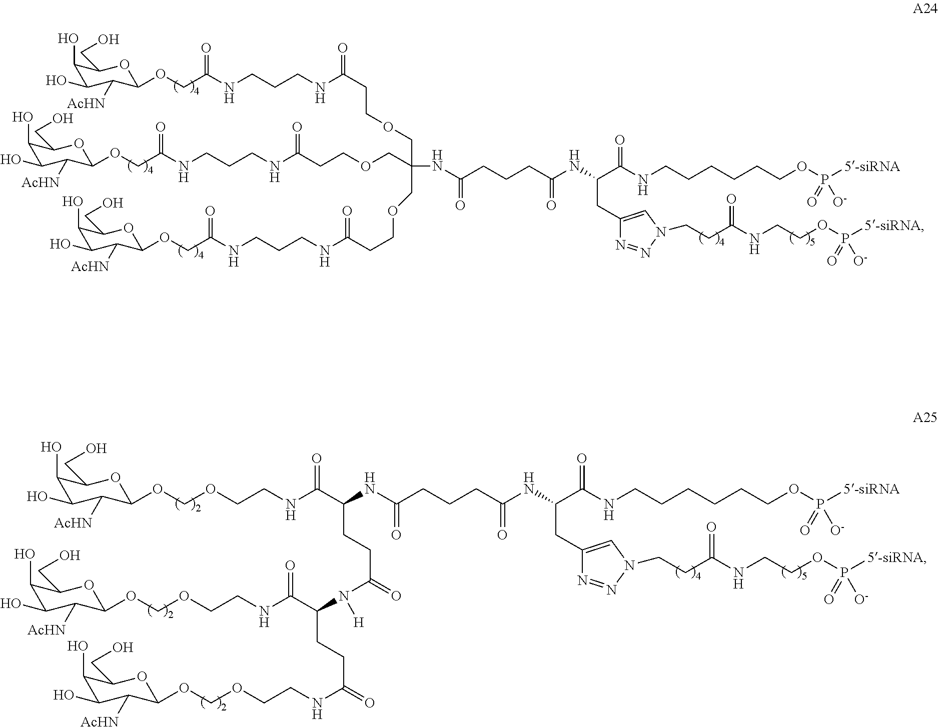 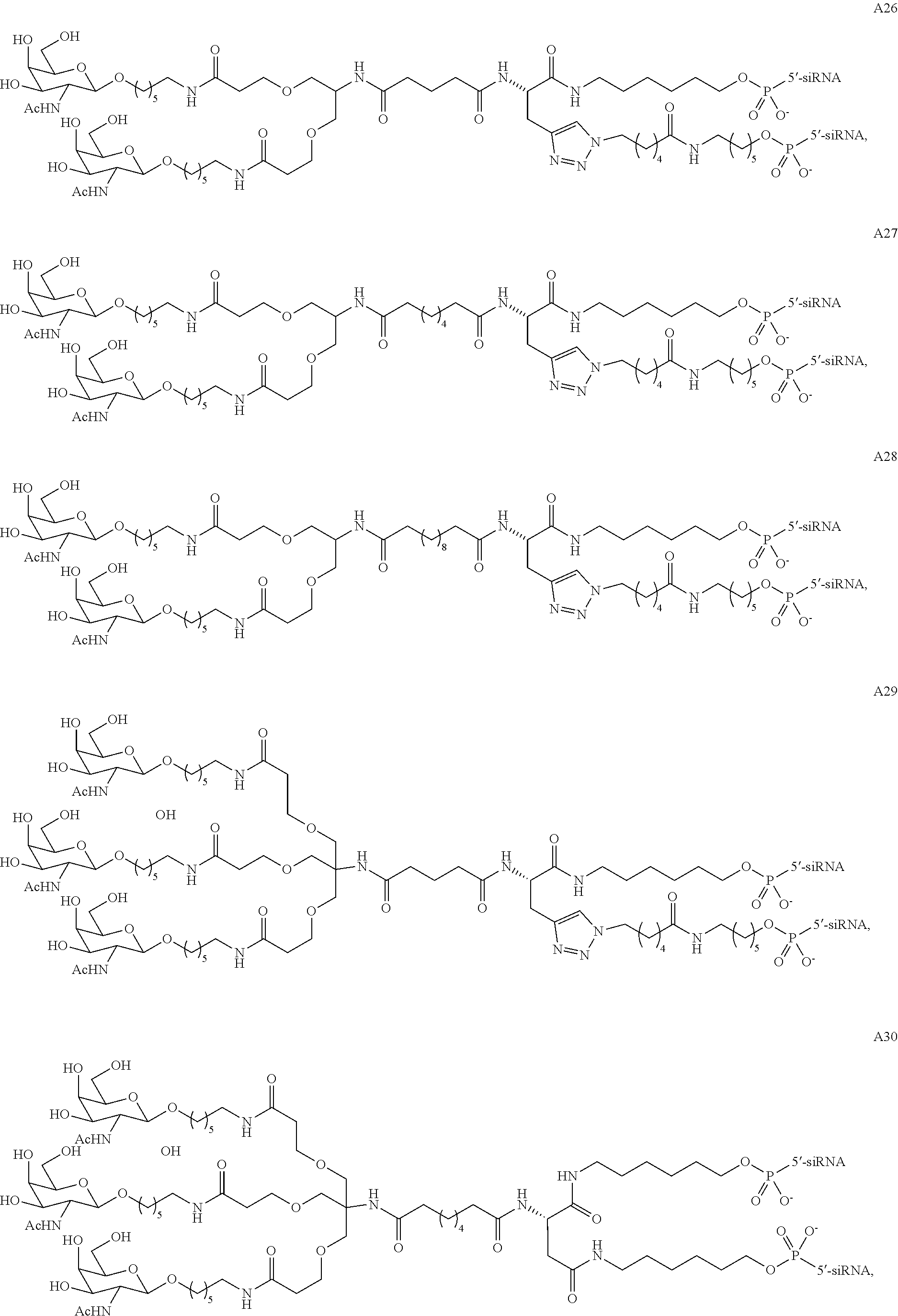 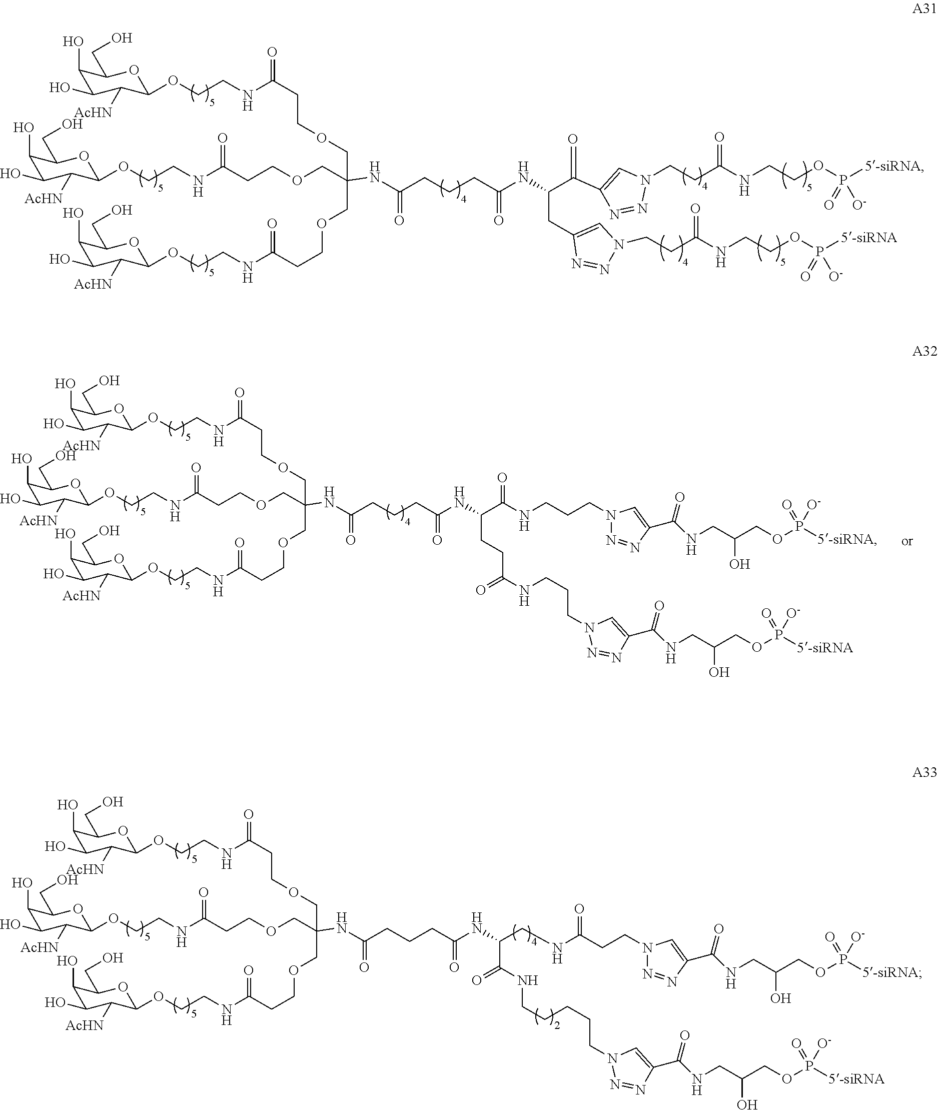
wherein each 3′-siRNA independently represents a siRNA with one of its 3′-termini connected to a trivalent linker; each 5′-siRNA represents a siRNA with one of its 5′-termini connected to a trivalent linker; and 5′-ssDNA represents a single stranded DNA with its 5′-terminus connected to a trivalent linker.

81. The carbohydrate-oligonucleotide conjugate of claim 1, having the structure of: 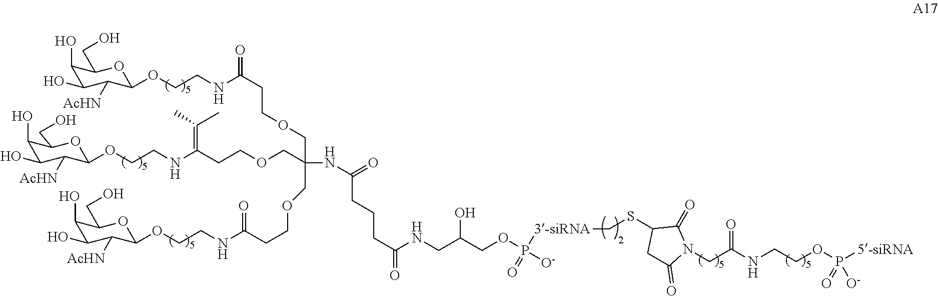
wherein 3′-siRNA represents a siRNA with one of its 3′-termini connected to a first divalent linker and one of its 5′-termini connected to a second divalent linker; and 5′-siRNA represents a siRNA with one of its 5′-termini connected to the second divalent linker.

82. The carbohydrate-oligonucleotide conjugate of claim 80 or 81, wherein each double-stranded oligonucleotide independently comprises a pair of nucleotide sequences of SEQ ID NOs: 1 and 2, 3 and 4, 5 and 6, 7 and 8, 9 and 10, 11 and 12, 13 and 14, 15 and 16, 17 and 18, 19 and 20, 21 and 22, or 23 and 24.

83. The carbohydrate-oligonucleotide conjugate of claim 80, wherein each single-stranded oligonucleotide comprises a nucleotide sequence of SEQ ID NO: 25.

84. A pharmaceutical composition comprising the carbohydrate-oligonucleotide conjugate of any one of claims 1 to 83 and a pharmaceutically acceptable excipient.

85. The pharmaceutical composition of claim 84, wherein the composition is in single dosage form.

86. The pharmaceutical composition of claim 84 or 85, wherein the composition is in a parenteral or intravenous dosage form.

87. The pharmaceutical composition of claim 86, wherein the composition is formulated in an intravenous dosage form.


SEQUENCE LISTING (25 sequences)

SEQ ID NO: 1 - RNA, 21 nt, Synthetic construct
  source 1..21: mol_type = other RNA
ctagacctgttttgcttttgt

SEQ ID NO: 2 - RNA, 23 nt, Synthetic construct
  source 1..23: mol_type = other RNA
acaaaagcaaaacaggtctagaa

SEQ ID NO: 3 - RNA, 21 nt, Synthetic construct
  source 1..21: mol_type = other RNA
gctcaacatatttgatcagta

SEQ ID NO: 4 - RNA, 21 nt, Synthetic construct
  source 1..21: mol_type = other RNA
tactgatcaaatatgttgagc

SEQ ID NO: 5 - RNA, 21 nt, Synthetic construct
  source 1..21: mol_type = other RNA
atgttgtcctttttatctgat

SEQ ID NO: 6 - RNA, 21 nt, Synthetic construct
  source 1..21: mol_type = other RNA
cagataaaaaggacaacatgc

SEQ ID NO: 7 - RNA, 21 nt, Synthetic construct
  source 1..21: mol_type = other RNA
gactttcatcctggaaatata

SEQ ID NO: 8 - RNA, 23 nt, Synthetic construct
  source 1..23: mol_type = other RNA
tatatttccaggatgaaagtcca

SEQ ID NO: 9 - RNA, 21 nt, Synthetic construct
  source 1..21: mol_type = other RNA
aagcaagatatttttataata

SEQ ID NO: 10 - RNA, 25 nt, Synthetic construct
  source 1..25: mol_type = other RNA
tattataaaaatatcttgctttttt

SEQ ID NO: 11 - RNA, 21 nt, Synthetic construct
  source 1..21: mol_type = other RNA
tgtgttcaaagtcaaggatat

SEQ ID NO: 12 - RNA, 23 nt, Synthetic construct
  source 1..23: mol_type = other RNA
atatccttgactttgaacacatg

SEQ ID NO: 13 - RNA, 21 nt, Synthetic construct
  source 1..21: mol_type = other RNA
cttcaagttcatcattcccaa

SEQ ID NO: 14 - RNA, 23 nt, Synthetic construct
  source 1..23: mol_type = other RNA
ttgggaatgatgaacttgaagat

SEQ ID NO: 15 - RNA, 21 nt, Synthetic construct
  source 1..21: mol_type = other RNA
gcagatgaacttgctcttgtt

SEQ ID NO: 16 - RNA, 23 nt, Synthetic construct
  source 1..23: mol_type = other RNA
aacaagagcaagttcatctgcca

SEQ ID NO: 17 - RNA, 21 nt, Synthetic construct
  source 1..21: mol_type = other RNA
gcatgtattacttgacaaaga

SEQ ID NO: 18 - RNA, 23 nt, Synthetic construct
  source 1..23: mol_type = other RNA
tctttgtcaagtaatacatgctg

SEQ ID NO: 19 - RNA, 21 nt, Synthetic construct
  source 1..21: mol_type = other RNA
aacaagaagaacaaactcaca

SEQ ID NO: 20 - RNA, 23 nt, Synthetic construct
  source 1..23: mol_type = other RNA
tgtgagtttgttcttcttgttca

SEQ ID NO: 21 - RNA, 21 nt, Synthetic construct
  source 1..21: mol_type = other RNA
tgtgttcaaagtcaaggatat

SEQ ID NO: 22 - RNA, 23 nt, Synthetic construct
  source 1..23: mol_type = other RNA
atatccttgactttgaacacatg

SEQ ID NO: 23 - RNA, 19 nt, Synthetic construct
  source 1..19: mol_type = other RNA
gggtttttcttgttgacaa

SEQ ID NO: 24 - RNA, 21 nt, Synthetic construct
  source 1..21: mol_type = other RNA
ttgtcaacaagaaaaaccctt

SEQ ID NO: 25 - DNA, 22 bp, Synthetic construct
  source 1..22: mol_type = other DNA
tgactgtgaacgttcgagatga